(19) United States
(12) Patent Application Publication
(10) Pub. No.: US 2020/0383960 A1
(43) Pub. Date: Dec. 10, 2020

(54) Pyridine and Pyrazine derivative for the Treatment of CF, COPD, and Bronchiectasis

(71) Applicant: NOVARTIS AG, Basel (CH)

(72) Inventors: Sarah Schmidt GRANT, Basel (CH); Bettina HEDERER, Wollerau (CH); Thomas LANGENICKEL, Bottmingen (CH); David J. ROWLANDS, Cambridge, MA (US); Robert Martin STRIETER, Falmouth, MA (US); Xianbin TIAN, East Hanover, NJ (US)

(21) Appl. No.: 16/895,583

(22) Filed: Jun. 8, 2020

Related U.S. Application Data
(60) Provisional application No. 62/859,442, filed on Jun. 10, 2019.
(60) Provisional application No. 63/025,567, filed on May 15, 2020.

(51) Int. Cl. A61K 31/444; A61K 9/00; A61K 45/06; A61K 31/44; A61K 31/443; A61K 31/5377; A61K 31/497; A61K 31/4965; A61K 31/4439; A61P 31/04

(52) U.S. Cl. CPC A61K 31/444; A61K 9/0053; A61K 45/06; A61K 31/44; A61P 31/04; A61K 31/5377; A61K 31/497; A61K 31/4965; A61K 31/4439; A61K 31/443

(57) ABSTRACT

The present invention provides pyridine and pyrazine derivatives which restore or enhance the function of mutant and/or wild type CFTR to treat bronchiectasis, cystic fibrosis, primary ciliary dyskinesia, chronic bronchitis, chronic obstructive pulmonary disease, asthma, respiratory tract infections, lung carcinoma, xerostomia and keratoconjunctivitis sire, or constipation (IBS, IBD, opioid induced). Pharmaceutical compositions comprising such derivatives are also encompassed.

### 1. CROSS REFERENCE TO RELATED APPLICATIONS

This application is claims the benefit of priority to U.S. Provisional Application No. 62/859,442, filed Jun. 10, 2019 and U.S. Provisional Application No. 63/025,567, filed May 15, 2020, the disclosure of each of which is incorporated by reference herein in its entirety.

### 2. FIELD OF THE INVENTION

This invention relates to pyridine and pyrazine compounds, their preparation and use as pharmaceuticals. The present invention also relates to their use as medicaments for the treatment of bronchiectasis, Chronic Obstructive Pulmonary Disorder (COPD), Cystic Fibrosis (CF), chronic bronchitis, primary ciliary dyskinesia, respiratory tract infections or asthma.

### 3. BACKGROUND

Cystic fibrosis (CF) is a fatal genetic disease caused by mutations in the gene encoding the CF transmembrane conductance regulator (CFTR), a protein kinase A (PKA)-activated epithelial anion channel involved in salt and fluid transport in multiple organs, including the lung. Most CF mutations either reduce the number of CFTR channels at the cell surface (e.g., synthesis or processing mutations) or impair channel function (e.g., gating or conductance mutations) or both. The present invention discloses compounds which restore or enhance the function of mutant and/or wild type CFTR to treat bronchiectasis, cystic fibrosis, primary ciliary dyskinesia, chronic bronchitis, chronic obstructive pulmonary disease, asthma, respiratory tract infections, lung carcinoma, xerostomia and keratoconjunctivitis sire, or constipation (e.g., IBS, IBD, opioid induced).

Bronchiectasis is a chronic disease characterized by abnormal and permanent dilation of the bronchi resulting in chronic cough, sputum production, and recurrent bacterial infections of the airways (Martinez-Garcia et al., Chest. 2005 August; 128(2):739-45; Wilson et al., Eur Respir J. 1997 August; 10(8):1754-60). Bronchiectasis is generally classified into either cystic fibrosis bronchiectasis or non-cystic fibrosis bronchiectasis (King et al., Intern Med J. 2006 36(11):729-737). Patients with bronchiectasis suffer from a high morbidity due to frequent exacerbations impairing quality of life and facilitating resistance to antibiotics, leading to reduced lung function. There is also a high socioeconomic impact through frequent use of primary and secondary healthcare with an economic burden estimated to be similar to COPD (Polverino et al., Eur Respir J. 2017 Sep. 9; 50(3)). The age-adjusted mortality of patients with bronchiectasis compared to the general population is approximately two-fold higher (Quint et al., Eur Respir J. 2016 January; 47(1):186-93). Patients with bronchiectasis have some similarities to those with CF such as radiological dilatation of airways, bronchial wall thickening, mucus plugging, and hyperinflation.

There is a significant unmet medical need for treatment of bronchiectasis and no approved therapy for the reduction of exacerbations is currently available. The European Respiratory Society (ERS) 2017 guidelines for the management of adult bronchiectasis suggests that apart from antibiotics to treat acute exacerbations, no other treatment can be recommended (Polverino et al., Eur Respir J. 2017 Sep. 9; 50(3)).

A reduction from baseline in colony forming units of potentially pathogenic microorganisms in spontaneous sputum by one log unit has been associated with a significant reduction in risk of exacerbation by approximately 20% in patients with bronchiectasis, which is considered to be clinically relevant (Chalmers et al., Am J Respir Crit Care Med. 2012 Oct. 1; 186(7):657-65).

Recent evidence also suggests the molecular mechanism for reduced mucociliary clearance (MCC) in bronchiectasis may relate to dysfunction of wild-type and mutated CFTR (Amaral, et al., Trends Pharmacol Sci. 2007 July; 28(7):334-41; see also Schafer et al., BMC Pulm Med. 2018; 18:79). Bronchiectasis patients may also have a component of ion channel dysfunction, including CFTR (Amaral, et al., Trends Pharmacol Sci. 2007 July; 28(7):334-41). 3-Amino-6-methoxy-5-trifluoromethyl-pyridine-2-carboxylic acid ((S)-3,3,3-trifluoro-2-hydroxy-2-methyl-propyl)-amide, herein referred to as Compound A, is an effective CFTR potentiator, has been shown to reduce MCC, resulting in decreased bacterial colonization, decreased small airway inflammation, improved lung function (FEV1), and ultimately fewer exacerbations in COPD patients. In addition, data suggests that Compound A decreases bacterial colonization and small airway inflammation by reducing fibrinogen levels in COPD patients. Compound A has also demonstrated significant improvement in lung function (FEV1) in patients with CF following two weeks of treatment and in patients with COPD following four weeks of treatment.

The present invention discloses compounds which restore or enhance the function of mutant and/or wild type CFTR to treat bronchiectasis. Further, the present invention discloses compounds that provide improved MCC resulting in decreased bacterial colonization, decreased small airway inflammation, improved forced expiratory volume in one second (FEV1), and ultimately fewer exacerbations to treat bronchiectasis.

### 4. SUMMARY

In one aspect, the invention provides methods of treating bronchiectasis comprising administering at least one compound according to Formula (I):

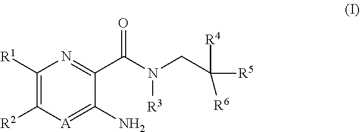

or a pharmaceutically acceptable salt thereof, to a subject in need thereof, wherein:

A is N or CR^{4a};

R¹ is H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; halogen; SO₂NR⁸R⁹; SO₂R¹⁰; S—C₁-C₈alkyl optionally substituted by one or more halogen atoms; S—C₆-C₁₄ aryl; CN; NR¹¹R¹²; C(O)NR¹³R¹⁴; NR¹³SO₂R¹⁵; NR¹³C(O)R¹⁵, CO₂R¹⁵, —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, aryl and heterocyclyl groups are each optionally substituted by one or more Z substituents;

R² is C₁-C₄ haloalkyl;

R³ and R^{4a} are each independently H or C₁-C₈ alkyl optionally substituted by one or more halogen atoms;

R⁴ is H, or C₁-C₈ alkyl optional substituted with one or more halogen; R⁵ is —(CH₂)ₘ—NR¹⁷R¹⁸, —(CH₂)ₘ—OR′; C₁-C₈ alkoxy optionally substituted by one or more halogen atoms; —(C₀-C₄ alkyl)-CO₂R¹⁵; —(C₀-C₄ alkyl)-C₆-C₁₄ aryl or −3 to 14 membered heterocyclic group, wherein the heterocyclic group contains at least one heteroatom selected from N, O and S; wherein the —(C₀-C₄ alkyl)-C₆-C₁₄ aryl and —(C₀-C₄ alkyl)-3 to 14 membered heterocyclic group are each optionally substituted by one or more Z substituents;

R⁶ is C₁-C₈ alkyl optionally substituted by one or more halogen atoms; C₃-C₁₀ cycloalkyl; —C₁-C₄ alkyl-C₃-C₈ cycloalkyl; C₁-C₈ alkoxy optionally substituted by one or more halogen atoms; OH; CN; halogen; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, —(C₀-C₄ alkyl)-C₆-C₁₄ aryl and —(C₀-C₄ alkyl)-3 to 14 membered heterocyclic group are each optionally substituted by one or more Z substituents; or

R⁶ is H, and R⁵ is —(CH₂)ₘ—NR¹⁷R¹⁸, —(CH₂)ₘ—OR′, C₁-C₈ alkoxy optionally substituted by one or more halogen atoms; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; or —(C₀-C₄ alkyl)-CO₂R¹⁵, wherein —(C₀-C₄ alkyl)-C₆-C₁₄ aryl and —(C₀-C₄ alkyl)-3 to 14 membered heterocyclic group groups are each optionally substituted by one or more Z substituents; or

R⁴ and R⁶ together with the carbon atoms to which they are bound form a 3 to 8 membered carbocyclic ring system; or

R⁴ and R⁵ together form an oxo group (C═O) and R⁶ is C₁-C₄ alkyl optionally substituted by one or more halogen atoms; C₁-C₄ alkoxy optionally substituted by one or more halogen atoms; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the aryl and heterocyclyl groups are each optionally substituted by one or more Z substituents; or

R⁵ and R⁶ together with the carbon atoms to which they are bound a 5 to 8 membered heterocyclic ring system containing one or more heteroatoms selected from N, O and S, wherein the ring system is optionally substituted by one or more Z substituents; or

R⁴ and R⁵ and R⁶ together with the carbon atoms to which they are bound form a 5 to 8 membered heterocyclic ring system containing one or more heteroatoms selected from N, O and S, wherein the ring system is optionally substituted by one or more Z substituents;

R′ is H, or C₁-C₈ alkyl optional substituted with one or more halogen;

m is 0, 1, 2 or 3;

R⁸, R¹¹, R¹³, and R¹⁷ are each independently H, C₁-C₈ alkyl optionally substituted by one or more halogen atoms, C₃-C₁₀ cycloalkyl or —(C₁-C₄ alkyl)-C₃-C₈ cycloalkyl;

R⁹, R¹⁰, R¹², R¹⁴, R¹⁵, R¹⁶ and R¹⁸ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the cycloalkyl, cycloalkenyl, aryl and heterocyclyl groups are each optionally substituted by one or more Z substituents; or

R⁸ and R⁹, R¹¹ and R¹², R¹³ and R¹⁴, and R¹⁷ and R¹⁸ together with the nitrogen atom to which they are attached may form a 4 to 14 membered heterocyclic group optionally substituted by one or more Z substituents;

Z is independently OH, aryl, O-aryl, benzyl, O-benzyl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁸(SO₂)R²¹, (SO₂)NR¹⁹R²¹, (SO₂)R²¹, NR¹⁸C(O)R²¹, C(O)NR¹⁹R²¹, NR¹⁸C(O)NR¹⁹R²¹, NR¹⁸C(O)OR¹⁹, NR¹⁹R²¹, C(O)OR¹⁹, C(O)R¹⁹, SR¹⁹, OR¹⁹, oxo, CN, NO₂, halogen or a 3 to 14 membered heterocyclic group, wherein the heterocyclic group contains at least one heteroatom selected from N, O and S;

R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy.

Various embodiments of the invention are described herein. It will be recognized that features specified in each embodiment may be combined with other specified features to provide further embodiments.

In an embodiment of the invention as described anywhere herein, A is N.

In an embodiment of the invention as described anywhere herein, A is CR⁴a.

In an embodiment of the invention as described anywhere herein, R¹ is selected from H; C₁-C₈ alkyl optionally substituted by one or more halogen atoms; C₁-C₈ alkoxy optionally substituted by one or more halogen atoms; halogen; C₆-C₁₄ aryl; —(C₀-C₄ alkyl)-3 to 14 membered heterocyclic group, wherein the heterocyclic group contains at least one heteroatom selected from N, O and S; and NR¹¹R¹², wherein the aryl and heterocyclic groups are each optionally substituted by one or more Z substituents.

In an embodiment of the invention as described anywhere herein, R¹ is C₁-C₄ alkyl optional substituted by one or more halogen atoms. For example, —CH₃ or CF₃.

In an embodiment of the invention as described anywhere herein, R¹ is C₁-C₄ alkoxy optional substituted by one or more halogen atoms. For example, —OCH₃ or —OCF₃.

In an embodiment of the invention as described anywhere herein, R¹ is aryl, wherein aryl is phenyl optionally substituted by one or more Z substituents, specific example are 4-fluorophenyl, 4-chloro-2-methylphenyl, or 2,4-dichlorophenyl.

In an embodiment of the invention as described anywhere herein, R¹ is 6 membered heterocyclyl group, wherein 6 membered heterocyclyl group is pyridyl optionally substituted by one or more Z substituents, specific example is 1-methyl-4-pyridyl.

In an embodiment of the invention as described anywhere herein, R¹ is Br, —CH₃, —CF₃, —OCH₃, —OCF₃, 4-fluorophenyl, 4-chloro-2-methylphenyl, or 2,4-dichlorophenyl.

In an embodiment of the invention as described anywhere herein, R² is CF₃CF₂—, (CF₃)₂CH—, CH₃—CF₂—, CF₃CF₂—, CF₃, CF₂H—, CH₃—CCl₂—, CF₃CFCClH—, CBr₃, CBr₂H—CF₃CF₂CHCF₃ or CF₃CF₂CF₂CF₂—.

In an embodiment of the invention as described anywhere herein, R² is CF₃.

In an embodiment of the invention as described anywhere herein, R³ is H or methyl.

In a further embodiment of the invention as described anywhere herein, R^{4a} is H.

An embodiment of the invention as defined above provides compounds according to Formula (I), wherein R⁵ provides a heteroatom two carbons from the amide nitrogen, wherein the heteroatom is oxygen or nitrogen.

An embodiment of the invention as defined above provides compounds according to Formula (I), wherein

R⁴ is H, C₁-C₄ alkyl optionally substituted by one or more halogen atoms or not present;

R⁵ is C₁-C₄ alkoxy optionally substituted by one or more halogen atoms; —(CH₂)ₘ—NR¹⁷R¹⁸; —(CH₂)ₘ—OR″, or OH;

m is 0, or 1;

R⁶ is C₁-C₄ alkyl optionally substituted by one or more halogen atoms; C₁-C₄ alkoxy optionally substituted by one or more halogen atoms; OH; CN; halogen; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the aryl and heterocyclyl groups are each optionally substituted by one or more Z substituents; or

R⁴ and R⁵ together form an oxo group (C═O); or

R⁵ and R⁶ together with the carbon atoms to which they are bound form a 5 to 8 membered heterocyclic ring system containing one or more heteroatoms selected from N, O and S, wherein the ring system is optionally substituted by one or more Z substituents;

R¹⁷ and R¹⁸ are each independently H; or C₁-C₄ alkyl optionally substituted by one or more halogen atoms.

An embodiment of the invention as defined above provides compounds according to Formula (I), wherein

A is CR^{4a};

R¹ is halogen, C₁-C₄ alkyl optionally substituted by one or more halogen atoms, or C₁-C₄ alkoxy optionally substituted by one or more halogen atoms;

R² is C₁-C₄ haloalkyl;

R³ is H;

R⁴ is H or Me;

R^{4a} is H;

R⁵ is —(CH₂)^{m}—NR¹⁷R¹⁸; —(CH₂)ₘ—OR′; or OH;

m is 0, or 1;

R⁶ is C₁-C₄ alkyl optionally substituted by one or more halogen atoms; or

R⁵ and R⁶ together with the carbon atoms to which they are bound form a 5 to 6 membered heterocyclic ring system containing one or more heteroatoms selected from N, O and S, wherein the ring system is optionally substituted by one or more Z substituents; and

R¹⁷ and R¹⁸ are each independently H; or C₁-C₄ alkyl optionally substituted by one or more halogen atoms.

An embodiment of the invention as defined above provides compounds according to Formula (I), wherein

A is CR^{4a};

R¹ is halogen, C₁-C₄ alkyl optionally substituted by one or more halogen atoms, or C₁-C₄ alkoxy optionally substituted by one or more halogen atoms;

R² is C₁-C₄ haloalkyl;

R³ is H;

R^{4a} is H;

R⁴ and R⁵ together form an oxo group (C═O); and

R⁶ is C₁-C₄ alkyl optionally substituted by one or more halogen atoms; C₁-C₄ alkoxy optionally substituted by one or more halogen atoms; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the aryl and heterocyclyl groups are each optionally substituted by one or more Z substituents.

An embodiment of the invention as defined above provides compounds according to Formula (I), wherein

A is CR^{4a};

R¹ is C₁-C₄ alkyl optionally substituted by one or more halogen atoms;

R² is C₁-C₄ haloalkyl;

R³ is H;

R⁴ is H or Me;

R^{4a} is H;

R⁵ is —(CH₂)^{m}—NR¹⁷R¹⁸; —(CH₂)ₘ—OR′; or OH;

m is 0, or 1;

R⁶ is C₁-C₄ alkyl optionally substituted by one or more halogen atoms; or

R⁵ and R⁶ together with the carbon atoms to which they are bound form a 5 to 6 membered heterocyclic ring system containing one or more heteroatoms selected from N, O and S, wherein the ring system is optionally substituted by one or more Z substituents; and

R¹⁷ and R¹⁸ are each independently H; or C₁-C₄ alkyl optionally substituted by one or more halogen atoms.

An embodiment of the invention as defined above provides compounds according to Formula (I), wherein

A is CR^{4a};

R¹ is C₁-C₄ alkoxy optionally substituted by one or more halogen atoms;

R² is C₁-C₄ haloalkyl;

R³ is H;

R⁴ is H or Me;

R^{4a} is H;

R⁵ is —(CH₂)ₘ—NR¹⁷R¹⁸; —(CH₂)ₘ—OR; or OH;

m is 0, or 1;

R⁶ is C₁-C₄ alkyl optionally substituted by one or more halogen atoms; or

R⁵ and R⁶ together with the carbon atoms to which they are bound form a 5 to 6 membered heterocyclic ring system containing one or more heteroatoms selected from N, O and S, wherein the ring system is optionally substituted by one or more Z substituents; and

R¹⁷ and R¹⁸ are each independently H; or C₁-C₄ alkyl optionally substituted by one or more halogen atoms.

An embodiment of the invention as defined above provides compounds according to Formula (I), wherein

A is CR^{4a};

R¹ is C₁-C₄ alkoxy optionally substituted by one or more halogen atoms;

R² is C₁-C₄ haloalkyl;

R³ is H;

R⁴ is H or Me;

R^{4a} is H;

R⁵ is —NR¹⁷R¹⁸; or OH;

R⁶ is C₁-C₄ alkyl optionally substituted by one or more halogen atoms; or

R⁵ and R⁶ together with the carbon atoms to which they are bound form a 5 to 6 membered heterocyclic ring system containing one or more heteroatoms selected from N, O and S, wherein the ring system is optionally substituted by one or more Z substituents; and

R¹⁷ and R¹⁸ are each independently H; or C₁-C₄ alkyl optionally substituted by one or more halogen atoms.

An embodiment of the invention as defined above provides compounds according to Formula (I), wherein

A is CR^{4a};

R¹ is C₁-C₄ alkyl optionally substituted by one or more halogen atoms;

R² is C₁-C₄ haloalkyl;

R³ is H;

R⁴ is H or Me;

R^{4a} is H;

R⁵ is —NR¹⁷R¹⁸; or OH;

R⁶ is C₁-C₄ alkyl optionally substituted by one or more halogen atoms; and

R¹⁷ and R¹⁸ are each independently H; or C₁-C₄ alkyl optionally substituted by one or more halogen atoms.

An embodiment of the invention as defined above provides compounds according to Formula (I), wherein

A is CR^{4a};

R¹ is C₁-C₄ alkoxy optionally substituted by one or more halogen atoms;

R² is C₁-C₄haloalkyl;

R³ is H;

R⁴ is H or Me;

R^{4a} is H;

R⁵ is —NR¹⁷R¹⁸; or OH;

R⁶ is C₁-C₄ alkyl optionally substituted by one or more halogen atoms; and

R¹⁷ and R¹⁸ are each independently H; or C₁-C₄ alkyl optionally substituted by one or more halogen atoms.

In an embodiment of the invention as described anywhere herein, wherein

Z is independently OH, C₁-C₄ alkyl optionally substituted by one or more OH groups or NH₂ groups, C₁-C₄ alkyl optionally substituted by one or more halogen atoms, C₁-C₄ alkoxy optionally substituted by one or more OH groups or C₁-C₄ alkoxy, NR¹⁹R²¹, C(O)OR¹⁹, C(O)R¹⁹, SR¹⁹, OR¹⁹, CN, NO₂, or halogen;

R¹⁹ and R²¹ are each independently H; C₁-C₄ alkyl; C₃-C₆ cycloalkyl; or C₁-C₄ alkoxy-C₁-C₄ alkyl, wherein all alkyls are optionally substituted with halogens.

In an embodiment of the invention as described anywhere herein, wherein

Z is independently OH, C₁-C₄ alkyl optionally substituted by one or more OH groups or NH₂ groups, C₁-C₄ alkyl optionally substituted by one or more halogen atoms, C₁-C₄ alkoxy optionally substituted by one or more OH groups or C₁-C₄ alkoxy, C(O)OR¹⁹, C(O)R¹⁹, CN, or halogen;

R¹⁹ is H; C₁-C₄ alkyl; C₃-C₆ cycloalkyl; or C₁-C₄ alkoxy-C₁-C₄ alkyl, wherein all alkyl are optionally substituted with halogens.

In an embodiment of the invention as described anywhere herein, wherein

Z is independently, C₁-C₄ alkyl optionally substituted by one or more halogen atoms, C₁-C₄ alkoxy or halogen.

Another embodiment of the invention as defined above provides compounds of Formula (I) in the form of substantially pure enantiomers having the R configuration.

Another embodiment of the invention as defined above provides compounds of Formula (I) in the form of substantially pure enantiomers having the S configuration.

Another embodiment of the invention as defined above provides that the bronchiectasis is cystic fibrosis bronchiectasis or non-cystic fibrosis bronchiectasis. In certain embodiments, the compound of Formula (I) (e.g., Compound A or a pharmaceutically acceptable salt thereof) is administered in combination with an additional therapy. In certain embodiments, the additional therapy comprises: a) a long-acting beta-agonist (LABA); b) a long-acting muscarinic antagonists (LAMA); c) an inhaled corticosteroid (ICS); d) macrolides; e) an antibiotic; f) a short-acting muscarinic antagonist (SAMA); or g) any combination thereof. In certain embodiments, the bronchiecstasis is characterized by deterioration in three or more symptoms for at least 48 hours. In certain embodiments, the symptoms are selected from the group consisting of: cough, sputum volume and/or consistency, sputum purulence, breathlessness and/or exercise tolerance, fatigue and/or malaise, and haemoptysis. In certain embodiments, the method further comprises: a) reducing the use of rescue medication (e.g., salbutamol/albuterol or systemic antibiotics) in a subject when compared to a subject who is not administered the compound; b) reducing the severity of exacerbations in a subject when compared to a subject who is not administered the compound; c) increasing one or more of improved lung function or forced vital capacity in a subject, e.g., as measured by spirometry, when compared to a patient who is not administered the compound; or d) any combination thereof.

Another embodiment of the invention as defined above provides that the compound of Formula (I) (e.g., Compound A or a pharmaceutically acceptable salt thereof) is administered to the subject in an amount of between about 300 mg b.i.d. and about 450 mg b.i.d., e.g., is administered in an amount of 300 mg b.i.d. or 450 mg b.i.d. In a particular embodiment, the compound of Formula (I) (e.g., Compound A or a pharmaceutically acceptable salt thereof) is administered to the subject in an amount of about 300 mg b.i.d. In yet another embodiment, the compound of Formula (I) (e.g., Compound A or a pharmaceutically acceptable salt thereof) is administered orally. In another embodiment, the compound of Formula (I) (e.g., Compound A or a pharmaceutically acceptable salt thereof) is administered to the subject without a high fat meal.

Another embodiment of the invention provides for methods of treating bronchiectasis comprising administering at least one compound of Formula (II):

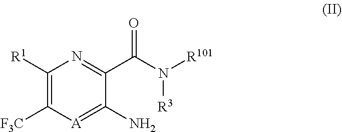

or a pharmaceutically acceptable salt thereof, to a subject in need thereof, wherein A, R¹, R² and R³ have the definitions of Formula (I) and

R¹⁰¹ is

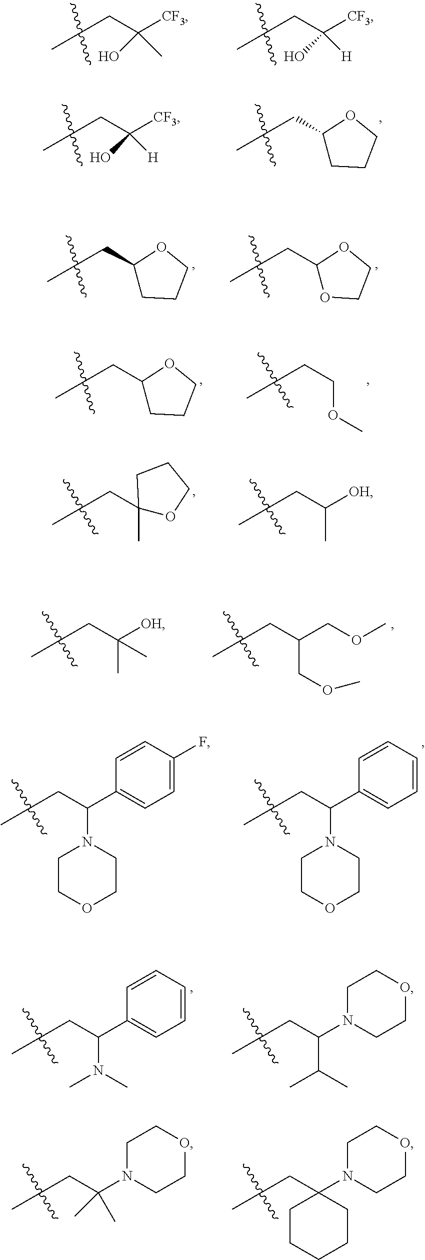

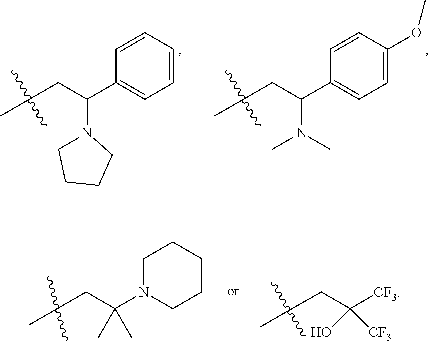 [SMILES: *CC(c1ccccc1)N1CCCC1 ; *CC(c1ccc(OC)cc1)N(C)C ; *CC(C)(C)N1CCCCC1 ; *CC(O)(C(F)(F)F)C(F)(F)F]

In a further embodiment of the invention as described above provides compounds according to Formula (II), wherein A is CR^{4a}, wherein R^{4a} is H.

In a further embodiment of the invention as described above provides compounds according to Formula (II), wherein R′ is selected from H; C₁-C₄ alkyl optionally substituted by one or more halogen atoms; C₁-C₄ alkoxy optionally substituted by one or more halogen atoms; halogen; C₆-C₁₄ aryl; —(C₀-C₄ alkyl)-3 to 14 membered heterocyclic group, wherein the heterocyclic group contains at least one heteroatom selected from N, O and S; and NR¹¹R¹², wherein the aryl and heterocyclic groups are each optionally substituted by one or more Z substituents.

In a further embodiment of the invention as described above provides compounds according to Formula (II), wherein R¹ is C₁-C₄ alkyl optional substituted by one or more halogen atoms, C₁-C₄ alkoxy optionally substituted by one or more halogen atoms; halogen; C₆ aryl; or 6 membered heterocyclic group, wherein the heterocyclic group contains at least one heteroatom selected from N, O and S, wherein the aryl and heterocyclic groups are each optionally substituted by one or more Z substituents.

In a further embodiment of the invention as described above provides compounds according to Formula (II), wherein R¹ is C₁-C₄ alkyl optional substituted by one or more halogen atoms, C₁-C₄ alkoxy optionally substituted by one or more halogen atoms; or halogen.

In a further embodiment of the invention as described above provides compounds according to Formula (II), wherein R³ is H or methyl.

An embodiment of the invention as defined above provides compounds according to Formula (II), wherein

A is CR^{4a};

R¹ is halogen;

R³ is H;

R^{4a} is H;

R¹⁰¹ is

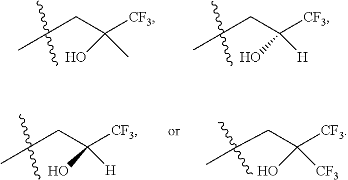

An embodiment of the invention as defined above provides compounds according to Formula (II), wherein

A is CR^{4a};

R¹ is C₁-C₄ alkyl optionally substituted by one or more halogen atoms;

R³ is H;

R^{4a} is H;

R¹⁰¹ is

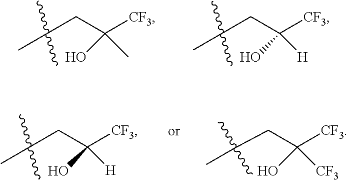

An embodiment of the invention as defined above provides compounds according to Formula (II), wherein

A is CR^{4a};

R¹ is C₁-C₄ alkoxy optionally substituted by one or more halogen atoms;

R³ is H;

R^{4a} is H;

R¹⁰¹ is

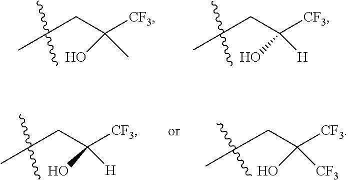

An embodiment of the invention as defined above provides compounds according to Formula (II), wherein

A is CR^{4a};

R¹ is halogen, C₁-C₄ alkyl optionally substituted by one or more halogen atoms, or C₁-C₄ alkoxy optionally substituted by one or more halogen atoms;

R³ is H;

R^{4a} is H;

R¹⁰¹ is

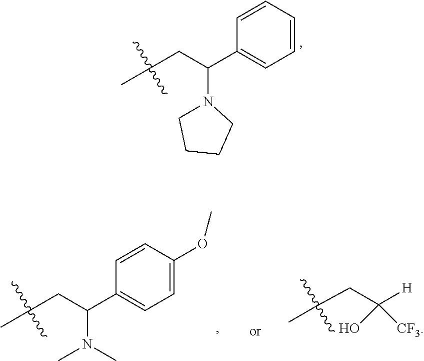 [SMILES: *CC(c1ccccc1)N1CCCC1 ; *CC(c1ccc(OC)cc1)N(C)C ; *CC(O)C(F)(F)F]

An embodiment of the invention as defined above provides compounds according to Formula (II), wherein

A is CR^{4a};

R¹ is halogen, C₁-C₄ alkyl optionally substituted by one or more halogen atoms, or C₁-C₄ alkoxy optionally substituted by one or more halogen atoms;

R³ is H;

R^{4a} is H;

R¹⁰¹ is

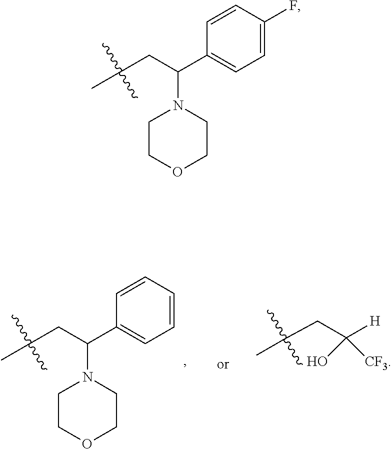 [SMILES: *CC(c1ccc(F)cc1)N1CCOCC1 ; *CC(c1ccccc1)N1CCOCC1 ; *CC(O)C(F)(F)F]

An embodiment of the invention as defined above provides compounds according to Formula (II), wherein

A is CR^{4a};

R¹ is halogen, C₁-C₄ alkyl optionally substituted by one or more halogen atoms, or C₁-C₄ alkoxy optionally substituted by one or more halogen atoms;

R³ is H;

R^{4a} is H;

R¹⁰¹ is

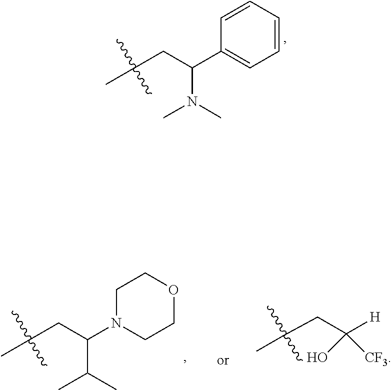 [SMILES: *CC(c1ccccc1)N(C)C ; *CC(C(C)C)N1CCOCC1 ; *CC(O)C(F)(F)F]

Another embodiment of the invention as defined above provides compounds of Formula (II) in the form of substantially pure enantiomers having the R configuration.

Another embodiment of the invention as defined above provides compounds of Formula (II) in the form of substantially pure enantiomers having the S configuration.

Another embodiment of the invention as defined above provides that the bronchiectasis is cystic fibrosis bronchiectasis or non-cystic fibrosis bronchiectasis. In certain embodiments, the compound of Formula (II) (e.g., Compound A or a pharmaceutically acceptable salt thereof) is administered in combination with an additional therapy. In certain embodiments, the additional therapy comprises: a) a long-acting beta-agonist (LABA); b) a long-acting muscarinic antagonists (LAMA); c) an inhaled corticosteroid (ICS); d) macrolides; e) an antibiotic; f) a short-acting muscarinic antagonist (SAMA); or g) any combination thereof. In certain embodiments, the bronchiecstasis is characterized by deterioration in three or more symptoms for at least 48 hours. In certain embodiments, the symptoms are selected from the group consisting of: cough, sputum volume and/or consistency, sputum purulence, breathlessness and/or exercise tolerance, fatigue and/or malaise, and haemoptysis. In certain embodiments, the method further comprises: a) reducing the use of rescue medication (e.g., salbutamol/albuterol or systemic antibiotics) in a subject when compared to a subject who is not administered the compound; b) reducing the severity of exacerbations in a subject when compared to a subject who is not administered the compound; c) increasing one or more of improved lung function or forced vital capacity in a subject, e.g., as measured by spirometry, when compared to a patient who is not administered the compound; or d) any combination thereof.

Another embodiment of the invention as defined above provides that the compound of Formula (II) (e.g., Compound A or a pharmaceutically acceptable salt thereof) is administered to the subject in an amount of between about 300 mg b.i.d. and about 450 mg b.i.d., e.g., is administered in an amount of 300 mg b.i.d. or 450 mg b.i.d. In a particular embodiment, the compound of Formula (II) (e.g., Compound A or a pharmaceutically acceptable salt thereof) is administered to the subject in an amount of about 300 mg b.i.d. In yet another embodiment, the compound of Formula (II) (e.g., Compound A or a pharmaceutically acceptable salt thereof) is administered orally. In another embodiment, the compound of Formula (II) (e.g., Compound A or a pharmaceutically acceptable salt thereof) is administered to the subject without a high fat meal.

Another embodiment of the invention provides for methods of treating bronchiectasis comprising administering at least one compound of Formula (III),

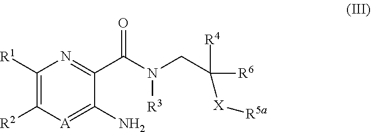

or a pharmaceutically acceptable salt thereof, to a subject in need thereof, wherein:

A is N or CR^{4a};

X is NR^{y} or O;

R¹ is C₁-C₈ alkyl optionally substituted by one or more halogen atoms; C₃-C₁₀ cycloalkyl; —C₁-C₄ alkyl-C₃-C₈ cycloalkyl; C₁-C₈ alkoxy optionally substituted by one or more halogen atoms; halogen; CN; NR¹¹R¹²; C(O)NR¹³R¹⁴; NR¹³C(O)R₁₅, CO₂R¹⁵, —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, aryl and heterocyclyl groups are each optionally substituted by one or more Z substituents;

R² is C₁-C₄ haloalkyl;

R³ and R^{4a} are each independently H or C₁-C₈ alkyl optionally substituted by one or more halogen atoms;

R⁴ is H, or C₁-C₈ alkyl optional substituted with one or more halogen;

R^{5a} is H, C₁-C₈ alkyl optional substituted with one or more halogen, —(C₀-C₄ alkyl)-C₆-C₁₄ aryl or −3 to 14 membered heterocyclic group, wherein the heterocyclic group contains at least one heteroatom selected from N, O and S; wherein the —(C₀-C₄ alkyl)-C₆-C₁₄ aryl and —(C₀-C₄ alkyl)-3 to 14 membered heterocyclic group are each optionally substituted by one or more Z substituents;

R^{y} is H, C₁-C₈ alkyl optional substituted with one or more halogen, —(C₀-C₄ alkyl)-C₆-C₁₄ aryl or −3 to 14 membered heterocyclic group, wherein the heterocyclic group contains at least one heteroatom selected from N, O and S; wherein the —(C₀-C₄ alkyl)-C₆-C₁₄ aryl and —(C₀-C₄ alkyl)-3 to 14 membered heterocyclic group are each optionally substituted by one or more Z substituents;

R⁶ is C₁-C₈ alkyl optionally substituted by one or more halogen atoms; C₃-C₁₀ cycloalkyl; —C₁-C₄ alkyl-C₃-C₈ cycloalkyl; C₁-C₈ alkoxy optionally substituted by one or more halogen atoms; OH; CN; halogen; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, —(C₀-C₄ alkyl)-C₆-C₁₄ aryl and —(C₀-C₄ alkyl)-3 to 14 membered heterocyclic group are each optionally substituted by one or more Z substituents; or

R⁴ and R⁶ together with the carbon atoms to which they are bound form a 3 to 8 membered carbocyclic ring system; or

R^{5a} and R⁶ together with the atoms to which they are bound a 5 to 8 membered heterocyclic ring system containing one or more heteroatoms selected from N, O and S, wherein the ring system is optionally substituted by one or more Z substituents; or

R^{5a} and R^{y} together with the atoms to which they are bound a 5 to 8 membered heterocyclic ring system containing one or more heteroatoms selected from N, O and S, wherein the ring system is optionally substituted by one or more Z substituents;

R¹¹ and R¹³ are each independently H, C₁-C₈ alkyl optionally substituted by one or more halogen atoms, C₃-C₁₀ cycloalkyl or —(C₁-C₄ alkyl)-C₃-C₈ cycloalkyl;

R¹², R¹⁴, and R¹⁵ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the cycloalkyl, cycloalkenyl, aryl and heterocyclyl groups are each optionally substituted by one or more Z substituents; or

R¹¹ and R¹², and R¹³ and R¹⁴ together with the nitrogen atom to which they are attached may form a 4 to 14 membered heterocyclic group optionally substituted by one or more Z substituents;

Z is independently OH, aryl, O-aryl, benzyl, O-benzyl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁸(SO₂)R²¹, (SO₂)NR¹⁹R²¹, (SO₂)R²¹, NR¹⁸C(O)R²¹, C(O)NR¹⁹R²¹, NR¹⁸C(O)NR¹⁹R²¹, NR¹⁸C(O)OR¹⁹, NR¹⁹R²¹, C(O)OR¹⁹, C(O)R¹⁹, SR¹⁹, OR¹⁹, oxo, CN, NO₂, halogen or a 3 to 14 membered heterocyclic group, wherein the heterocyclic group contains at least one heteroatom selected from N, O and S;

R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂-C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy.

Another embodiment of the invention as defined above provides compounds of Formula (III) in the form of substantially pure enantiomers having the R configuration.

Another embodiment of the invention as defined above provides compounds of Formula (III) in the form of substantially pure enantiomers having the S configuration.

Another embodiment of the invention as defined above provides that the bronchiectasis is cystic fibrosis bronchiectasis or non-cystic fibrosis bronchiectasis. In certain embodiments, the compound of Formula (III) (e.g., Compound A or a pharmaceutically acceptable salt thereof) is administered in combination with an additional therapy. In certain embodiments, the additional therapy comprises: a) a long-acting beta-agonist (LABA); b) a long-acting muscarinic antagonists (LAMA); c) an inhaled corticosteroid (ICS); d) macrolides; e) an antibiotic; f) a short-acting muscarinic antagonist (SAMA); or g) any combination thereof. In certain embodiments, the bronchiecstasis is characterized by deterioration in three or more symptoms for at least 48 hours. In certain embodiments, the symptoms are selected from the group consisting of: cough, sputum volume and/or consistency, sputum purulence, breathlessness and/or exercise tolerance, fatigue and/or malaise, and haemoptysis. In certain embodiments, the method further comprises: a) reducing the use of rescue medication (e.g., salbutamol/albuterol or systemic antibiotics) in a subject when compared to a subject who is not administered the compound; b) reducing the severity of exacerbations in a subject when compared to a subject who is not administered the compound; c) increasing one or more of improved lung function or forced vital capacity in a subject, e.g., as measured by spirometry, when compared to a patient who is not administered the compound; or d) any combination thereof.

Another embodiment of the invention as defined above provides that the compound of Formula (III) (e.g., Compound A or a pharmaceutically acceptable salt thereof) is to the subject administered in an amount of between about 300 mg b.i.d. and about 450 mg b.i.d., e.g., is administered in an amount of 300 mg b.i.d. or 450 mg b.i.d. In a particular embodiment, the compound of Formula (III) (e.g., Compound A or a pharmaceutically acceptable salt thereof) is administered to the subject in an amount of about 300 mg b.i.d. In yet another embodiment, the compound of Formula (III) (e.g., Compound A or a pharmaceutically acceptable salt thereof) is administered orally. In another embodiment, the compound of Formula (III) (e.g., Compound A or a pharmaceutically acceptable salt thereof) is administered to the subject without a high fat meal.

Another embodiment of the invention as defined above provides a method of treating bronchiectasis comprising administering at least one compound according to Formula (I) and/or Formula (II) to a subject in need thereof, selected from the group consisting of:
3-amino-6-bromo-N-(imidazo[1,2-a]pyridin-2-ylmethyl)-5-(trifluoromethyl)pyrazine-2-carboxamide;
3-amino-6-bromo-N-((1-methyl-1H-imidazol-4-yl)methyl)-5-(trifluoromethyl)pyrazine-2-carboxamide;
3-amino-6-bromo-N-((1-methyl-1H-pyrazol-3-yl)methyl)-5-(trifluoromethyl)pyrazine-2-carboxamide;
3-amino-N-(2-(4-fluorophenyl)-2-oxoethyl)-6-(1-methyl-1H-indol-6-yl)-5-(trifluoromethyl)picolinamide;
3-amino-6-bromo-N-((1-methyl-1H-imidazol-2-yl)methyl)-5-(trifluoromethyl)pyrazine-2-carboxamide;
3-amino-6-(6-(3-(dimethylamino)propoxy)pyridin-3-yl)-N-(2-(4-fluorophenyl)-2-oxoethyl)-5-(trifluoromethyl)pyrazine-2-carboxamide;
(R)-3-amino-6-bromo-N-((4-methylpiperazin-2-yl)methyl)-5-(trifluoromethyl)pyrazine-2-carboxamide;
3-amino-6-bromo-N-((1-methyl-1H-imidazol-5-yl)methyl)-5-(trifluoromethyl)pyrazine-2-carboxamide;
3-amino-6-(3-(N,N-dimethylsulfamoyl)phenyl)-N-(2-(4-fluorophenyl)-2-oxoethyl)-5-(trifluoromethyl)picolinamide;
3-amino-6-bromo-N-isobutyl-N-methyl-5-(trifluoromethyl)pyrazine-2-carboxamide; 3-amino-6-bromo-N-((1-methyl-1H-pyrazol-5-yl)methyl)-5-(trifluoromethyl)pyrazine-2-carboxamide;
(3-amino-6-bromo-5-(trifluoromethyl)pyrazin-2-yl)(4-methylpiperazin-1-yl)methanone;
3-amino-6-bromo-N-(2-(pyridin-4-yl)ethyl)-5-(trifluoromethyl)pyrazine-2-carboxamide;
3-amino-N-(2-(4-fluorophenyl)-2-oxoethyl)-6-(1-oxo-1,2,3,4-tetrahydroisoquinolin-6-yl)-5-(trifluoromethyl)picolinamide;
3-amino-6-(4-carbamoyl-2-methylphenyl)-N-(2-(4-fluorophenyl)-2-oxoethyl)-5-(trifluoromethyl)picolinamide;
3-amino-6-bromo-N-(2-(pyridin-3-yl)ethyl)-5-(trifluoromethyl)pyrazine-2-carboxamide;
3-amino-6-(3,4-dimethylphenyl)-N-(2-(4-fluorophenyl)-2-oxoethyl)-5-(trifluoromethyl)picolinamide;
3-amino-N-benzyl-6-bromo-N-methyl-5-(trifluoromethyl)pyrazine-2-carboxamide;
(S)-3-amino-6-bromo-N-((1-ethylpyrrolidin-2-yl)methyl)-5-(trifluoromethyl)pyrazine-2-carboxamide; and
3-amino-6-bromo-N-(imidazo[1,5-a]pyridin-1-ylmethyl)-5-(trifluoromethyl)pyrazine-2-carboxamide; or
   a pharmaceutically acceptable salt thereof.

Another embodiment of the invention as defined above provides a method of treating bronchiectasis comprising administering at least one compound according to Formula (I) and/or Formula (II), to a subject in need thereof, selected from the group consisting of:
3-Amino-6-methoxy-5-trifluoromethyl-pyridine-2-carboxylic acid ((S)-3,3,3-trifluoro-2-hydroxy-2-methyl-propyl)-amide;
3-Amino-6-methoxy-5-trifluoromethyl-pyridine-2-carboxylic acid ((R)-3,3,3-trifluoro-2-hydroxy-2-methyl-propyl)-amide;
Methyl 3-(3-amino-6-bromo-5-(trifluoromethyl)picolinamido)propanoate;
3-Amino-N-(benzo[d]isoxazol-3-ylmethyl)-6-bromo-5-(trifluoromethyl) picolinamide;
3-Amino-6-(oxazol-2-yl)-N-(3,3,3-trifluoro-2-hydroxy-2-methylpropyl)-5-(trifluoromethyl)picolinamide;
3-Amino-6-bromo-N-(3,3,3-trifluoro-2-methoxy-2-methylpropyl)-5-(trifluoromethyl)picolinamide;
3-amino-N-(2-hydroxy-3-methyl-2-(trifluoromethyl)butyl)-6-methoxy-5-(trifluoromethyl)picolinamide;
3-Amino-6-cyclopropyl-N-(3,3,3-trifluoro-2-hydroxy-2-methylpropyl)-5-(trifluoromethyl)picolinamide;
3-Amino-6-methoxy-N-(3,3,3-trifluoro-2-hydroxy-2-(trifluoromethyl)propyl)-5-(trifluoro methyl) picolinamide;
5-amino-N-(3,3,3-trifluoro-2-hydroxy-2-methylpropyl)-3-(trifluoromethyl)-2,4′-bipyridine-6-carboxamide;
3-Amino-6-bromo-5-trifluoromethyl-pyridine-2-carboxylic acid (3-methyl-2-oxo-butyl)-amide;
3-Amino-6-bromo-5-trifluoromethyl-pyrazine-2-carboxylic acid [2-(4-fluoro-phenyl)-2-oxo-ethyl]-amide;
3-Amino-6-furan-2-yl-5-trifluoromethyl-pyrazine-2-carboxylic acid [2-(2-methoxy-phenyl)-ethyl]-amide;
3-Amino-6-(1-methyl-1H-pyrazol-4-yl)-N-(3,3,3-trifluoro-2-hydroxy-2-methylpropyl)-5-(trifluoromethyl)picolinamide;
3-amino-N-(3,3,3-trifluoro-2-hydroxy-2-methylpropyl)-5,6-bis(trifluoromethyl) pyrazine-2-carboxamide;
N-(2-(1H-imidazol-2-yl)propyl)-3-amino-6-bromo-5-(trifluoromethyl)pyrazine-2-carboxamide;
3-Amino-6-bromo-N-(2-morpholinoethyl)-5-(trifluoromethyl)pyrazine-2-carboxamide;
(S)-3-amino-6-ethoxy-N-(3,3,3-trifluoro-2-hydroxy-2-methylpropyl)-5-(trifluoro methyl)picolinamide;
3-Amino-6-(pyrrolidin-1-yl)-N-(3,3,3-trifluoro-2-hydroxy-2-methylpropyl)-5-(trifluoromethyl)picolinamide;
3-Amino-N-(2-amino-3,3,3-trifluoro-2-methylpropyl)-6-methoxy-5-(trifluoromethyl) picolinamide; and
3-Amino-6-methoxy-N-(3,3,3-trifluoro-2-(4-methoxybenzylamino)-2-methylpropyl)-5-(trifluoromethyl)picolinamide; or
   a pharmaceutically acceptable salt thereof.

Another embodiment of the invention as defined above provides a method for treating bronchiectasis comprising administering an effective amount of a compound selected from the group consisting of: 3-Amino-6-methoxy-5-trifluoromethyl-pyridine-2-carboxylic acid ((5)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to a subject in need thereof.

Another embodiment of the invention as defined above provides a method for inhibiting or reducing the level of colonization of at least one pathogenic bacteria in the lungs of a subject in need thereof comprising administering an effective amount of a compound selected from the group consisting of: 3-Amino-6-methoxy-5-trifluoromethyl-pyridine-2-carboxylic acid ((5)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to the subject, optionally wherein the level of at least one pathogenic bacteria is measured from a sputum sample obtained from the subject, optionally wherein the level of at least one pathogenic bacteria is measured by 16S rRNA PCR. In certain embodiments, the pathogenic bacteria is a non-fermenting Gram negative bacteria. In still further embodiments, the pathogenic bacteria is selected from the group consisting of M. catarrhalis, S. aureus, Enterobacteriaceae, Stenotrophomonous Maltophilia Hemophilus parainfluenza, Hemophilus influenza, Pseudomonas aeruginosa, Moraxella, and Streptococcus pneumonia. In other embodiments, the level of colonization of pathogenic bacteria is reduced by at least one log.

Another embodiment of the invention as defined above provides a method for reducing the level of fibrinogen in the blood of a subject in need thereof, e.g., a bronchiectasis subject, comprising administering an effective amount of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to the subject.

In particular embodiments, the compound is 3-Amino-6-methoxy-5-trifluoromethyl-pyridine-2-carboxylic acid ((S)-3,3,3-trifluoro-2-hydroxy-2-methyl-propyl)-amide or a pharmaceutically acceptable salt thereof. In certain embodiments, the subject in need thereof is a bronchiectasis subject. In other embodiments, the compound is administered to the subject in an amount of between about 300 mg b.i.d. and about 450 mg b.i.d., e.g., is administered in an amount of 300 mg b.i.d. or 450 mg b.i.d. In a particular embodiment, the compound is administered to the subject in an amount of about 300 mg b.i.d. In other embodiments, the compound is administered orally. In some embodiments, the compound is administered to the subject without a high fat meal. In certain embodiments, the compound is administered in combination with an additional therapy. In certain embodiments, the additional therapy comprises: a) a long-acting beta-agonist (LABA); b) a long-acting muscarinic antagonists (LAMA); c) an inhaled corticosteroid (ICS); d) macrolides; e) an antibiotic; f) a short-acting muscarinic antagonist (SAMA); or g) any combination thereof. In other embodiments, the bronchiecstasis is characterized by deterioration in three or more symptoms for at least 48 hours. In some embodiments, the symptoms are selected from the group consisting of: cough, sputum volume and/or consistency, sputum purulence, breathlessness and/or exercise tolerance, fatigue and/or malaise, and haemoptysis. In certain embodiments, the method further comprises: a) reducing the use of rescue medication (e.g., salbutamol/albuterol or systemic antibiotics) in a subject when compared to a subject who is not administered the compound; b) reducing the severity of exacerbations in a subject when compared to a subject who is not administered the compound; c) increasing one or more of improved lung function or forced vital capacity in a subject, e.g., as measured by spirometry, when compared to a patient who is not administered the compound; or d) any combination thereof.

It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments. It is understood by those skilled in the art that combinations of substituents where not possible are not an aspect of the present invention.

Especially preferred specific compounds of Formula (I) or Formula (II) are those described hereinafter in the Examples.

### 5. BRIEF DESCRIPTION OF THE DRAWINGS

FIG. 1. The mean change from baseline (SE) over time for fibrinogen per treatment with Compound A in COPD patients. The numbers presented in FIG. 1 adjacent to legends at each visit represent the number of patients.

FIG. 2. Flowchart describing a randomized, subject- and investigator-blinded, placebo-controlled, parallel-group study investigating the preliminary efficacy and safety of Compound A administered orally for 12 weeks in subjects with bronchiectasis.

### 6. DETAILED DESCRIPTION

### 6.1. Definitions

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

“Compound A” means 3-amino-6-methoxy-5-trifluoromethyl-pyridine-2-carboxylic acid ((S)-3,3,3-trifluoro-2-hydroxy-2-methyl-propyl)-amide or a pharmaceutically accceptable salt thereof having the following structure:

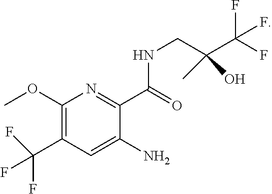 [SMILES: COc1nc(C(=O)NC[C@](C)(O)C(F)(F)F)c(N)cc1C(F)(F)F]

“Administration” and “administering” and “administer” refer to the manner in which a compound described herein (e.g., Compound A) is presented to a subject.

“Optionally substituted” means the group referred to can be substituted at one or more positions by any one or any combination of the radicals listed thereafter.

“Optionally substituted by one or more Z groups” denotes that the relevant group may include one or more substituents, each independently selected from the groups included within the definition of Z. Thus, where there are two or more Z group substituents, these may be the same or different.

“Halo” or “halogen”, as used herein, may be fluorine, chlorine, bromine or iodine.

“C₁-C₈-Alkyl”, as used herein, denotes straight chain or branched alkyl having 1-8 carbon atoms. If a different number of carbon atoms is specified, such as C₆ or C₃, then the definition is to be amended accordingly, such as “C₁-C₄-Alkyl” will represent methyl, ethyl, propyl, isopropyl, butyl, isobutyl, sec-butyl and tert-butyl.

“C₁-C₈-Alkoxy”, as used herein, denotes straight chain or branched alkoxy having 1-8 carbon atoms. If a different number of carbon atoms is specified, such as C₆ or C₃, then the definition is to be amended accordingly, such as “C₁-C₄-Alkoxy” will represent methoxy, ethoxy, propoxy, isopropoxy, butoxy, isobutoxy, sec-butoxy and tert-butoxy.

“C₁-C₄-Haloalkyl”, as used herein, denotes straight chain or branched alkyl having 1-4 carbon atoms with at least one hydrogen substituted with a halogen. If a different number of carbon atoms is specified, such as C₆ or C₃, then the definition is to be amended accordingly, such as “C₁-C₄-Haloalkyl” will represent methyl, ethyl, propyl, isopropyl, butyl, isobutyl, sec-butyl and tert-butyl that have at least one hydrogen substituted with halogen, such as where the halogen is fluorine: CF₃CF₂—, (CF₃)₂CH—, CH₃—CF₂—, CF₃CF₂—, CF₃, CF₂H—, CF₃CF₂CHCF₃ or CF₃CF₂CF₂CF₂—.

“C₃-C₁₅-Cycloalkyl group”, as used herein, denotes a cycloalkyl group having 3- to 15-ring carbon atoms that is saturated or partially saturated, such as a C₃-C₈-cycloalkyl. Examples of C₃-C₁₅-cycolalkyl groups include but are not limited to cyclopropyl, cyclobutyl, cyclopentyl, cyclohexyl, cycloheptyl or cyclooctyl or a bicyclic group, such as bicyclooctyl, bicyclononyl including indanyl and indenyl and bicyclodecyl. If a different number of carbon atoms is specified, such as C₆, then the definition is to be amended accordingly.

“Aryl” or “C₆-C₁₅-Aromatic carbocyclic group”, as used herein, denotes an aromatic group having 6- to 15-ring carbon atoms. Examples of C₆-C₁₅-aromatic carbocyclic groups include, but are not limited to, phenyl, phenylene, benzenetriyl, naphthyl, naphthylene, naphthalenetriyl or anthrylene. If a different number of carbon atoms is specified, such as C₁₀, then the definition is to be amended accordingly.

“4- to 8-Membered heterocyclic group”, “5- to 6-membered heterocyclic group”, “3- to 10-membered heterocyclic group”, “3- to 14-membered heterocyclic group”, “4- to 14-membered heterocyclic group” and “5- to 14-membered heterocyclic group”, refers, respectively, to 4- to 8-membered, 5- to 6-membered, 3- to 10-membered, 3- to 14-membered, 4- to 14-membered and 5- to 14-membered heterocyclic rings containing at least one ring heteroatom selected from the group consisting of nitrogen, oxygen and sulphur, which may be saturated, partially saturated or unsaturated (aromatic). The heterocyclic group includes single ring groups, fused ring groups and bridged groups. Examples of such heterocyclic groups include, but are not limited to, furan, pyrrole, pyrrolidine, pyrazole, imidazole, triazole, isotriazole, tetrazole, thiadiazole, isothiazole, oxadiazole, pyridine, piperidine, pyrazine, oxazole, isoxazole, pyrazine, pyridazine, pyrimidine, piperazine, pyrrolidine, pyrrolidinone, morpholine, triazine, oxazine, tetrahyrofuran, tetrahydrothiophene, tetrahydrothiopyran, tetrahydropyran, 1,4-dioxane, 1,4-oxathiane, indazole, quinoline, indazole, indole, 8-aza-bicyclo[3.2.1]octane or thiazole.

“Subject” refers to a living organism suffering from one or more of the diseases or disorders described here (e.g., bronchiectasis, COPD, CF, chronic bronchitis, primary ciliary dyskinesia, respiratory tract infections or asthma) that can be treated by administration of a pharmaceutical composition described herein. Examples of subjects include mammals (e.g., humans and animals such as dogs, cows, horses, monkeys, pigs, sheep, goats, cats, mice, rabbits, rats, and transgenic non-human animals). In certain embodiments, the subject is a human, e.g., a human suffering from, at risk of suffering from, or potentially capable of suffering from a disease described herein (e.g., bronchiectasis, COPD, CF, chronic bronchitis, primary ciliary dyskinesia, respiratory tract infections or asthma). “Treat”, “treating”, or “treatment” includes prophylactic (preventive) and therapeutic treatment as well as the delay of progression of a disease or disorder described herein (e.g., bronchiectasis, COPD, CF, chronic bronchitis, primary ciliary dyskinesia, respiratory tract infections or asthma). The term “delay of progression” as used herein means administration of the pharmaceutical composition to patients being in a pre-stage or in an early phase of the disease or disorder described herein (e.g., bronchiectasis, COPD, CF, chronic bronchitis, primary ciliary dyskinesia, respiratory tract infections or asthma) to be treated, in which patients, for example a pre-form of the corresponding disease, are diagnosed or which patients are in a condition, e.g., during a medical treatment, under which it is likely that a corresponding disease will develop.

Throughout this specification and in the claims that follow, unless the context requires otherwise, the word “comprise”, or variations such as “comprises” or “comprising”, should be understood to imply the inclusion of a stated integer or step or group of integers or steps but not the exclusion of any other integer or step or group of integers or steps.

“Pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, and sulfosalicylic acid.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Furthermore, the compounds of the present invention, including their salts, can also be obtained in the form of their hydrates, or include other solvents used for their crystallization.

Compounds of the invention, i.e., compounds of Formula (I), (II) or (III) that contain groups capable of acting as donors and/or acceptors for hydrogen bonds may be capable of forming co-crystals with suitable co-crystal formers. These co-crystals may be prepared from compounds of Formula (I), (II) or (III) by known co-crystal forming procedures. Such procedures include grinding, heating, co-subliming, co-melting, or contacting in solution compounds of Formula (I), (II) or (III) with the co-crystal former under crystallization conditions and isolating co-crystals thereby formed. Suitable co-crystal formers include those described in WO 2004/078163. Hence the invention further provides co-crystals comprising a compound of Formula (I), (II) or (III).

“Isomers” refers to different compounds that have the same molecular formula but differ in arrangement and configuration of the atoms.

“An optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term is used to designate a racemic mixture where appropriate.

“Diastereoisomers” are stereoisomers that have at least two asymmetric atoms, but which are not mirror-images of each other. The absolute stereochemistry is specified according to the Cahn-lngold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers or axes and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

Compounds of the present invention are either obtained in the free form, as a salt thereof, or as prodrug derivatives thereof. Drug dosages disclosed herein are calculated using the free base form of a compound of Formula (I), (II), or (III) (e.g., Compound A or a pharmaceutically acceptable salt thereof), e.g., an amount of between about 300 mg twice daily (b.i.d.) and about 450 mg b.i.d., e.g., is administered in an amount of 300 mg b.i.d. or 450 mg b.i.d. In a particular embodiment, the compound of Formula (I), (II), or (III) (e.g., Compound A or a pharmaceutically acceptable salt thereof) is administered to the subject in an amount of about 300 mg b.i.d. In a more particular embodiment, Compound A is administered to the subject in an amount of about 300 mg b.i.d.

When both a basic group and an acid group are present in the same molecule, the compounds of the present invention may also form internal salts, e.g., zwitterionic molecules.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous fluorine and chlorine such as ²H, ³H, ¹¹C, ¹³C, ¹⁴C, ¹⁵N, ¹⁸F, ³¹P, ³²P, ³⁵S, ³⁶Cl, ¹²⁵I respectively. The invention includes various isotopically labeled compounds as defined herein, for example those into which radioactive isotopes, such as ³H, ¹³C, and ¹⁴C are present. Such isotopically labeled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically labeled compounds of this invention can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the Formula (I), (II) or (III). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically-labeled compounds of Formula (I), (II) or (III) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d_{c6}-acetone, d₆-DMSO.

If there is a discrepancy between a depicted structure and a chemical name given to that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure of portion of the structure.

“High fat meal” refers to the definition by the U.S. Food and Drug Administration in the draft guidance on Assessing the Effects of Food on Drugs in INDs and NDAs (FDA 2019) (see also Assessing the Effects of Food on Drugs in Investigational New Drug Applications and New Drug Applications-Clinical Pharmacology Considerations; Draft Guidance for Industry; Availability, 84 Fed. Reg. 6151 (Feb. 26, 2019)) and the corresponding EMA guideline (EMA 2012), wherein the high fat meal contains at least 1000 kcal (4184 kJ) and at least 50% of that energy content is derived from fat. An example of a high-fat meal would be:

Total nutritional energy value: 1000 kcal
of which from proteins: 150 kcal
of which from carbohydrates: 250 kcal
of which from fats: 600 kcal.

“Without a high fat meal” is defined to mean the condition of not having consumed a high fat meal together with administration of a compound of formula (I) or a pharmaceutically effective salt thereof (e.g., Compound A or a pharmaceutically effective salt thereof) or the condition of not having consumed a high fat meal within a certain time prior to the administration of a compound of formula (I) or a pharmaceutically effective salt thereof to a certain time after the administration of a compound of formula (I) or a pharmaceutically effective salt thereof. In some embodiments, the high fat meal has not been consumed for about 10 hours, about 8 hours, about 6 hours, about 4 hours, about 2 hours, about 1 hour, about 30 minutes, about 15 minutes, about 5 minutes, or about 1 minute prior to administration of a compound of formula (I) or a pharmaceutically effective salt thereof. In some embodiments, the high fat meal has not been consumed for about 10 hours, about 8 hours, about 6 hours, about 4 hours, about 2 hours, about 1 hour, about 30 minutes, about 15 minutes, about 5 minutes, or about 1 minute after the administration of a compound of formula (I) or a pharmaceutically effective salt thereof. In other embodiments, the high fat meal has not been consumed together with administration of a compound of formula (I) or a pharmaceutically effective salt thereof. In certain embodiments, the high fat meal has not been consumed for about 30 minutes prior to administration of a compound of formula (I) or a pharmaceutically effective salt thereof.

“Exacerbation(s)” refers to a deterioration in three or more of the following key symptoms for at least 48 hours:
cough
sputum volume and/or consistency;
sputum purulence;
breathlessness and/or exercise tolerance;
fatigue and/or malaise;
haemoptysis

### AND

A clinician determines that a change in bronchiectasis treatment is required (e.g. requiring systemic glucocorticosteroid treatment and/or systemic or inhaled antibiotics).

A worsening of symptoms that either does not meet the above symptom definition but is treated by the investigator with antibiotics, or that meets the symptom definition but is not treated with antibiotics, is not considered a pulmonary exacerbation for the study.

### 6.2. Pharmaceutical Compositions and Methods of Use

### 6.2.1. Inflammatory or Allergic Condition

An aspect of the invention provides a compound of Formula (I), (II) or (III) (e.g., Compound A or a pharmaceutically acceptable salt thereof) as defined anywhere herein for use as a pharmaceutical.

A further aspect of the invention provides a method of treating an inflammatory or allergic condition or infection, particularly an inflammatory or obstructive airways disease or mucosal hydration comprising administering an effective amount of a compound of Formula (I), (II) or (III) (e.g., Compound A or a pharmaceutically acceptable salt thereof) to a subject in need thereof.

A further aspect of the invention provides a compound of Formula (I), (II) or (III) (e.g., Compound A or a pharmaceutically acceptable salt thereof) for use in the treatment of an inflammatory or allergic condition or infection, particularly an inflammatory or obstructive airways disease or mucosal hydration in a subject in need thereof.

A still further aspect of the present invention provides for the use of a compound of Formula (I), (II) or (III) (e.g., Compound A or a pharmaceutically acceptable salt thereof), as defined in any of the aforementioned embodiments, in free or pharmaceutically acceptable salt form, in the manufacture of a medicament for the treatment of an inflammatory or allergic condition or infection, particularly an inflammatory or obstructive airways disease or mucosal hydration in a subject in need thereof.

A still further aspect of the present invention provides for the use of a compound of Formula (I), (II) or (III) (e.g., Compound A or a pharmaceutically acceptable salt thereof), as defined in any of the aforementioned embodiments, in free or pharmaceutically acceptable salt form, for the treatment of an inflammatory or allergic condition or infection, particularly an inflammatory or obstructive airways disease or mucosal hydration in a subject in need thereof.

A still further aspect of the present invention provides for a pharmaceutical composition comprising a compound of Formula (I), (II) or (III) (e.g., Compound A or a pharmaceutically acceptable salt thereof), as defined in any of the aforementioned embodiments, in free or pharmaceutically acceptable salt form, for the treatment of an inflammatory or allergic condition or infection, particularly an inflammatory or obstructive airways disease or mucosal hydration in a subject in need thereof.

An aspect of the invention provides a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for use as a pharmaceutical.

A further aspect of the invention provides a method of treating an inflammatory or allergic condition or infection, particularly an inflammatory or obstructive airways disease or mucosal hydration comprising administering an effective amount of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to a subject in need thereof.

A further aspect of the invention provides a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for use in the treatment of an inflammatory or allergic condition or infection, particularly an inflammatory or obstructive airways disease or mucosal hydration in a subject in need thereof.

A still further aspect of the present invention provides for the use of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, in the manufacture of a medicament for the treatment of an inflammatory or allergic condition or infection, particularly an inflammatory or obstructive airways disease or mucosal hydration in a subject in need thereof.

A still further aspect of the present invention provides for the use of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for the treatment of an inflammatory or allergic condition or infection, particularly an inflammatory or obstructive airways disease or mucosal hydration in a subject in need thereof.

A still further aspect of the present invention provides for a pharmaceutical composition comprising a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for the treatment of an inflammatory or allergic condition or infection, particularly an inflammatory or obstructive airways disease or mucosal hydration in a subject in need thereof.

In some embodiments, the compound is a compound of Formula (I), (II), or (III) or a pharmaceutically aacceptable salt thereof. In particular embodiments, the compound is 3-Amino-6-methoxy-5-trifluoromethyl-pyridine-2-carboxylic acid ((S)-3,3,3-trifluoro-2-hydroxy-2-methyl-propyl)-amide or a pharmaceutically acceptable salt thereof. In certain embodiments, the bronchiectasis is cystic fibrosis bronchiectasis or non-cystic fibrosis bronchiectasis. In certain embodiments, the compound is administered in combination with at least one additional therapy. In certain embodiments, the additional therapy comprises: a) a long-acting beta-agonist (LABA); b) a long-acting muscarinic antagonists (LAMA); c) an inhaled corticosteroid (ICS); d) macrolides; e) an antibiotic (e.g., a macrolide antibiotic); f) a short-acting muscarinic antagonist (SAMA); or g) any combination thereof. When administered in combination, the two or more agents may be administered sequentially or concurrently and may be administered in one or more compositions. In certain embodiments, the bronchiecstasis is characterized by deterioration in three or more symptoms for at least 48 hours. In certain embodiments, the symptoms are selected from the group consisting of: cough, sputum volume and/or consistency, sputum purulence, breathlessness and/or exercise tolerance, fatigue and/or malaise, and haemoptysis. Another embodiment of the invention as defined above provides that the compound is administered to the subject in an amount of between about 300 mg b.i.d. and about 450 mg b.i.d., e.g., is administered in an amount of 300 mg b.i.d. or 450 mg b.i.d. In a particular embodiment, the compound is administered to the subject in an amount of about 300 mg b.i.d. In yet another embodiment, the compound is administered orally. In another embodiment, the compound is administered to the subject without a high fat meal. In certain embodiments, the method further comprises: a) reducing the use of rescue medication (e.g., salbutamol/albuterol or systemic antibiotics) in a subject when compared to a subject who is not administered the compound; b) reducing the severity of exacerbations in a subject when compared to a subject who is not administered the compound; c) increasing one or more of improved lung function or forced vital capacity in a subject, e.g., as measured by spirometry, when compared to a patient who is not administered the compound; or d) any combination thereof.

### 6.2.2. Bronchiectasis

An embodiment of the present invention provides for a method of treating bronchiectasis comprising administering an effective amount of a compound of Formula (I), (II), or (III) (e.g., Compound A or a pharmaceutically acceptable salt thereof), as defined in any of the aforementioned embodiments, in free or pharmaceutically acceptable salt form, to a subject in need thereof.

A further embodiment of the present invention provides for the use of a compound of Formula (I), (II) or (III) (e.g., Compound A or a pharmaceutically acceptable salt thereof), as defined in any of the aforementioned embodiments, in free or pharmaceutically acceptable salt form, in the manufacture of a medicament for the treatment of bronchiectasis in a subject in need thereof.

A further embodiment of the present invention provides for the use of a compound of Formula (I), (II) or (III) (e.g., Compound A or a pharmaceutically acceptable salt thereof), as defined in any of the aforementioned embodiments, in free or pharmaceutically acceptable salt form, for the treatment of bronchiectasis in a subject in need thereof.

Another embodiment of the present invention provides a compound of Formula (I), (II), or (III) (e.g., Compound A or a pharmaceutically acceptable salt thereof), as defined in any of the aforementioned embodiments, in free or pharmaceutically acceptable salt form, for use in the treatment of bronchiectasis in a subject in need thereof.

Another embodiment of the invention provides for a pharmaceutical composition comprising a compound of Formula (I), (II) or (III) (e.g., Compound A) for use in the treatment of bronchiectasis in a subject in need thereof.

Another particular embodiment of the invention as defined above provides a method for treating bronchiectasis comprising administering an effective amount of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to a subject in need thereof.

Another embodiment of the invention as defined above provides for the use of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, in the treatment of bronchiectasis in a subject in need thereof.

Another embodiment of the invention as defined above provides for the use of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, in the manufacture of a medicament for the treatment of bronchiectasis in a subject in need thereof.

Another embodiment of the invention as defined above provides a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for use in the treatment of bronchiectasis in a subject in need thereof.

Another embodiment of the invention provides for pharmaceutical composition comprising a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for use in the treatment of bronchiectasis in a subject in need thereof.

In some embodiments, the compound is a compound of Formula (I), (II), or (III) or a pharmaceutically aacceptable salt thereof. In particular embodiments, the compound is 3-Amino-6-methoxy-5-trifluoromethyl-pyridine-2-carboxylic acid ((S)-3,3,3-trifluoro-2-hydroxy-2-methyl-propyl)-amide or a pharmaceutically acceptable salt thereof. In certain embodiments, the bronchiectasis is cystic fibrosis bronchiectasis or non-cystic fibrosis bronchiectasis. In other embodiments, the compound is administered in combination with at least one additional therapy. In some embodiments, the additional therapy comprises: a) a long-acting beta-agonist (LABA); b) a long-acting muscarinic antagonists (LAMA); c) an inhaled corticosteroid (ICS); d) macrolides; e) an antibiotic (e.g., a macrolide antibiotic); f) a short-acting muscarinic antagonist (SAMA); or g) any combination thereof. When administered in combination, the two or more agents may be administered sequentially or concurrently and may be administered in one or more compositions. In some embodiments, the bronchiecstasis is characterized by deterioration in three or more symptoms for at least 48 hours. In some embodiments, the symptoms are selected from the group consisting of: cough, sputum volume and/or consistency, sputum purulence, breathlessness and/or exercise tolerance, fatigue and/or malaise, and haemoptysis. In other embodiments, the compound is administered to the subject in an amount of between about 300 mg b.i.d. and about 450 mg b.i.d., e.g., is administered in an amount of 300 mg b.i.d. or 450 mg b.i.d. In a particular embodiment, the compound is administered to the subject in an amount of about 300 mg b.i.d. In yet another embodiment, the compound is administered orally. In another embodiment, the compound is administered to the subject without a high fat meal. In certain embodiments, the method further comprises: a) reducing the use of rescue medication (e.g., salbutamol/albuterol or systemic antibiotics) in a subject when compared to a subject who is not administered the compound; b) reducing the severity of exacerbations in a subject when compared to a subject who is not administered the compound; c) increasing one or more of improved lung function or forced vital capacity in a subject, e.g., as measured by spirometry, when compared to a patient who is not administered the compound; or d) any combination thereof.

### 6.2.3. Inhibiting/Reducing Colonization of Pathogenic Bacteria

An embodiment of the present invention provides for a method for inhibiting or reducing the level of colonization of at least one pathogenic bacteria in the lungs of a subject comprising administering a compound of Formula (I), (II), or (III) (e.g., Compound A or a pharmaceutically acceptable salt thereof), as defined in any of the aforementioned embodiments, in free or pharmaceutically acceptable salt form, to a subject in need thereof.

Another embodiment of the present invention provides for the use of a compound of Formula (I), (II), or (III) (e.g., Compound A or a pharmaceutically acceptable salt thereof), as defined in any of the aforementioned embodiments, in free or pharmaceutically acceptable salt form, in the manufacture of a medicament for inhibiting or reducing the level of colonization of at least one pathogenic bacteria in the lungs of a subject in need thereof.

Another embodiment of the present invention provides for a compound of Formula (I), (II), or (III) (e.g., Compound A or a pharmaceutically acceptable salt thereof), as defined in any of the aforementioned embodiments, in free or pharmaceutically acceptable salt form, for use in inhibiting or reducing the level of colonization of at least one pathogenic bacteria in the lungs of a subject in need thereof.

Another embodiment of the invention provides for pharmaceutical composition comprising a compound of Formula (I), (II), or (III) (e.g., Compound A) for use in inhibiting or reducing the level of colonization of at least one pathogenic bacteria in the lungs of a subject in need thereof.

A particular embodiment of the invention as defined above provides a method for inhibiting or reducing the level of colonization of at least one pathogenic bacteria in the lungs of a subject in need thereof comprising administering an effective amount of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to the subject.

Another embodiment of the present invention provides for the use of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, in the manufacture of a medicament for inhibiting or reducing the level of colonization of at least one pathogenic bacteria in the lungs of a subject in need thereof.

Another embodiment of the present invention provides for the use of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for inhibiting or reducing the level of colonization of at least one pathogenic bacteria in the lungs of a subject in need thereof.

Another embodiment of the present invention provides for a compound selected from the group consisting of: 3-Amino-6-methoxy-5-trifluoromethyl-pyridine-2-carboxylic acid ((5)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for use in inhibiting or reducing the level of colonization of at least one pathogenic bacteria in the lungs of a subject in need thereof.

Another embodiment of the invention provides for a pharmaceutical composition comprising a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for use in inhibiting or reducing the level of colonization of at least one pathogenic bacteria in the lungs of a subject in need thereof.

In some embodiments, the compound is a compound of Formula (I), (II), or (III) or a pharmaceutically aacceptable salt thereof. In particular embodiments, the compound is 3-Amino-6-methoxy-5-trifluoromethyl-pyridine-2-carboxylic acid ((S)-3,3,3-trifluoro-2-hydroxy-2-methyl-propyl)-amide or a pharmaceutically acceptable salt thereof. In certain embodiments, the level of at least one pathogenic bacteria is measured from a sputum sample obtained from the subject. In some embodiments, the level of at least one pathogenic bacteria is measured by 16S rRNA PCR. In certain embodiments, the pathogenic bacteria is a non-fermenting Gram negative bacteria. In certain embodiments, the pathogenic bacteria is selected from the group consisting of M. catarrhalis, S. aureus, Enterobacteriaceae, Stenotrophomonous Maltophilia Hemophilus parainfluenza, Hemophilus influenza, Pseudomonas aeruginosa, Moraxella, and Streptococcus pneumonia. In certain embodiments, the level of colonization of pathogenic bacteria is reduced to a desired level (e.g., at least one log, at least two log, at least three log, at least four log, at least five log, or more). In particular embodiments, the level of colonization of pathogenic bacteria is reduced by at least one log. In other embodiments, the compound is administered in combination with at least one additional therapy. In some embodiments, the additional therapy comprises: a) a long-acting beta-agonist (LABA); b) a long-acting muscarinic antagonists (LAMA); c) an inhaled corticosteroid (ICS); d) macrolides; e) an antibiotic (e.g., a macrolide antibiotic); f) a short-acting muscarinic antagonist (SAMA); or g) any combination thereof. When administered in combination, the two or more agents may be administered sequentially or concurrently and may be administered in one or more compositions. In certain embodiments, the subject in need thereof is a bronchiectasis subject. In other embodiments, the bronchiecstasis is characterized by deterioration in three or more symptoms for at least 48 hours. In some embodiments, the symptoms are selected from the group consisting of: cough, sputum volume and/or consistency, sputum purulence, breathlessness and/or exercise tolerance, fatigue and/or malaise, and haemoptysis. In other embodiments, the compound is administered in an amount of between about 300 mg b.i.d. and about 450 mg b.i.d., e.g., is administered to the subject in an amount of 300 mg b.i.d. or 450 mg b.i.d. In a particular embodiment, the compound is administered to the subject in an amount of about 300 mg b.i.d. In other embodiments, the compound is administered orally. In some embodiments, the compound is administered to the subject without a high fat meal. In certain embodiments, the method further comprises: a) reducing the use of rescue medication (e.g., salbutamol/albuterol or systemic antibiotics) in a subject when compared to a subject who is not administered the compound; b) reducing the severity of exacerbations in a subject when compared to a subject who is not administered the compound; c) increasing one or more of improved lung function or forced vital capacity in a subject, e.g., as measured by spirometry, when compared to a patient who is not administered the compound; or d) any combination thereof.

### 6.2.4. Fibrinogen

An embodiment of the invention as defined above provides a method for reducing the level of fibrinogen in the blood of a subject in need thereof, comprising administering an effective amount of a compound of Formula (I), (II), or (III) (e.g., Compound A or a pharmaceutically acceptable salt thereof), as defined in any of the aforementioned embodiments, in free or pharmaceutically acceptable salt form, to the subject.

Another embodiment of the invention as defined above provides for the use of a compound of Formula (I), (II), or (III) (e.g., Compound A or a pharmaceutically acceptable salt thereof), as defined in any of the aforementioned embodiments, in free or pharmaceutically acceptable salt form, in the manufacture of a medicament for reducing the level of fibrinogen in the blood of a subject in need thereof.

Another embodiment of the invention as defined above provides for the use of a compound of Formula (I), (II), or (III) (e.g., Compound A or a pharmaceutically acceptable salt thereof), as defined in any of the aforementioned embodiments, in free or pharmaceutically acceptable salt form, for reducing the level of fibrinogen in the blood of a subject in need thereof.

Another embodiment of the invention as defined above provides for a compound of Formula (I), (II), or (III) (e.g., Compound A or a pharmaceutically acceptable salt thereof), as defined in any of the aforementioned embodiments, in free or pharmaceutically acceptable salt form, for use in reducing the level of fibrinogen in the blood of a subject in need thereof.

Another embodiment of the invention as defined above provides for a pharmaceutical composition comprising a compound of Formula (I), (II), or (III) (e.g., Compound A or a pharmaceutically acceptable salt thereof), as defined in any of the aforementioned embodiments, in free or pharmaceutically acceptable salt form, for use in reducing the level of fibrinogen in the blood of a subject in need thereof.

A particular embodiment of the invention as defined above provides a method for reducing the level of fibrinogen in the blood of a subject in need thereof, comprising administering an effective amount of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to the subject.

Another embodiment of the invention as defined above provides for the use of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, in the manufacture of a medicament for reducing the level of fibrinogen in the blood of a subject in need thereof.

Another embodiment of the invention as defined above provides for the use of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for reducing the level of fibrinogen in the blood of a subject in need thereof.

Another embodiment of the invention as defined above provides for a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for use in reducing the level of fibrinogen in the blood of a subject in need thereof.

Another embodiment of the invention as defined above provides for a pharmaceutical composition comprising a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, for use in reducing the level of fibrinogen in the blood of a subject in need thereof.

In some embodiments, the compound is a compound of Formula (I), (II), or (III) or a pharmaceutically aacceptable salt thereof. In particular embodiments, the compound is 3-Amino-6-methoxy-5-trifluoromethyl-pyridine-2-carboxylic acid ((S)-3,3,3-trifluoro-2-hydroxy-2-methyl-propyl)-amide or a pharmaceutically acceptable salt thereof. In certain embodiments, the subject in need thereof is a bronchiectasis subject. In other embodiments, the bronchiecstasis is characterized by deterioration in three or more symptoms for at least 48 hours. In some embodiments, the symptoms are selected from the group consisting of: cough, sputum volume and/or consistency, sputum purulence, breathlessness and/or exercise tolerance, fatigue and/or malaise, and haemoptysis. In other embodiments, the compound is administered to the subject in an amount of between about 300 mg b.i.d. and about 450 mg b.i.d., e.g., is administered in an amount of 300 mg b.i.d. or 450 mg b.i.d. In a particular embodiment, the compound is administered to the subject in an amount of about 300 mg b.i.d. In other embodiments, the compound is administered orally. In some embodiments, the compound is administered to the subject without a high fat meal. In certain embodiments, the compound is administered in combination with at least one additional therapy. In certain embodiments, the additional therapy comprises: a) a long-acting beta-agonist (LABA); b) a long-acting muscarinic antagonists (LAMA); c) an inhaled corticosteroid (ICS); d) macrolides; e) an antibiotic (e.g., a macrolide antibiotic); f) a short-acting muscarinic antagonist (SAMA); or g) any combination thereof. When administered in combination, the two or more agents may be administered sequentially or concurrently and may be administered in one or more compositions. In certain embodiments, the method further comprises: a) reducing the use of rescue medication (e.g., salbutamol/albuterol or systemic antibiotics) in a subject when compared to a subject who is not administered the compound; b) reducing the severity of exacerbations in a subject when compared to a subject who is not administered the compound; c) increasing one or more of improved lung function or forced vital capacity in a subject, e.g., as measured by spirometry, when compared to a patient who is not administered the compound; or d) any combination thereof.

### 6.3. Preparation of Compounds

Generally, compounds according to Formula (I), (II) or (III) (e.g., Compound A or a pharmaceutically acceptable salt thereof) can be synthesized by the routes described in Scheme 1, 2 and 3 and the Examples.

When A is CH the pyridinyl moiety may be synthesized according to the general scheme 1 shown below.

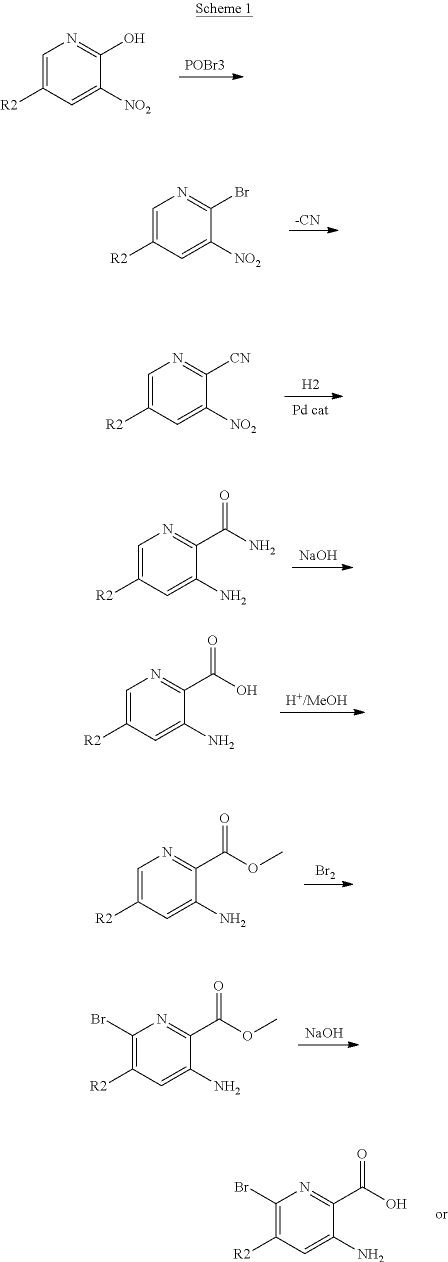

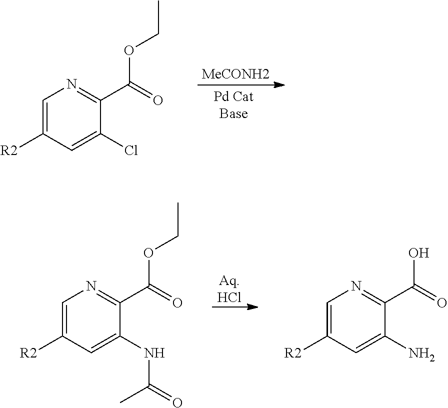

When A is nitrogen, the pyrazine moiety may be synthesized according to the general scheme 2 shown below.

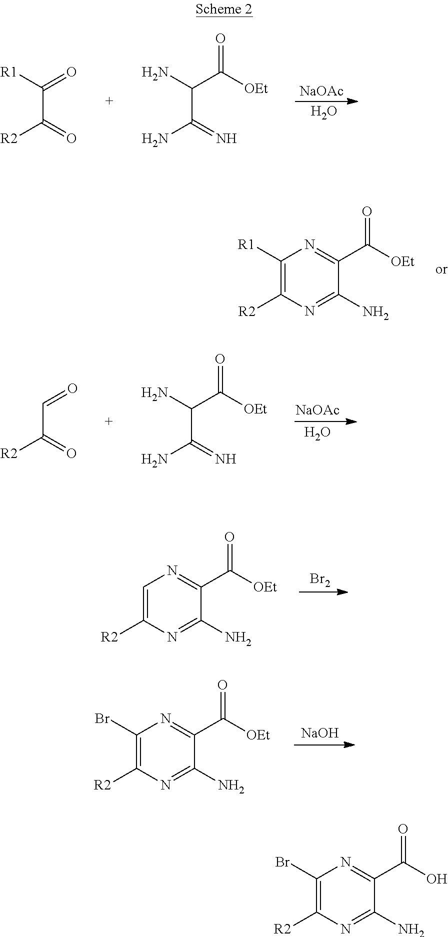

The right hand side of the moiety is typically added via an amide formation reaction as shown below in general scheme 3.

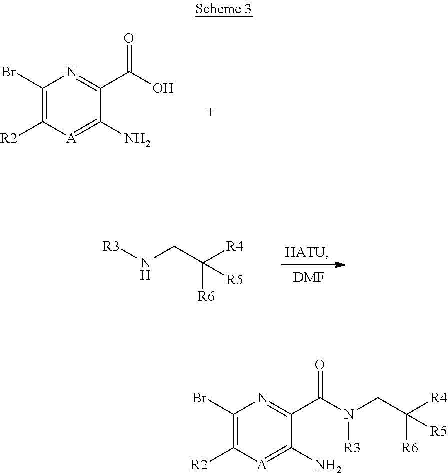

HATU (2-(1H-7-Azabenzotriazol-1-yl)-1,1,3,3-tetramethyl uronium hexafluorophosphate Methanaminium) is a peptide coupling agent. A skilled artisan would understand that other coupling agents cold possibly work. The halogen group in the above schemes can be replaced with other groups by choosing the appropriate nucleophile and catalyst. Protection of the Aryl NH₂ group may be required and is represented by P. The schemes 4-7 below are some representative examples.

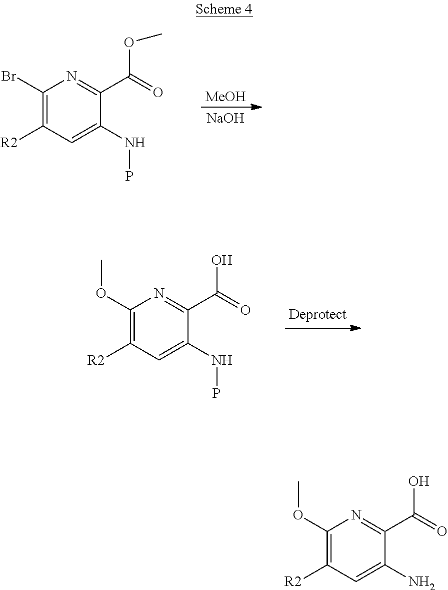

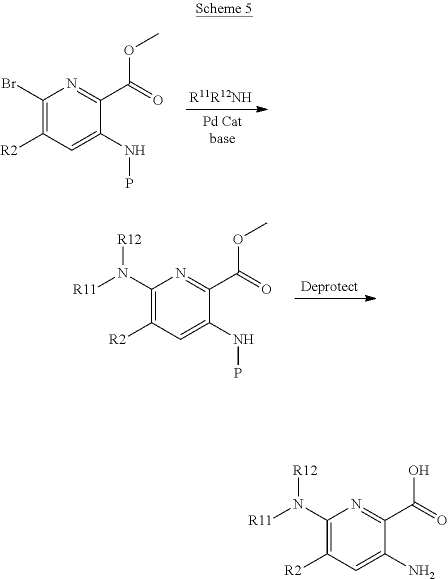

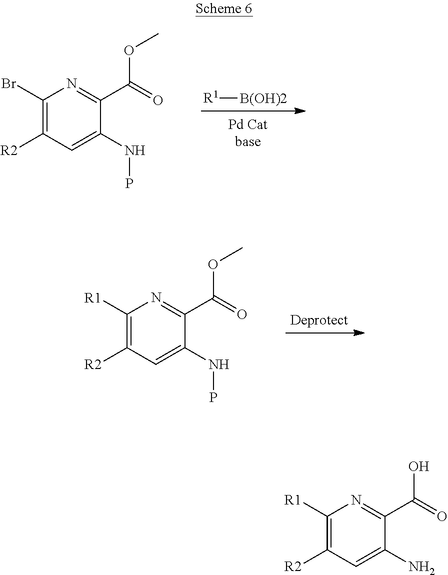

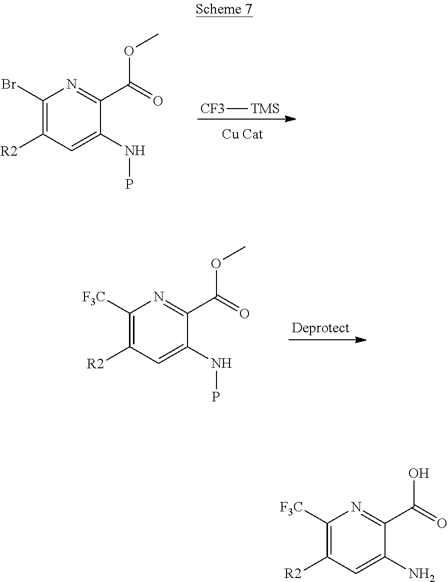

The skilled person will appreciate that the general synthetic routes detailed above show common reactions to transform the starting materials as required. The specific reaction conditions are not provided, but these are well known to those skilled in the art and appropriate conditions considered to be within the skilled person's common general knowledge.

The starting materials are either commercially available compounds or are known compounds and can be prepared from procedures described in the organic chemistry art.

Compounds of Formula (I), (II) or (III) (e.g., Compound A), in free form, may be converted into salt form, and vice versa, in a conventional manner understood by those skilled in the art. The compounds in free or salt form can be obtained in the form of hydrates or solvates containing a solvent used for crystallisation. In one embodiment, the compounds are present as a besylate salt, a mesylate salt, a tosylate salt, a hydrochloride salt, or a sulfate salt. In a preferred embodiment, the compounds are present as the besylate salt. Compounds of Formula (I), (II) or (III) (e.g., Compound A or a pharmaceutically acceptable salt thereof) can be recovered from reaction mixtures and purified in a conventional manner. Isomers, such as stereoisomers, may be obtained in a conventional manner, e.g., by fractional crystallisation or asymmetric synthesis from correspondingly asymmetrically substituted, e.g., optically active, starting materials.

Compounds of Formula (I), (II) or (III) (e.g., Compound A or a pharmaceutically acceptable salt thereof) can be prepared, e.g., using the reactions and techniques described below and in the Examples. The reactions may be performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

The various substituents on the synthetic intermediates and final products shown in the following reaction schemes can be present in their fully elaborated forms, with suitable protecting groups where required as understood by one skilled in the art, or in precursor forms which can later be elaborated into their final forms by methods familiar to one skilled in the art. The substituents can also be added at various stages throughout the synthetic sequence or after completion of the synthetic sequence. In many cases, commonly used functional group manipulations can be used to transform one intermediate into another intermediate, or one compound of Formula (I), (II) or (III) (e.g., Compound A or a pharmaceutically acceptable salt thereof) into another compound of Formula (I), (II) or (III) (e.g., Compound A or a pharmaceutically acceptable salt thereof). Examples of such manipulations are conversion of an ester or a ketone to an alcohol; conversion of an ester to a ketone; interconversions of esters, acids and amides; alkylation, acylation and sulfonylation of alcohols and amines; and many others. Substituents can also be added using common reactions, such as alkylation, acylation, halogenation or oxidation. Such manipulations are well-known in the art, and many reference works summarize procedures and methods for such manipulations. Some reference works which gives examples and references to the primary literature of organic synthesis for many functional group manipulations, as well as other transformations commonly used in the art of organic synthesis are March's Organic Chemistry, 5th Edition, Wiley and Chichester, Eds. (2001); Comprehensive Organic Transformations, Larock, Ed., VCH (1989); Comprehensive Organic Functional Group Transformations, Katritzky et al. (series editors), Pergamon (1995); and Comprehensive Organic Synthesis, Trost and Fleming (series editors), Pergamon (1991). It will also be recognized that another major consideration in the planning of any synthetic route in this field is the judicious choice of the protecting group used for protection of the reactive functional groups present in the compounds described in this invention. Multiple protecting groups within the same molecule can be chosen such that each of these protecting groups can either be removed without removal of other protecting groups in the same molecule, or several protecting groups can be removed using the same reaction step, depending upon the outcome desired. An authoritative account describing many alternatives to the trained practitioner is Greene and Wuts, Protective Groups in Organic Synthesis, Wiley and Sons (1999).

### 6.4. Pharmacological Activity

Having regard to their modulation of CFTR activity, compounds of Formula (I), (II) or (III) (e.g., Compound A or a pharmaceutically acceptable salt thereof), in free or pharmaceutically acceptable salt form, are useful in the treatment of conditions which respond to the modulation of CFTR activity, particularly conditions benefiting from mucosal hydration such as cystic fibrosis. Compounds of Formula (I), (II) or (III) (e.g., Compound A or a pharmaceutically acceptable salt thereof), in free or pharmaceutically acceptable salt form, are also useful in the treatment of bronchiectasis, wherein the bronchiectasis is cystic fibrosis bronchiectasis or non-cystic fibrosis bronchiectasis.

Diseases mediated by modulation of CFTR activity, include diseases associated with the regulation of fluid volumes across epithelial membranes. For example, the volume of airway surface liquid is a key regulator of mucociliary clearance and the maintenance of lung health. The modulation of CFTR activity will promote fluid accumulation on the mucosal side of the airway epithelium thereby promoting mucus clearance and preventing the accumulation of mucus and sputum in respiratory tissues (including lung airways). Such diseases include respiratory diseases, such as bronchiectasis, cystic fibrosis, primary ciliary dyskinesia, chronic bronchitis, chronic obstructive pulmonary disease (COPD), asthma, respiratory tract infections (acute and chronic; viral and bacterial) and lung carcinoma. Diseases mediated by modulation of CFTR activity also include diseases other than respiratory diseases that are associated with abnormal fluid regulation across an epithelium, perhaps involving abnormal physiology of the protective surface liquids on their surface, e.g., Sjögren's Syndrome, xerostomia (dry mouth) or keratoconjunctivitis sire (dry eye). Furthermore, modulation of CFTR activity in the kidney could be used to promote diuresis and thereby induce a hypotensive effect.

Treatment in accordance with the invention may be symptomatic or prophylactic.

Asthma includes intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. For convenience this particular asthmatic condition is referred to as “wheezy-infant syndrome”.

Prophylactic efficacy in the treatment of asthma will be evidenced by reduced frequency or severity of symptomatic attack, e.g., of acute asthmatic or bronchoconstrictor attack, improvements in lung function or improved airways hyperreactivity. It may further be evidenced by reduced requirement for other, symptomatic therapy, i.e., therapy for or intended to restrict or abort symptomatic attack when it occurs, e.g., anti-inflammatory (e.g., cortico-steroid) or bronchodilatory. Prophylactic benefit in asthma may, in particular, be apparent in subjects prone to “morning dipping”. “Morning dipping” is a recognized asthmatic syndrome, common to a substantial percentage of asthmatics and characterized by asthma attack, e.g., between the hours of about 4-6 am, i.e., at a time normally substantially distant from any previously administered symptomatic asthma therapy.

Chronic obstructive pulmonary disease includes chronic bronchitis or dyspnea associated therewith, emphysema, as well as exacerbation of airways hyperreactivity consequent to other drug therapy, in particular, other inhaled drug therapy. The invention is also applicable to the treatment of bronchitis of whatever type or genesis including, e.g., acute, arachidic, catarrhal, croupus, chronic or phthinoid bronchitis.

Dry eye disease is characterized by a decrease in tear aqueous production and abnormal tear film lipid, protein and mucin profiles. There are many causes of dry eye, some of which include age, laser eye surgery, arthritis, medications, chemical/thermal burns, allergies and diseases, such as cystic fibrosis and Sjögren's Syndrome. Increasing anion secretion via CFTR would enhance fluid transport from the corneal endothelial cells and secretory glands surrounding the eye to increase corneal hydration. This would help to alleviate the symptoms associated with dry eye disease.

Sjögren's Syndrome is an autoimmune disease in which the immune system attacks moisture-producing glands throughout the body, including eye, mouth, skin, respiratory tissue, liver, vagina and gut. Symptoms include dry eye, dry mouth and dry vagina, as well as lung disease. The disease is also associated rheumatoid arthritis, systemic lupus, systemic sclerosis and polymypositis/dermatomyositis. Defective protein trafficking is believed to cause the disease, for which treatment options are limited. Modulators of CFTR activity may hydrate the various organs affected by the disease and help to alleviate the associated symptoms.

The suitability of CFTR activity modulators as a treatment of a disease benefiting from mucosal hydration may be tested by determining the movement of chloride ions in a suitable cell-based assay. For example single cells or confluent epithelia, endogenously expressing or engineered to overexpress CFTR can be used to assess channel function using electrophysiological techniques or ion flux studies. See methods described in: Hirsh et al., J Pharm Exp Ther (2004); Moody et al., Am J Physiol Cell Physiol (2005).

CFTR activity modulators, including compounds of Formula (I), (II), or (III), are also useful as co-therapeutic agents for use in combination with other drug substances, such as anti-inflammatory, bronchodilatory, antihistamine or anti-tussive drug substances, particularly in the treatment of bronchiectasis, cystic fibrosis or obstructive or inflammatory airways diseases such as those mentioned hereinbefore, e.g., as potentiators of therapeutic activity of such drugs or as a means of reducing required dosaging or potential side effects of such drugs.

Compounds of Formula (I), (II) or (III) (e.g., Compound A or a pharmaceutically acceptable salt thereof) may be mixed with the other drug substance in a fixed pharmaceutical composition or it may be administered separately, before, simultaneously with or after the other drug substance.

Accordingly, the invention includes as a further aspect a combination of a CFTR activity modulator with osmotic agents (hypertonic saline, dextran, mannitol, Xylitol), ENaC blockers, an anti-inflammatory, bronchodilatory, antihistamine, anti-tussive, antibiotic and/or DNase drug substance, wherein the CFTR activity modulator and the further drug substance may be in the same or different pharmaceutical composition.

Suitable antibiotics include macrolide antibiotics, e.g., tobramycin (TOBI™).

Suitable DNase drug substances include dornase alfa (Pulmozyme™), a highly-purified solution of recombinant human deoxyribonuclease I (rhDNase), which selectively cleaves DNA. Dornase alfa is used to treat cystic fibrosis.

Other useful combinations of CFTR activity modulators with anti-inflammatory drugs are those with antagonists of chemokine receptors, e.g., CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists, such as Schering-Plough antagonists SC-351125, SCH-55700 and SCH-D; Takeda antagonists, such as N-[[4-[[[6,7-dihydro-2-(4-methyl-phenyl)-5H-benzo-cyclohepten-8-yl]carbonyl]amino]phenyl]-methyl]tetrahydro-N,N-dimethyl-2H-pyran-4-amin-ium chloride (TAK-770); and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

Suitable anti-inflammatory drugs include steroids, in particular, glucocorticosteroids, such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920; non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248; LTD4 antagonists, such as montelukast and zafirlukast; PDE4 inhibitors, such as cilomilast (Ariflo GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID™CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; adenosine A2B receptor antagonists such as those described in WO 02/42298; and beta-2 adrenoceptor agonists, such as albuterol (salbutamol), metaproterenol, terbutaline, salmeterol fenoterol, procaterol, and especially, formoterol, carmoterol and pharmaceutically acceptable salts thereof, and compounds (in free or salt or solvate form) of Formula (I) of WO 0075114, which document is incorporated herein by reference, preferably compounds of the Examples thereof, especially indacaterol and pharmaceutically acceptable salts thereof, as well as compounds (in free or salt or solvate form) of Formula (I) of WO 04/16601, and also compounds of EP 1440966, JP 05025045, WO 93/18007, WO 99/64035, USP 2002/0055651, WO 01/42193, WO 01/83462, WO 02/66422, WO 02/70490, WO 02/76933, WO 03/24439, WO 03/42160, WO 03/42164, WO 03/72539, WO 03/91204, WO 03/99764, WO 04/16578, WO 04/22547, WO 04/32921, WO 04/33412, WO 04/37768, WO 04/37773, WO 04/37807, WO 04/39762, WO 04/39766, WO 04/45618, WO 04/46083, WO 04/80964, WO 04/108765 and WO 04/108676.

Suitable bronchodilatory drugs include anticholinergic or antimuscarinic agents, in particular, ipratropium bromide, oxitropium bromide, tiotropium salts and CHF 4226 (Chiesi), and glycopyrrolate, but also those described in EP 424021, U.S. Pat. Nos. 3,714,357, 5,171,744, WO 01/04118, WO 02/00652, WO 02/51841, WO 02/53564, WO 03/00840, WO 03/33495, WO 03/53966, WO 03/87094, WO 04/018422 and WO 04/05285.

Suitable dual anti-inflammatory and bronchodilatory drugs include dual beta-2 adrenoceptor agonist/muscarinic antagonists such as those disclosed in USP 2004/0167167, WO 04/74246 and WO 04/74812.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine, as well as those disclosed in JP 2004107299, WO 03/099807 and WO 04/026841.

In accordance with the foregoing, the invention also provides as a further aspect a method for the treatment of a condition responsive to modulation of CFTR activity, e.g., diseases associated with the regulation of fluid volumes across epithelial membranes, particularly an obstructive airways disease, which comprises administering to a subject, particularly a human subject, in need thereof a compound of Formula (I), (II) or (III) (e.g., Compound A), in free form or in the form of a pharmaceutically acceptable salt.

In another aspect the invention provides a compound of Formula (I), (II) or (III) (e.g., Compound A), in free form or in the form of a pharmaceutically acceptable salt, for use in the manufacture of a medicament for the treatment of a condition responsive to modulation of CFTR activity, particularly an obstructive airways disease, e.g., bronchiectasis, cystic fibrosis, and COPD.

Compounds of Formula (I), (II), or (III) (e.g., Compound A or a pharmaceutically acceptable salt thereof) may be administered by any appropriate route, e.g., orally, e.g., in the form of a tablet or capsule; parenterally, e.g., intravenously; by inhalation, e.g., in the treatment of an obstructive airways disease; intranasally, e.g., in the treatment of allergic rhinitis; topically to the skin; or rectally. In a further aspect, the invention also provides a pharmaceutical composition comprising a compound of Formula (I), (II), or (III) (e.g., Compound A) in free form or in the form of a pharmaceutically acceptable salt, optionally together with a pharmaceutically acceptable diluent or carrier therefor. The composition may contain a co-therapeutic agent, such as an anti-inflammatory, broncho-dilatory, antihistamine or anti-tussive drug as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g., patches. Compositions for inhalation may comprise aerosol or other atomizable formulations or dry powder formulations.

When the composition comprises an aerosol formulation, it preferably contains, e.g., a hydro-fluoro-alkane (HFA) propellant, such as HFA134a or HFA227 or a mixture of these, and may contain one or more co-solvents known in the art, such as ethanol (up to 20% by weight), and/or one or more surfactants, such as oleic acid or sorbitan trioleate, and/or one or more bulking agents, such as lactose. When the composition comprises a dry powder formulation, it preferably contains, e.g., the compound of Formula (I), (II) or (III) (e.g., Compound A or a pharmaceutically acceptable salt thereof) having a particle diameter up to 10 microns, optionally together with a diluent or carrier, such as lactose, of the desired particle size distribution and a compound that helps to protect against product performance deterioration due to moisture, e.g., magnesium stearate. When the composition comprises a nebulised formulation, it preferably contains, e.g., the compound of Formula (I), (II) or (III) (e.g., Compound A or a pharmaceutically acceptable salt thereof) either dissolved, or suspended, in a vehicle containing water, a co-solvent, such as ethanol or propylene glycol and a stabilizer, which may be a surfactant.

Further aspects of the invention include methods for treating bronchiectasis comprising administering to a subject in need thereof at least one of the following:

(a) a compound of Formula (I), (II) or (III) (e.g., Compound A or a pharmaceutically acceptable salt thereof) in inhalable form, e.g., in an aerosol or other atomisable composition or in inhalable particulate, e.g., micronised form;
(b) an inhalable medicament comprising a compound of Formula (I), (II) or (III) (e.g., Compound A or a pharmaceutically acceptable salt thereof) in inhalable form;
(c) a pharmaceutical product comprising a compound of Formula (I), (II), or (III) (e.g., Compound A or a pharmaceutically acceptable salt thereof) in inhalable form in association with an inhalation device; or
(d) an inhalation device containing a compound of Formula (I), (II) or (III) (e.g., Compound A or a pharmaceutically acceptable salt thereof) in inhalable form.

### 6.5. Pharmaceutical Use and Assay

Compounds of Formula (I), (II) or (III) (e.g., Compound A or a pharmaceutically acceptable salt thereof) and their pharmaceutically acceptable salts are useful as pharmaceuticals. In particular, the compounds are suitable CFTR activity modulators and may be tested in the following assays.

### 6.5.1. Membrane Potential Assay

CFTR activity can be quantified by measuring the transmembrane potential. The means for measuring the transmembrane potential in a biological system can employ a number of methods including electrophysiological and optical fluorescence-based membrane potential assays.

The optical membrane potential assay utilises a negatively charged potentiometric dye, such as the FLIPR membrane potential dye (FMP) (see Baxter D F, Kirk M, Garcia A F, Raimondi A, Holmqvist M H, Flint K K, Bojanic D, Distefano P S, Curtis R, Xie Y. ‘A novel membrane potential-sensitive fluorescent dye improves cell-based assays for ion channels.’ J Biomol Screen. 2002 February; 7(1):79-85) which when extracellular is bound to a quenching agent. Upon cellular depolarisation the negatively charged dye redistributes to the intracellular compartment, unbinding from the membrane impermeant quench agent, yielding an increase in fluorescence. This change in fluorescence is proportional to the change in transmembrane potential which can result from the activity of CFTR. The changes in fluorescence can be monitored in real time by an appropriately equipped fluorescence detector such as the FLIPR (fluorometric imaging plate reader) in 96 or 384-well microtitre plates.

Cell culture:

Chinese hamster ovary (CHO) cells stably expressing the ΔF508-CFTR channel were used for membrane potential experiments. Cells were maintained at 37° C. in 5% v/v CO₂ at 100% humidity in Modified Eagles medium (MEM) supplemenetd with 8% v/v foetal calf serum, 100 μg/ml methotrexate and 100 U/ml penicillin/streptomycin. Cells were grown in 225 cm² tissue culture flasks. For membrane potential assays cells were seeded into 96 well plates at 40,000 cells per well, allowed to adhere and then maintained at 26° C. for 48 h to facilitate channel insertion.

### 6.5.2. Potentiator Assay

The membrane potential screening assay utilised a low chloride ion containing extracellular solution (˜5 mM) combined with a double addition protocol. The first addition was of buffer with or without test compound followed 5 minutes later by an addition of forskolin (1-20 μM)—this protocol favours maximum chloride efflux in response to ΔF508-CFTR activation. The ΔF508-CFTR mediated chloride ion efflux leads to a membrane depolarisation which is optically monitored by the FMP dye.

### Solutions:

Low chloride extracellular (mM): 120 Na-gluconate, 1.2 CaCl₂, 3.3 KH₂PO₄, 0.8 K₂HPO₄, 1.2 MgCl₂, 10.0 D-glucose, 20.0 HEPES, pH 7.4 with NaOH

FMP dye: made up as per manufacturers' instructions in low chloride extracellular solution detailed above, at 10× final concentration, and stored as 1 mL aliquots at −20° C.

6.5.3. IonWorks Quattro Assay

CFTR activity can also be quantified electrophysiologically using the whole-cell configuration of the patch clamp technique (Hamill et al Pflugers Acrhive 1981). This assay directly measures the currents associated with chloride flow through CFTR channels whilst either maintaining or adjusting the transmembrane voltage. This assay can use either single glass micropipettes or parallel planar arrays to measure CFTR activity from native or recombinant cell systems. Currents measured using parallel planar arrays can be quantified using an appropriately equipped instrument such as the IonWorks Quattro (Molecular Devices) or the Qpatch (Sophion). The Quattro system can measure CFTR currents from either a single cell per recording well (HT configuration) or alternatively from a population of 64 cells per well (Population Patch Clamp PPC) (Finkel A, Wittel A, Yang N, Handran S, Hughes J, Costantin J. ‘Population patch clamp improves data consistency and success rates in the measurement of ionic currents.’ J Biomol Screen. 2006 August; 11(5):488-96).

Cell culture:

Chinese hamster ovary (CHO) cells stably expressing the ΔF508-CFTR channel were used for IonWorks Quattro experiments. Cells were maintained at 37° C. in 5% v/v CO₂ at 100% humidity in D-MEM supplemented with 10% (v/v) FCS, 100 U/mL Penicillin/Streptomycin, 1% (v/v) NEAA, 1 mg/ml Zeocin and 500 ug/ml Hygromycin B. For experiments cells were grown in 225 cm² tissue culture flasks until near confluence and then cultured at 26° C. for 48-72 h to facilitate channel insertion. Cells were removed from the flask and resuspended in either extracellular recording solution for immediate experimentation or alternatively in growth medium supplemented with 10% v/v DMSO and frozen to −80° C. as 1-2 mL aliquots for use at a later date.

6.5.4. Potentiator Assay

Cells, at a density of 1.5-3 million per mL, were placed on the Quattro system, added to the planar patch array and seals allowed to establish for 5-10 mins. After assessing seal resistances (commonly >50 MΩ), whole-cell access was obtained by perforation with 100 μg/mL amphotericin B. Baseline currents were measured by a pre-compound scan obtained by application of a voltage ramp from −100 to +100 mV. This was followed by addition of either buffer or test compound diluted in the extracellular solution supplemented with 20 μM forskolin, to each of the 384 wells of the planar parch array. After incubation step (5-20 minutes) the post-compound currents were measured again by application of a voltage ramp from −100 to +100 mV. The difference in currents between the pre- and post-compound scans defined the efficacy of CFTR potentiation.

### Solutions:

Extracellular solution (ECS): 145 mM NaCl, 4 mM CsCl, 5 mM D-glucose, 10 mM TES, 1 mM CaCl₂), 1 mM MgCl₂, pH 7.4 NaOH

Intracellular buffer (ICS): 113 mM L-Aspartic acid, 113 mM CsOH, 27 mM CsCl, 1 mM NaCl, 1 mM MgCl₂, 1 mM EGTA, 10 mM TES. pH 7.2 with CsOH. Filter sterilized before use.

6.5.5. Ion Transport Assay

Another method to measure CFTR function is Ussings chamber short circuit current measurement. Engineered or native epithelial cells are grown to confluent monolayer on a semi-permeable filter and sandwiched between two perspex blocks. The flow of chloride ions via CFTR from one side of the epithelia to the other can be quantified by measuring the flow of current whilst maintaining the transepithelial potential at 0 mV. This is achieved using KCl filled agar-based electrodes to both clamp the cellular monolayer and measure the flow of currents.

Cell culture:

FRT cells stably expressing ΔF508-CFTR were cultured on plastic in Coon's modified F-12 medium supplemented with 32 mM NaHCO₃, 10% v/v fetal bovine serum, 2 mM L-glutamine, 100 U/mL penicillin, 100 μg/mL streptomycin and 30 μg/mL hygromycin B as the growth medium. For Ussing chamber experiments, the cells were grown as polarized epithelia on Snapwell permeable support inserts (500000 cells/insert in growth medium) and cultured for 7 to 9 days. The inserts were fed with fresh Coon's modified F-12 growth medium every 48 hours, and 24 hours prior to Ussing chamber experiment. To increase the ΔF508 CFTR protein expression at the cell surface, plates were incubated at 27° C. for 48 h before performing an Ussing chamber experiment.

6.5.6. Potentiator Assay

Fischer Rat Thyroid (FRT) epithelial cells, stably expressing human ΔF508-CFTR were used as monolayer cultures on permeable supports. Cl⁻ current was measured using the short circuit current technique, under an imposed basolateral to apical Cl⁻ gradient in Ussing chambers. To measure stable Cl⁻ currents, FRT cells were cultured for 48 h at 27° C. to facilitate the insertion of ΔF508 CFTR into the plasma membrane. Ussing chamber studies were likewise conducted at 27° C. Under these conditions, the effects of cumulative additions of test compounds on ΔF508 CFTR currents could be quantitated with both potency and efficacy endpoints. Compounds were added to both the apical and basloalteral sides subsequent to addition of 10 μM forskolin. Efficacy of compounds was compared to a known potentiator such as gensitein.

### Solutions:

Basolateral Ringer solution (mM): 126 NaCl, 24 NaHCO₃, 0.38 KH₂PO₄, 2.13 K₂HPO₄, 1 MgSO₄, 1 CaCl₂) and 10 glucose.

Apical Ringer solution (mM): 140 Na-gluconate, 1 MgSO₄, 2 CaCl₂), 1 HCl, 10 glucose and 24 NaHCO₃.

Compounds can also be tested for their ability to stimulate insertion of ΔF508 CFTR into the cell membrane using the above assays. For these assays the protocols were identical other than cells were not cultured at low temperature (26° C. or 27° C.) but instead incubated with test compounds for 12-24 h prior to assay.

Compounds of the Examples, herein below, generally have EC₅₀ values in the data measurements described above below 10 μM. Table 1 provides a list of representative compounds with their EC₅₀ value.

TABLE 1
Representative Compounds

| Example No. | EC₅₀ μM | Example No. | EC₅₀ |
|---|---|---|---|
| 2 | 0.015 | 9 | 0.090 |
| 3 | 0.055 | 10 | 0.112 |
| 4 | 0.076 | 11 | 0.037 |
| 5 | 0.05 | 12 | 0.035 |
| 6 | 0.426 | 14 | 0.115 |
| 7 | 0.040 | 15 | 0.051 |
| 8 | 0.060 | 16 | 0.008 |
| | | 17 | 0.010 |

Compounds listed below are within the scope of the broadest claim and the CFTR EC₅₀ values in the data measurements described above were above 5 μM:
3-amino-6-bromo-N-(imidazo[1,2-a]pyridin-2-ylmethyl)-5-(trifluoromethyl)pyrazine-2-carboxamide;
3-amino-6-bromo-N-((1-methyl-1H-imidazol-4-yl)methyl)-5-(trifluoromethyl)pyrazine-2-carboxamide;
2-(3-amino-6-bromo-5-(trifluoromethyl)picolinamido)acetic acid;
3-amino-6-bromo-N-((1-methyl-1H-pyrazol-3-yl)methyl)-5-(trifluoromethyl)pyrazine-2-carboxamide;
3-amino-N-(2-(4-fluorophenyl)-2-oxoethyl)-6-(1-methyl-1H-indol-6-yl)-5-(trifluoromethyl)picolinamide;
3-amino-6-bromo-N-((1-methyl-1H-imidazol-2-yl)methyl)-5-(trifluoromethyl)pyrazine-2-carboxamide;
6-((3H-[1,2,3]triazolo[4,5-b]pyridin-3-yl)oxy)-3-(2,5-dimethyl-1H-pyrrol-1-yl)-N-(3,3,3-trifluoro-2-hydroxy-2-methylpropyl)-5-(trifluoromethyl)picolinamide;
3-amino-6-(6-(3-(dimethylamino)propoxy)pyridin-3-yl)-N-(2-(4-fluorophenyl)-2-oxoethyl)-5-(trifluoromethyl)pyrazine-2-carboxamide;
(R)-3-amino-6-bromo-N-((4-methylpiperazin-2-yl)methyl)-5-(trifluoromethyl)pyrazine-2-carboxamide;
3-amino-6-bromo-N-((1-methyl-1H-imidazol-5-yl)methyl)-5-(trifluoromethyl)pyrazine-2-carboxamide;
3-amino-6-(3-(N,N-dimethylsulfamoyl)phenyl)-N-(2-(4-fluorophenyl)-2-oxoethyl)-5-(trifluoromethyl)picolinamide;
3-amino-6-bromo-N-isobutyl-N-methyl-5-(trifluoromethyl)pyrazine-2-carboxamide;
3-amino-6-bromo-N-((1-methyl-1H-pyrazol-5-yl)methyl)-5-(trifluoromethyl)pyrazine-2-carboxamide;
6-bromo-3-(methylamino)-N-(3,3,3-trifluoro-2-hydroxy-2-methylpropyl)-5-(trifluoromethyl)picolinamide;
(3-amino-6-bromo-5-(trifluoromethyl)pyrazin-2-yl)(4-methylpiperazin-1-yl)methanone;
3-amino-6-bromo-N-(2-(pyridin-4-yl)ethyl)-5-(trifluoromethyl)pyrazine-2-carboxamide;
3-amino-N-(2-(4-fluorophenyl)-2-oxoethyl)-6-(1-oxo-1,2,3,4-tetrahydroisoquinolin-6-yl)-5-(trifluoromethyl)picolinamide;
3-amino-6-(4-carbamoyl-2-methylphenyl)-N-(2-(4-fluorophenyl)-2-oxoethyl)-5-(trifluoromethyl)picolinamide;
3-amino-6-bromo-N-(2-(pyridin-3-yl)ethyl)-5-(trifluoromethyl)pyrazine-2-carboxamide;
3-amino-6-(3,4-dimethylphenyl)-N-(2-(4-fluorophenyl)-2-oxoethyl)-5-(trifluoromethyl)picolinamide;
3-amino-N-benzyl-6-bromo-N-methyl-5-(trifluoromethyl)pyrazine-2-carboxamide;
3-amino-6-hydroxy-N-(3,3,3-trifluoro-2-hydroxy-2-methylpropyl)-5-(trifluoromethyl)picolinamide;
3-amino-6-hydroxy-N-(3,3,3-trifluoro-2-hydroxy-2-methylpropyl)-5-(trifluoromethyl)picolinamide;
(3-amino-6-bromo-5-(trifluoromethyl)pyrazin-2-yl)(4-methyl-3-phenylpiperazin-1-yl)methanone;
(S)-3-amino-6-bromo-N-((l-ethylpyrrolidin-2-yl)methyl)-5-(trifluoromethyl)pyrazine-2-carboxamide; and
3-amino-6-bromo-N-(imidazo[1,5-a]pyridin-1-ylmethyl)-5-(trifluoromethyl)pyrazine-2-carboxamide.

### 7. EXAMPLES

The invention is illustrated by the following Examples.

### 7.1. Synthesis of Compounds

### General Conditions:

Mass spectra were run on LC-MS systems using electrospray ionization. These were either Agilent 1100 HPLC/Micromass Platform Mass Spectrometer combinations or Waters Acquity UPLC with SQD Mass Spectrometer. [M+H]⁺ refers to mono-isotopic molecular weights.

NMR spectra were run on open access Bruker AVANCE 400 NMR spectrometers using ICON-NMR. Spectra were measured at 298K and were referenced using the solvent peak. Optical rotations were measured at 589 nm and 546 nm using an Optical activity AA-1000 polarimeter at 21° C.

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, and NMR. Abbreviations used are those conventional in the art. If not defined, the terms have their generally accepted meanings.

### Abbreviations

app apparent
ATP adenosine 5′-triphosphate
BINAP racemic 2,2′-bis(diphenylphosphino)-1,1′-binaphthyl
BOC tertiary butyl carboxy
br broad
d doublet
dd doublet of doublets
DCM dichloromethane
DIEA diethylisopropylamine
DIPEA diisopropylethylamine
DMF N,N-dimethylformamide
DMSO dimethylsulfoxide
DTT dithiothreitol
ESI electrospray ionization
EtOAc ethyl acetate
eq equivalent
h hour(s)
HATU 2-(7-Aza-1H-benzotriazole-1-yl)-1,1,3,3-tetramethyluronium hexafluorophosphate
HPLC high pressure liquid chromatography
IR infrared spectroscopy
LCMS liquid chromatography and mass spectrometry
MeOH methanol
MS mass spectrometry
MW microwave
m multiplet
min minutes
ml milliliter(s)
m/z mass to charge ratio
NMR nuclear magnetic resonance
ppm parts per million
PS polymer supported
rac racemic
RT room temperature
Rt retention time
s singlet
SCX-2 strong cation exchange (e.g., Isolute® SCX-2 columns from Biotage)
t triplet
TEA triethylamine
TFA trifluoroacetic acid
THF tetrahydrofuran

Referring to the examples that follow, compounds of the preferred embodiments were synthesized using the methods described herein, or other methods, which are known in the art.

The various starting materials, intermediates, and compounds of the preferred embodiments may be isolated and purified, where appropriate, using conventional techniques such as precipitation, filtration, crystallization, evaporation, distillation, and chromatography. Unless otherwise stated, all starting materials are obtained from commercial suppliers and used without further purification. Salts may be prepared from compounds by known salt-forming procedures.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

If not indicated otherwise, the analytical HPLC conditions are as follows:

Method 10minLC_v001

| | |
|---|---|
| Column | Waters BEH C18 100 × 2.1 mm, 1.7 μm |
| Column Temp. | 50° C. |
| Eluents | A: H₂O, B: acetonitrile, both containing 0.1% TFA |
| Flow Rate | 0.7 ml/min |
| Gradient | 0.25 min 5% B; 5% to 95% B in 7.75 min, |
| | 1.00 min 95% B |

Method 10minLC_v002

| | |
|---|---|
| Column | Waters BEH C18 50 × 2.1 mm, 1.7 μm |
| Column | 50° C. |
| Temperature | |
| Eluents | A: H₂O, B: methanol, both containing 0.1% TFA |
| Flow Rate | 0.8 ml/min |
| Gradient | 0.20 min 5% B; 5% to 95% B in 7.80 min, |
| | 1.00 min 95% B |

Method 10minLC_v003

| | |
|---|---|
| Column | Waters BEH C18 50 × 2.1 mm, 1.7 μm |
| Column | 50° C. |
| Temperature | |
| Eluents | A: H₂O, B: acetonitrile, both containing 0.1% TFA |
| Flow Rate | 0.8 ml/min |
| Gradient | 0.20 min 5% B; 5% to 95% B in 7.80 min, |
| | 1.00 min 95% B |

Method 2minLC_v001

| | |
|---|---|
| Column | Waters BEH C18 100 × 2.1 mm, 1.7 μm |
| Column Temp. | 50° C. |
| Eluents | A: H₂O, B: acetonitrile, both containing 0.1% TFA |
| Flow Rate | 0.7 ml/min |
| Gradient | 0.25 min 5% B; 5% to 95% B in 1.00 min, |
| | 0.25 min 95% B |

Method 2minLC_v002

| | |
|---|---|
| Column | Waters BEH C18 50 × 2.1 mm, 1.7 μm |
| Column | 50° C. |
| Temperature | |
| Eluents | A: H₂O, B: methanol, both containing 0.1% TFA |
| Flow Rate | 0.8 ml/min |
| Gradient | 0.20 min 5% B; 5% to 95% B in 1.30 min, |
| | 0.25 min 95% B |

Method 2minLC_v003

| | |
|---|---|
| Column | Waters BEH C18 50 × 2.1 mm, 1.7 μm |
| Column | 50° C. |
| Temperature | |
| Eluents | A: H₂O, B: acetonitrile, both containing 0.1% TFA |
| Flow Rate | 0.8 ml/min |
| Gradient | 0.20 min 5% B; 5% to 95% B in 1.30 min, |
| | 0.25 min 95% B |

Method 10minC18

| | |
|---|---|
| Column: | Gemini C18 100 × 3 mm, 3 micron |
| Column | 50 ° C. |
| Temperature | |
| Eluents: | A: H₂O, B: Methanol, 0.1% formic acid |
| Flow rate: | 1 ml/min |
| Gradient: | 0.00 min 0% B, 10.00 min 95% B |

Method AD25IPA_EA

| | |
|---|---|
| Mobile Phase: | 25% isopropanol + 0.1% v/v DEA/75% CO₂ |
| Column: | Chiralpak AD-H, 250 × 10 mm id, 5 μm |
| Detection: | UV @ 220 nm |
| Flow rate: | 10 ml/min |

### Example 1: 3-Amino-6-bromo-5-trifluoromethyl-pyridine-2-carboxylic acid (3,3,3-trifluoro-2-hydroxy-2-methyl-propyl)-amide

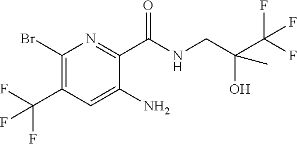 [SMILES: CC(O)(CNC(=O)c1nc(Br)c(C(F)(F)F)cc1N)C(F)(F)F]

3-Amino-6-bromo-5-trifluoromethyl-pyridine-2-carboxylic acid (Intermediate A) (397 mg, 1.392 mmol), 3-amino-1,1,1-trifluoro-2-methyl-propan-2-ol hydrochloride (250 mg, 1.392 mmol) and HATU (529 mg, 1.392 mmol) were dissolved in DMF (10 ml) and stirred at RT for 2 min. 4-Methylmorpholine (0.413 ml, 4.18 mmol) was added and stirring continued at RT for 3 h. The reaction mixture was poured onto ice/water (100 ml) and extracted with EtOAc (250 ml). The organic extract was washed with sat NH₄C₁ solution (˜50 ml), dried over MgSO₄ and concentrated in vacuo to give a pale brown oil. The oil was dissolved in CHCl₃ (˜3 ml) and loaded onto a 24 g ISCO (silica) column eluting with iso-hexane:EtOAc to afford the title product; LC-MS Rt=1.46 mins; [M+H]⁺ 410.1, Method 2 minLC_v002. ¹H NMR (400 MHz, DMSO-d6) δ 8.30 (NH, t), 7.72 (1H, s), 7.29 (NH2, b s), 6.28 (OH, s), 3.68 (1H, dd), 3.47 (1H, dd), 1.24 (3H, s). ¹⁹F NMR (400 MHz, DMSO-d6) δ −62.71 (CF₃, s), −80.48 (CF₃, s).

The compounds of the following tabulated Examples (Table 2) were prepared by a similar method to that of Example 1 from the appropriate starting compound and amine. Single enantiomers were prepared by using chiral amines or by separation of the product by Supercritical Fluid Chromatography. The preparations of the starting compounds and amines are described in the Intermediates section, unless they are commercially available. DIPEA or TEA may have been used in place of 4-methylmorpholine in some reactions.

TABLE 2
Exemplary Compounds

| | | | Retention Time, |
|---|---|---|---|
| Ex. | Structure | Name | [M + H]⁺, 1H NMR |
| 1.1 | 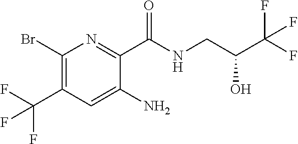 [SMILES: Nc1cc(C(F)(F)F)c(Br)nc1C(=O)NC[C@@H](O)C(F)(F)F] | 3-Amino-6-bromo- 5-trifluoromethyl- pyridine-2- carboxylic acid ((R)- 3,3,3-trifluoro-2- hydroxy-propyl)- amide (Separated by SFC, second eluted peak) | R.t 3.26 mins; Method = AD25IPA_DEA 1H NMR (DMSO) δ 3.4 (1H, m), 3.6 (1H, m), 4.3 (1H, m), 6.5 (1H, s), 7.3 (2H, s), 7.7 (1H, s), 8.6 (1H, t), |
| 1.2 | 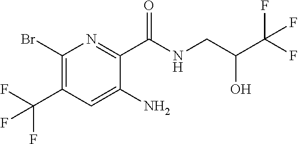 [SMILES: Nc1cc(C(F)(F)F)c(Br)nc1C(=O)NCC(O)C(F)(F)F] | 3-Amino-6-bromo- 5-trifluoromethyl- pyridine-2- carboxylic acid (3,3,3-trifluoro-2- hydroxy-propyl)- amide (Racemate) | Rt 1.42 mins; [M + H]⁺ 398 Method: 2 minLC_v002 1H NMR (DMSO) δ 3.4 (1H, m), 3.6 (1H, m), 4.3 (1H, m), 6.5 (1H, d), 7.3 (2H, s), 7.7 (1H, s), 8.6 (1H, t), |
| 1.3 | 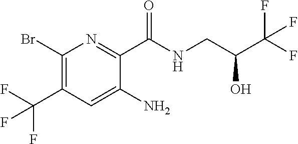 [SMILES: Nc1cc(C(F)(F)F)c(Br)nc1C(=O)NC[C@H](O)C(F)(F)F] | 3-Amino-6-bromo- 5-trifluoromethyl- pyridine-2- carboxylic acid ((S)- 3,3,3-trifluoro-2- hydroxy-propyl)- amide Prepared using (S)- 3-Amino-1,1,1- trifluoro-propan-2-ol | Rt 1.41 mins; [M + H]⁺ 398 Method: 2 minLC_v002 1H NMR (DMSO) δ 3.4 (1H, m), 3.6 (1H, m), 4.3 (1H, m), 6.5 (1H, d), 7.3, (2H, s), 7.7 (1H, s), 8.6 (1H, t) |
| 1.4 | 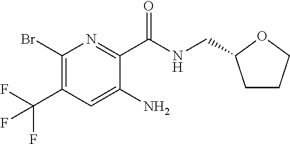 [SMILES: Nc1cc(C(F)(F)F)c(Br)nc1C(=O)NC[C@H]1CCCO1] | 3-Amino-6-bromo- 5-trifluoromethyl- pyridine-2- carboxylic acid [(R)- 1-(tetrahydro-furan- 2-yl)methyl]-amide Prepared using (R)- (−)- tetrahydrofurfuryl- | Rt 1.51 mins; [M + H]⁺ 370 Method: 2 minLC_v002 1H NMR (DMSO) δ 1.6 (1H, m), 1.9, (3H, m), 3.4, (2H, m), 3.7, (1H, m), 3.8, (1H, m), 4.1, (1H, m), 7.3, (2H, s), 7.7, (1H, s), 8.4, (1H, t) |
| | | amine | |
| 1.5 | 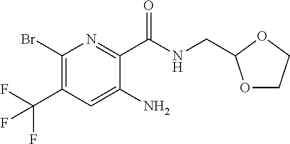 [SMILES: Nc1cc(C(F)(F)F)c(Br)nc1C(=O)NCC1OCCO1] | 3-Amino-6-bromo- 5-trifluoromethyl- pyridine-2- carboxylic acid ([1,3]dioxolan-2- ylmethyl)-amide | Rt 1.42 mins; [M + H]⁺ 370 Method: 2 minLC_v002 1H NMR (DMSO) δ3.4, (2H, t), 3.8, (2H, m), 3.9, (2H, m), 5.0, (1H, t), 7.3, (2H, br), 7.7, (1H, s), 8.4, (1H, t), |
| 1.6 | 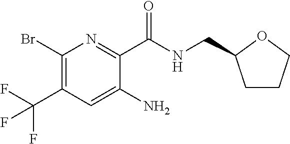 [SMILES: Nc1cc(C(F)(F)F)c(Br)nc1C(=O)NC[C@@H]1CCCO1] | 3-Amino-6-bromo- 5-trifluoromethyl- pyridine-2- carboxylic acid [(S)- 1-(tetrahydro-furan- 2-yl)methyl]-amide Prepared using (S)- (+)- Tetrahydrofurfuryl- | Rt 1.52 mins; [M + H]⁺ 370 Method: 2 minLC_v002. 1H NMR (DMSO) 1.6, (1H, m), 1.9, (3H, m), 3.3, (2H, m), 3.6, (1H, m), 3.8, (1H, m), 4.0, (1H, m), 7.4, (2H, s), 7.7, (1H, s), 8.4, (1H, d) |
| | | amine | |
| 1.7 | 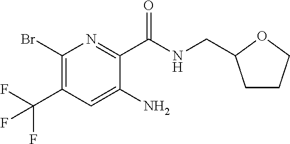 [SMILES: Nc1cc(C(F)(F)F)c(Br)nc1C(=O)NCC1CCCO1] | 3-Amino-6-bromo- 5-trifluoromethyl- pyridine-2- carboxylic acid (tetrahydro-furan-2- ylmethyl)-amide | Rt 1.49 mins; [M + H]⁺ 368 Method: 2 minLC_v002. 1H NMR (DMSO) δ1.55, (1H, m), 1.8, (3H, m), 3.3, (2H, m), 3.6, (1H, m), 3.8, (1H, m), 4.0, (1H, m), 7.3, (2H, s), 7.7, (1H, s), 8.4, (1H, t) |
| 1.8 | 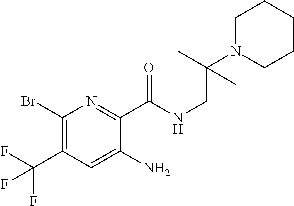 [SMILES: CC(C)(CNC(=O)c1nc(Br)c(C(F)(F)F)cc1N)N1CCCCC1] | 3-Amino-6-bromo- 5-trifluoromethyl- pyridine-2- carboxylic acid (2- methyl-2-piperidin- 1-yl-propyl)-amide | Rt 1.14 mins; [M + H]⁺ 423 Method: 2 minLC_v002 1H NMR (DMSO) δ 1.3, (6H, s), 1.4, (1H, m), 1.7, (3H, m), 1.9, (2H, m), 2.9, (2H, m), 3.6, (2H, m), 3.7, (2H, m), 7.3, (2H, s), 7.7, (1H, s), 8.7, (1H, t) |
| 1.9 | 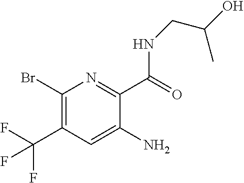 [SMILES: CC(O)CNC(=O)c1nc(Br)c(C(F)(F)F)cc1N] | 3-Amino-6-bromo- 5-trifluoromethyl- pyridine-2- carboxylic acid (2- hydroxy-propyl)- amide | Rt 4.06 mins; [M + H]⁺ 344. Method: 10 minLC_v002 1H NMR. ([400 MHz], [DMSO-d6]) δ 8.30 (1H, t), 7.69 (1H, s), 7.28 (2H, br s), 4.84 (1H, d), 3.78 (1H, m), 3.29 (1H, m), 3.14 (1H, m), 1.05 (3H, d). |
| 1.10 | 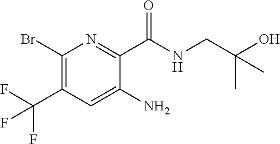 [SMILES: CC(C)(O)CNC(=O)c1nc(Br)c(C(F)(F)F)cc1N] | 3-Amino-6-bromo- 5-trifluoromethyl- pyridine-2- carboxylic acid (2- hydroxy-2-methyl- propyl)-amide | Rt 4.35 mins; [M + H]⁺ 338. Method: 10 minLC_v002 1H NMR. ([400 MHz], [DMSO-d6]) δ 8.14 (1H, t), 7.70 (1H, s), 7.29 (2H, br s), 4.70 (1H, s), 3.24 (1H, d), 1.10 (6H, s). |
| 1.11 | 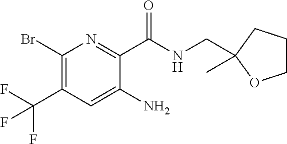 [SMILES: CC1(CNC(=O)c2nc(Br)c(C(F)(F)F)cc2N)CCCO1] | 3-Amino-6-bromo- 5-trifluoromethyl- pyridine-2- carboxylic acid (2- methyl-tetrahydro- furan-2-ylmethyl)- amide | Rt 1.53 mins; [M + H]⁺ 384 Method: 2 minLC_v002. 1H NMR. ([400 MHz], [DMSO-d6]) δ 8.12 (1H, t), 7.70 (1H, s), 7.28 (2H, br s), 3.76 (2H, t), 3.33 (2H, d), 1.88 (2H, m), 1.80 (1H, m), 1.60 (1H, m), 1.13 (3H, s). |
| 1.12 | 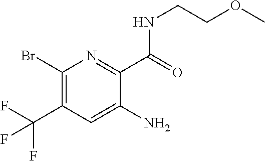 [SMILES: COCCNC(=O)c1nc(Br)c(C(F)(F)F)cc1N] | 3-Amino-6-bromo- 5-trifluoromethyl- pyridine-2- carboxylic acid (2- methoxy-ethyl)- amide | Rt 1.43 mins; [M + H]⁺ 342; Method: 2 minLC_v002. 1H NMR. ([400 MHz], [DMSO-d6]) δ 8.41 (1H, t), 7.69 (1H, s), 7.28 (2H, br s), 3.44 (4H, m), 3.27 (3H, s). |
| 1.13 | 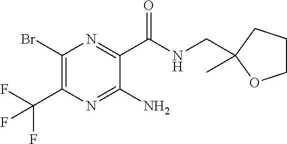 [SMILES: CC1(CNC(=O)c2nc(Br)c(C(F)(F)F)nc2N)CCCO1] | 3-Amino-6-bromo- 5-trifluoromethyl- pyrazine-2- carboxylic acid (2- methyl-tetrahydro- furan-2-ylmethyl)- amide | Rt 1.52 mins; [M + H]⁺ 385; Method: 2 minLC_v002. 1H NMR. ([400 MHz], [DMSO-d6]) δ 8.35 (1H, t), 8.09 (2H, br s), 3.76 (2H, t), 3.34 (2H, d), 1.86 (3H, m), 1.58 (1H, m), 1.13 (3H, s). |
| 1.14 | 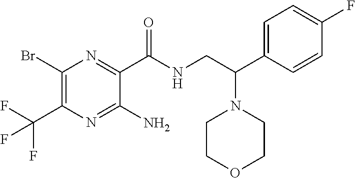 [SMILES: Nc1nc(C(F)(F)F)c(Br)nc1C(=O)NCC(c1ccc(F)cc1)N1CCOCC1] | 3-Amino-6-bromo- 5-trifluoromethyl- pyrazine-2- carboxylic acid [2- (4-fluoro-phenyl)-2- morpholin-4-yl- ethyl]-amide | Rt 4.29 mins; [M + H]⁺ 492. Method: 10 minLC_v002. ¹H NMR (400 MHz, MeOD) δ_{H} 7.61-7.64 (2H, m, 2 × Ar′H (AA′BB′X system)), 7.28-7.32 (2H, m, 2 × Ar′H (AA′BB′X system)), 4.65-4.73 (1H, br m, NHCH_{A}H_{B}CH_{X}N (Ar′)), 4.31 (1H, dd_{ABX}, J = 6.5/14.4 Hz, |
| | | | NHCH_{A}H_{B}CH_{X}N(Ar′)), |
| | | | 3.70-4.14 (6H, m, |
| | | | NHCH_{A}H_{B}CH_{X}N (Ar′) + |
| | | | 5 × morpholine CH), 3.00- |
| | | | 3.30 (3H, m, |
| | | | 3 × morpholine CH). |
| 1.15 | 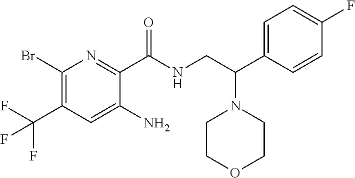 [SMILES: Nc1cc(C(F)(F)F)c(Br)nc1C(=O)NCC(c1ccc(F)cc1)N1CCOCC1] | 3-Amino-6-bromo- 5-trifluoromethyl- pyridine-2- carboxylic acid [2- (4-fluoro-phenyl)-2- morpholin-4-yl- ethyl]-amide | Rt 4.39 mins; [M + H]⁺ 491 Method: 10 minLC_v002. ¹H NMR (400 MHz, MeOD) δ_{H} 8.37-8.38 (1H, m, ArC(O)NHCH₂), 7.68 (1H, s, ArH-1), 7.35 (2H, m (AA′BB′X system), 2 × Ar′H-2), 7.17-7.25 (4H, br s + m (AA′BB′X system), ArNH₂ + 2 × Ar′H-3), 3.73- |
| | | | 3.81 (2H, m, |
| | | | NHCH_{A}H_{B}CH(N)Ar′ + |
| | | | NHCH_{A}H_{B}CH(N)Ar′), |
| | | | 3.53-3.58 (5H, morpholine |
| | | | 2 × CH₂ + |
| | | | NHCH_{A}H_{B}CH(N)Ar′), |
| | | | 2.34-2.43 (4H, m, |
| | | | 2 × morpholine CH₂). |
| 1.16 | 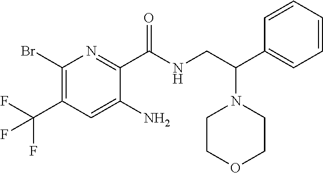 [SMILES: Nc1cc(C(F)(F)F)c(Br)nc1C(=O)NCC(c1ccccc1)N1CCOCC1] | 3-Amino-6-bromo- 5-trifluoromethyl- pyridine-2- carboxylic acid (2- morpholin-4-yl-2- phenyl-ethyl)-amide | Rt 4.20 mins; [M + H]⁺ 473 Method 10 minLC_v002. 1H NMR (400 MHz, DMSO-d6) δ 8.46 (1H, s broad), 7.70 (1H, s), 7.32 (7H, m broad), 3.75 (2H, m broad), 3.59 (4H, broad), 2.40 (2H, broad), 2.30 (2H, broad). |
| 1.17 | 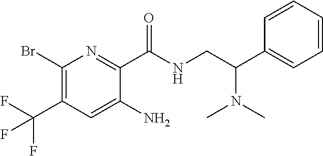 [SMILES: CN(C)C(CNC(=O)c1nc(Br)c(C(F)(F)F)cc1N)c1ccccc1] | 3-Amino-6-bromo- 5-trifluoromethyl- pyridine-2- carboxylic acid (2- dimethylamino-2- phenyl-ethyl)-amide | Rt 4.25 mins; [M + H]⁺ 431; Method; 10 minLC_v002. 1H NMR (400 MHz, DMSO-d6) δ 10.20 (1H, s), 8.72 (1H, t), 7.71 (1H, s), 7.60 (2H, s broad), 7.50 (3H, s broad), 7.30 (2H, s broad), 4.67 (1H, m), 4.19 |
| | | | (1H, qui), 3.83 (m), 2.80 |
| | | | (3H, d), 2.61 (3H, d). |
| 1.18 | 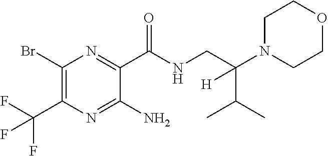 [SMILES: CC(C)C(CNC(=O)c1nc(Br)c(C(F)(F)F)nc1N)N1CCOCC1] | 3-Amino-6-bromo- 5-trifluoromethyl- pyrazine-2- carboxylic acid (3- methyl-2- morpholin-4-yl- butyl)-amide | Rt 3.39 mins; [M + H]⁺ 442. Method 10 minLC_v002. 1H NMR (400 MHz, DMSO-d6) δ 9.03 (1H, s), 9.10 (1H, s), 8.12 (2H, s), 4.00-3.20 (12H, broad), 1.10 (3H, broad), 1.00 (3H, broad). |
| 1.19 | 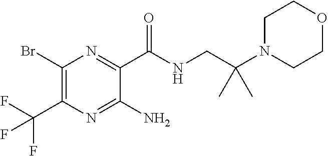 [SMILES: CC(C)(CNC(=O)c1nc(Br)c(C(F)(F)F)nc1N)N1CCOCC1] | 3-Amino-6-bromo- 5-trifluoromethyl- pyrazine-2- carboxylic acid (2- methyl-2- morpholin-4-yl- propyl)-amide | Rt 2.83 mins; [M + H]⁺ 428; Method 10 minLC_v002. 1H NMR (400 MHz, DMSO-d6) δ 9.04 (1H, s), 8.95 (1H, s), 8.10 (2H, s), 4.04 (2H, d), 3.75 (2H, t), 3.60 (4H + broad water signal underneath), 3.20 (2H, q), 1.35 (6H, s). |
| 1.20 | 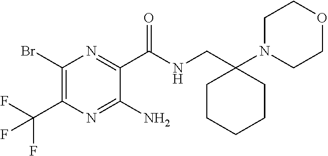 [SMILES: Nc1nc(C(F)(F)F)c(Br)nc1C(=O)NCC1(N2CCOCC2)CCCCC1] | 3-Amino-6-bromo- 5-trifluoromethyl- pyrazine-2- carboxylic acid (1- morpholin-4-yl- cyclohexylmethyl)- amide | Rt 3.59 mins; [M + H]⁺ 466. Method 10 minLC_v002. 1H NMR (400 MHz, DMSO-d6) δ 9.00 (1H, broad), 8.90 (1H, broad), 8.10 (1H, s), 4.02-3.35 (20H, very broad) |
| 1.21 | 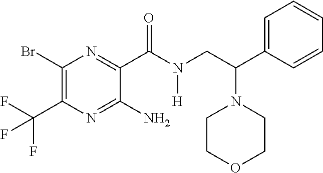 [SMILES: Nc1nc(C(F)(F)F)c(Br)nc1C(=O)NCC(c1ccccc1)N1CCOCC1] | 3-Amino-6-bromo- 5-trifluoromethyl- pyrazine-2- carboxylic acid (2- morpholin-4-yl-2- phenyl-ethyl)-amide | Rt 6.09 mins; [M + H]⁺ 474 Method 10 minC18 |
| 1.22 | 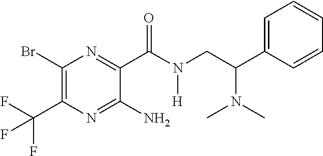 [SMILES: CN(C)C(CNC(=O)c1nc(Br)c(C(F)(F)F)nc1N)c1ccccc1] | 3-Amino-6-bromo- 5-trifluoromethyl- pyrazine-2- carboxylic acid (2- dimethylamino-2- phenyl-ethyl)-amide | Rt 5.42 mins; [M + H]⁺ 432 Method 10 minC18 |
| 1.23 | 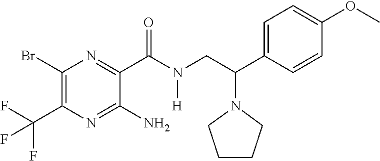 [SMILES: COc1ccc(C(CNC(=O)c2nc(Br)c(C(F)(F)F)nc2N)N2CCCC2)cc1] | 3-Amino-6-bromo- 5-trifluoromethyl- pyrazine-2- carboxylic acid [2- (4-methoxy-phenyl)- 2-pyrrolidin-1-yl- ethyl]-amide | Rt 5.64 mins; [M + H]⁺ 488 Method 10 minC18 |
| 1.24 | 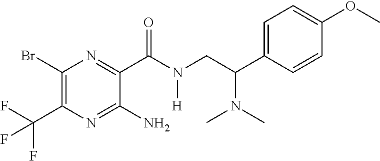 [SMILES: COc1ccc(C(CNC(=O)c2nc(Br)c(C(F)(F)F)nc2N)N(C)C)cc1] | 3-Amino-6-bromo- 5-trifluoromethyl- pyrazine-2- carboxylic acid [2- dimethylamino-2-(4- methoxy-phenyl)- ethyl]-amide | Rt 5.51 mins; [M + H]⁺ 462; Method 10 minC18 |
| 1.25 | 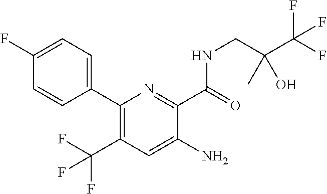 [SMILES: CC(O)(CNC(=O)c1nc(-c2ccc(F)cc2)c(C(F)(F)F)cc1N)C(F)(F)F] | 3-Amino-6-(4- fluoro-phenyl)-5- trifluoromethyl- pyridine-2- carboxylic acid (3,3,3-trifluoro-2- hydroxy-2-methyl- propyl)-amide | Rt 5.41 mins; [M + H]⁺ 426; Method 10 minLC_v002. ¹H NMR δ 8.42 (1H, m), 7.72 (1H, s), 7.5 (2H, m), 7.3 (2H, t), 7.22 (2H, br s), 6.24 (1H, s), 3.68 (1H, m), 3.46 (1H, m), 1.24 (3H, s) |
| 1.26 | 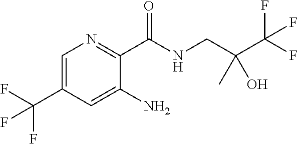 [SMILES: CC(O)(CNC(=O)c1ncc(C(F)(F)F)cc1N)C(F)(F)F] | 3-Amino-5- trifluoromethyl- pyridine-2- carboxylic acid (3,3,3-trifluoro-2- hydroxy-2-methyl- propyl)-amide | Rt 4.18 mins; [M + H]⁺ 332; Method 10 minLC_v002. ¹H NMR δ 8.58 (1H, t), 8.1 (1H, s), 7.56 (1H, s), 7.2 (2H, br s), 6.29 (1H, s), 3.61-3.7 (1H, m), 3.42- 3.5 (1H, m), 1.26 (3H, s). |
| 1.27 | 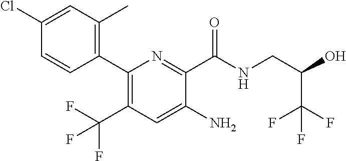 [SMILES: Cc1cc(Cl)ccc1-c1nc(C(=O)NC[C@@H](O)C(F)(F)F)c(N)cc1C(F)(F)F] | 3-Amino-6-(4- chloro-2-methyl- phenyl)-5- trifluoromethyl- pyridine-2- carboxylic acid ((R)- 3,3,3-trifluoro-2- hydroxy-propyl)- amide Prepared using (R)- | Rt 1.59 mins; [M + H]⁺ 442 Method: 2 minLC_v002. 1H NMR. ([400 MHz], [DMSO-d6]) δ 8.54 (1H, br), 7.69 (1H, s), 7.41 (1H, d), 7.30 (1H, dd), 7.24 (2H, br s), 7.20 (1H, d), 6.40 (1H, br), 4.19 (1H, m), 3.54 (1H, m), 3.36 (1H, m), 2.01 (3H, s). |
| | | 3-amino-1,1,1- | |
| | | trifluoropropan-2-ol | |
| 1.28 | 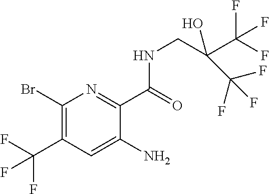 [SMILES: Nc1cc(C(F)(F)F)c(Br)nc1C(=O)NCC(O)(C(F)(F)F)C(F)(F)F] | 3-Amino-6-bromo- 5-trifluoromethyl- pyridine-2- carboxylic acid (3,3,3-trifluoro-2- hydroxy-2- trifluoromethyl- propyl)-amide | Rt 1.59 mins; [M + H]⁺ 466; Method 2 minLC_v002. ¹H NMR (400 MHz, DMSO-d6) δ 8.50 (1H, t), 8.30 (1H, s), 7.72 (1H, s), 7.30 (2H, s), 4.00 (2H, d) |
| 1.29 | 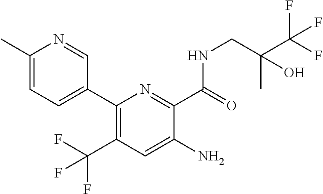 [SMILES: Cc1ccc(-c2nc(C(=O)NCC(C)(O)C(F)(F)F)c(N)cc2C(F)(F)F)cn1] | 5-Amino-6′-methyl- 3-trifluoromethyl- [2,3′]bipyridinyl-6- carboxylic acid (3,3,3-trifluoro-2- hydroxy-2-methyl- propyl)-amide | Rt 1.16 mins; [M + H]⁺ 423; Method 2 minLC_v002. ¹H NMR (400 MHz, DMSO-d6) δ 8.53 (1H, d), 8.45 (1H, t), 7.75 (1H, d), 7.71 (1H, s), 7.34 (1H, d), 7.25 (2H, s), 6.21 (1H, s), 3.69 (1H, dd), 3.42 (1H, dd), 2.54 (3H, s), 1.22 (3H, s). |
| 1.30 | 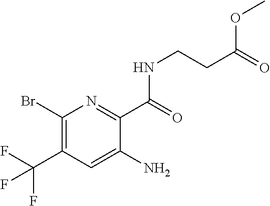 [SMILES: COC(=O)CCNC(=O)c1nc(Br)c(C(F)(F)F)cc1N] | Methyl 3-(3-amino- 6-bromo-5- (trifluoromethyl) picolinamido) propanoate | Rt 4.64 mins; [M + H]⁺ 372.1; Method 10 minLC_v002. ¹H NMR (400 MHz, DMSO-d6) δ 8.56 (1H, t), 7.68 (1H, s), 7.27 (2H, br s), 3.61 (3H, s) 3.50 (2H, q), 2.60 (2H, t). |
| 1.31 | 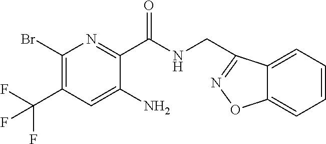 [SMILES: Nc1cc(C(F)(F)F)c(Br)nc1C(=O)NCc1noc2ccccc12] | 3-Amino-N- (benzo[d]isoxazol-3- ylmethyl)-6-bromo- 5-(trifluoromethyl) picolinamide | Rt 4.18 mins; [M + H]⁺ 417.1; Method 10 minLC_v002. ¹H NMR (400 MHz, DMSO-d6) δ 9.32 (NH, t), 7.96 (1H, dt), 7.74 (1H, dt), 7.70 (1H, s), 7.65-7.62 (1H, m), 7.40-7.36 (1H, m), 7.29 (NH2, b s), 4.88 (2H, d) |
| 1.32 | 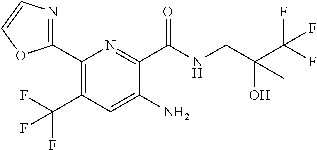 [SMILES: CC(O)(CNC(=O)c1nc(-c2ncco2)c(C(F)(F)F)cc1N)C(F)(F)F] | 3-Amino-6-(oxazol- 2-yl)-N-(3,3,3- trifluoro-2-hydroxy- 2-methylpropyl)-5- (trifluoromethyl) picolinamide | Rt 3.44 mins; [M + H]⁺ 399.1; Method 10 minLC_v003. |
| 1.33 | 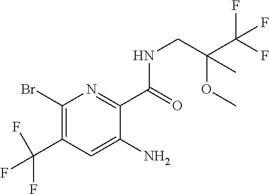 [SMILES: COC(C)(CNC(=O)c1nc(Br)c(C(F)(F)F)cc1N)C(F)(F)F] | Single enantiomer of 3-Amino-6-bromo- N-(3,3,3-trifluoro-2- methoxy-2- methylpropyl)-5- (trifluoromethyl) picolinamide (Separated by SFC, second eluted peak) | 1H NMR (400 MHz, DMSO-d6) δ 8.24 (1H, t), 7.72 (1H, s), 7.29 (2H, s), 3.65 (2H, m), 3.37 (3H, s), 1.35 (3H, s) |
| 1.34 | 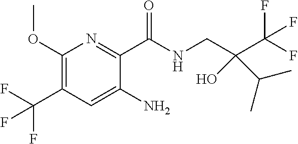 [SMILES: COc1nc(C(=O)NCC(O)(C(C)C)C(F)(F)F)c(N)cc1C(F)(F)F] | Single enantiomer of 3-amino-N-(2- hydroxy-3-methyl- 2-(trifluoromethyl) butyl)-6-methoxy-5- (trifluoromethyl) picolinamide (Separated by SFC, first eluted peak) | Rt 4.48 mins; [M + H]⁺ 390.3; Method 10 minLC_v003. ¹H NMR (400 MHz, DMSO-d6) δ 8.21 (1H, m), 7.69 (1H, s), 6.59 (2H, s), 6.26 (1H, s), 3.91 (3H, s), 3.68 (2H, m), 2.02 (1H, m), 1.02 (6H, m) |
| 1.35 | 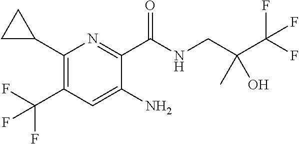 [SMILES: CC(O)(CNC(=O)c1nc(C2CC2)c(C(F)(F)F)cc1N)C(F)(F)F] | 3-Amino-6- cyclopropyl-N- (3,3,3-trifluoro-2- hydroxy-2- methylpropyl)-5- (trifluoromethyl) picolinamide | Rt 1.23 mins; [M + H]⁺ 372.2; Method 2 minLC_v003. ¹H NMR (400 MHz, DMSO-d6) δ 8.29 (1H, m), 7.56 (1H, s), 6.9 (2H, br s), 6.3 (1H, s), 3.62 (1H, m), 3.48 (1H, m), 2.1 (1H, m), 1.24 (3H, s), 0.9-1.1 (4H, m) |
| 1.36 | 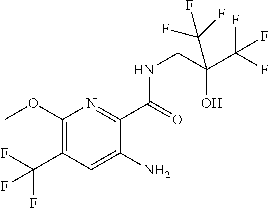 [SMILES: COc1nc(C(=O)NCC(O)(C(F)(F)F)C(F)(F)F)c(N)cc1C(F)(F)F] | 3-Amino-6- methoxy-N-(3,3,3- trifluoro-2-hydroxy- 2- (trifluoromethyl) propyl)-5- (trifluoromethyl) picolinamide | Rt 4.27 mins; [M + H]⁺ 416.3; Method 10 minLC_v003. ¹H NMR (400 MHz, DMSO-d6) δ 8.39 (1H, t), 8.32 (1H, br s), 7.69 (1H, s), 6.70 (2H, br s), 3.99 (2H, d), 3.93 (3H, s), |
| 1.37 | 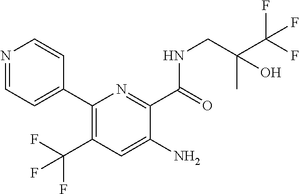 [SMILES: CC(O)(CNC(=O)c1nc(-c2ccncc2)c(C(F)(F)F)cc1N)C(F)(F)F] | Single enantiomer of 5-amino-N-(3,3,3- trifluoro-2-hydroxy- 2-methylpropyl)-3- (trifluoromethyl)- 2,4′-bipyridine-6- carboxamide (Separated by SFC, first eluted peak) | Rt 0.86 mins; [M + H]⁺ 409.1; Method 2 minLC_v003. ¹H NMR (400 MHz, DMSO-d6) δ 8.70 (2H, d), 8.45 (1H, t), 7.75 (1H, s), 7.50 (2H, d), 7.33 (2H, s), 6.22 (1H, s), 3.69 (1H, dd), 3.43 (1H, dd), 1.21 (3H, s). |
| 1.38 | 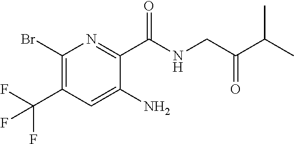 [SMILES: CC(C)C(=O)CNC(=O)c1nc(Br)c(C(F)(F)F)cc1N] | 3-Amino-6-bromo- 5-trifluoromethyl- pyridine-2- carboxylic acid (3- methyl-2-oxo- butyl)-amide | Rt 1.18 mins; [M + H]⁺ 368; Method 2 minLC_v003. ¹H NMR (400 MHz, DMSO-d6) δ 8.64 (1H, t), 7.71 (1H, s), 7.29 (2H, broad s), 4.2 (2H, d), 2.7- 2.8 (1H, m), 1.08 (6H, d, 2 × CH3). |
| 1.39 | 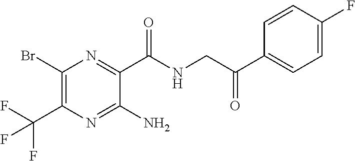 [SMILES: Nc1nc(C(F)(F)F)c(Br)nc1C(=O)NCC(=O)c1ccc(F)cc1] | 3-Amino-6-bromo- 5-trifluoromethyl- pyrazine-2- carboxylic acid [2- (4-fluoro-phenyl)-2- oxo-ethyl]-amide | ¹H NMR (400 MHz, DMSO-d6) δ 9.0 (1H, t, NH), 8.1 (4H, m, NH2, Ar- H), 7.4 (2H, t, Ar-H), 4.8 (2H, 5, CH2) |

### Examples 2 and 3: 3-Amino-6-bromo-5-trifluoromethyl-pyridine-2-carboxylic acid ((R)-3,3,3-trifluoro-2-hydroxy-2-methyl-propyl)-amide and 3-Amino-6-bromo-5-trifluoromethyl-pyridine-2-carboxylic acid ((S)-3,3,3-trifluoro-2-hydroxy-2-methyl-propyl)-amide

Example 2: 3-Amino-6-bromo-5-trifluoromethyl-pyridine-2-carboxylic acid ((R)-3,3,3-trifluoro-2-hydroxy-2-methyl-propyl)-amide,

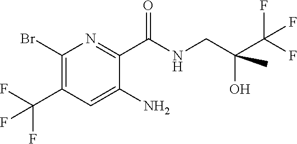 [SMILES: C[C@@](O)(CNC(=O)c1nc(Br)c(C(F)(F)F)cc1N)C(F)(F)F]

was prepared by chiral separation of 3-amino-6-bromo-5-trifluoromethyl-pyridine-2-carboxylic acid (3,3,3-trifluoro-2-hydroxy-2-methyl-propyl)-amide (Example 1) using Supercritical Fluid Chromatography under the following conditions:
Mobile Phase: 12% isopropanol+0.1% DEA/88% CO₂

### Column: Chiralpak OJ-H, 250×10 mm id, 5 μm

### Detection: UV @ 220 nm

Flow rate: 10 ml/min
Sample concentration: 347 mg in 5 ml EtOH.
Injection volume: 50 μl

First eluted peak: 3-Amino-6-bromo-5-trifluoromethyl-pyridine-2-carboxylic acid ((R)-3,3,3-trifluoro-2-hydroxy-2-methyl-propyl)-amide.

LC-MS: Rt=4.97 min [M+H]+ 410.1/412.2 (Method 10 minLC_v002).

¹H NMR (400 MHz, DMSO-d6) δ 8.30 (NH, t), 7.72 (1H, s), 7.29 (NH₂, b s), 6.28 (OH, s), 3.68 (1H, dd), 3.47 (1H, dd), 1.24 (3H, s)

¹⁹F NMR (400 MHz, DMSO-d6) d −62.70 (CF₃, s), −80.47 (CF₃, s)

Optical rotation [α]²¹_{D} at 589 nm+14.4° (c=0.522, MeOH).

Example 3: 3-Amino-6-bromo-5-trifluoromethyl-pyridine-2-carboxylic acid ((S)-3,3,3-trifluoro-2-hydroxy-2-methyl-propyl)-amide,

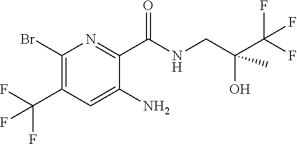 [SMILES: C[C@](O)(CNC(=O)c1nc(Br)c(C(F)(F)F)cc1N)C(F)(F)F]

was prepared by chiral separation of 3-amino-6-bromo-5-trifluoromethyl-pyridine-2-carboxylic acid (3,3,3-trifluoro-2-hydroxy-2-methyl-propyl)-amide (Example 1) using Supercritical Fluid

Chromatography under the following conditions:

Mobile Phase: 12% isopropanol+0.1% DEA/88% CO₂

Column: Chiralpak OJ-H, 250×10 mm id, 5 μm

Detection: UV @ 220 nm

Flow rate: 10 ml/min

Sample concentration: 347 mg in 5 ml EtOH.

Injection volume: 50 μl

Second eluted peak: 3-Amino-6-bromo-5-trifluoromethyl-pyridine-2-carboxylic acid ((S)-3,3,3-trifluoro-2-hydroxy-2-methyl-propyl)-amide.

LC-MS Rt=4.94 min [M+H]+ 412.1 (Method 10 minLC_v002).

¹H NMR (400 MHz, DMSO-d6) δ 8.30 (NH, t), 7.72 (1H, s), 7.29 (NH₂, b s), 6.28 (OH, s), 3.68 (1H, dd), 3.47 (1H, dd), 1.24 (3H, s)

¹⁹F NMR (400 MHz, DMSO-d6) d −62.70 (CF₃, s), −80.48 (CF₃, s).

The stereochemistry of this compound was confirmed by X-ray crystallography.

### Examples 4, 5 and 6: 3-Amino-6-methoxy-5-trifluoromethyl-pyridine-2-carboxylic acid (3,3,3-trifluoro-2-hydroxy-2-methyl-propyl)-amide and its enantiomers

Example 4: 3-Amino-6-methoxy-5-trifluoromethyl-pyridine-2-carboxylic acid (3,3,3-trifluoro-2-hydroxy-2-methyl-propyl)-amide,

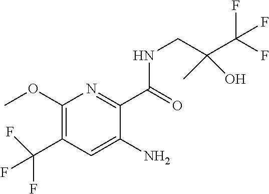 [SMILES: COc1nc(C(=O)NCC(C)(O)C(F)(F)F)c(N)cc1C(F)(F)F]

was prepared according to the following procedure:

A solution comprising 3-amino-6-methoxy-5-trifluoromethyl-pyridine-2-carboxylic acid (Intermediate D)(4 g, 16.94 mmol) and 3-amino-1,1,1-trifluoro-2-methylpropan-2-ol hydrochloride (Intermediate R) (3.04 g, 16.94 mmol) in NMP (188 ml) was treated with HATU (7.73 g, 20.33 mmol) followed by dropwise addition (2 ml portions) of DIPEA (8.88 ml, 50.8 mmol) over 1 hour. After stirring for a further hour, the reaction mixture was poured into water (450 ml) and EtOAc (450 ml). The aqueous phase was acidified with 5M HCl (50 ml) and the layers were separated. The organic portion was washed with 2M NaOH (200 ml), water (4×200 ml), brine (2×100 ml), dried over MgSO₄, filtered and concentrated in vacuo to afford a brown solid. Purification of the solid by chromatography on silica (220 g pre-packed silica cartridge) eluting with 0-50% EtOAc in iso-hexane afforded the racemate, 3-amino-6-methoxy-5-trifluoromethyl-pyridine-2-carboxylic acid (3,3,3-trifluoro-2-hydroxy-2-methyl-propyl)-amide (Ex. 4) as a yellow solid;

1H NMR (400 MHz, DMSO-d6) δ 8.3 (1H, t), 7.7 (1H, s), 6.7 (2H, s), 6.2 (1H, s), 3.9 (3H, s), 3.7 (1H, m), 3.5 (1H, m), 1.2 (3H, s).

LC-MS: Rt 1.24 min; MS m/z 362.4 [M+H]+; Method 2 minLC_v003.

Chiral separation of the racemate by Supercritical Fluid Chromatography was carried out using the following conditions to afford the compounds listed hereinafter:

Mobile Phase: 12% 2-propanol+0.1% DEA/50% CO₂

Column: Chiralcel OD-H, 250×10 mm id, 5 μm (2 columns linked in series)

Detection: UV @ 220 nm

Flow rate: 10 ml/min

Sample concentration: 3.5 g in 30 ml EtOH

Injection volume: 100 μl

### Examples 5 and 6 are Entantiomers

Example 5: First eluted peak Rt=7.30 minutes. 3-Amino-6-methoxy-5-trifluoromethyl-pyridin e-2-carboxylic acid ((S)-3,3,3-trifluoro-2-hydroxy-2-methyl-propyl)-amide (“Compound A”):

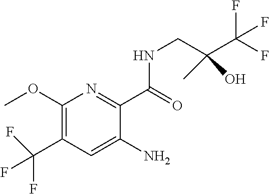 [SMILES: COc1nc(C(=O)NC[C@](C)(O)C(F)(F)F)c(N)cc1C(F)(F)F]

1H NMR (400 MHz, DMSO-d6) δ 8.3 (1H, t), 7.6 (1H, s), 6.6 (2H, broad), 6.2 (1H, s), 3.9 (3H, s), 3.6 (1H, m), 3.5 (1H, m), 1.3 (3H, s);

LC-MS Rt=1.15 mins, [M+H]+ 362.4 (Method 2 minLC_v003).

Optical rotation [α]²¹_{D} at 589 nm −20.83° (c=0.513, MeOH).

The stereochemistry of this compound was confirmed by X-ray crystallography.

Example 6: Second eluted peak Rt=8.29 minutes. 3-Amino-6-methoxy-5-trifluoromethyl-pyridine-2-carboxylic acid ((R)-3,3,3-trifluoro-2-hydroxy-2-methyl-propyl)-amide

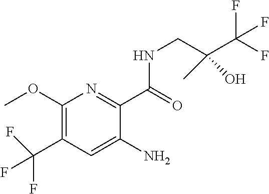 [SMILES: COc1nc(C(=O)NC[C@@](C)(O)C(F)(F)F)c(N)cc1C(F)(F)F]

1H NMR (400 MHz, DMSO-d6) δ 8.3 (1H, t), 7.6 (1H, s), 6.6 (2H, broad), 6.2 (1H, s), 3.9 (3H, s), 3.6 (1H, m), 3.5 (1H, m), 1.3 (3H, s);

LC-MS Rt=1.15 mins [M+H]+ 362.4 (Method 2 minLC_v003).

Alternatively, Example 5 may be prepared according to the following method: To a solution of 3-amino-6-methoxy-5-trifluoromethyl-pyridine-2-carboxylic acid (Intermediate D) (10 g, 42.3 mmol) and (S)-3-amino-1,1,1-trifluoro-2-methylpropan-2-ol hydrochloride (Intermediate RA)(7.60 g, 42.3 mmol) in NMP (400 ml) was added HATU (19.3 g, 50.8 mmol) followed by dropwise addition of DIPEA (22.19 ml, 127 mmol) over ˜1 hr. After stirring at room temperature for 30 min, the mixture was added to EtOAc (2 L), washed with 1M NaOH (2×1 L), water (1 L), brine (1 L), dried (MgSO₄) and evaporated under reduced pressure to give the crude product as a dark brown oil. Purification by chromatography on silica eluting with a gradient of 1 to-25% of EtOAc in iso-hexane afforded a yellow oil. Recrystallisation of the oil from iso-hexane/DCM afforded 3-amino-6-methoxy-5-trifluoromethyl-pyridine-2-carboxylic acid ((S)-3,3,3-trifluoro-2-hydroxy-2-methyl-propyl)-amide as a crystalline solid;

¹H NMR (400 MHz, DMSO-d6) δ 8.28 (1H, t), 7.66 (1H, s), 6.67 (2H, s), 6.27 (1H, s), 3.91 (3H, s), 3.65 (1H, m), 3.45 (1H, m), 1.24 (3H, s).

¹⁹F NMR (376 MHz, DMSO-d6) −62.58 ppm (s), −80.43 ppm (s)

### Example 7: 3-Amino-6-(4-fluoro-phenyl)-5-trifluoromethyl-pyridine-2-carboxylic acid ((S)-3,3,3-trifluoro-2-hydroxy-2-methyl-propyl)-amide

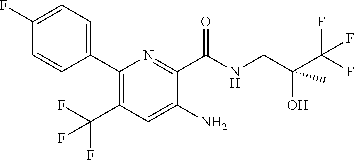 [SMILES: C[C@](O)(CNC(=O)c1nc(-c2ccc(F)cc2)c(C(F)(F)F)cc1N)C(F)(F)F]

A mixture comprising 3-amino-6-bromo-5-trifluoromethyl-pyridine-2-carboxylic acid ((S)-3,3,3-trifluoro-2-hydroxy-2-methyl-propyl)-amide (Ex. 3) (100 mg, 0.244 mmol), 4-fluorophenylboronic acid (37.5 mg, 0.268 mmol) and 1,1′bis(diphenylphosphoshio) ferrocenepalladium dichloride (19.90 mg, 0.024 mmol) was suspended in THF (2 ml) and 1M Cs₂CO₃ (0.667 ml). The vial was flushed with N₂, sealed and heated at 160° C. using microwave radiation for 15 minutes. The mixture was partitioned between EtOAc (50 ml) and water (50 ml). The organic portion was separated and washed with brine (30 ml), dried (MgSO₄), filtered through Celite® (filter material) and concentrated in vacuo. The crude residue was dissolved in DMSO (2 ml) and purified by mass directed LCMS using MeCN/Water/0.1% TFA eluent to afford clean product. The product fraction obtained as MeCN/Water/0.1% TFA solution was poured into EtOAc (50 ml) and washed with saturated NaHCO₃ (50 ml) to free base the product. The organic portion were combined, dried (MgSO₄) and concentrated in vacuo to afford the title compound as a pale orange crystalline solid; 1H NMR (400 MHz, DMSO-d6) δ 8.4 (1H, m), 7.7 (1H, s), 7.49 (2H, m), 7.29 (2H, t), 7.2 (2H, br s), 6.22 (1H, s), 3.68 (1H, m), 3.44 (1H, m), 1.22 (3H, s); LC-MS Rt 4.41 mins [M+H]+ 426 (Method 10 minLC_v003).

### Example 8: 3-Amino-6-(4-fluoro-phenyl)-5-trifluoromethyl-pyridine-2-carboxylic acid ((R)-3,3,3-trifluoro-2-hydroxy-2-methyl-propyl)-amide

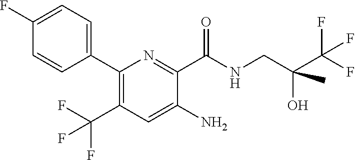 [SMILES: C[C@@](O)(CNC(=O)c1nc(-c2ccc(F)cc2)c(C(F)(F)F)cc1N)C(F)(F)F]

This compound was prepared from 3-Amino-6-bromo-5-trifluoromethyl-pyridine-2-carboxylic acid ((R)-3,3,3-trifluoro-2-hydroxy-2-methyl-propyl)-amide (Ex. 2) analogously to Example 8. 1H NMR (400 MHz, DMSO-d6) δ 8.42 (1H, m), 7.7 (1H, s), 7.5 (2H, m), 7.3 (2H, t), 7.21 (2H, br s), 6.24 (1H, s), 3.68 (1H, m), 3.44 (1H, m), 1.22 (3H, s); LC-MS Rt=4.39 mins [M+H]+ 426 (Method 10 minLC_v003).

### Examples 9 and 10: Enantiomers of 3-amino-6-(2,4-dichloro-phenyl)-5-trifluoromethyl-pyridine-2-carboxylic acid (3,3,3-trifluoro-2-hydroxy-2-methyl-propyl)-amide

The enantiomers of 3-amino-6-(2,4-dichloro-phenyl)-5-trifluoromethyl-pyridine-2-carboxylic acid (3,3,3-trifluoro-2-hydroxy-2-methyl-propyl)-amide were prepared from 3-Amino-6-(2,4-dichloro-phenyl)-5-trifluoromethyl-pyridine-2-carboxylic acid (Intermediate H) and 3-amino-1,1,1-trifluoro-2-methylpropan-2-ol hydrochloride analogously to Example 1 and separated by chiral separation using Supercritical Fluid Chromatography:

### Example 9: First eluted peak. Entantiomer 1 of 3-Amino-6-(2,4-dichloro-phenyl)-5-trifluoromethyl-pyridine-2-carboxylic acid (3,3,3-trifluoro-2-hydroxy-2-methyl-propyl)-amide

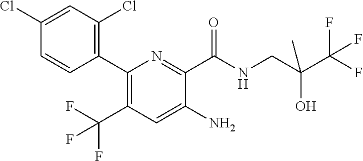 [SMILES: CC(O)(CNC(=O)c1nc(-c2ccc(Cl)cc2Cl)c(C(F)(F)F)cc1N)C(F)(F)F]

1H NMR (400 MHz, DMSO-d6) δ 8.38 (t, 1H), 7.83 (s, 1H), 7.78 (s, 1H), 7.60 (d, 1H), 7.54 (d, 1H), 7.39 (br s, 2H), 6.25 (br s, 1H). 3.71 (dd, 1H), 3.48 (dd, 1H), 1.26 (s, 3H); LC-MS Rt=1.65 mins [M+H]+ 476 (Method 2 minLC_v002).

Example 10: Second Eluted peak. Enantiomer 2 of 3-Amino-6-(2,4-dichloro-phenyl)-5-trifluoromethyl-pyridine-2-carboxylic acid (3,3,3-trifluoro-2-hydroxy-2-methyl-propyl)-amide.

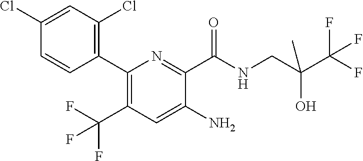 [SMILES: CC(O)(CNC(=O)c1nc(-c2ccc(Cl)cc2Cl)c(C(F)(F)F)cc1N)C(F)(F)F]

1H NMR (400 MHz, DMSO-d6) δ 8.38 (t, 1H), 7.83 (s, 1H), 7.78 (s, 1H), 7.60 (d, 1H), 7.54 (d, 1H), 7.39 (br s, 2H), 6.25 (br s, 1H). 3.71 (dd, 1H), 3.48 (dd, 1H), 1.26 (s, 3H); LC-MS Rt 1.65 mins [M+H]+=476.1 (Method 2 minLC_v002).

### Example 11: 3-Amino-6-(4-fluoro-phenyl)-5-trifluoromethyl-pyridine-2-carboxylic acid (2-hydroxy-2-methyl-propyl)-amide

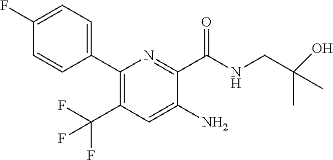 [SMILES: CC(C)(O)CNC(=O)c1nc(-c2ccc(F)cc2)c(C(F)(F)F)cc1N]

To a stirred suspension of 3-amino-6-bromo-5-trifluoromethyl-pyridine-2-carboxylic acid (2-hydroxy-2-methyl-propyl)-amide (Ex. 1.10) (180 mg, 0.505 mmol) and 4-fluorophenylboronic acid (106 mg, 0.758 mmol) in a 2:1 mixture of toluene:EtOH (12 ml) under nitrogen was added 2M Na₂CO₃(aq) (1.011 ml, 2.022 mmol) followed by Pd(dppf)Cl₂.CH₂Cl₂ adduct (41 mg, 0.051 mmol). The reaction mixture was heated using microwave radiation at 140° C. for 1 hour and then allowed to cool to RT. The mixture was diluted with EtOAc (100 ml) and washed with water (100 ml). The organic phase was separated, filtered through Celite® (filter material) dried (MgSO₄) and concentrated in vacuo to yield a brown oil/solid. Purification by chromatography on silica eluting with MeOH/DCM yielded a yellow oil/solid. This was passed through a 500 mg Isolute® Si-TMT cartridge (2,4,6-trimercaptotriazine silica, pre-wetted with DCM) eluting with 30% MeOH/DCM (50 ml) to afford a yellow oil/solid. The crude product was dried in vacuo and slurried in ˜0.5 ml DCM. The resulting suspension was removed by filtration and the filtrate was evaporated to yield the title compound as a light yellow/brown foam-like solid; LC-MS Rt=5.30 mins [M+H]+ 372 (Method 10 minLC_v002). 1H NMR (400 MHz, DMSO-d6), δ 8.29 (1H, t), 7.69 (1H, s), 7.49 (2H, t), 7.29 (2H, t), 7.22 (2H, s), 4.63 (1H, s), 3.24 (2H, d), 1.08 (6H, s).

### Example 12: 3-Amino-6-methoxy-5-trifluoromethyl-pyridine-2-carboxylic acid (3,3,3-trifluoro-2-hydroxy-propyl)-amide

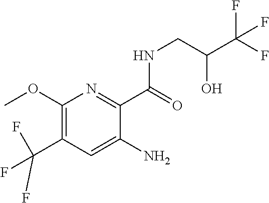 [SMILES: COc1nc(C(=O)NCC(O)C(F)(F)F)c(N)cc1C(F)(F)F]

### Step 1: 3-(2,5-Dimethyl-pyrrol-1-yl)-6-methoxy-5-trifluoromethyl-pyridine-2-carboxylic acid (3,3,3-trifluoro-2-hydroxy-propyl)-amide

This compound was prepared from 3-(2,5-Dimethyl-pyrrol-1-yl)-6-methoxy-5-trifluoromethyl-pyridine-2-carboxylic acid (Intermediate D2) and 3-amino-1,1,1-trifluoropropan-2-ol analogously to Example 1; LC-MS Rt=1.50 mins [M+H]+ 426 (Method 2 minLC_v002).

### Step 2: 3-Amino-6-methoxy-5-trifluoromethyl-pyridine-2-carboxylic acid (3,3,3-trifluoro-2-hydroxy-propyl)-amide

3-(2,5-Dimethyl-pyrrol-1-yl)-6-methoxy-5-trifluoromethyl-pyridine-2-carboxylic acid (3,3,3-trifluoro-2-hydroxy-propyl)-amide (350 mg, 0.823 mmol) was dissolved in EtOH (14 ml) and water (7 ml). Hyroxylamine hydrochloride (572 mg, 8.23 mmol) was added followed by TEA (167 mg, 1.646 mmol) and the mixture was heated at reflux overnight. After cooling the RT, the mixture was purified by reverse phase chromatography eluting with MeOH; water (0.1% TFA) to afford the title compound as a pale yellow solid; LC-MS Rt=4.20 min [M+H]+ 348.2 (Method 10 minLC_v002). 1H NMR (400 MHz, DMSO-d6) δ 8.47 (NH, t), 7.66 (1H, s), 6.68 (NH₂, b s), 6.51 (OH, d), 4.27-4.20 (1H, m), 3.93 (3H, s), 3.64-3.58 (1H, m), 3.44-3.37 (1H, m) 19F NMR (400 MHz, DMSO-d6) d −62.67 (CF₃, s), −77.05 (CF₃, s), Trace TFA.

### Example 14: 5-Amino-6′-methyl-3-trifluoromethyl-[2,3′]bipyridinyl-6-carboxylic acid (3,3,3-trifluoro-2-hydroxy-2-trifluoromethyl-propyl)-amide

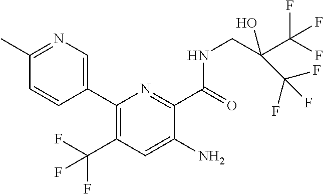 [SMILES: Cc1ccc(-c2nc(C(=O)NCC(O)(C(F)(F)F)C(F)(F)F)c(N)cc2C(F)(F)F)cn1]

This compound was prepared from 3-Amino-6-bromo-5-trifluoromethyl-pyridine-2-carboxylic acid (3,3,3-trifluoro-2-hydroxy-2-trifluoromethyl-propyl)-amide (Ex. 1.28) and 2-methylpyridine-5-boronic acid analogously to Example 8. LC-MS Rt 1.28 min; 477[M+H]+; (Method 2 minLC_v002); ¹H NMR (400 MHz, MeOD) δ 8.50 (1H, s), 7.85 (1H, dd), 7.69 (1H, s), 7.40 (1H, d), 4.00 (2H, s), 2.62 (3H, s).

### Example 15: 5-Amino-6′-methyl-3-trifluoromethyl-[2,3′]bipyridinyl-6-carboxylic acid (3,3,3-trifluoro-2-hydroxy-2-methyl-propyl)-amide

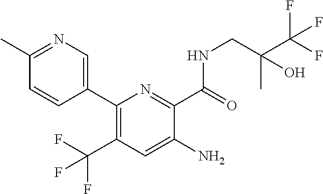 [SMILES: Cc1ccc(-c2nc(C(=O)NCC(C)(O)C(F)(F)F)c(N)cc2C(F)(F)F)cn1]

This compound was prepared by chiral separation of 5-amino-6′-methyl-3-trifluoromethyl-[2,3′]bipyridinyl-6-carboxylic acid (3,3,3-trifluoro-2-hydroxy-2-methyl-propyl)-amide (Example 1.29) using Supercritical Fluid Chromatography; LC-MS Rt 3.15 min [M+H]+ 423; (Method 10 minLC_v002); 1H NMR (400 MHz, DMSO-d6) δ 8.53 (1H, s), 8.49 (1H, t), 7.75 (1H, d), 7.71 (1H, s), 7.35 (1H, d), 7.25 (2H, s), 6.22 (1H, s), 3.69 (1H, dd), 3.42 (1H, dd), 2.54 (3H, s), 1.22 (3H, s). SFC Retention Time: 4.87 min.

### Examples 16 and 17: 3-Amino-5,6-bis-trifluoromethyl-pyridine-2-carboxylic acid ((S)-3,3,3-trifluoro-2-hydroxy-2-methyl-propyl)-amide and 3-Amino-5,6-bis-trifluoromethyl-pyridine-2-carboxylic acid ((R)-3,3,3-trifluoro-2-hydroxy-2-methyl-propyl)-amide

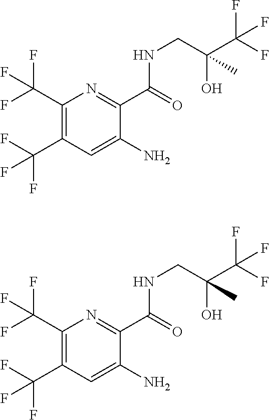 [SMILES: C[C@](O)(CNC(=O)c1nc(C(F)(F)F)c(C(F)(F)F)cc1N)C(F)(F)F ; C[C@@](O)(CNC(=O)c1nc(C(F)(F)F)c(C(F)(F)F)cc1N)C(F)(F)F]

### Step 1: 3-(2,5-Dimethyl-pyrrol-1-yl)-5,6-bis-trifluoromethyl-pyridine-2-carboxylic acid (3,3,3-trifluoro-2-hydroxy-2-methyl-propyl)-amide

To a stirred solution of 3-(2,5-dimethyl-pyrrol-1-yl)-5,6-bis-trifluoromethyl-pyridine-2-carboxylic acid (Intermediate M) (1.16 g, 3.29 mmol) in NMP (32 ml) was added 3-Amino-1,1,1-trifluoro-2-methyl-propan-2-ol hydrochloride (commercially available) (591 mg, 3.29 mmol) followed by HATU (1.25 g, 3.29 mmol) and NEt₃ (918 ul, 6.59 mmol) and the reaction mixture was left to stir at RT. After 1 h a further 0.2 equiv. NEt₃ was added. After 15 min a further 0.4 equiv. NEt₃ and 0.2 equiv. amine were added. After 30 min a further 0.1 equiv HATU was added. After 30 min most of the starting material had been consumed. The reaction mixture was added to EtOAc (50 ml), washed with 0.1M NaOH and the aqueous layer was back extracted with EtOAc (2×50 ml). The combined organic extracts were washed with water (2×150 ml), brine (100 ml), dried (MgSO₄) and concentrated in vacuo to give the crude product as an orange oil.

The crude material was purified by chromatography on silica eluting with 0-15% EtOAc in iso-hexane to afford the title product as a yellow solid; LC-MS Rt 1.32 min; MS m/z 478.2 [M+H]+; Method 2 minLC_v003.

### Step 2: 3-Amino-5,6-bis-trifluoromethyl-pyridine-2-carboxylic acid (3,3,3-trifluoro-2-hydroxy-2-methyl-propyl)-amide

To a stirred solution of 3-(2,5-dimethyl-pyrrol-1-yl)-5,6-bis-trifluoromethyl-pyridine-2-carboxylic acid (3,3,3-trifluoro-2-hydroxy-2-methyl-propyl)-amide (985 mg, 2.064 mmol) in 2:1 EtOH/H₂O (7.5 ml) was added hydroxylamine hydrochloride (1.43 g, 20.64 mmol) followed by NEt₃ (575 ml, 4.13 mmol). The reaction mixture was heated to reflux (˜98° C.) for 11.5 hours and then allowed to cool to RT. The solvent was removed in vacuo and the resulting residue was partitioned between EtOAc (25 ml) and water (25 ml). The aqueous layer was separated and extracted with EtOAc (2×25 ml) and the combined organic extracts were washed with brine (50 ml), dried (MgSO₄) and concentrated in vacuo. The crude material was purified by chromatography on silica eluting with 0-25% EtOAc in iso-hexane to afford the title product as a pale yellow solid; LC-MS: Rt 1.24 min; MS m/z 400.0 [M+H]+; Method 2 minLC_v003.

### Step 3: 3-Amino-5,6-bis-trifluoromethyl-pyridine-2-carboxylic acid ((S)-3,3,3-trifluoro-2-hydroxy-2-methyl-propyl)-amide and 3-Amino-5,6-bis-trifluoromethyl-pyridine-2-carboxylic acid ((R)-3,3,3-trifluoro-2-hydroxy-2-methyl-propyl)-amide

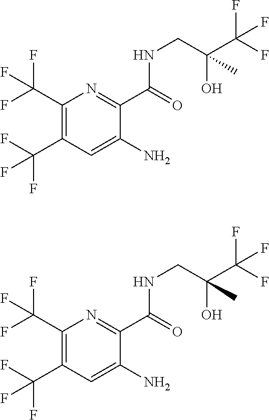 [SMILES: C[C@](O)(CNC(=O)c1nc(C(F)(F)F)c(C(F)(F)F)cc1N)C(F)(F)F ; C[C@@](O)(CNC(=O)c1nc(C(F)(F)F)c(C(F)(F)F)cc1N)C(F)(F)F]

These compounds were prepared by chiral separation of 3-amino-5,6-bis-trifluoromethyl-pyridine-2-carboxylic acid (3,3,3-trifluoro-2-hydroxy-2-methyl-propyl)-amide; Enantiomer 1: LC-MS Rt 1.23 min; MS m/z 400.0 [M+H]+; Method 2 minLC_v003. SFC Retention Time 5.07 min.

Enantiomer 2: LC-MS Rt 1.23 min; MS m/z 400.0 [M+H]+; Method 2 minLC_v003. SFC Retention Time 5.13 min.

### Example 18: 3-Amino-6-methoxy-N-(3,3,3-trifluoro-2-(4-methoxybenzylamino)-2-methylpropyl)-5-(trifluoromethyl)picolinamide

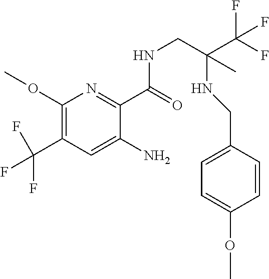 [SMILES: COc1ccc(CNC(C)(CNC(=O)c2nc(OC)c(C(F)(F)F)cc2N)C(F)(F)F)cc1]

The title compound was prepared analogously to Example 1 from 3-amino-6-methoxy-5-trifluoromethyl-pyridine-2-carboxylic acid (Intermediate D) and 3,3,3-trifluoro-N2-(4-methoxybenzyl)-2-methylpropane-1,2-diamine (Intermediate N). DIPEA was used in this reaction. 1H NMR (400 MHz, DMSO-d6) δ 8.27 (1H, m), 7.68 (1H, s), 7.25 (2H, d), 6.83 (2H, d), 6.70 (2H, s), 3.85 (3H, s), 3.75 (2H, m), 3.72 (3H, s), 3.70 (1H, m), 3.47 (1H, m), 2.80 (1H, t), 1.24 (3H, s)

### Example 19: 3-Amino-N-(2-amino-3,3,3-trifluoro-2-methylpropyl)-6-methoxy-5-(trifluoromethyl) picolinamide

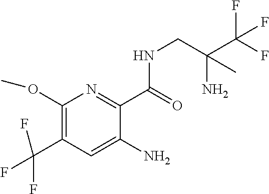 [SMILES: COc1nc(C(=O)NCC(C)(N)C(F)(F)F)c(N)cc1C(F)(F)F]

A mixture comprising 3-amino-6-methoxy-N-(3,3,3-trifluoro-2-(4-methoxybenzylamino)-2-methylpropyl)-5-(trifluoromethyl)picolinamide (Ex. 18) (0.9 g, 1.873 mmo) in TFA (50 ml) was heated to 50° C. for 2 h. After cooling to RT, the pH was adjusted to pH 12 using 2M NaOH. The product was extracted with DCM and the organic extract was washed with water, dried over MgSO₄ and concentrated in vacuo. The crude product was loaded onto a SCX-2 cartridge eluting with MeOH followed by 2M NH₃ in MeOH. The methanolic ammonia fractions were concentrated in vacuo and dried under vacuum to afford the title compound. 1H NMR (400 MHz, DMSO-d6) δ 8.35 (1H, m), 7.67 (1H, s), 6.67 (2H, s), 3.93 (3H, s), 3.58 (1H, m), 3.40 (1H, m), 2.22 (2H, s), 1.14 (3H, s). LC-MS Rt 0.94 min; MS m/z 361.2 [M+H]+; Method 2 minLC_v003.

### Example 20: 3-Amino-6-(pyrrolidin-1-yl)-N-(3,3,3-trifluoro-2-hydroxy-2-methylpropyl)-5-(trifluoromethyl)picolinamide

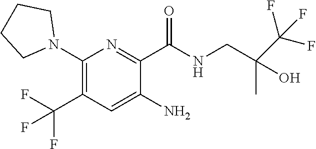 [SMILES: CC(O)(CNC(=O)c1nc(N2CCCC2)c(C(F)(F)F)cc1N)C(F)(F)F]

### Step 1: 3-(2,5-Dimethyl-1H-pyrrol-1-yl)-6-(pyrrolidin-1-yl)-N-(3,3,3-trifluoro-2-hydroxy-2-methylpropyl)-5-(trifluoromethyl)picolinamide

The title compound was prepared from Intermediate DA analogously to Example 1; LC-MS Rt 1.42 min; MS m/z 479.3 [M+H]+; Method 2 minLC_v003.

### Step 2: 3-Amino-6-(pyrrolidin-1-yl)-N-(3,3,3-trifluoro-2-hydroxy-2-methylpropyl)-5-(trifluoromethyl)picolinamide

This compound was prepared from 3-(2,5-dimethyl-1H-pyrrol-1-yl)-6-(pyrrolidin-1-yl)-N-(3,3,3-trifluoro-2-hydroxy-2-methylpropyl)-5-(trifluoromethyl)picolinamide analogously to Intermediate D (final step). The resulting racemate was separated by SFC to afford the title compound; First eluted peak: 1H NMR (400 MHz, DMSO-d6) δ 8.24 (1H, m), 7.6 (1H, s), 6.4 (2H, br s), 6.32 (1H, s), 3.64 (1H, m), 3.48 (1H, m), 3.35 (4H), 1.88 (4H, m), 1.25 (3H, s); LC-MS Rt 3.87 min; MS m/z 401.3 [M+H]+; Method 10 minLC_v003.

### Example 21: (S)-3-amino-6-ethoxy-N-(3,3,3-trifluoro-2-hydroxy-2-methylpropyl)-5-(trifluoro methyl)picolinamide

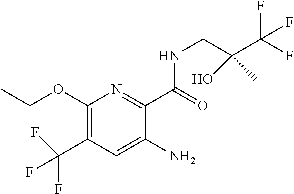 [SMILES: CCOc1nc(C(=O)NC[C@](C)(O)C(F)(F)F)c(N)cc1C(F)(F)F]

The title compound was prepared from Intermediate DB and Intermediate R analogously to Example 20; 1H NMR (400 MHz, DMSO-d6) δ 8.3 (1H, t), 7.7 (1H, s), 6.6 (2H, broad), 6.3 (1H, s), 4.4 (2H, q), 3.6 (1H, mult), 3.5 (1H, mult), 1.3 (3H, t), 1.2 (3H, s).

LC-MS Rt 1.20 min; MS m/z 376.2 [M+H]+; Method 2 minLC_v003.

### Example 22: 3-Amino-6-bromo-N-(2-morpholinoethyl)-5-(trifluoromethyl)pyrazine-2-carboxamide

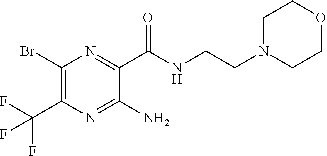 [SMILES: Nc1nc(C(F)(F)F)c(Br)nc1C(=O)NCCN1CCOCC1]

To a stirred solution of 3-amino-6-bromo-5-trifluoromethyl-pyrazine-2-carboxylic acid (Intermediate C) (250 mg, 0.874 mmol) in NMP (8 ml) was added 4-(2-aminoethyl)morphonline (138 ul, 1.049 mmol) followed by DIPEA (763 ul, 4.37 mmol). To this solution was then added HATU (499 mg, 1.311 mmol) in portions and the reaction mixture was left to stir at RT for 1 hour. A further 1 equiv. of 4-(2-aminoethyl)morphonline was added. After a further 1.5 hr, 0.5 equiv. HATU (166 mg, 0.425 mmol) was added and the RM was left to stir for a further 30 min. The mixture was added to EtOAc (50 ml) and washed with 0.1M NaOH (50 ml). The aqueous layer was back extracted with EtOAc (50 ml). The combined organics were washed with water (50 ml), brine (50 ml), dried over magnesium sulfate and evaporated under reduced pressure to give a brown oil (418 mg). The crude product was purified by chromatography (Biotage-silica 20 g/70 ml column, 3:1 EtOAc/iso-hexane). The resulting yellow residue was loaded onto an SCX-2 cartridge (10 g) that had been pre-wetted with MeOH. The cartridge was washed with MeOH (140 ml) and eluted with 3.5M ammonia in methanol solution (70 ml). The appropriate fractions were evaporated under reduced pressure to give a solid. This solid was dissolved in EtOAc and filtered under vacuum. The filtrate was evaporated under reduced pressure and then dried in vacuo to afford the title compound as a yellow solid; LC-MS: Rt 2.61 min; MS m/z 398.2 [M+H]+; Method 10 minLC_v002; 1H NMR (400 MHz, DMSO-d6) δ 8.70 (1H, s), 8.10 (2H, s), 3.58 (4H. t), 3.40 (2H, q), 2.45 (2H, m), 2.40 (4H, s).

### Example 23: N-(2-(1H-imidazol-2-yl)propyl)-3-amino-6-bromo-5-(trifluoromethyl)pyrazine-2-carboxamide

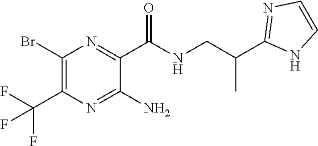 [SMILES: CC(CNC(=O)c1nc(Br)c(C(F)(F)F)nc1N)c1ncc[nH]1]

The title compound was prepared from 3-amino-6-bromo-5-trifluoromethyl-pyrazine-2-carboxylic acid (Intermediate C) and 2-(1H-imidazol-2-yl)propan-1-amine (prepared according to the procedure of Steffens, Robert; Schunack, Walter. Histamine analogs, XXVI. Racemic histamine H1-agonists. Archiv der Pharmazie (Weinheim, Germany) (1984), 317(9), 771-6; 1H NMR (400 MHz, DMSO-d6) δ 11.8 (1H, s), 9.0 (1H, t), 8.1 (2H, s), 7.0 (1H, s), 6.8 (1H, s), 3.55 (2H, m), 3.15 (1H, m), 1.2 (3H, d). LC-MS [M+H]+ 393.0/395.1

### Examples 24a and 24b: Enantiomers of 3-amino-N-(3,3,3-trifluoro-2-hydroxy-2-methylpropyl)-5,6-bis(trifluoromethyl) pyrazine-2-carboxamide

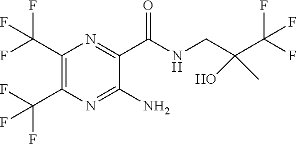 [SMILES: CC(O)(CNC(=O)c1nc(C(F)(F)F)c(C(F)(F)F)nc1N)C(F)(F)F]

The title compound was prepared from Intermediate BA and 3-amino-1,1,1-trifluoro-2-methylpropan-2-ol analogously to Example 4. Chiral separation of the racemate by Supercritical Fluid Chromatography afforded the title compound.

Example 24a: First eluted peak: Enantiomer 1 of 3-amino-N-(3,3,3-trifluoro-2-hydroxy-2-methylpropyl)-5,6-bis(trifluoromethyl) pyrazine-2-carboxamide; 1H NMR (400 MHz, DMSO-d6) δ 8.61-8.74 (1H, broad hump), 8.5-8.61 (1H, broad hump), 8.46 (1H, t), 6.3 (1H, s), 3.69 (1H, m), 3.5 (1H, m), 1.29 (3H, s)

LC-MS: Rt 4.23 min; MS m/z 401.2 [M+H]+; Method 10 minLC_v003.

Example 24b: Second eluted peak: Enantiomer 2 of 3-amino-N-(3,3,3-trifluoro-2-hydroxy-2-methylpropyl)-5,6-bis(trifluoromethyl) pyrazine-2-carboxamide; 1H NMR (400 MHz, DMSO-d6) δ 8.61-8.76 (1H, broad hump), 8.5-8.60 (1H, broad hump), 8.46 (1H, t), 6.3 (1H, s), 3.69 (1H, m), 3.5 (1H, m), 1.29 (3H, s); LC-MS: Rt 4.24 min; MS m/z 401.2 [M+H]+; Method 10 minLC_v003. Optical rotation [α]²¹_{D} at 589 nm+22.0° (c=0.517, MeOH).

### Example 25: 3-Amino-6-(1-methyl-1H-pyrazol-4-yl)-N-(3,3,3-trifluoro-2-hydroxy-2-methylpropyl)-5-(trifluoromethyl)picolinamide

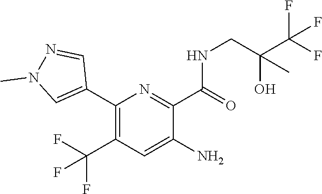 [SMILES: Cn1cc(-c2nc(C(=O)NCC(C)(O)C(F)(F)F)c(N)cc2C(F)(F)F)cn1]

### Step 1: 3-Amino-6-(1-methyl-1H-pyrazol-4-yl)-5-(trifluoromethyl)picolinic acid

3-Amino-6-bromo-5-trifluoromethyl-pyridine-2-carboxylic acid methyl ester (Intermediate A4) (500 mg, 1.672 mmol), PdCl₂(dppf).CH₂Cl₂ adduct (205 mg, 0.251 mmol), 1-methyl-4-(4,4,5,5-tetramethyl-1,3,2-dioxaborolan-2-yl)-1H-pyrazole (383 mg, 1.839 mmol) and Cs₂CO₃ (6.69 ml, 6.69 mmol) in THF (12 ml) under N2, was heated using microwave radiation at 150° C. for 10 minutes. 2M NaOH (5 ml) was added and the mixture was stirred at RT overnight. The mixture was filtered through Celite® (filter material) and the organic solvent was removed. The resulting aqueous layer was washed with EtOAc and acidified to pH1. The product was extracted with DCM and concentrated in vacuo to afford the title compound.

### Step 2: 3-Amino-6-(1-methyl-1H-pyrazol-4-yl)-N-(3,3,3-trifluoro-2-hydroxy-2-methylpropyl)-5-(trifluoromethyl)picolinamide

The title compound was prepared from 3-amino-6-(1-methyl-1H-pyrazol-4-yl)-5-(trifluoro methyl)picolinic acid and 3-amino-1,1,1-trifluoro-2-methylpropan-2-ol analogously to Example 4 1H NMR (400 MHz, Methanol-d4) δ 7.97 (1H, s), 7.85 (1H, s), 7.60 (1H, s), 3.97 (3H, s), 3.77 (1H, m), 3.56 (1H, m), 1.37 (3H, s); LC-MS: Rt 3.22 min; MS m/z 412.3 [M+H]+; Method 10 minLC_v003.

### Example 26: 3-Amino-6-furan-2-yl-5-trifluoromethyl-pyrazine-2-carboxylic acid [2-(2-methoxy-phenyl)-ethyl]-amide

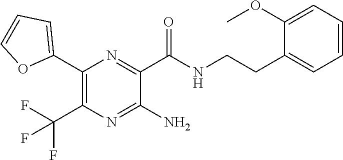 [SMILES: COc1ccccc1CCNC(=O)c1nc(-c2ccco2)c(C(F)(F)F)nc1N]

The title compound was prepared from 3-amino-6-furan-2-yl-5-trifluoromethyl-pyrazine-2-carboxylic acid (Intermediate PA) and the appropriate amine; MS m/z 406.93 [M+H]+

### Preparation of Intermediates

### Intermediate A: 3-Amino-6-bromo-5-trifluoromethyl-pyridine-2-carboxylic acid

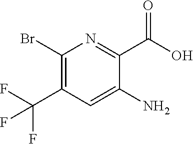 [SMILES: Nc1cc(C(F)(F)F)c(Br)nc1C(=O)O]

### Intermediate A1: 2-Bromo-3-nitro-5-trifluoromethyl-pyridine

3-Nitro-5-(trifluoromethyl)pyridin-2-ol (31.00 g, 149 mmol) was dissolved in acetonitrile (250 ml) to give a dark brown solution. Phosphorus(V) oxybromide (85 g, 298 mmol) was added and the mixture was heated at reflux for 4.5 hours and then stirred at RT overnight. The reaction mixture was quenched by pouring into vigorously stirring water (600 ml) containing sodium hydrogencarbonate (110 g). The dark brown mixture was extracted with DCM (3×200 ml) and the organic phase was washed with water (200 ml) and brine (100 ml), dried (MgSO₄) and concentrated in vacuo to afford the title product as a brown oil. ¹H-NMR: [400 MHz, CDCl₃, δ_{H} 8.87 (1H, d, J=1.4 Hz, ArH), 8.39 (1H, d, J=1.9 Hz, ArH).

### Intermediate A2: 3-Nitro-5-trifluoromethyl-pyridine-2-carbonitrile

2-Bromo-3-nitro-5-trifluoromethyl-pyridine (10.00 g, 36.87 mmol) was dissolved in toluene (250 ml) with stirring to give a pale yellow solution. Tetrabutylammonium bromide (11.90 g, 36.9 mmol) was added followed by copper(I) cyanide (9.92 g, 111 mmol) and the mixture was heated at reflux for 10 h. After cooling to RT, the reaction mixture was partitioned between water (750 ml) and EtOAc (750 ml). The organic fractions were combined, washed with water (2×250 ml) and brine (100 ml), dried (MgSO₄) and concentrated in vacuo to afford the title product. ¹H-NMR: [400 MHz, DMSO-d₆ δ_{H} 9.55 (1H, m, ArH), 9.24 (1H, m, ArH).

### Intermediate A3: 3-Amino-5-trifluoromethyl-pyridine-2-carboxylic acid methyl ester

3-Nitro-5-trifluoromethyl-pyridine-2-carbonitrile (6.5 g, 29.9 mmol) was dissolved in EtOAc (150 ml) to give a pale yellow solution and placed under an atmosphere of nitrogen. 10% Palladium on activated carbon (3.19 g, 2.99 mmol) was added and the reaction mixture stirred under an atmosphere of hydrogen for 18 hours. The reaction mixture was filtered and concentrated in vacuo. The crude residue was dissolved in HCl conc. (45 ml) and heated to reflux for 24 hours. The reaction mixture was allowed to cool to RT and concentrated in vacuo. The solid was dissolved in MeOH (300 ml) and sulfuric acid (14.4 ml) was added. The resulting solution was heated at reflux for 48 hours. The reaction was allowed to cool to RT, then neutralised by addition of 10% NaHCO_{3(aq)} (600 ml). The product was extracted into DCM (3×200 ml) and the combined organic phases were washed with water (200 ml), brine (50 ml), (MgSO₄) and concentrated in vacuo. The resulting solid was purified by chromatography on silica: Eluant gradient: isohexane (500 ml), 10% EtOAc in isohexane (1000 ml), 20% EtOAc in isohexane (1500 ml) to afford the titled compound as a pale yellow solid ¹H-NMR: [400 MHz, DMSO-d₆, δ_{H} 8.13 (1H, d, J=1.7 Hz, ArH), 7.60 (1H, d, J=1.3 Hz, ArH), 7.01 (2H, br, NH₂), 3.85 (3H, s, ArOCH₃), m/z 221.1 [M+H]⁺

### Intermediate A4: 3-Amino-6-bromo-5-trifluoromethyl-pyridine-2-carboxylic acid methyl ester

3-Amino-5-trifluoromethyl-pyridine-2-carboxylic acid methyl ester (9.49 g, 43.16 mmol) was suspended in water (300 ml). Sulfuric acid (4.60 ml, 86 mmol) was added followed by dropwise addition over 30 minutes of a solution of bromine (2.222 ml, 43.1 mmol) in acetic acid (29.6 ml, 517 mmol). The reaction mixture was stirred at RT for 18 hours. A further 100 ml of water was added, followed by a further 0.25 equivalents of the bromine/AcOH mixture (550 μL bromine in 7.4 ml AcOH) and the reaction mixture stirred at RT for an additional 90 minutes. The reaction mixture was diluted with 500 ml water and neutralised by addition of solid NaHCO₃ (˜85 g). The suspension was extracted with DCM (3×300 ml) and the combined organic phases washed with sat.NaHCO_{3(aq)} (250 ml), water (250 ml) and brine (100 ml), dried (MgSO₄) and concentrated in vacuo. The crude material was recrystallised from boiling MeOH (˜300 ml) to give the title product as a pale orange solid m/z 301.0 [M+H]^{+ 1}H-NMR: [400 MHz, DMSO-d₆ δ_{H} 7.77 (1H, s, ArH), 7.17 (2H, s, NH₂), 3.86 (3H, s, ArCO₂CH₃).

### Intermediate A: 3-Amino-6-bromo-5-trifluoromethyl-pyridine-2-carboxylic acid

3-Amino-6-bromo-5-trifluoromethyl-pyridine-2-carboxylic acid methyl ester (1.40 g, 4.68 mmol) was suspended in MeOH (15 ml); Sodium hydroxide (2.0 M aqueous solution) (14.04 ml, 28.1 mmol) was added and the suspension was stirred at RT overnight. The mixture was concentrated in vacuo and the resulting residue was dissolved in water (100 ml) and then acidifed by the addition of 5.0M HCl(aq). The product was extracted into ethyl acetate (2×75 ml) and the combined organic extracts were washed with water (50 ml), brine (25 ml), dried (MgSO₄) and concentrated in vacuo to afford the title product as a yellow solid. ¹H-NMR: [400 MHz, DMSO-d6, 6H 13.24 (1H, br s, CO₂H), 7.74 (1H, s, ArH), 7.17 92H, br s ArNH₂). m/z 285.1, 287.1 [M+H]⁺

### Intermediate B: 3-Amino-5-trifluoromethyl-pyrazine-2-carboxylic acid ethyl ester

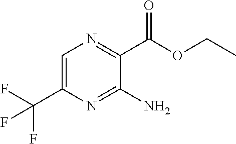 [SMILES: CCOC(=O)c1ncc(C(F)(F)F)nc1N]

### Intermediate B1: Carbamimidoyl-nitroso-acetic acid ethyl ester

To a solution of 2M ammonia in Ethanol (152 ml, 0.304 mmol) at 0° C. to 5° C., ethyl ethoxycarbonylacetimidate HCl (25 g, 0.127 mmol) was added over 30 minutes. The reaction was stirred vigorously at this temperature for 3 hours, after which a solution of sodium nitrite in water (9.63 g, 0.139 mmol) was added in a single portion. The pH of the mixture was adjusted to pH6 with the addition of 5N HCl. The reaction mixture was left to stir at RT overnight. The yellow precipitate formed was filtered under vacuum, washed with water and dried to give the title compound. ¹H NMR (400 MHz, DMSO-d6) δ 10.1 (2H, br s), 7.6 (2H, br s), 4.3 (2H, q), 1.3 (3H, t).

### Intermediate B2: Amino-Carbamimidoyl-Acetic Acid Ethyl Ester

To a solution of carbamimidoyl-nitroso-acetic acid ethyl ester (5.5 g, 31.4 mmol) in ethanol/5M HCl (1:1 ratio, 250 ml) was added 10% Pd/C (1.3 g). The reaction mixture was hydrogenated (H_{2(g)}) at low pressure over 2 nights. The Pd/C was filtered through Celite®(filter material) and the filtrate reduced in vacuo to give the title compound as a white solid. This was taken through to the next step as crude.

### Intermediate B: 3-Amino-5-trifluoromethyl-pyrazine-2-carboxylic Acid Ethyl Ester

To a mixture of amino-carbamimidoyl-acetic acid ethyl ester (2 g, 9.22 mmol) and water (50 ml), a 20% aqueous solution of trifluoropyruvic aldehyde (2.32 g, 18.43 mmol) was added. To this mixture, sodium acetate (5.29 g, 64.52 mmol) was added (pH of the reaction mixture was pH5). The reaction mixture was left to stir at RT overnight. The resultant precipitate was filtered under vacuum purification by chromatography on silica eluting with iso-hexane: EtOAc (gradient of 0 to 10% EtOAc) afforded the title compound. ¹H NMR (400 MHz, DMSO-d6) δ 8.4 (1H, s), 7.8 (2H, br s), 4.4 (2H, q), 1.4 (3H, t).

### Intermediate BA: 3-Amino-5,6-bis(trifluoromethyl)pyrazine-2-carboxylic acid

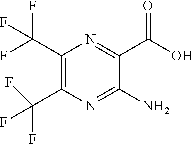 [SMILES: Nc1nc(C(F)(F)F)c(C(F)(F)F)nc1C(=O)O]

### Step 1: Ethyl 3-amino-5,6-bis(trifluoromethyl)pyrazine-2-carboxylate

The title compound was prepared from amino-carbamimidoyl-acetic acid ethyl ester (Intermediate B2) and 1,1,1,4,4,4-hexafluorobutane-2,3-dione analogously to Intermediate B; 10 LCMS Rt=4.72 minutes, [M+H]+ 304.2/326.1 Method 10 minLC_v002.

### Step 2: 3-Amino-5,6-bis(trifluoromethyl)pyrazine-2-carboxylic acid

To a stirring solution of ethyl 3-amino-5,6-bis(trifluoromethyl)pyrazine-2-carboxylate (300 mg, 0.990 mmol) in EtOH (10 ml), 2M NaOH (0.495 ml, 0.990 mmol) was added dropwise over 1 minute. After stirring at RT for 30 minutes the reaction mixture was poured into water (30 ml) and the pH was adjusted to pH 4 by addition of 1M HCl. The mixture was extracted with EtOAc (2×50 ml) and the combined organic extracts were washed with brine (30 ml), dried over MgSO₄ (5 g), filtered and concentrated in vacuo to afford the title compound as an off white crystalline solid. ¹H NMR (400 MHz, DMSO-d6) δ 8.6-9.2 (2H, broad hump), 7.8-8.3 (2H, broad hump), 4.4 (2H, q), 1.32 (3H, t).

### Intermediate C: 3-Amino-6-bromo-5-trifluoromethyl-pyrazine-2-carboxylic acid

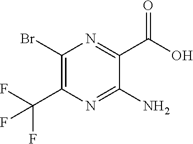 [SMILES: Nc1nc(C(F)(F)F)c(Br)nc1C(=O)O]

### Intermediate C1: 3-Amino-6-bromo-5-trifluoromethyl-pyrazine-2-carboxylic acid ethyl ester

To a solution of 3-amino-5-trifluoromethyl-pyrazine-2-carboxylic acid ethyl ester (Intermediate B) (30 mg, 0.13 mmol) in acetic acid (5 ml), sodium carbonate (15 mg, 0.14 mmol) was added. To this mixture, half the contents of a solution of bromine (7 μL, 0.13 mmol) in acetic acid (5 ml) were added, followed by the addition of sodium carbonate ((15 mg, 0.14 mmol). The remaining solution of bromine in acetic acid was added and the reaction mixture was left to stir at RT for 2 hours. The mixture was diluted with water and the resulting yellow precipitate was filtered under vacuum to afford the title compound.

### Intermediate C: 3-Amino-6-bromo-5-trifluoromethyl-pyrazine-2-carboxylic acid

To a stirring solution of 3-amino-5-trifluoromethyl-pyrazine-2-carboxylic acid ethyl ester (10 g, 31.8 mmol) in ethanol (20 ml), 2M NaOH (20 ml, 31.8 mmol) was added. The resulting solution was stirred at RT for 5 minutes and poured into water (50 ml). The pH was adjusted to pH6 with the addition of 1M HCl. The resulting suspension was filtered under vacuum, washed with water (20 ml) and dried to afford the title compound. MS m/z 287[M+H]t ¹H NMR (400 MHz, DMSO-d6) δ 7.98 (2H, s).

### Intermediate D: 3-Amino-6-methoxy-5-trifluoromethyl-pyridine-2-carboxylic acid

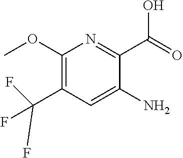 [SMILES: COc1nc(C(=O)O)c(N)cc1C(F)(F)F]

### Intermediate D1: 6-Bromo-3-(2,5-dimethyl-pyrrol-1-yl)-5-trifluoromethyl-pyridine-2-carboxylic acid methyl ester

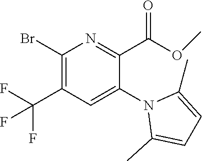 [SMILES: COC(=O)c1nc(Br)c(C(F)(F)F)cc1-n1c(C)ccc1C]

3-Amino-6-bromo-5-trifluoromethyl-pyridine-2-carboxylic acid methyl ester (Intermediate A4) (2 g, 6.69 mmol) was suspended in toluene (8 ml), and treated with p-toluenesulfonic acid (TsOH) (0.115 g, 0.669 mmol) and acetonylacetone (0.941 ml, 8.03 mmol). The reaction mixture was heated at reflux for 2 hours (using Dean-Stark apparatus) and allowed to cool to RT overnight. The resulting dark red/black solution was concentrated in vacuo to remove toluene and the crude residue diluted with EtOAc (200 ml), washed with NaHCO₃ (50 ml), dried (MgSO₄) and concentrated in vacuo to give a brown solid. Purification of the solid by chromatography on silica eluting with EtOAc/iso-hexane afforded the title compound; LC-MS Rt=5.58 min [M+H]+ 377/379 (Method 10 minLC_v002). 1H NMR (400 MHz, DMSO-d6) δ 8.50 (1H, s), 7.77 (2H, s), 5.83 (3H, s), 1.90 (6H, s); 19F NMR (400 MHz, DMSO-d6) δ −62.26 (CF₃, s).

### Intermediate D2: 3-(2,5-Dimethyl-pyrrol-1-yl)-6-methoxy-5-trifluoromethyl-pyridine-2-carboxylic acid

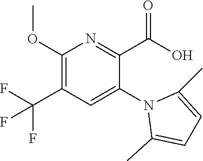 [SMILES: COc1nc(C(=O)O)c(-n2c(C)ccc2C)cc1C(F)(F)F]

6-Bromo-3-(2,5-dimethyl-pyrrol-1-yl)-5-trifluoromethyl-pyridine-2-carboxylic acid methyl ester (2 g, 5.30 mmol) was dissolved in MeOH (40 ml) and treated with 2M NaOH (20 ml) to give a suspension which was stirred at RT for 1 h to afford a clear solution. The solvent was removed in vacuo and the resulting residue was acidified to pH1 with 5M HCl. The mixture was extracted with EtOAc (200 ml) and the organic extract was dried (MgSO₄) and concentrated in vacuo to afford the title compound as a dark brown solid which was used in the next step without further purification; LC-MS Rt=1.50 min [M+H]+ 315.2.1/316.2 (Method 2 minLC_v002); 1H NMR (400 MHz, DMSO-d6) δ14.42-12.61 (COOH, b), 8.25 (1H, s), 5.84 (2H, s), 4.13 (3H, s), 1.97 (6H, s); 19F NMR (400 MHz, DMSO-d6) δ −62.43 (CF₃, s).

### Intermediate D: 3-Amino-6-methoxy-5-trifluoromethyl-pyridine-2-carboxylic acid

3-(2,5-Dimethyl-pyrrol-1-yl)-6-methoxy-5-trifluoromethyl-pyridine-2-carboxylic acid (2.1 g, 6.68 mmol) was dissolved in EtOH (40 ml) and water (20 ml). To this mixture was added TEA (2.79 ml, 20.05 mmol) followed by hydroxylamine hydrochloride (4.64 g, 66.8 mmol). The resulting mixture was heated at reflux for 5 hours. After cooling to RT, the mixture was diluted with EtOAc (100 ml) and washed with aqueous HCl (1M, 100 ml). The aqueous phase was back extracted with EtOAc (100 ml) and the combined organic phases washed with brine (100 ml), dried (MgSO₄) and concentrated in vacuo to afford the product as an orange solid. The material can be used crude or recrystallised from isohexane-EtOAc (10:1) LC-MS Rt=1.0 min [M+H]+ 237 (Method 2 minLC_v003)

1H NMR (400 MHz, DMSO-d6) δ 8.5 (NH₂, b), 7.70 (1H, s), 3.89 (3H, s).

### Intermediate DA: 3-(2,5-Dimethyl-1H-pyrrol-1-yl)-6-(pyrrolidin-1-yl)-5-(trifluoromethyl)picolinic acid

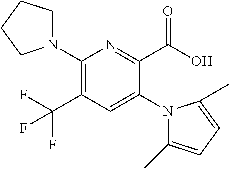 [SMILES: Cc1ccc(C)n1-c1cc(C(F)(F)F)c(N2CCCC2)nc1C(=O)O]

### Step 1: 6-Bromo-3-(2,5-dimethyl-1H-pyrrol-1-yl)-5-(trifluoromethyl)picolinic acid

6-Bromo-3-(2,5-dimethyl-pyrrol-1-yl)-5-trifluoromethyl-pyridine-2-carboxylic acid methyl ester (1.9 g, 5.04 mmol) and 2M NaOH (2.52 ml, 5.04 mmol) in THF (10 ml) was stirred at RT for 1 hour. The reaction mixture was poured into water (50 ml) and the pH was adjusted to pH 4 by addition of 1M HCl. The mixture was extracted with EtOAc (2×50 ml) and the organic portion was washed with brine (30 ml), dried over MgSO₄ (5 g), filtered and concentrated to give the title compound as a crystalline orange solid; LC MS Rt=1.21 min [M+H]+ 363.1 (Method 2 minLC_v003).

### Step 2: 3-(2,5-Dimethyl-1H-pyrrol-1-yl)-6-(pyrrolidin-1-yl)-5-(trifluoromethyl)picolinic acid

To a stirring solution of 6-bromo-3-(2,5-dimethyl-1H-pyrrol-1-yl)-5-(trifluoromethyl) picolinic acid (300 mg, 0.826 mmol) in THF (1 ml), pyrrolidine (0.136 ml, 1.652 mmol) was added. The orange solution was stirred at RT overnight. The reaction mixture was partitioned between 0.5M HCl (30 ml) and EtOAc (30 ml) and shaken. The organic portion was separted and washed with brine (30 ml), dried over MgSO₄, filtered and concentrated in vacuo to give a red oil. The crude product was purified on silica eluting with 0-40% EtOAc in iso-hexane to afford the title product. 1H NMR (400 MHz, DMSO d6) δ 13.45 (1H, br s), 7.88 (1H, s), 5.74 (2H, s), 3.58 (5H, br s), 1.88-2.0 (11H, unresolved peaks).

### Intermediate DB: 3-(2,5-Dimethyl-1H-pyrrol-1-yl)-6-ethoxy-5-(trifluoromethyl)picolinic acid

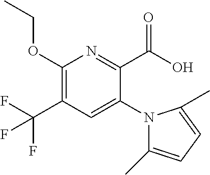 [SMILES: CCOc1nc(C(=O)O)c(-n2c(C)ccc2C)cc1C(F)(F)F]

### Step 1: Methyl 3-(2,5-dimethyl-1H-pyrrol-1-yl)-6-methoxy-5-(trifluoromethyl)picolinate

3-(2,5-Dimethyl-pyrrol-1-yl)-6-methoxy-5-trifluoromethyl-pyridine-2-carboxylic acid (Intermediate D2)(500 mg, 1.591 mmol) in methanol (15.91 ml) was treated with H2504 (0.0424 ml, 0.795 mmol) and the solution was heated at reflux for overnight.

The solvent removed was removed in vacuo and the resulting brown oil was neutralised to pH 7 using saturated sodium bicarbonate. The mixture was extracted with EtOAc (20 ml) and the combined organic extracts were washed with water (20 ml), brine (20 ml), passed though a phase separator and concentrated in vacuo. Purification of the crude product by chromatography on silica eluting with iso-hexane: EtOAc (gradient of 0 to 10% EtOAc) afforded the title compound as an off-white powder. 1H NMR (400 MHz, DMSO-d6) δ 8.3 (1H, s), 5.8 (2H, s), 4.1 (3H, s), 3.6 (3H, s), 1.9 (6H, s).

### Step 2: Methyl 3-(2,5-dimethyl-1H-pyrrol-1-yl)-6-hydroxy-5-(trifluoromethyl)picolinate

Methyl 3-(2,5-dimethyl-1H-pyrrol-1-yl)-6-methoxy-5-(trifluoromethyl)picolinate (100 mg, 0.305 mmol) in acetonitrile (3.05 ml) was treated with KI (202 mg, 1.218 mmol) and TMS-Chloride (0.156 ml, 1.221 mmol) and heated at reflux for 6 hours. The solvent removed was in vacuo and the crude product was dissolved in EtOAc (20 ml) and washed with water (2×10 ml) and brine (10 ml), dried over a phase separator and concentrated in vacuo. Purification of the crude product by chromatography on silica eluting with iso-hexane: EtOAc (gradient of 0 to 30% EtOAc) afforded the title compound as an yellow powder. LC-MS Rt=1.11 mins [M+H]+ 315.4 (Method 2 minLC_v003).

### Step 3: Methyl 3-(2,5-dimethyl-1H-pyrrol-1-yl)-6-ethoxy-5-(trifluoromethyl)picolinate

Methyl 3-(2,5-dimethyl-1H-pyrrol-1-yl)-6-hydroxy-5-(trifluoromethyl)picolinate (62 mg, 0.168 mmol) in 1,4-dioxane (1.5 ml) (dry) was treated with EtOH (0.020 ml, 0.335 mmol) and triphenylphosphine (88 mg, 0.335 mmol) and the solution stirred. DEAD (0.053 ml, 0.335 mmol) was added dropwise and the reaction mixture stirred at room temperature for 2 hours. The solvent was removed in vacuo and purification of the crude product by chromatography on silica eluting with iso-hexane: EtOAc (gradient of 0 to 10% EtOAc) afforded the title compound. 1H NMR (400 MHz, DMSO-d6) δ 8.3 (1H, s), 5.8 (2H, s), 4.5 (2H, q), 3.6 (3H, s), 1.9 (6H, s), 1.4 (3H, t).

### Step 4: 3-(2,5-Dimethyl-1H-pyrrol-1-yl)-6-ethoxy-5-(trifluoromethyl)picolinic acid

Methyl 3-(2,5-dimethyl-1H-pyrrol-1-yl)-6-ethoxy-5-(trifluoromethyl)picolinate (140 mg, 0.409 mmol) was dissolved in THF (2.045 ml). NaOH (0.613 ml, 1.226 mmol) was added and heated at reflux for 6 hours. The solvent was removed in vacuo and the resulting mixture was diluted with EtOAc (25 ml) was acidified to pH 1 using HCl (5M). The organic portion washed with brine, dried using a phase separator and concentrated in vacuo to afford the title compound as a yellow oil. LC-MS Rt=1.26 mins [M+H]+ 329.2 Method 2 minLC_v003.

### Intermediate E: 3-Amino-5-trifluoromethyl-pyridine-2-carboxylic acid

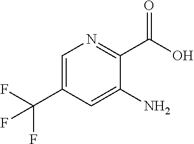 [SMILES: Nc1cc(C(F)(F)F)cnc1C(=O)O]

To a stirring solution of 3-Amino-5-trifluoromethyl-pyridine-2-carboxylic acid methyl ester (Intermediate A3) (1 g, 4.54 mmol) in MeOH (20 ml) was added 2M NaOH (0.182 g, 4.54 mmol). The orange solution was stirred at RT for 1 minute and then into water (10 ml). The solution was acidified to pH1 with the addition of 1M HCl and the product was extracted with EtOAc (150 ml). The organic portions were combined, washed with brine (50 ml), dried over MgSO₄ and concentrated in vacuo to afford the title compound as an orange solid; LC-MS Rt=0.82 mins [M+H]+ 207.1 (Method 2 minLC_v002); 1H NMR (400 MHz, DMSO-d6) δ 13.9 (1H, broad hump), 8.11 (1H, s), 7.59 (1H, s), 7.08 (2H, broad hump) (trace of EtOAc present but correlates to proposed structure).

### Intermediate G: 3-Amino-6-(4-fluoro-phenyl)-5-trifluoromethyl-pyridine-2-carboxylic acid

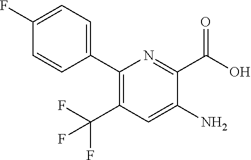 [SMILES: Nc1cc(C(F)(F)F)c(-c2ccc(F)cc2)nc1C(=O)O]

A mixture comprising 3-amino-6-bromo-5-trifluoromethyl-pyridine-2-carboxylic acid (Intermediate A) (1 g, 3.51 mmol), 4-fluorophenylboronic acid (0.736 g, 5.26 mmol) and 1,1′Bis(diphenylphosphoshio)ferrocene palladium dichloride (0.286 g, 0.351 mmol) and 1.0M Cs₂CO₃ (3.3 ml) in THF (10 ml) was heated to reflux for 10 hours. After cooling to RT, the mixture was partitioned between DCM (100 ml) and 1 M NaOH (2×100 ml). The aqueous phase was acidified with 5M HCl and the resulting milky solution was extracted into DCM (2×100 ml). The organic portion was separated, dried (MgSO₄) and concentrated in vacuo to afford the product as a crude oil. The crude material was purified by flash chromatography on silica cartridge eluting with a gradient of DCM:MeOH from 0% to 10% MeOH to afford the title product as a pale yellow solid. ¹H NMR (DMSO-d6, 400 MHz) δ 12.9 (1H, br s, COOH), 7.7 (1H, s, CH, Ar—H), 7.4 (2H, m, Ar—H), 7.25 (2H, m, Ar—H), 7.1 (2H, br s, NH2).

### Intermediate GA: 3-Amino-6-cyclopropyl-5-(trifluoromethyl)picolinic acid

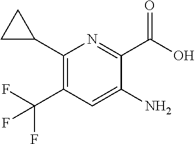 [SMILES: Nc1cc(C(F)(F)F)c(C2CC2)nc1C(=O)O]

### Step 1: 3-Amino-6-cyclopropyl-5-(trifluoromethyl)picolinic acid

A microwave vial was charged with amino-6-bromo-5-trifluoromethyl-pyridine-2-carboxylic acid methyl ester (Intermediate A4) (0.5 g, 1.754 mmol), cyclopropylboronic acid (0.753 g, 8.77 mmol), and 1,1′Bis(diphenylphosphosphino) ferrocene palladium dichloride (0.143 g, 0.175 mmol). The mixture was taken up as a solution in THF (6 ml) and flushed with N2, sealed and heated using microwave radiation at 150° C. for 20 minutes. The reaction mixture was filtered through Celite® (filter material) and washed through with EtOAc (20 ml). The filtrate was partitioned between EtOAc (30 ml) and water (50 ml). The phases were separated and the organic portion was washed with brine (30 ml), dried over MgSO₄, filtered and concentrated under vacuum.

The crude material was taken up in EtOAc (20 ml) and dry loaded onto silica (2-3 g). Material then purified on the Combiflash Rf Teledyne ISCO System 100% Isohexane to 60% EtOAc:Isohexane to afford semi pure material which was used without further purification.

### Step 2: 3-Amino-6-cyclopropyl-5-(trifluoromethyl)picolinic acid

To a stirring solution of 3-amino-6-cyclopropyl-5-(trifluoromethyl)picolinic acid (472 mg, 1.814 mmol) in THF (10 ml), 2M NaOH (10 ml, 20.00 mmol) was added. The orange solution was stirred at RT for 2 days. The reaction mixture was poured into water (30 ml) and the pH adjusted to pH6 with the addition of 1M HCl. The product was extracted with EtOAc (50 ml) and the organic portion was dried over MgSO₄, filtered and concentrated in vacuo to give the title compound as a red/orange oil. LC-MS Rt=1.10 mins [M+H]+ 247.1 (Method 2 minLC_v003);

### Intermediate H: 3-Amino-6-(2,4-dichloro-phenyl)-5-trifluoromethyl-pyridine-2-carboxylic acid

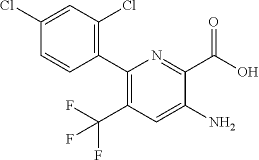 [SMILES: Nc1cc(C(F)(F)F)c(-c2ccc(Cl)cc2Cl)nc1C(=O)O]

### Intermediate H1: 3-Amino-6-(2,4-dichloro-phenyl)-5-trifluoromethyl-pyridine-2-carboxylic acid methyl ester

3-Amino-6-bromo-5-trifluoromethyl-pyridine-2-carboxylic acid methyl ester (Intermediate A4) (3 g, 10.03 mmol), 2,4-dichlorophenylboronic acid (2.297 g, 12.04 mmol), potassium phosphate (4.26 g, 20.06 mmol) and Fibrecat® 1034A (Johnson Matthey, polymer supported palladium complex) (500 mg, 10.03 mmol) were suspended in toluene (50 ml) and water (15 ml). The reaction mixture was heated to 110° C. under vigorous stirring for 3 hours. The mixture was allowed to cool to RT and EtOAc (100 ml) was added. The organic layer was separated and washed with brine (15 ml). MP-TMT (macroporous polystyrene-bound trimercaptotriazine, 3 g, Polymern labs) was added and stirred for 1 hour at RT. MgSO₄ was added and the suspension filtered off. The filtrate was concentrated in vacuo and purification of the residue by reverse phase chromatography (130 g C₁₈ column) eluting with water/MeOH afforded the title compound as a white solid; LS-MS Rt=1.55 mins [M+H]+ 365 (Method 2 minLC_v002).

### Intermediate H: 3-Amino-6-(2,4-dichloro-phenyl)-5-trifluoromethyl-pyridine-2-carboxylic acid

3-Amino-6-(2,4-dichloro-phenyl)-5-trifluoromethyl-pyridine-2-carboxylic acid methyl ester (0.9 g, 2.465 mmol) was suspended in MeOH (15 ml) and NaOH 2M (2.465 ml, 4.93 mmol) was added under stirring. 1,4-Dioxane (15.00 ml) was added and the solution was left standing at RT over night. The solvent was removed in vacuo and the resulting residue was dissolved in water (10 ml) and carefully acidified to pH4 with slow addition of 2M HCl (2 ml) whilst stirring. The mixture was extracted with EtOAc (20 ml) and the organic portion was washed with brine and concentrated in vacuo. The residue was purified by reverse phase chromatography (130 g C₁₈ column) eluting with water/MeOH to afford the title compound; LS-MS Rt=1.57 mins [M+H]+ 351.0 (Method 2 minLC_v002).

### Intermediate I: 3-Amino-6-(4-chloro-2-methyl-phenyl)-5-trifluoromethyl-pyridine-2-carboxylic acid methyl ester

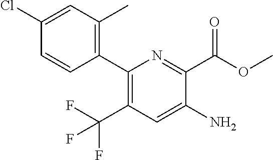 [SMILES: COC(=O)c1nc(-c2ccc(Cl)cc2C)c(C(F)(F)F)cc1N]

This compound was prepared from 3-Amino-6-bromo-5-trifluoromethyl-pyridine-2-carboxylic acid methyl ester (Intermediate A4) and 4-chloro-2-methylphenylboronic acid analogously to Intermediate H; LC-MS Rt=1.53 mins, [M+H]+ 331 (Method 2 minLC_v002).

### Intermediate J: 2-Aminomethyl-1,1,1,3,3,3-hexafluoro-propan-2-ol

To a stirred mixture of 35% ammonium solution (1 ml) and diethyl ether (1 ml) was added 3,3,3-trifluoro-2-(trifluoromethyl)-1,2-propenoxide (500 mg, 2.78 mmol) dropwise and the reaction mixture was left to stir at RT for 3 hours. The reaction mixture was separated and the aqueous layer was extracted with diethyl ether (2×3 ml). The combined organic portions were dried (MgSO₄) and concentrated in vacuo to give a white crystalline solid. ¹H NMR (400 MHz, DMSO-d6) δ 4.20 (broad), 3.30 (broad), 3.15 (s), 3.02 (s), 2.50 (s, DMSO). 19F NMR (400 MHz, DMSO-d6) δ −85 (CF₃), −84.5 (CF₃).

### Intermediate K: 5-Amino-6′-methyl-3-(trifluoromethyl)-2,3′-bipyridine-6-carboxylic acid

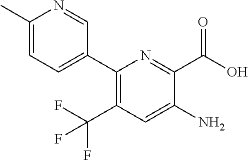 [SMILES: Cc1ccc(-c2nc(C(=O)O)c(N)cc2C(F)(F)F)cn1]

### Intermediate K1: 5-Amino-6′-methyl-3-trifluoromethyl-[2,3′]bipyridinyl-6-carboxylic acid methyl ester

This compound was prepared from 3-Amino-6-bromo-5-trifluoromethyl-pyridine-2-carboxylic acid methyl ester (Intermediate A4) and 2-methylpyridine-5-boronic acid analogously to 3-amino-6-(4-fluoro-phenyl)-5-trifluoromethyl-pyridine-2-carboxylic acid (Intermediate G); LC-MS Rt 0.96 min [M+H]+ 312 (Method 2 minLC_v002). ¹H NMR (400 MHz, DMSO-d6) δ 8.41 (1H, s), 7.79 (1H, s), 7.69 (1H, dd), 7.32 (1H, d), 7.10 (2H, s), 3.82 (3H, s), 2.52 (3H, s).

### Intermediate K: 5-Amino-6′-methyl-3-(trifluoromethyl)-2,3′-bipyridine-6-carboxylic acid

This compound was prepared from 5-Amino-6′-methyl-3-trifluoromethyl-[2,3′]bipyridinyl-6-carboxylic acid methyl ester analogously to 3-amino-6-(4-chloro-2-methyl-phenyl)-5-trifluoromethyl-pyridine-2-carboxylic acid methyl ester (Intermediate I); LC-MS Rt 0.90 min; [M+H]+ 298 (Method 2 minLC_v002). ¹H NMR (400 MHz, DMSO-d6) δ 12.90 (1H, broad), 8.45 (1H, s), 7.72 (2H), 7.32 (1H, d), 7.12 (2H, broad), 2.51 (3H).

### Intermediate KA: 5-Amino-3-(trifluoromethyl)-2,4′-bipyridine-6-carboxylic acid

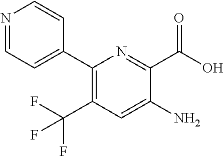 [SMILES: Nc1cc(C(F)(F)F)c(-c2ccncc2)nc1C(=O)O]

The title compound was prepared analogously to Intermediate K using the appropriate boronic acid in step 1. ¹H NMR (400 MHz, DMSO-d6) δ 13.00 (1H, broad), 8.65 (2H, d), 7.65 (1H, s), 7.43 (2H, d), 7.18 (2H, broad).

### Intermediate M: 3-(2,5-Dimethyl-pyrrol-1-yl)-5,6-bis-trifluoromethyl-pyridine-2-carboxylic acid

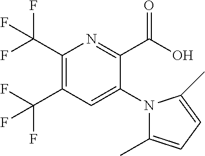 [SMILES: Cc1ccc(C)n1-c1cc(C(F)(F)F)c(C(F)(F)F)nc1C(=O)O]

### Intermediate M1: 3-(2,5-Dimethyl-pyrrol-1-yl)-5,6-bis-trifluoromethyl-pyridine-2-carboxylic acid methyl ester

A stirred mixture of KF (2.12 g, 5.62 mmol) and CuI (0.490 g, 8.43 mmol) was heated in a sealed 10.0-20.0 ml microwave vial under vacuum until a slight greenish colour began to appear. The vial was then placed under nitrogen to cool. A solution 6-bromo-3-(2,5-dimethyl-pyrrol-1-yl)-5-trifluoromethyl-pyridine-2-carboxylic acid methyl ester (Intermediate D) (2.64 ml, 16.86 mmol) in 1:1 dry DMF/dry NMP (14 ml) was then added, followed by TMS-CF₃ (2.64 ml, 16.86 mmol). A new septum was then used to seal the vial and the reaction mixture was heated using microwave radiation with stirring at 100° C. for 3 h and allowed to cool. The mixture was added to 5M NH₃ solution (50 ml) and then extracted with diethyl ether (4×50 ml). The combined organic extracts were washed with 5M NH₃ solution (3×20 ml), 1M HCl (50 ml), sat. sodium bicarbonate solution (2×50 ml), brine (50 ml), dried (MgSO₄) and concentrated in vacuo to give a brown oil. The crude material was purified by chromatography on silica eluting with Iso-hexane/EtOAc, 0-10% to afford the title compound as an orange solid. LC-MS Rt 1.37 min; MS m/z 367.1 [M+H]+; Method 2 minLC_v003.

### Intermediate M: 3-(2,5-Dimethyl-pyrrol-1-yl)-5,6-bis-trifluoromethyl-pyridine-2-carboxylic acid

To a stirred solution 3-(2,5-dimethyl-pyrrol-1-yl)-5,6-bis-trifluoromethyl-pyridine-2-carboxylic acid methyl ester (1.28 g, 3.49 mmol) in methanol (25 ml) was added 1M NaOH (7 ml, 6.99 mmol) and the reaction mixture was left to stir at RT for 30 min. The solvent was removed in vacuo and water (20 ml) was added to the remaining residue. The pH was adjusted to pH 4/5 by the addition of 1M HCl. The mixture was extracted with EtOAc (3×20 ml) and the combined organic extracts were washed with brine (30 ml), dried (MgSO₄) and concentrated in vacuo and dried in a vacuum oven (50° C.) overnight to give the crude title product as an orange solid which was used without further purification; LC-MS: Rt 1.23 min; MS m/z 353.1 [M+H]+; Method 2 minLC_v003.

### Intermediate N: 3,3,3-Trifluoro-N2-(4-methoxybenzyl)-2-methylpropane-1,2-diamine

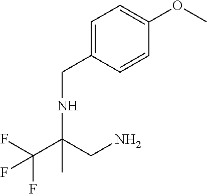 [SMILES: COc1ccc(CNC(C)(CN)C(F)(F)F)cc1]

### Step 1: 1-(4-methoxyphenyl)-N-(1,1,1-trifluoropropan-2-ylidene)methanamine

To a stirring solution of trifluoroacetone (7.75 g, 69.2 mmol) in diethyl ether (60 ml) at −40° C. was added 4-methoxybenzyl amine (9.49 g, 69.2 mmol) and triethylamine (14 g, 138 mmol) in diethyl ether (40 ml). A cooled (0° C.) mixture of TiCl₄ (6.56 g, 34.6 mmol) in hexane (40 ml) at was added dropwise over 10 minutes and the resulting mixture was allowed to warm up to ambient temperature over 20 mins and stirred at 50° C. for 2.5 h. The inorganic precipitate was removed by filtration and washed with diethyl ether. The filtrate was concentrated in vacuo to afford a yellow oil. Purification of the crude product by chromatography on silica eluting with 0% to 25% EtOAc in iso-hexane afforded the title product.

### Step 2: 3,3,3-trifluoro-2-(4-methoxybenzylamino)-2-methyl propanenitrile

To a cooled (0° C.) solution of 1-(4-methoxyphenyl)-N-(1,1,1-trifluoropropan-2-ylidene)methanamine (4.41 g, 19.07 mmol) in DCM (100 ml) was added cyanotrimethylsilane (2.84 g, 28.6 mmol) and magnesium bromide. The mixture was stirred at RT for 90 h and then diluted with sat. NaHCO₃ (200 ml). After stirring at RT for 1 h, the organic phase was separated, washed with a further portion of sat. NaHCO₃ (100 ml), dried over MgSO₄ and concentrated in vacuo to afford the title compound.

### Step 3: 3,3,3-trifluoro-N2-(4-methoxybenzyl)-2-methylpropane-1,2-diamine

To a cooled (0° C.) solution of 3,3,3-trifluoro-2-(4-methoxybenzylamino)-2-methyl propanenitrile (1.5 g, 5.81 mmol) in dry diether ether (50 ml) was added LiAlH4 (11.62 ml of a 2M solution in THF) and the resulting mixture was stirred at RT overnight. The reaction mixture was hydrolyzed by successive addition of water 15% KOH, and water. The resulting precipitate was filtered on Celite® (filter material) and the organic portion was washed with water, dried over MgSO₄ and concentrated under reduced pressure to afford the title product. ¹H NMR (400 MHz, Methanol-d4) δ 7.97 (1H, s), 7.85 (1H, s), 7.60 (1H, s), 3.97 (3H, s), 3.77 (1H, m), 3.56 (1H, m), 1.37 (3H, s). LC-MS: Rt 3.22 min; MS m/z 412.3 [M+H]+; Method 10 minLC_v003.

### Intermediate 0: Benzo[d]isoxazol-3-ylmethanamine

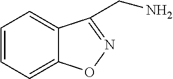 [SMILES: NCc1noc2ccccc12]

The title compound was prepared according to the procedure of Pigini, Maria; Giannella, Mario; Gualtieri, Fulvio; Melchiorre, Carlo; Bolle, Paola; Angelucci, Luciano. Analogs with a 1,2-benzisoxazole nucleus of biologically active indole derivatives. III. Tryptamine and gramine isosteres. European Journal of Medicinal Chemistry (1975), 10(1), 29-32 (Compound 11 page 31-32).

### Intermediate P: Methyl 3-amino-6-(oxazol-2-yl)-5-(trifluoromethyl)picolinate

A solution of 3-amino-6-bromo-5-trifluoromethyl-pyridine-2-carboxylic acid methyl ester (Intermediate A4) (500 mg, 1.672 mmol), 2-(tributylstannyl)oxazole (0.704 ml, 3.34 mmol) and tetrakis(triphenylphosphine)palladium(0) (193 mg, 0.167 mmol) in dioxane (10 ml) was heated at reflux for 13 hours. After cooling to room temperature over 8 hours, the solvent was evaporated and the resulting residue triturated with hot methanol to remove a yellow solid impurity. The remaining crude material was used without further purification. LC-MS: Rt 0.95 min; MS m/z 288 [M+H]+; Method 2 minLC_v003.

### Intermediate PA: 3-Amino-6-furan-2-yl-5-trifluoromethyl-pyrazine-2-carboxylic acid

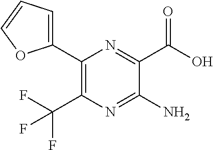 [SMILES: Nc1nc(C(F)(F)F)c(-c2ccco2)nc1C(=O)O]

### Step 1: 3-Amino-6-furan-2-yl-5-trifluoromethyl-pyrazine-2-carboxylic acid

The title compound was prepared from 3-amino-6-bromo-5-trifluoromethyl-pyrazine-2-carboxylic acid ethyl ester (Intermediate C1) and tributyltin-2-furylstannane analogously to methyl 3-amino-6-(oxazol-2-yl)-5-(trifluoromethyl)picolinate (Intermediate P)

### Step 2: 3-Amino-6-furan-2-yl-5-trifluoromethyl-pyrazine-2-carboxylic acid

The title compound was prepared from 3-amino-6-furan-2-yl-5-trifluoromethyl-pyrazine-2-carboxylic acid and 6M NaOH analogously to 3-amino-6-bromo-5-trifluoromethyl-pyrazine-2-carboxylic acid (Intermediate C, final step).

### Intermediate Q: 2-Hydroxy-3-methyl-2-(trifluoromethyl)butan-1-aminium chloride

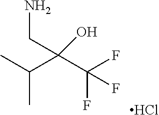

### Step 1: 1,1,1-Trifluoro-3-methyl-2-(nitromethyl)butan-2-ol

A cooled (0° C.) solution of lithium hydroxide (0.048 g, 2.015 mmol) in water (20 ml) was stirred and treated with nitromethane (1.23 g, 20.15 mmol), 1,1,1-trifluoro-3-methylbutan-2-one (3.11 g, 22.17 mmol), cetyltrimethylammonium Chloride (0.871 g, 2.72 mmol) and MgSO₄ (0.485 g, 4.03 mmol). The white suspension was stirred at 0° C. for 1 hr, then at RT for 2 days. The resulting biphasic mixture was separated and the more dense lower layer was collected and dissolved in diethyl ether (30 ml). The mixture was dried over MgSO₄, filtered and concentrated in vacuo to give a pale yellow oil. The oil was taken up in diethyl ether (10 ml) and passed through a pre-packed SCX-2 cartridge eluting with 100% diethyl ether. The filtrate was concentrated in vacuo to afford the title compound as a colourless oil. ¹H NMR (400 MHz, CDCl₃):

δ 4.74 (1H, d), 4.59 (1H, d), 4.29 (1H, s), 2.29 (1H, m), 1.1 (6H, two sets of unresolved doublets)

### Step 2: 2-Hydroxy-3-methyl-2-(trifluoromethyl)butan-1-aminium chloride

To a solution of 1,1,1-trifluoro-3-methyl-2-(nitromethyl)butan-2-ol (753 mg, 3.74 mmol) in EtOH (10 ml) in a 25 ml medium pressure glass hydrogenation vessel under N2, 10% Pd on carbon (39.8 mg, 0.374 mmol) was added. The vessel was flushed with N2, followed by H2 (22.64 mg, 11.23 mmol) at 5 bar pressure and stirred at RT for 6 days. The mixture was filtered through Celite® and washed through with EtOH (30 ml), followed by DCM (10 ml). The filtrate was concentrated under vacuum to give a colourless oil. The crude product was taken up in methanol (20 ml) and treated with a 1.25M HCl in methanol solution. The resulting colourless solution was stirred at RT for 1 hour and concentrated under vacuum to afford the title compound. ¹H NMR (400 MHz, DMSO-d6) δ 8.04 (3H, broad peak), 6.74 (1H, s), 3.58 (broad peak), 3.6 (2H, m), 2.12 (1H, m), 0.99 (6H).

### Intermediate R: 3-Amino-1,1,1-trifluoro-2-methylpropan-2-ol hydrochloride

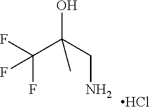

### Step 1: 1,1,1-trifluoro-2-methyl-3-nitropropan-2-ol

To LiOH (0.193 g, 8.06 mmol) in a 3-neck roundbottom flask was added water (25 ml), nitromethane (3.76 ml, 81 mmol) and trifluoroacetone (7.95 ml, 89 mmol).

Cetyltrimethylammonium chloride (3.8 g, 10.88 mmol) and MgSO₄ (1.9 g, 16.12 mmol) were added and the resulting yellow solution stirred at 20-25° C. for 2 days. The reaction mixture was poured into diethyl ether (120 ml) and washed with water (3×200 ml) and brine (lx 100 ml). The organic portion was dried over MgSO₄ and concentrated in vacuo to afford the title compound as a yellow liquid. 41 NMR (CDCl₃, 400 MHz): δ 4.7 (1H d), δ 4.5 (1H, d), δ 3.7 (1H, broad), δ 1.6 (3H, s).

### Step 2: 3-Amino-1,1,1-trifluoro-2-methylpropan-2-ol hydrochloride

Pd/C was added (1 g) to a 200 ml glass vessel. Ethanol (50 ml, dry) was added cautiously under an atmosphere of CO_{2.} 1,1,1-Trifluoro-2-methyl-3-nitropropan-2-ol (10 g, 57.8 mmol) was dissolved in ethanol (50 ml, dry) and added to the glass vessel. The reaction mixture was put under a positive pressure of hydrogen (5 bar) at room temperature and hydrogenated for 2 days. The reaction mixture was filtered through Celite® (filter material) and washed with excess ethanol. The solvent was removed in vacuo to yield a colourless oil. The oil was dissolved in MeOH (50 ml) and treated dropwise with HCl (1M) in MeOH (30 ml). The solution was left to stir for 30 minutes and concentrated in vacuo azeotroping with MeCN to afford the title compound as a waxy white solid. ¹H NMR (DMSO-d6, 400 MHz) δ 8.3 (3H, broad s), 6.9 (1H, broad), 3.0 (2H, q), 1.4 (3H, s).

### Intermediate RA: (S)-3-Amino-1,1,1-trifluoro-2-methylpropan-2-ol hydrochloride

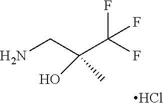

### Step 1: Benzyl 3,3,3-trifluoro-2-hydroxy-2-methylpropylcarbamate

To a stirring suspension of amino-1,1,1-trifluoro-2-methylpropan-2-ol hydrochloride (Intermediate R) (1.5 g, 8.35 mmol) in DCM (50 ml) was added TEA 93.54 g, 35.0 mmol) followed by benzyl 2,5-dioxopyrrolidin-1-yl carbonate (1.983 g, 7.96 mmol). The mixture was stirred at RT for 6 hours and then diluted with water. The organic portion was separated using a phase separator and concentrated in vacuo. Purification by chromatography on silica eluting with 0-70% EtOAc in iso-hexane afforded the title product. ¹H NMR (400 MHz, DMSO-d6) δ 7.34 (6H, m), 5.98 (1H, s), 5.05 (2H, s), 3.31 (1H, m), 3.18 (1H, m), 1.21 (3H. s) LC-MS: Rt 1.05 min; MS m/z 278.1 [M+H]+; Method 2 minLC_v003.

### Step 2: Separation of Enantiomers of benzyl 3,3,3-trifluoro-2-hydroxy-2-methyl propylcarbamate

Benzyl 3,3,3-trifluoro-2-hydroxy-2-methylpropylcarbamate (1.7 g) was dissolved in 2-propanol (10 ml) and purified using the following chromatographic conditions:

Mobile Phase: 10% 2-propanol/90% CO₂
Column: 2×Chiralcel OJ-H, 250×10 mm id, 5 μm (columns coupled in series)

### Detection: UV @ 220 nm

Flow rate: 10 ml/min
Sample concentration: 1.7 g in 10 ml 2-propanol
Injection volume: 75 μl

First eluted peak: Rt=6.94 minutes (R)-benzyl 3,3,3-trifluoro-2-hydroxy-2-methyl propylcarbamate

Second eluted peak: Rt=8.04 minutes (S)-benzyl 3,3,3-trifluoro-2-hydroxy-2-methyl propylcarbamate (Stereochemistry confirmed by analysis of final compound prepared by subsequent steps)

### Step 3: (S)-3-Amino-1,1,1-trifluoro-2-methylpropan-2-ol hydrochloride

A mixture comprising (S)-benzyl 3,3,3-trifluoro-2-hydroxy-2-methyl propylcarbamate in EtOH (165 ml) was pumped through a H-Cube (hydrogenation reactor, 1-2 ml/min, 1 bar pressure, RT) for 8 hours using a 10% palladium on carbon catalyst cartridge. 1.25 M HCl in methanol (130 ml) was added to the mixture was stirred for 30 mins. The solvent was removed in vacuo azeotroping with MeCN to afford the title product as a white powder. ¹H NMR (400 MHz, DMSO-d6) δ 8.3 (3H, broad), 6.8 (1H, s), 3.0 (2H, s), 1.5 (3H, s).

Alternatively, racemic 3-Amino-1,1,1-trifluoro-2-methylpropan-2-ol can be resolved into separate enantiomers by recrystallistion with either (S)-Mandelic acid or L-tartaric acid in isopropanol or ethanol.

### Intermediate S: 2-Aminomethyl-1,1,1,3,3,3-hexafluoro-propan-2-ol

3,3,3-Trifluoro-2-(trifluoromethyl)-1,2-propenoxide (1 g, 5.55 mmol) was added to a stirred solution of aqueous ammonia solution (0.88 g/ml, 3 ml) and diethyl ether (3 ml). The resulting colourless solution was stirred at room temperature for 3 hours. The biphasic mixture was separated and and the aqueous portion was further extracted with diethyl ether (2×5 ml). The combined organic layers were dried over MgSO₄ and concentrated in vacuo (no heating) to afford the title compound as a white crystalline solid which was used without further purification. ¹H NMR (400 MHz, DMSO-d6) signals unassigned δ 4.20 (broad), 3.15 (s).

### Intermediate T: 3,3,3-Trifluoro-2-methoxy-2-methylpropan-1-amine

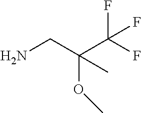

### Step 1: 2-(3,3,3-Trifluoro-2-hydroxy-2-methylpropyl)isoindoline-1,3-dione

A mixture comprising 3,3,3-trifluoro-2-hydroxy-2-methyl-propyl-ammonium (0.9 g), phthalic anhydride (1.039 g) and DIPEA (2.188 ml) in chloroform (30 ml) was heated at 70° C. for 5 hours. After cooling to RT, the mixture was washed with water and passed through a phase separator. The organic phase was reduced to dryness. The crude product was purified by chromatography on silica, eluting in a 0% to 30% iso-hexane:EtOAc removed to give the title product. ¹H NMR (400 MHz, Methanol-d4) δ 7.92 (2H, m), 7.85 (2H, m), 3.95 (2H, m), 1.36 (3H, s).

### Step 2: 2-(3,3,3-Trifluoro-2-methoxy-2-methylpropyl)isoindoline-1,3-dione

To a stirring solution of 2-(3,3,3-trifluoro-2-hydroxy-2-methylpropyl)isoindoline-1,3-dione (250 mg, 0.915 mmol)) at 0° C. in THF (8 ml), NaH (80 mg, 2 mmol) was added. After 30 minutes methyl iodide (1.299, 9.15 mmol) was added. The reaction mixture was left stirring in a ice-bath and allowed to warm to 25° C. over 3.5 hours. The reaction was quenched with sat. NH₄Cl and the mixture extracted with DCM. The organic extract was separated using a phase seperator and purification by chromatography on silica, eluting in a 0% to 30% iso-hexane:EtOAc afforded the title product. ¹H NMR (400 MHz, Methanol-d4) δ 7.91 (2H, m), 7.85 (2H, m), 3.97 (2H, m), 3.44 (3H, s), 1.42 (3H, s). LC-MS: Rt 1.17 min; MS m/z 288.10 [M+H]+; Method 2 minLC_v003.

### Step 3: 3,3,3-Trifluoro-2-methoxy-2-methylpropan-1-amine

A mixture comprising 2-(3,3,3-trifluoro-2-methoxy-2-methylpropyl)isoindoline-1,3-dione (272 mg, 0.95 mmol) and hydrazine (0.033 ml, 1.045 mmol) was stirred at 75° C. for 4 hours. After cooling to RT, the mixture was filtered and the filtrate was concentrated in vacuo to afford the title product which was used without further purification (no characterisation data available).

From the foregoing it will be appreciated that, although specific embodiments of the invention have been described herein for purposes of illustration, various modifications may be made without deviating from the spirit and scope of the invention. Accordingly, the invention is not limited except as by the appended claims.

7.2. Phase II Study of Compound A

A double-blinded, randomized, placebo-controlled, non-confirmatory, and multi-center, fixed-dose study of Compound A in COPD patients was conducted to assess efficacy, safety, tolerability, pharmacokinetics (PK), and pharmacodynamics (PD). Patients were randomized and stratified by smoking status (former vs current smokers). The double-blind placebo-controlled treatment period was 28 days (Day 1 to Day 28) and included visits on Days 1, 14 and 28. Eligible patients were randomized in a 2:1 ratio to receive 300 mg b.i.d. of Compound A or matching placebo for 28 consecutive days. Of note, the first 4 patients enrolled randomized in this study received 450 mg b.i.d. of Compound A or placebo, prior to an amendment reducing the dose and thereafter the dose was reduced to 300 mg b.i.d. of Compound A. The single-blind placebo-controlled follow-up period was 28 days. The follow-up period included a visit on Day 29 for key primary and secondary objective efficacy assessments, weekly follow-up contact via telephone, and an End-of-Study (EOS) visit on Day 56. Number of patients (planned and analyzed): Ninety two (92) patients were randomized into the study. The majority of the patients (N=78, 84.8%) completed the study as planned. A total of 14 patients discontinued the study (N=2 from placebo group and N=12 from Compound A group). The reasons for discontinuations include adverse events, protocol deviations (failure to meet inclusion/exclusion at Baseline), patient decision, abnormal laboratory values (positive drug screen) and administrative problems (patient unable to return to site for visits).

7.2.1. Fibrinogen Levels

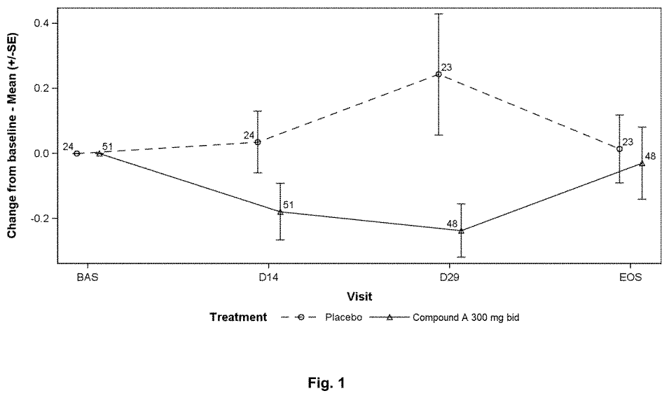

Blood samples were collected from patients for the analysis of fibrinogen. A decrease in fibrinogen levels indicates improvement and suggests reduction in small airway inflammation. The mean change from baseline (SE) over time for fibrinogen per treatment with Compound A is shown in FIG. 1. The plot displays decrease of fibrinogen levels on Day 14 and 29 during treatment period, indicating improvement.

The summary of fibrinogen levels in patients is summarized in Table 3. On Day 29, the estimated difference (Compound A to placebo) in mean change from baseline fibrinogen was −0.40 g/L (90% CI: −0.65, −0.14; p-value=0.006), indicating improvement. The treatment effect of Compound A is statistically significant as compared to placebo. Fibrinogen on Day 29 showed a mean 40 mg/dL improvement over placebo which was statistically significant (p-value=0.006). Fibrinogen has been approved as a prognostic biomarker of COPD patients at high risk for exacerbations and/or all-cause mortality. The change in fibrinogen supports a potential indirect anti-inflammatory effect for the Compound A mechanism of action.

TABLE 3
Summary of fibrinogen levels.¹

| | | | 300 mg bid of | |
|---|---|---|---|---|
| | | Placebo | Compound A | Total |
| Visit | Statistics | N = 25 | N = 53 | N = 78 |
| SCR | n | 25 | 50 | 75 |
| | mean | 3.15 | 3.03 | 3.07 |
| | SD | 0.612 | 0.660 | 0.643 |
| | minimum | 2.3 | 1.1 | 1.1 |
| | median | 3.05 | 3.00 | 3.03 |
| | maximum | 4.3 | 4.9 | 4.9 |
| BAS | n | 24 | 51 | 75 |
| | mean | 3.11 | 3.28 | 3.23 |
| | SD | 0.763 | 0.699 | 0.719 |
| | minimum | 1.4 | 2.0 | 1.4 |
| | median | 3.22 | 3.16 | 3.18 |
| | maximum | 4.3 | 4.9 | 4.9 |
| D14 | n | 25 [24] | 52 [51] | 77 [75] |
| | mean | 3.13 [0.04] | 3.10 [−0.18] | 3.11 [−0.11] |
| | SD | 0.663 [0.464] | 0.616 [0.621] | 0.627 [0.581] |
| | minimum | 2.0 [−1.0] | 1.6 [−1.7] | 1.6 [−1.7] |
| | median | 3.18 [0.00] | 3.03 [−0.20] | 3.05 [−0.08] |
| | maximum | 4.4 [1.1] | 4.4 [1.7] | 4.4 [1.7] |
| D29 | n | 24 [23] | 49 [48] | 73 [71] |
| | mean | 3.34 [0.24] | 3.02 [−0.24] | 3.13 [−0.08] |
| | SD | 0.802 [0.893] | 0.641 [0.569] | 0.709 [0.720] |
| | minimum | 2.1 [−2.1] | 1.6 [−1.8] | 1.6 [−2.1] |
| | median | 3.22 [0.13] | 2.98 [−0.17] | 3.06 [−0.08] |
| | maximum | 5.8 [2.6] | 4.4 [1.3] | 5.8 [2.6] |
| EOS | n | 24 [23] | 49 [48] | 73 [71] |
| | mean | 3.08 [0.01] | 3.24 [−0.03] | 3.19 [−0.02] |
| | SD | 0.608 [0.500] | 0.772 [0.769] | 0.722 [0.690] |
| | minimum | 1.9 [−1.4] | 2.0 [−1.7] | 1.9 [−1.7] |
| | median | 3.25 [−0.12] | 3.16 [−0.11] | 3.16 [−0.12] |
| | maximum | 4.0 [1.0] | 6.6 [3.0] | 6.6 [3.0] |

¹Results in brackets indicate change from baseline. Two subjects received a 450 mg b.i.d. dose of Compound A.
Day 29 assessment only was excluded from the analysis due to missing dosing near to the assessment for one subject dosed with placebo and two subjects treated with Compound A.

7.2.2. Sputum Microbiology

Spontaneous sputum was collected from patients and used to quantify colony forming units (CFUs) as a potential efficacy marker. The collected sputum samples were cultured for the following specific bacteria: Hemophilus parainfluenza, Hemophilus influenza, Pseudomonas aeruginosa, Moraxella, and Streptococcus pneumonia. In the event of bacterial growth for one or more of the above five pathogens, then the test result was reported, including the quantitative growth. In order to be included in the sputum analysis, a patient must have provided a sufficient sputum sample at baseline and at least one other visit (Day 14, Day 29 or EOS).

A decrease in airway bacterial colonization as detected in the sputum is considered improvement. The summary of sputum microbiology data for all patients included in the sputum analysis is presented in Table 4. In Table 4, the frequency (n, %) of patients was reported by treatment with Compound A and type of bacteria. Within each treatment group, the percentage was calculated as number of patients who were colonized with bacteria divided with total number of patients who produced a sputum sample at that time point.

TABLE 4
Categorical summary of sputum microbiology.²

| | | Placebo | | Compound A | |
|---|---|---|---|---|---|
| Visit | Type of Organism | N | n (%) | N | n (%) |
| Baseline | Any Pathogen | 23 | 15 (65.2) | 45 | 26 (57.8) |
| | Haemophilus influenzae | 23 | 15 (65.2) | 45 | 25 (55.6) |
| | Pseudomonas aeruginosa Biotype 2 - dry | | | 45 | 2 (4.4) |
| | Pseudomonas aeruginosa small colony variant | | | 45 | 1 (2.2) |
| D14 | Any Pathogen | 21 | 12 (57.1) | 43 | 23 (53.5) |
| | Haemophilus influenzae | 21 | 12 (57.1) | 43 | 20 (46.5) |
| | Pseudomonas aeruginosa Biotype 2 - dry | | | 43 | 4 (9.3) |
| D29 | Any Pathogen | 20 | 14 (70.0) | 42 | 23 (54.8) |
| | Haemophilus influenzae | 20 | 13 (65.0) | 42 | 22 (52.4) |
| | Pseudomonas aeruginosa Biotype 2 - dry | 20 | 1 (5.0) | 42 | 2 (4.8) |
| EOS | Any Pathogen | 19 | 14 (73.7) | 42 | 17 (40.5) |
| | Haemophilus influenzae | 19 | 14 (73.7) | 42 | 16 (38.1) |
| | Pseudomonas aeruginosa Biotype 2 - dry | | | 42 | 1 (2.4) |

²To be included in the sputum analysis, subjects had to have a sputum sample sufficient for testing at baseline and at least one other visit.
N = the number of subjects analyzed at each visit.
Day 29 assessment only was excluded from the analysis due to missing dosing near to the assessment for one subject dosed with placebo and two subjects treated with Compound A.

The summary of sputum microbiology data for patients with a positive baseline sputum result is presented in Table 5. For the treatment effect a summary table, the frequency (n, %) of patients with a positive culture was reported by each treatment. Only patients with a positive culture at baseline are included in this analysis. Percentage was calculated as number of patients who are colonized (i.e., have at least one culture resulted) divided with total number of patients who produced a sputum sample at each visit, by treatment.

TABLE 5
Categorical summary of sputum microbiology on subjects with
a pathogen at baseline.³

| | | Placebo | | Compound A | |
|---|---|---|---|---|---|
| Visit | Type of Organism | N | n (%) | N | n (%) |
| D14 | Any Pathogen | 14 | 8 (57.1) | 25 | 19 (76.0) |
| D29 | Any Pathogen | 13 | 11 (84.6) | 24 | 18 (75.0) |
| EOS | Any Pathogen | 13 | 10 (76.9) | 24 | 14 (58.3) |

³To be included in the sputum analysis, subjects had to have a sputum sample sufficient for testing at baseline and at least one other visit.
N = the number of subjects with a pathogen at baseline, analyzed at each visit.
Day 29 assessment only was excluded from the analysis due to missing dosing near to the assessment for one subject dosed with placebo and two subjects treated with Compound A.

At Baseline, 60% of patients (N=41) in the sputum analysis set (N=68) summarized in Table 4 had a positive sputum culture result with at least one of the five investigated pathogens. Baseline bacterial colonization was balanced between Compound A (58%) and placebo (65%) arms. For all patients included in the sputum analysis set, there is a numerical trend for decreased colonization on Day 29 and EOS for patients randomized to Compound A versus placebo. On Day 29, 55% of patients treated with Compound A were colonized versus 70% of patients dosed with placebo. At EOS, 41% of patients treated with Compound A were colonized versus 74% of patients dosed with placebo.

Among the 41 patients with a culture positive sputum at Baseline, there is a numerical trend for increased colonization clearance at EOS for patients treated with Compound A. On Day 29, 25% of patients treated with Compound A cleared colonization versus 15% of patients dosed with placebo. At EOS, 42% of patients treated with Compound A cleared colonization versus 23% of patients treated with placebo.

7.3. A Randomized, Subject- and Investigator-Blinded, Placebocontrolled Parallel Group Study to Assess the Safety, Tolerability, Pharmacokinetics and Pharmacodynamics of Compound a in Patients with Bronchiectasis

7.3.1. Study Design

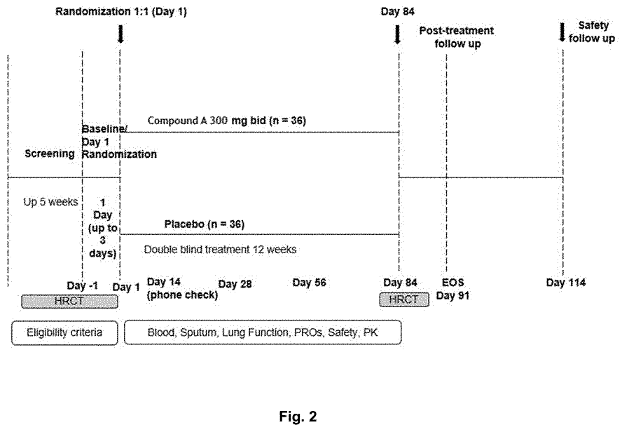

This is a randomized, subject- and investigator-blinded, placebo-controlled, parallel-group study investigating the preliminary efficacy and safety of Compound A administered orally for 12 weeks in subjects with bronchiectasis. The study consists of the following periods: Screening, baseline/Day 1, treatment period, and end of study assessments (EOS) visit followed by an additional post-treatment safety follow up via phone call. The total duration for each patient in the study is up to approximately 18 weeks. The study design is described in FIG. 2. A summary of study objectives is described in Table 6.

TABLE 6
Summary of Study Objectives

| Primary Objective | Primary Endpoint |
|---|---|
| To assess the change on sputum bacterial | Change from baseline in bacterial load of |
| colonization | colony forming units ( CFU/mL) of potentially |
| | pathogenic microorganisms in spontaneous |
| | sputum with Compound A compared to placebo |
| | at week 12 |
| Secondary Objectives | Secondary Endpoints |
| To assess the change of Compound A | Proportion of subjects with absence of any CFU |
| compared to placebo on sputum bacterial | of potentially pathogenic bacteria in sputum |
| clearance | culture after 12 weeks of treatment |
| To assess the change on patient reported | Changes from baseline in Quality of Life |
| outcomes on bronchiectasis symptom | Questionnaire for Bronchiectasis (QOL-B) |
| assessment | (Respiratory symptoms domain) after 12 weeks |
| | of treatment |
| To assess the change of fibrinogen plasma | Change from baseline in fibrinogen plasma |
| concentration | concentration after 12 weeks of treatment |
| To assess the change in rescue medication | Change from baseline in rescue medication use |
| use | (salbutamol/albuterol) after 12 weeks of treatment |
| To assess the change on lung function | Changes from baseline in pre- and |
| | postbronchodilator FEV1, FVC after 12 weeks |
| | of treatment, measured by spirometry |
| To assess the change in airway structure and | Change from baseline in airway wall and lumen |
| function | parameters along with extent of global and |
| | regional air trapping after 12 weeks of |
| | treatment, as measured by HRCT |
| To assess the pharmacokinetics of Compound | Assessment of drug exposure (Cmax, AUC) on |
| A in patients with bronchiectasis | Days 1 and 28; pre- and post-dose concentration |
| | (Ctrough and Cmax) on Days 56 and 84 (for a |
| | subset of patients at selected sites). Pre- and |
| | post-dose concentration (Ctrough and Cmax) |
| | on Days 1, 28, 56 and 84 (for all other patients) |
| To assess the safety and tolerability of | All safety endpoints (including adverse events, |
| Compound A in patients with bronchiectasis | vital signs, ECG, and safety laboratory changes) |
| | during the study |

### Screening Visit (Day −35 to Pay −1):

Screening assessments can be performed over a 5-week period maximum (up to 35 days). Inclusion and exclusion criteria will be checked to confirm patient's eligibility. This check includes medical history, maintenance therapy of LABA/ICS or LABA/LAMA or LABA/LAMA/ICS and/or macrolides, physical examination, ECG, vital signs, oxygen saturation, and clinical laboratory evaluations (hematology, blood chemistry, urinalysis). Sputum will be collected once within the screening window to confirm bacterial load with at least one strain of potentially pathogenic bacteria (refer inclusion criteria). The sputum microbiology results at screening will need to be available prior to randomization.

At screening, all subjects will be provided with an electronic diary (eDiary) and be trained on its use on how to record information about their rescue medication (salbutamol/albuterol), other concomitant medication use, how to complete questionnaires, how to record symptoms as well as study medication intake (from Day 1 onwards).

A HRCT assessment will be performed at screening. For patients who have historical HRCT reports with confirmation of bronchiectasis diagnosis, do not need to wait until the results are available for eligibility check.

### Baseline/Randomization Day 1:

Subjects who meet the eligibility criteria will be admitted to baseline/Day 1 safety and efficacy evaluations before randomization. During baseline, sputum samples will be collected at the same time of the day (sputum collection procedure and timing will be detailed in the SOM and laboratory manual) for biomarker assessments (bacterial load and colonization). Subjects will be also asked to complete various scales and questionnaires.

There is no antibiotic intervention allowed between screening and baseline except for the use of macrolides for subjects who are on this medication before enrolment. In this case, macrolides are to be continued at the same dose and regimen during the study.

Once all baseline assessments have been completed and subjects are again confirmed as being eligible for the study, they can be randomized on the same day (baseline/randomization Day 1). In the case that sputum sample cannot be collected or other assessments cannot be completed at baseline visit for various reasons, the site must not randomize the subject on the same day. An unscheduled visit needs to be planned for sputum collection prior to randomization and treatment allocation. Once sputum is collected and required assessments are completed, the subject can be randomized on the same day of the unscheduled visit

### Treatment Period (Day 1 to Day 84):

The treatment period will be 84 days (Day 1 to Day 84), with dosing occurring on Days 1 through 84, and will include a Day 1 Visit, a Day 14 visit (via telephone check), a Day 28 Visit, a Day 56 Visit, and a Day 84 Visit. On Day 1, after completion of all pre-dose assessments, eligible subjects will be randomized in a 1:1 ratio to receive Compound A 300 mg b.i.d. or matching placebo for 84 consecutive days.

At randomization, stratification will be done according to the status of macrolides use and geographic region (sites from China/sites outside China) in order to balance patients distribution in treatment and placebo group. The first study medication for this treatment period should be administered in the clinic in the morning of Day 1, following a pre-dose pharmacokinetic blood sample collection (NOTE: all PK sampling times are relative to the first dose of the treatment day).

During treatment period, subjects will return to the site for scheduled visits for biomarker blood/sputum sample collections, PK, safety and efficacy assessments including completion of the questionnaires. On visit days, subjects will take their morning dose in the clinic after completion of predose assessments.

On scheduled sputum collection visits, sputum specimens should be collected in the morning at pre-dose timepoint and before breakfast. In case subjects can't produce enough sputum on the individual scheduled visits, they can come back to site up to 3 days after the scheduled visit to try to produce a sputum sample. If two spontaneous sputum collection attempts are still not satisfactory, investigator may take decision to collect sputum sample after induction by the inhalation of saline.

At Day 14, site will call the subject to evaluate the compliance and to check patient well-being. Pharmacokinetic blood sampling (pre dose and 3 hr post dose) will be done at Day 1, Day 28, Day 56 and Day 84 visits. Additionally, a subset of approximately 30-40 subjects will undergo serial PK sampling at pre- and up to 8 hours post-dose on Day 1 and Day 28.

As a formal thorough QT assessment of Compound A has not been completed, triplicate ECGs preand post-dose at Tmax will be performed at visits on Day 1, Day 28, Day 56 and Day 84 (end of treatment). These assessments will be complemented by PK sampling (trough and Cmax) on the matching time-points, at visits on Day 1, Day 28, Day 56 and Day 84.

The morning dose on Day 84 will be the final dose administration for this treatment period. End of treatment assessment, e.g. safety assessment, lung function assessments and PROs will be performed on Day 84. If spontenous sputum collection is not possible at Day 84, site needs to reschedule the visit within 3 days after Day 84 and subject has to continue to take study medication. Other assessments that have been completed on Day 84 do not need to be repeated.

A second HRCT will be performed at Day 84 after the morning dose of study medication at site. If the HRCT can't be performed at Day 84 for any reason, the assessment needs to be planned within the 3 coming days and patient must continue to take study medication.

In case of symptom deterioration (via e-diary alert), subjects have to visit their study center to determine whether exacerbation criteria have been met and an immediate antibiotic treatment may be necessary (CRP increase over normal laboratory level). In addition, other markers of inflammation such as fibrinogen in blood will be taken to gain more information on systemic inflammation and sputum sample collection in order to determine if there are changes in pathogen or bacterial load that may have resulted in the exacerbation. Subjects experiencing an exacerbation during the treatment period will continue with the study treatment along with the standard of care (SOC) therapy for an exacerbation (i.e. antibiotics).

### End of Study (EoS) Visit (Day 91):

Approximately one week upon completion of the treatment period, subjects will be invited to the center for study visit completion (EOS) assessments.

### Safety Follow-Up Call (Day 114):

A follow-up phone call for safety will occur 30 days after the last dose administration. The safety follow-up includes adverse events safety monitoring.

7.3.2. Rationale

### Rationale for Study Design:

This is a non-confirmatory, multi-center, randomized, placebo-controlled, subject- and investigator-blinded, parallel-group with a 12 week treatment period. Key efficacy endpoints will be evaluated over the time during the study period.

The design of this study addresses the primary objective to assess the effect of Compound A compared to placebo administered for 84 days on sputum bacterial colonization. A reduction from baseline in colony forming units of potentially pathogenic microorganisms in spontaneous sputum by one log unit was associated with a significant reduction in risk of exacerbation by approximately 20% in patients with bronchiectasis, which is considered to be clinincally relevant (Chalmers J D, Smith M P, McHugh B J, et al (2012) Short- and Long-Term Antibiotic Treatment Reduces Airway and Systemic Inflammation in Non-Cystic Fibrosis Bronchiectasis. Am J Respir Crit Care Med; 186(7): 657-665).

In order to optimize the rigor and integrity of the study and minimize bias, a randomized, subject- and investigator-blinded parallel group is used. The design is well-established in respiratory clinical trials and enables the study treatment to be given for an appropriate and practical length of time to assess the efficacy and safety of the treatment. A parallel study design was chosen because a crossover design assumes patients will return to their own baseline levels of CFU in each period and this may not be the case in the study. It is more versatile in that a stable disease state is not a pre-requisite which is beneficial as also newly diagnosed patients with bronchiectasis may be included.

Compound A, an effective Cystic Fibrosis Transmembrane Conductance Regulator (CFTR) potentiator, is hypothesized to be efficacious in patients with bronchiectasis based on improved mucociliary clearance (MCC), resulting in decreased bacterial colonization, decreased small airway inflammation, improved FEV1 and ultimately fewer exacerbations. Recent evidence suggests the molecular mechanism for reduced mucociliary clearance in bronchiectasis may relate to dysfunction of wild-type and mutated CFTR. Bronchiectasis patients may also have a component of ion channel dysfunction, including CFTR. COPD data suggests that Compound A may decrease bacterial colonization and small airway inflammation (reduced fibrinogen). In addition, Compound A demonstrated statistically important improvement in lung function (FEV1) in cystic fibrosis (CF) following 2 weeks of treatment and in COPD following 4 weeks of treatment.

Therefore, in addition to the primary endpoint, several complementary endpoints will be used in this study to assess efficacy, including spirometry and patient reported outcomes (PROs) as well as pharmacokinetic parameters. These evaluations provide a comprehensive view of airway structure and function as well as an assessment of patients' well-being in addition to the safety and tolerability evaluation. The study will also include the measurement of soluble biomarkers to provide additional information relevant to the endpoints.

### Rationale for Dose/Regimen and Duration of Treatment:

There is no preclinical in vivo model of CFTR potentiation. As such, the assumption is made that maintaining steady state unbound trough concentrations which are commensurate with the in vitro EC50 against the wild type CTFR channel (72 nM) will be sufficient to potentiate CFTR.

Clinical activity was observed in CF and COPD patients in clinical study 1 (150 and 450 mg b.i.d.) and study 2 (300 mg b.i.d. for 4 weeks), respectively. The COPD PoC study (study 2) provided evidence of efficacy (FEV1 improvement) with a 300 mg b.i.d. regimen. Following 28 days of Compound A 300 mg bid in patients with COPD, the difference in mean change from baseline (Compound A-placebo) on Day 29 are as follows:
The posterior difference in mean change from baseline LCI was 0.28 (90% CrI: −0.24, 0.79) with a 19% probability that the Compound A treatment effect is better than placebo.
The posterior difference in mean change from baseline pre-bronchodilator FEV1 was 0.05 L (90% CrI: −0.03, 0.13) with an 84% probability that the Compound A treatment effect is better than placebo.
The estimated difference in mean change from baseline post-bronchodilator FEV1 was 0.06 L (90% CI: −0.02, 0.14; p-value=0.100) with a statistically significant improvement in Compound A group as compared to placebo.
The estimated difference in mean change from baseline sweat chloride was −5.19 mmol/L (90% CI: −11.6, 1.25; p-value=0.091) with a statistically significant improvement in Compound A group as compared to placebo. This endpoint suggests Compound A engagement with the CFTR target.
The estimated difference in mean change from baseline fibrinogen was −0.40 g/L (90% CI: −0.65, −0.14; p-value=0.006), with a statistically significant improvement in Compound A group as compared to placebo. This endpoint suggests Compound A may have an indirect antiinflammatory effect.
Other spirometry and lung volume secondary and exploratory efficacy endpoints, including the pre- and post-bronchodilator forced vital capacity (FVC), the IC, and the % FEF25-75, provide supportive evidence of improvement with Compound A as compared to placebo.
A sub-group analysis suggests that former smokers may have had greater improvement with Compound A than current smokers. The subgroup analysis must be interpreted with caution, however, as the former and current smoker placebo populations did not behave similarly during the 28-day treatment period.
In addition to Day 29, efficacy results were assessed at Day 14 and EOS:
At Day 14, LCI, pre-bronchodilator FEV1 and post-bronchodilator FEV1 demonstrated evidence of improvement in the mean change from baseline.
On Day 29 and EOS, there is a numerical trend for decreased colonization for patients randomized to Compound A versus placebo (41% versus 74% at EOS). Among patients with a sputum culture positive at Baseline, there is a numerical trend for increased colonization clearance for patients on Compound A versus placebo (42% versus 23% at EOS).
At EOS, the efficacy results for FEV1, sweat chloride, fibrinogen as well as additional secondary and exploratory spirometry and lung volume measurements had returned to baseline, consistent with the EOS assessments occurring 28 days after the last dose of Compound A.

A PK monitoring plan was implemented to ensure that patients' exposures are generally consistent with an exposure threshold (AUCO-24 h=91,700 ng*h/ml), which was established based on the NOAEL (no observed adverse event level) exposure in a chronic monkey toxicology study. A statistical stopping rule was pre-specified to discontinue the Compound A 400 mg b.i.d. cohort due to a higher than anticipated proportion of patients within the treatment cohort who demonstrated stead-state daily exposure above the threshold (AUC>91′700 ng*h/ml).

The twice-daily dosing regimen was chosen based on the half-life of Compound A (10-16 hours) and the intent to have a sustained effect on the ion channel. Additionally a twice daily regimen is expected to provide a reduced Cmax/Ctrough fluctuation compared to once daily dosing. In comparison with a single dosing regimen designed to achieve the same trough concentrations, the proposed twice daily regimen is expected to yield higher average concentrations during the dosing interval. A twice daily regimen was therefore selected to maximize the opportunity of observing efficacy of Compound A in bronchiectasis patients.

A study duration of 12 weeks is expected to provide clinically significant changes in mucociliary clearance allowing the assessment of safety and tolerability. Moreover an adequate study duration of at least 3 months is especially important for patient reported outcomes such as quality of life to obtain significant treatment effects.

7.3.3. Population

### Inclusion Criteria:

1. Male or female patients aged ≥18 years at screening.
2. Proven diagnosis of bronchiectasis by chest HRCT
3. Evidence of sputum bacterial load of ≥106 CFU/mL with at least one potentially pathogenic microorganism at screening (H. Influenzae, M catarrhalis, S aureus, S pneumoniae, Enterobacteriaceae, P aeruginosa, Stenotrophomonous maltophilia, or any potential pathogenic non-fermenting Gram negative bacteria measured by dilution/outgrowth.) Any organism that is to be included and that is not included in the list of 7 protocol defined pathogens, requires approval by the steering committee in collaboraton with Novartis for inclusion
4. Documented history of at least one bronchiectasis exacerbation in the 12 months prior to screening.
5. Patients with bronchial hypersecretion, defined as productive cough that occurs on most days (defined as >50% days) for at least three consecutive months within 12 months prior to screening, as assessed by documentation of patient recollection (anamnesis) or documented in patients' record.
6. Patients are allowed to stay on fixed or free combinations of LABA/LAMA or LABA/ICS or LABA/LAMA/ICS as maintenance therapy if they are treated with them at a stable dose for the last 3 months prior to screening. Patients are also allowed to stay on macrolides as maintenance therapy if they are treated with them at a stable dose 3 months before screening. If prescribed, patients are included in the study with unchanged chest physiotherapy for at least 4 weeks prior to screening.
7. Clinically stable pulmonary status in the opinion of the investigator and unlikely to require any change in the standard regimen of care during the course of the study.
8. Able to perform reliable, reproducible pulmonary function test maneuvers per American Thoracic Society/European Respiratory Society (ATS/ERS) guidelines at screening. At screening, patients who have failed to meet ATS/ERS requirements for acceptability and reproducibility for spirometry will be allowed one additional repeat testing session during the screening period.

### Exclusion Criteria:

Subjects meeting any of the following criteria are not eligible for inclusion in this study.

1. Use of other investigational drugs at the time of enrollment, or within 5 half-lives of enrollment, or within 30 days, whichever is longer; or longer if required by local regulations. Current or planned participation to another clinical trial during this study.
2. History of hypersensitivity to the study drugs or to drugs of similar chemical classes or excipients.
3. Patients with a history of long-QT syndrome or the QTcF interval at Screening or baseline is prolonged (QTcF>450 ms in males, >460 ms in females).
4. Patients who have a clinically significant ECG abnormality before randomization Note: Clinically significant abnormalities may include but are not limited to the following: left bundle branch block, Wolff-Parkinson-White syndrome, clinically significant arrhythmias (e.g. atrial fibrillation, ventricular tachycardia).
5. Patients with a history or current treatment for hepatic disease including but not limited to acute or chronic hepatitis, cirrhosis or hepatic failure or an aspartate aminotransferase (AST) to alanine aminotransferase (ALT) ratio (AST/ALT) or prothrombin time international normalized ratio(PT/INR) of more than 1.5×ULN at screening.
6. History of lung transplant or malignancy of any organ system (other than localized basal cell carcinoma of the skin), treated or untreated, within the past 5 years, regardless of whether there is evidence of local recurrence or metastases, with the exception of localized basal cell carcinoma of the skin. Patients with segmentectomy for other reasons than cancer are allowed to be included in the study. Patients with a history of cancer and 5 years or more disease free survival time may be included in the study by agreement with Novartis Medical Monitor on a case-by-case basis.
7. Pregnant or nursing (lactating) women, where pregnancy is defined as the state of a female after conception and until the termination of gestation, confirmed by a positive hCG laboratory blood test.
8. Women of child-bearing potential, defined as all women physiologically capable of becoming pregnant, unless they are using acceptable effective methods of contraception during study participation. Acceptable effective contraception methods include:
Total abstinence (when this is in line with the preferred and usual lifestyle of the subject). Periodic abstinence (e.g., calendar, ovulation, symptothermal, postovulation methods) and withdrawal are not acceptable methods of contraception.
Female sterilization (have had surgical bilateral oophorectomy with or without hysterectomy), total hysterectomy or tubal ligation at least six weeks before taking investigational drug. In case of oophorectomy alone, only when the reproductive status of the woman has been confirmed by follow up hormone level assessment.
Male sterilization (at least 6 months prior to screening). For female subjects on the study, the vasectomized male partner should be the sole partner for that subject.
Barrier methods of contraception: condom or occlusive cap (diaphragm or cervical/vault caps). For the United Kingdom of Great Britain and Northern Ireland (UK): with spermicidal foam/gel/film/cream/vaginal suppository.
Placement of an intrauterine device (IUD) or intrauterine system (IUS)
   Note that systemic hormonal contraception (e.g. oral contraception or hormone vaginal ring) is not an acceptable means of contraception due to the potential influence of Compound A in decreasing the systemic levels of these hormones and therefore making them ineffective.

Women are considered post-menopausal and not of child bearing potential if they have had 12 months of natural (spontaneous) amenorrhea with an appropriate clinical profile (e.g. age appropriate history of vasomotor symptoms) or have had surgical bilateral oophorectomy (with or without hysterectomy), total hysterectomy or tubal ligation at least 6 weeks ago. In the case of oophorectomy alone, only when the reproductive status of the woman has been confirmed by follow up hormone level assessment is she considered not of child bearing potential.

9. Use of prescription drugs prohibited within 1 week prior to Day 1.
10. Clinical significant laboratory values abnormalities (including G-GT, AST, ALT, total bilirubin or creatinine) in the opinion of the investigator at screening. For additional guidance on hepatic parameters refer to exclusion criterion #5
11. Patients requiring long-term oxygen therapy for chronic hypoxemia. This is typically patients requiring oxygen therapy >12 h per day delivered by home oxygen cylinder or concentrator.

Note: Nocturnal oxygen therapy for transient oxygen desaturations during sleep is allowed.

12. Patients with bronchiectasis who have had a pulmonary exacerbation with a deterioration in three or more of the following key symptoms for at least 48 h:
cough;
sputum volume and/or consistency;
sputum purulence;
breathlessness and/or exercise tolerance;
fatigue and/or malaise;
haemoptysis
AND

A clinician determines that a change in bronchiectasis treatment is required (e.g. requiring systemic glucocorticosteroid treatment and/or systemic or inhaled antibiotics) within 4 weeks prior to screening.

In the event of an exacerbation occurring 4 weeks before screening, or between the screening and baseline (please see definition above), the patient must NOT be enrolled. The patient may be rescreened once, 4 weeks after the resolution of exacerbation.

13. Hemoptysis, requiring medical intervention at any time within 4 weeks prior to creening.
14. Bronchiectasis predominantly characterized by isolated cavitary lung lesions.
15. Patients with bronchiectasis requiring therapy that may interfere with the assessment of Compound A efficiency or that are unlikely to respond to Compound A as follows:
Patients with suspected active pulmonary tuberculosis or currently being treated for active pulmonary tuberculosis are not allowed. Note: Patients with a history of pulmonary tuberculosis can be enrolled if they meet the following requirements: history of appropriate drug treatment followed by negative imaging results within 12 months prior to baseline visit suggesting low probability of recurrent active tuberculosis
Patients with active allergic bronchopulmonary aspergillosis and asthma as primary diagnosis.
Patients with cystic fibrosis
   16. Current or ex-smokers with severe emphysema.
   Bidi or other similar non-filtered cigarette may be considered applicable to smoking history. They should be counted in the same way as standard cigarettes. Occasional smoking of cigars, pipes, e-cigarettes, or inhaled nicotinwe products are not relevant to smoking history (Dinakar C, O'Connor GT (2016) The Health Effects of Electronic Cigarettes. N Engl J Med; 375(14):1372-1381).
   Note: An ex-smoker may be defined as a subject who has not smoked for ≥6 months at screening or at the time of assessment.
   17. Patients with another concomitant pulmonary disease according to the definition of the International ERS/ATS guidelines, including but not limited to COPD, asthma, interstitial pulmonary fibrosis (IPF), sarcoidosis or other granulomatous or infectious process. Concomitant COPD and asthma with characteristics of airway hyperresponsiveness as well as COPD-Asthma overlap syndrome are allowed as long as it is not the main, primary diagnosis.
   18. Patients currently receiving treatment for nontuberculous mycobacterial (NTM) pulmonary disease. If performed, patients with one or more positive cultures in the last 12 months for M. avium complex, M. abscessus complex, M. kansasii, M. malmoense, M. xenopi, M. simiae or M. chelonae, unless all subsequent NTM cultures (at least two) are negative and in the opinion of the investigator the patient does not meet ATS criteria for NTM-pulmonary disease. Patients receiving any medication that may influence the response to treatment within 4 weeks prior to screening including systemic or inhaled steroids (ICS alone), other systemic immunomodulators, mucolytics or hyperosmolar agents, recombinant human DNAse, any systemic or inhaled antibiotics. Patients with a body mass index (BMI) of more than 40 kg/m2 at screening
   19. Recent (within three years of screening) and/or recurrent history of autonomic dysfunction (e.g., recurrent episodes of fainting, palpitations, etc.).
   20. Patients with a major vascular surgery in the 6 months prior to the screening visit.
   21. Patients who have clinically significant renal, cardiovascular (such as but not limited to unstable ischemic heart disease, NYHA Class III/IV left ventricular failure, myocardial infarction), neurological, endocrine, immunological, psychiatric, gastrointestinal, or hematological abnormalities, which could interfere with the assessment of the efficacy and safety of the study treatment, or patients with Type I diabetes or uncontrolled Type II diabetes. Note: Clinically significant is defined as any disease that, in the opinion of the investigator, would put the safety of the patient at risk through participating, or which would affect the efficacy or safety analysis if the disease/condition exacerbated during the study, or would compromise patient compliance or preclude completion of the study.
   22. Known or suspected history of ongoing, chronic or recurrent infectious disease of HIV, Hepatitis B/C.

7.3.4. Treatment

The study treatment includes:
Investigational drug Compound A dose of 300 mg
Matching placebo

Details on the requirements for storage and management of study treatment, and instructions to be followed for subject numbering, prescribing/dispensing, and taking study treatment are outlined in the SOM.

### Investigational and Control Drugs

Table 7 presents details of the investigational drug and its control.

TABLE 7
Investigational and control drug¹

| Investigational/Control | | |
|---|---|---|
| Drug | Pharmaceutical | Route of |
| (Name and Strength) | Dosage Form | Administration |
| Compound A, 300 mg | Capsule | Oral use |
| b.i.d. | | |
| Compound A placebo | Capsule | Oral use |
| b.i.d. | | |

¹All capsules are of identical appearance to ensure blinding

### Rescue Medication

Rescue medication for pulmonary exacerbations (including systemic antibiotics) are allowed. All subjects will also be provided with a short-acting beta agonist (SABA) (salbutamol 100 μg or albuterol 90 μg or equivalent dose). Patients will be instructed to use it throughout the study on a “needed basis” (No other rescue medication is permitted during the study). Sites will be instructed to record the short-acting beta agonist rescue medications dispension in the eDiary.

### Treatment Arms/Group

On Day 1, subjects will be randomized to one of the following 2 treatment groups in a ratio of 1:1
Compound A 300 mg b.i.d.
Matching placebo to Compound A b.i.d.

All subjects will receive their respective Compound A or placebo capsules for 12 weeks (from Day 1 through Day 84).

7.3.5. Other Treatments

Permitted Concomitant Therapy Requiring Caution and/or Action

Table 8 provides an overview of medications permitted under certain conditions, including bronchodilator medications which need to be withheld for certain timeframes prior to spirometry assessments on visit days and an overview regarding actions to be taken for antibiotics.

TABLE 8
Medications permitted under certain conditions

| Rationale/Group | Medication | Prohibition Period | Action Taken |
|---|---|---|---|
| LABA/LAMA/ICS | e.g. Vilanterol, | Hold treatment for 24 | In case the washout criterion is |
| for maintenance | Umeclidinium, | hours (+/−2 hours) | not fulfilled, reschedule |
| therapy | Indacaterol | prior to each | spirometry accordingly. |
| | Glycopyrronium | FEV1/spirometry | Trough spirometry (−45 min |
| | | measurement on visit | and −15 min assessments) |
| | | days for once daily | should be done within 24 hours |
| | | maintenance inhaled | for once daily maintenance |
| | | therapy and 12 hours | inhaled therapy such as |
| | | (+/−2 hours) for twice | LABA/LAMA or LABA/ICS |
| | | daily maintenance | or LABA/LAMA/ICS from |
| | | inhaled therapy | dosing on the previous |
| | | | morning and approximately 12 |
| | | | hours for twice daily |
| | | | LABA/LAMA or LABA/ICS |
| | | | or LABA/LAMA/ICS from |
| | | | dosing on the previous |
| | | | evening. Otherwise, postpone |
| | | | visit to the next day where |
| | | | washout criteria can be |
| | | | fulfilled |
| Rescue medication | Short-Acting | Hold treatment at | If possible, postpone |
| only | Beta-2 Agonists | least 6 hours prior to | spirometry measurement on the |
| | (SABA) | each | same day until the washout |
| | | FEV1/spirometry | criterion is fulfilled. Trough |
| | | measurement | spirometry (−45 min and −15 |
| | | | min assessments) should be |
| | | | done within 6 hours after last |
| | | | rescue medication intake |
| Antibiotics for | Antibiotics, e.g. | Systemic and/or | NA |
| treatment of | amoxicillin; | inhaled antibiotics are | |
| exacerbations and | Macrolides, e.g. | allowed for treatment | |
| macrolides as | erythromycin | of acute infections, or | |
| maintenance | | are allowed for the | |
| medication | | treatment of acute | |
| | | exacerbations (but | |
| | | not as permanent | |
| | | prophylactic | |
| | | treatment). Topical | |
| | | treatment (e.g. use of | |
| | | intraocular, | |
| | | intraconjunctival | |
| | | antibiotic treatments, | |
| | | topical use of creams, | |
| | | etc.) is permitted. | |

Patients are allowed to have macrolides at stable doses as maintenance therapy throughout the study. If a patient experiences a pulmonary exacerbation and/or worsening of the disease condition, he/she will be treated as deemed appropriate by the investigator. Antibiotics (systemic or inhaled) are allowed for the treatment of pulmonary exacerbations as dictated by the patient's condition.

Compound A may inhibit the metabolic clearance of co-medications mainly metabolized by CYP1A2 or induce those mainly metabolized by CYP2B6. Thus, drugs that are sensitive substrates of CYP1A2 may have potential for an increase in exposure by Compound A, and that sensitive substrates of CYP2B6 may decrease in exposure. In addition, Compound A is a time dependent inhibitor and inducer of CYP3A4/5. The net effect of Compound A on CYP3A4/5 is anticipated to be induction based on results of an oral contraceptive study that resulted in a decrease in exposure of the substrate.

Weak in vitro inhibition of BCRP, OAT1/3, OATP1B1, OATP1B3, UGT1A1 and UGT2B7 was also observed. Compound A may increase the exposure of drugs which are substrates of the transporters or enzymes. The above mentioned drugs are listed below in Table 9 and can be used when indicated and no alternative treatment is available. Safety and efficacy of drug should be monitored accordingly. The following lists are not considered exhaustive and labels for individual drugs should be referred to.

TABLE 9
Medications which may be co-administred with Compound A (if no alternative
treatment is available)
Medications that may have decreased exposure due to co-administration with Compound A

| | |
|---|---|
| Sensitive Substrates of CYP2B6 | bupropion, efavirenz |
| Narrow therapeutic index substrates of CYP3A | alfentanil¹, astemizole, cisapride, cyclosporine, |
| | dihydroergotamine, ergotamine, fentanyl, |
| | pimozide, quinidine, sirolimus¹, tacrolimus¹, |
| | terfenadine¹ |
| Sensitive substrates of CYP3A | alpha-dihydroergocryptine, alfentanil, |
| | almorexant, alisoporivir, aplaviroc, aprepitant, |
| | atazanavir, atorvastatin, avanafil, bosutinib, |
| | brecanavir, brotizolam, , buspirone, |
| | capravirine, casopitant, cobimetinib, |
| | conivaptan, danoprevir, darifenacin, darunavir, |
| | dasatinib, dronedarone, ebastine, eletriptan, |
| | elvitegravir, eplerenone, everolimus, |
| | felodipine, , grazoprevir, ibrutinib, indinavir, |
| | isavuconazole, ivabradine, ivacaftor, |
| | levomethadyl, lomitapide, lopinavir, lovastatin, |
| | lumefantrine, lurasidone, maraviroc, |
| | midazolam, midostaurin, naloxegol, neratinib, |
| | nisoldipine, paritaprevir, perospirone, |
| | quetiapine, ridaforolimus, saquinavir, |
| | sildenafil, simeprevir, simvastatin, sirolimus, |
| | tacrolimus, terfenadine, ticagrelor, |
| | tilidine, tipranavir, tolvaptan, triazolam, |
| | ulipristal, vardenafil, venetoclax, vicriviroc, |
| | voclosporin |

Medications that may have increased exposure due to co-administration with Compound A

| | |
|---|---|
| Sensitive Substrates of CYP1A2 | alosetron, caffeine, duloxetine, melatonin, |
| | pirfenidone, ramelteon, selegiline, tacrine, |
| | tasimelteon |
| BCRP substrates | atorvastatin daunorubicin, doxorubicin, ethinyl |
| | estradiol, hematoporphyrin, imatinib, |
| | methotrexate¹, mitoxantrone, pitavastatin¹, |
| | rosuvastatin¹, SN-38 (irinotecan), simvastatin, |
| | sulfasalazine, sofosbuvir¹, sulfasalazine¹, |
| | tenofovir¹, topotecan¹ |
| OAT Substrates | acyclovir, adefovir, anagliptin, beta-lactam |
| | antibiotics, bumetanide, captopril, cefonicid, |
| | cefaclor, cephradine, cimetidine, |
| | chlorothiazide, cidofovir, dapagliflozin, |
| | famotidine, furosemide, ganciclovir, ibuprofen, |
| | methotrexate, olmesartan, pemetrexed, |
| | pravastatin, pitavastatin, quinaprilat, ranitidine, |
| | rosuvastatin, tenofovir, tetracycline, |
| | topotecanhydroxyl acid, valsartan, zidovudine, |
| | zonampanel. |
| OATP substrates | aliskiren, ambrisentan, anacetrapib, |
| | asunaprevir, atenolol, atrasentan, atorvastatin, |
| | bosentan, bromociptine, caspofungin, |
| | cerivastatin, celiprolol, danoprevir, digoxin, |
| | docetaxel, eliglustat, empangliflozin, |
| | ezetimibe, fimasartan, fexofenadine, |
| | fluvastatin, glyburide, maraviroc, |
| | methotrexate, montelukast, olmesartan, |
| | paclitaxel, pirataprevir, pitavastatin, |
| | pravastatin, repaglinide, rifampin, rosuvastatin, |
| | saquinavir, simvastatin acid, simvastatin, SN- |
| | 38 (irinotecan), telmisartan, ticlopidine, |
| | thyroxine, valsartan |
| UGT1A1 Substrates | abacavir, acetaminophen, atorvastatin, axitinib, |
| | belinostat, buprenorphine, carvediol, |
| | diclofenac, dolutegravir, desvenlafaxine |
| | succinate, eltrombopag, elvitegravir, estradiol, |
| | etoposide, ezetimibe, ezogabine, febuxostat, |
| | flurbiprofen, fluvastatin, furosemide |
| | gemfibrozil, indacaterol, indomethacin, |
| | irinotecan, ketoconazole, levothyroxine, |
| | losartan, lovastatin, morphine, muraglitazar, |
| | mycophenolate mofetil, mycophenolic acid, |
| | naltrexone, naproxen, paracetamol, raloxifene, |
| | raltegravir, rosuvastatin, simvastatin, |
| | irinotencan, suprofen, telmisartan |
| UGT2B7 Substrates | almokalan, ambrisentan, atorvastatin, |
| | buprenorphine, canagliflozin, carabamazepine, |
| | carvediolol, chloramphenicol, clofibric acid, |
| | codeine, cyclosporine, dabigatran etexilate, |
| | dapagliflozin, diclofenac, empagliflozin, |
| | entacapone, epirubicin, etodolac, ezetimibe, |
| | febuxostat, fenofibrate, fenoprofen, |
| | flurbiprofen, fluvastatin, furosemide, |
| | gemfibrozil, hydromorphone, ibuprofen, |
| | indomethacin, ketoprofen, lamotrigine, |
| | lorazepam, lorazepam, losartan, lovastatin, |
| | methadone, midazolam, mitiglinide, morphine, |
| | mycophenolate mofetil, mycophenolic acid, |
| | nalorphine, naloxone naltrexone, naproxen, |
| | oxazepam, pitavastatin, sertraline, silodosin, |
| | simvastatin, suprofen, tacrolimus, tapentadol, |
| | temazepam, zaltoprofen, zidovudine |

Medications in this table were identified as substrates based on either in vivo or in vitro data.
¹Also considered sensitive CYP3A substrates. Budesonide and fluticasone are also sensitive substrates of CYP3A, but have not been listed here since these are prohibited medications). Furthermore, patients should be instructed not to take grapefruit, Seville oranges or their juice for 14 days prior to dosing, during the treatment and until 7 days following the last dose, due to an ingredient that is an inhibitor sensitive substrate of CYP3A.

### Prohibited Medication

Use of the treatments displayed in the below table is NOT allowed after the onset of the prohibition period as indicated in Table 10 and Table 11. Should administration of one of these drugs during the course of the treatment period be required, study treatment should be discontinued. Table 10 and Table 11 are not considered all-inclusive. Medications should be assessed for adherence to the indication and other inclusion/exclusion criteria. These medications are also prohibited if administered for other indications.

TABLE 10
Prohibited medication

| Rationale/Group | Medication | Prohibition Period |
|---|---|---|
| Medication with a narrow | Theophyline | Discontinue treatment |
| therapeutic range and potential | Tizanidine | at least 1 week |
| for increased exposure with | | prior to Day 1 |
| Compound A due to inhibition | | |
| of CYP1A2 | | |
| Medications lacking | Pirbuterol | Discontinue treatment |
| information on metabolizing | | at least 1 week |
| enzymes | | prior to Day 1 |
| Strong uridine diphosphate | Mefenamic acid | Discontinue treatment |
| Glucuronosyl transferase | Probenecid | at least 1 week |
| (UGT) inhibitors, which will | Valproic acid | prior to Day 1 |
| potentially increase systemic | | |
| concentrations of Compound | | |
| A | | |

TABLE 11
Prohibited respiratory related medications and washout period prior to Day 1

| | Minimum washout period prior to any |
|---|---|
| Class of Medication | spirometry assessments |
| Long-acting muscarinic antagonists (LAMA) | 12 hours for twice-daily LAMAs |
| (other than as ingredient of study maintenance | 24 hours for once-daily LAMAs |
| therapy) | |
| Short-acting muscarinic antagonists (SAMA) | 6 hours |
| Fixed combinations of long-acting (β2 agonists | 12 hours for twice-daily combinations |
| and inhaled corticosteroids (LABA/ICS) or | 24 hours for once-daily combinations |
| LABA/LAMA/ICS | |
| Long-acting (β₂ agonists (LABA) (other than as | 12 hours for twice-daily LABAs, |
| ingredient of study maintenance therapy) | 24 hours for once-daily LABAs |
| Short-acting (β₂ agonists (SABA) (other than | 6 hours |
| trial rescue medication) | |
| Oral phosphodiesterase-IV inhibitor | 7 days |
| Xanthines (any formulation) | 7 days |
| Systemic corticosteroids | 30 days |
| Inhaled corticosteroids | 12 hours |
| Intra-muscular depot corticosteroids | 3 months |
| Mucolytics | Discontinue treatment at least 5 weeks prior |
| Acetylcysteine | Day 1 |
| Ambroxol | |
| Bromohexine | |
| Carbocysteine | |
| Erdosteine | |

### Rescue Medication

Rescue medication for pulmonary exacerbations (including systemic antibiotics) and for bronchospasm is allowed. At Screening and whenever needed thereafter, patients will be provided with a short acting beta agonist (salbutamol 100 μg or albuterol 90μg) inhaler to use as rescue medication on an “as needed” basis throughout the study for bronchospasm. Nebulized salbutamol/albuterol is not allowed as rescue medication throughout the trial. No other rescue medication for bronchospasm is permitted.

### Dietary Restrictions

The following are the instructions for the investigational drug (Compound A/placebo):
It is recommended not to take the investigational drug in together with high-fat meals (refer to SOM for details). The definition of high-fat meals follow the definition suggested by FDA in the draft guidance on Assessing the Effects of Food on Drugs in INDs and NDAs (FDA 2019): a meal containing at least 1000 kcal (4184 kJ), and at least 50% of that energy content from fat.

An example of a high fat meal would be for a total nutritional energy value of 1000 kcal:
of which from proteins: 150 kcal
of which from carbohydrates: 250 kcal
of which from fats: 600 kcal.
Patients can drink water as needed.
Patients should be instructed not to take grapefruit, Seville oranges or their juice for 14 days prior to dosing, during treatment and until 7 days following the last dose as these products are considered as inhibitors of CYP3A.

### Smoking and Other Restrictions

Smoking is prohibited during the study. On study days when spirometry will be performed, patients should refrain from the following:
Coffee, tea, chocolate, cola and other caffeine-containing beverages and foods and ice cold beverage for 4 hours prior to spirometry
Alcohol for 4 hours prior to spirometry
Strenuous activity for 12 hours prior to spirometry
Exposure to environmental smoke, dust or areas with strong odors

7.3.6. Dose Escalation and Dose Modification

### Dose Interruptions

Study drug interruptions are not permitted unless the investigator considers a temporary interruption is necessary for the treatment of an adverse event. If the adverse event grade is severe and suspected to be related to the investigational study drug, the investigational study drug should be permanently discontinued. Any interruption of study medication for more than 5 consecutive days during the treatment period should be discussed with the local Novartis Medical Monitor to review the patient's eligibility to continue in the trial.

### Dose Modifications (Program Wide Data Level)

Study data (coming from Dose Range Finding study (study 2), Mode of Action study (study 3) and bronchiectasis study (study 4)) will be submitted to the Data Monitoring Committee (DMC) for the following considerations:

1. The proportion of subjects predicted to have exposure or show exposures above threshold (AUCO-24 h=159,000 ng*h/ml). Treatment will be permanently discontinued if more than one patient exhibits a projected AUCO-24 h above the upper range of the individual animal (monkey) exposures (159,000 ng×h/mL).
2. The proportion of subjects predicted to have exposures or show exposuress above threshold (AUCO-24 h=91,700 ng*h/ml).

Actual AUCO-24 h will be derived at Day 1 and Day 28. Additionally trough concentration data will be collected throughout the study at the study visits. A regression of Cmin,ss against AUCO-24 h,ss has shown that trough concentrations that are =3000 ng/ml correspond to an AUCO-24 h of =91,700 ng*h/ml. If at any time in the study a subject is predicted to or has data showing that the AUCO-24 h is above this cutoff, this subject will count in the proportion of subjects above the cutoff.

### Dose Modifications (Individual Data Level)

Safety data monitoring at the individual level through permanent discontinuation of study drug should follow the below conditions:

1. All patients with AEs suspected to be related to study drug and categorized as severe and/or serious will permanently discontinue study drug.
2. For all patients reporting any Serious Adverse Event (SAE), a PK sample will be taken as close as possible to the event. If this PK sample can't be collected for any reason, the most recent available PK sample collected will be analyzed. If PK result shows an exposure above the NOAEL threshold (AUCO-24 h=91,700 ng*h/ml), the patient will permanently discontinue study drug.
3. If a patient presents a verified exposure AUCO-24 h≥159,000 ng*h/ml, the investigator must permanently discontinue the investigational study drug as soon as possible upon receipt of the PK results.

### Dose Adjustments for QTcF Prolongation

In case of QTcF >500 msec, (or QTcF prolongation >60 msec from Baseline/Day 1)
Assess the quality of the ECG recording and the QT value and repeat, if needed
Interrupt study investigational treatment
Determine the serum electrolyte levels (in particular hypokalemia, hypomagnesemia). If abnormal, correct abnormalities
If possible, collect a time-matched PK sample on that visit and record time and date of last study treatment intake
   If QTcF interval >500 msec is confirmed:
Permanently discontinue the study treatment.
Take a blood sample for PK analysis. Timepoint should be as close as possible to the ECG recording in question
Consult with a cardiologist (or qualified specialist)
Increase cardiac monitoring as indicated, until the QTcF returns to ≤480 msec
Review concomitant medication use for other causes for QT prolongation (refer to http://www.qtdrugs.org for known QT prolonging drugs), and for drugs with the potential to increase the risk of drug exposure related QT prolongation
Check the dosing schedule and treatment compliance

### Instruction of Prescribing and Taking Study Medication

The following are the instructions for the investigational drug (Compound A/placebo):
Compound A/placebo is an oral capsule.
One capsule should be taken twice a day at approximately the same time each day, with about 12 hours between each dose administration (approximately in the morning between 7 and 10 a.m. and in the evening between 8 and 11 p.m.)
If vomiting occurs during the course of treatment, patients should be instructed not to take the study drug again before the next scheduled dose.
Patients should be instructed not to make up missed doses.
Subjects should be instructed to swallow whole capsules and not to chew or open them.

Instructions for the maintenance treatment and rescue medication should be according to the respective product label.

On study visit days, patients should be reminded not to take either the investigational drug (Compound A/placebo) or the maintenance therapy doses prior to the site visit to ensure compliance with the pre-dose PK sampling procedure and spirometry pre-dose measurements. The morning dose on the visit days should be taken after the pre-dose PK sampling and spirometry assessments have been completed within 15 min approximately.

Of note, spirometry on visit days shall be conducted:
10-14 hours after the last intake of investigational drug on the evening before for b.i.d. drugs, and
22-26 hours after the last inhalation of daily mainanance medication on the morning before o.d drugs.

7.3.7. Efficacy

Blood and sputum samples will be collected and evaluated in all patients at the following timepoints: Days −35 to −1 (screening), Day 1 (baseline), Day 28±3 (predose), Day 56±3 (predose), Day 84±3 (predose).

### Microbiological Assessment

Spontaneous sputum (if possible) will be collected for analysis of pathogenic bacterial colonization (CFU/mL) (e.g. H influenzae, M catarrhalis, S aureus, S pneumoniae, Enterobacteriaceae, P aeruginosa, Stenotrophomonous Maltophilia, or any potential pathogenic non-fermenting Gram negative bacteria). In addition, 16S rRNA PCR will be performed to measure the bacterial load. The analysis will be performed from remaining sputum samples collected for microbiology assessment.

Spontaneous sputum collection: On scheduled sputum collection visits, at least one sputum specimen should be collected in the morning at pre-dose timepoint and before breakfast (including drinks). In case patients cannot produce enough sputum on the individual scheduled visits, they can come back within 3 days after the scheduled visits to try to produce sputum sample. If two spontaneous sputum collection attempts were still not satisfactory, an investigator may take decision to collect sputum sample after induction by the inhalation of saline.

Treatment emerging pathogens will be determined at all visits. Microbiological analyses, including bacterial colonization and bacterial load profiling, will be performed centrally at a qualified microbiology laboratory. Furthermore, all patients with signs of an exacerbation will have to come to the study center where additional sputum sample will be collected. The analysis of this sample would help to determine whether the bacteria load and/or bacterial colonization may change with an exacerbation.

Finally, all sputum samples must be of good quality. If the sample is determined to be a suboptimal, the site's staff should be contacted and immediately a request should be made for a new sample.

### Spirometry

Spirometry testing will be performed with the MasterScope system (manufactured by eResearch Technology GmbH) according to the ATS/ERS guidelines (Miller M. R, Hankinson J. et al (2005)a Standardisation of spirometry. Eur Respir J. 26(2):319-38; Miller M. R, Crapo R. J. et al (2005)b General considerations for lung function testing. Eur Respir J. 26: 153-161) at screening to assess patient eligibility for the study and at the following timepoints on visits: Days −35 to −1 (screening), Day 1 (predose), Day 28±3 (predose), Day 56±3 (predose), Day 84±3 (predose).

The spirometry evaluation should be performed at the site prior to the morning investigational drug intake and the daily maintenance therapy, such as LABA, LAMA, LABA/LAMA, as pre and post-bronchodilator. In particular, spirometry on visit days shall be conducted 10-14 hours after the last intake of investigational drug on the evening before for bid drugs, and 22-26 hours for once daily drugs after the last inhalation of daily maintenance medication on the prior morning.

The spirometry equipment used during the trial must meet or exceed the minimal ATS/ERS recommendations for diagnostic spirometry equipment as defined in the guideline provided by the vendor. Calibration of the spirometry equipment is mandatory on all visit days and must be performed before the first patient spirometry test is assessed.

The same spirometry equipment should be used for all assessments performed by a subject. A limited number of qualified staff, as designated by the investigator, will evaluate all patients at all visits throughout the entire trial. Where possible the same technician should perform all maneuvers for an individual subject. All staff conducting the spirometry tests must have received appropriate training which must be documented.

All spirometry assessments will be undergoing review by a central overreader. Acceptability of a spirometric assessment attempt depends on the overreader's judgement for compliance with and acceptability according to the ATS/ERS criteria.

### Fibrinogen

Fibrinogen is a glycoprotein, which is the most abundant clotting factor in plasma. It is associated with severity of disease and quality of life in bronchiectasis (Saleh A D, Chalmers J D, Soyza D A et al (2017) The heterogeneity of systemic inflammation in bronchiectasis. Respir Med; 127:33-39). Plasma fibrinogen will be measured by photometric clot detection technique (Clauss method). Samples will be collected at the following timepoints on visits: Days −35 to −1 (screening), Day 1 (predose), Day 28±3 (predose), Day 56±3 (predose), Day 84±3 (predose).

### High Resolution Computed Tomography (HRCT)

High Resolution Computed Tomography (HRCT) will be performed at screening period/baseline and at Day 84 (12 weeks). At the following time points, an HRCT scan of the lung, without contrast agent, will be acquired: Days −35 to −1 (screening), Day 84±3 (predose). The acquisition will include inspiratory and expiratory image sets at both assessment time points. In all subjects, the baseline and follow-up HRCT scan should be performed where possible on the same scanner.

In summary, evaluation of the HRCT scans will be used to assess extent of:
Airway structure and function for evaluation of change from baseline compared to week 12 of HRCT
Exploratory assessments of mucus load and 12 week change from baseline

7.3.8. Safety

Safety assessments are specified in Table 12.

TABLE 12
Assessments and Specifications

| Assessment | Specification |
|---|---|
| Physical examination | A complete physical examination will include the examination of |
| | general appearance, skin, neck (including thyroid), eyes, ears, nose, |
| | throat, lungs, heart, abdomen, back, lymph nodes, extremities, |
| | vascular, and neurological. If indicated based on medical history |
| | and/or symptoms, rectal, external genitalia, breast, and pelvic exams |
| | will be performed. |
| | A short physical exam will include the examination of general |
| | appearance and vital signs (blood pressure [SBP and DBP] and |
| | pulse). A short physical exam will be at all visits starting from Day 1 |
| | visit except where a complete physical examination is required (see |
| | above). |
| Vital signs | Vital signs include blood pressure and pulse measurements. After the |
| | subject has been sitting for 5 minutes, with back supported and both |
| | feet placed on the floor, systolic and diastolic blood pressure will be |
| | measured three times using an automated validated device, e.g. |
| | OMRON, with an appropriately sized cuff. The repeat sitting |
| | measurements will be made at 1-2 minute intervals and the mean of |
| | the 3 measurements will be used. In case the cuff sizes available are |
| | not large enough for the subject's arm circumference, a |
| | sphygmomanometer with an appropriately sized cuff may be used. |
| Height and weight | Height in centimeters (cm) and body weight (to the nearest 0.1 |
| | kilogram (kg) in indoor clothing, but without shoes) will be |
| | measured, mass index (BMI) will be calculated as (Body weight (kg)/ |
| | [Height M)]²) (Screening only). |

### Laboratory Evaluations

A central laboratory will be used for analysis of all specimens collected. All abnormal lab results must be evaluated for criteria defining an adverse event and reported as such if the criteria are met. For those lab adverse events, repeated evaluations are mandatory until normalization of the result(s) or until the result is no longer considered to be clinically significant. Laboratory assessments are described in Table 13 and will be performed at the following timepoints: Days −35 to −1 (screening), Day 1 (predose), Day 28±3 (predose), Day 56±3 (predose), Day84±3 (predose). Pregnancy test will also be administered on Day 91±3 (end of study).

TABLE 13
Laboratory Evaluations

| Test Category | Test Name |
|---|---|
| Hematology | Hematocrit, Hemoglobin, Platelets, Red blood cells, White blood cells, |
| | Differential (Basophils, Eosinophils, Lymphocytes, Monocytes, (absolute |
| | value preferred, %s are acceptable) |
| Chemistry | fibrinogen |
| | Albumin, Alkaline phosphatase, ALT, AST, Gamma-glutamyltransferase |
| | (GGT), Lactate dehydrogenase (LDH), Bicarbonate, Calcium, Magnesium, |
| | Phosphorus, Chloride, Sodium, Potassium, Creatinine, Creatine kinase, |
| | Direct Bilirubin, Indirect Bilirubin, Total Bilirubin, Total Cholesterol, LDL, |
| | HDL, Total Protein, Triglycerides, Blood Urea Nitrogen (BUN) or Urea, |
| | Uric Acid, Amylase, Lipase, Glucose (fasting) |
| Urinalysis | Microscopic Panel (Red Blood Cells, White Blood Cells, Casts, Crystals) |
| | only if abnormalities on the macroscopic panel(dipstick) are detected) |
| | Macroscopic Panel (Dipstick) (Color, Bilirubin, Blood, Glucose, Ketones, |
| | Leukocytes esterase, Nitrite, pH, Protein, Specific Gravity, Urobilinogen) |
| Coagulation | Prothrombin time (PT), International normalized ratio [INR]), Partial |
| | thromboplastin time (PTT), Activated partial thromboplastin time (APTT) |
| Pregnancy Test | Serum/Urine pregnancy test |

### Electrocardiogram (ECG)

PR interval, QRS duration, heart rate, RR interval, QT interval, QTcF will be assessed. The Fridericia QT correction formula (QTcF) must be used for clinical decisions. Unless auto-calculated by the ECG machine, the investigator must calculate QTcF at the Screening to assess eligibility. Clinically significant abnormalities must be reported as adverse events.

### Pregnancy and Assessments of Fertility

All pre-menopausal women who are not surgically sterile (women of childbearing potential) will have a pregnancy testing. A positive pregnancy test at any time during the study requires the patient to be discontinued from the study treatment. Additional pregnancy testing might be performed if requested by local requirements. A serum or urine pregnancy test will be performed at the following timepoints: Days −35 to −1 (screening), Day 1 (predose), Day 28±3 (predose), Day 56±3 (predose), Day84±3 (predose), Day 91±3 (end of study).

### Bronchiectasis Exacerbation

Bronchiectasis exacerbations are defined as a deterioration in three or more of the following key symptoms for at least 48 hours:
cough
sputum volume and/or consistency;
sputum purulence;
breathlessness and/or exercise tolerance;
fatigue and/or malaise;
haemoptysis

### AND

A clinician determines that a change in bronchiectasis treatment is required (e.g. requiring systemic glucocorticosteroid treatment and/or systemic or inhaled antibiotics).

A worsening of symptoms that either does not meet the above symptom definition but is treated by the investigator with antibiotics, or that meets the symptom definition but is not treated with antibiotics, is not considered a pulmonary exacerbation for the study.

For the above reported signs and symptoms, additional information will be collected to document if the reported signs and symptoms last for more than 48 hours.

Patients should contact the site when experiencing a pulmonary exacerbation. An unscheduled visit should occur within 2 working days of the event to confirm the diagnosis, unless the patient is hospitalized and thus unable to attend the site. AEs/SAEs, concomitant medications, and safety laboratory exams should be captured, as appropriate.

The start date for a pulmonary exacerbation recorded in the CRF should be the first day of treatment with antibiotics, as defined above. The end of a pulmonary exacerbation episode is marked by the end of treatment with antibiotics. An exacerbation might result in missed or rescheduled visit(s) and missing associated CRF data in some circumstances. Patients who develop a pulmonary exacerbation between screening and prior to treatment will be screen failed but will be permitted to be re-screened once the inclusion/exclusion criteria have been met.

7.3.9. Additional Assessments

### Patient Reported Outcomes

The impact of bronchiectasis on a subject's health status will be assessed by the following patient-reported questionnaires (PROs):
The St. George Respiratory Questionnaire (SGRQ) providing the health status measurements
The Quality of Life Questionnaire for Bronchiectasis(QOL-B) (Respiratory Symptoms domain) assessing symptoms for patients with bronchiectasis
The European Quality of Life-5 Dimensions-3 level (EQ-5D-3L) measuring global health status
EXAcerbations of COPD Tool-Patient Reported Outcome (EXACT-PRO) evaluating frequency, severity, and duration of exacerbations.
   St. George's Respiratory Questionnaire (SGRQ)

The St. George Respiratory Questionnaire (SGRQ) will be used to provide the health status measurements in this study (Jones P W, Quirk F H, Baveystock C M et al (1992) A self-complete measure of health status for chronic airflow limitation. The St. George's Respiratory Questionnaire. Am Rev Respir Dis. 145(6):1321-7). The SGRQ will be electronically completed by the patient at the investigator's site at the following timepoints: Day 1 (baseline), Day 84±3 (predose).

The SGRQ questionnaire should always be completed before any other assessments (including any other questionnaires) are made to avoid influencing the responses. The SGRQ contains 50 items divided into two parts covering three aspects of health related to Bronchiectasis: Part I covers “Symptoms” and is concerned with respiratory symptoms, their frequency and severity; Part II covers “Activity” and is concerned with activities that cause or are limited to breathlessness; Part II is also concerned with “Impacts”, which covers a range of aspects concerned with social functioning and psychological disturbances resulting from airways disease. A score will be calculated for each of these three subscales and a “Total” score will also be calculated. In each case the lowest possible value is zero and the highest 100. Higher values correspond to greater impairment of health status.

### Quality of Life Questionnaire for Bronchiectasis (QOL-B) (Respiratory Symptoms)

The Quality of Life Questionnaire for Bronchiectasis (QOL-B) (Respiratory Symptoms scale) will be used to assess respiratory symptoms for patients in this study (Quittner A L, O'Donnell A E, Salathe M A et al (2015) Quality of Life Questionnaire-Bronchiectasis: final psychometric analyses and determination of minimal important difference scores. Thorax; 70:12-20). It is a self-administered patient-reported outcome (PRO) measure. QOL-B will be electronically completed by the patient at the investigator's site at the following timepoints: Day 1 (baseline), Day 28±3 (predose), Day 56±3 (predose), Day 84±3 (predose).

QOL-B will be electronically completed by the patient at the investigator's site. It should always be completed before any other assessments (including any other questionnaires) are made to avoid influencing the responses. The Respiratory Symptoms scale contains 9 items.

### European Quality of Life-5 Dimension-3 Level (EQ-5D-3L)

The European Quality of Life-5 Dimensions-3 Level (EQ-5D-3L) developed by the EuroQol Group provides a standardized self-reported measure of global health status. It is a simple, generic measure of health for clinical and economic appraisal (EuroQol Group (1990) EuroQol-a new facility for the measurement of health-related quality of life. Health Policy; 16:199-208). The EQ-5D-3L consists of two pages—the descriptive system and the EQ visual analogue scale (EQ VAS). The descriptive system comprises five dimensions (mobility, self-care, usual activities, pain/discomfort, and anxiety/depression), each with three levels: no problems, some problems, and extreme problems. The patient is asked to indicate his/her present health state according to the most appropriate statement for each of the five dimensions. The EQ VAS records the patients' self-rated health on a 20 cm vertical, visual analogue scale with endpoints labeled ‘the best health you can imagine’ and ‘the worst health you can imagine’. There is no recall period and patient responds to the present health status. EQ-5D-3L will be electronically completed by the patient at the investigator's site at the following timepoints: Day 1 (baseline), Day 28±3 (predose), Day 84±3 (predose). EXACT PRO will be electronically completed by the patient at the investigator's site at the following timepoints: Day 1 (predose), Day 28±3 (predose), Day 56±3 (predose).

The appropriate language version of the questionnaires will be used in each participating country. Subject questionnaires will be completed in the language most familiar to the subject. The same language should be used by a particular patient throughout the study. The site personnel administering the questionnaire should be familiar with the measures and the associated user guides and training materials provided. The patient should complete the questionnaires in a quiet area and be allowed to ask questions; however site personnel should take care not to influence the patient's responses. The patient will be instructed to provide the truest and best response for them.

A subject's refusal to complete all or any part of a PRO measure should be documented in the study EDC system and will not be considered a protocol deviation. The site personnel should check PRO measures for completeness and ask the subject to complete any missing responses. The responses stored electronically on the e-Diary will be considered the source file.

Completed measures and any unsolicited comments written by the subject should be reviewed and assessed by the investigator for responses which may indicate potential AEs or SAEs before any clinical study examinations. This assessment should be documented in the patient's source records. If AEs or SAEs are confirmed, the study investigator should not encourage the subject to change responses reported in the completed questionnaires. Study investigators must follow reporting instructions outlined in the study protocol EXACT Questionnaire.

The EXACT-PRO is a validated 14-item electronic questionnaire designed to detect the frequency, severity, and duration of exacerbations. It is to be completed by the patient at the end of every day at bedtime in order to measure the underlying day to day variability of disease, and detect worsening indicative of an exacerbation.

Within the 14-item EXACT-PRO tool, the Evaluating Respiratory Symptoms (E-RS™) scale is based on the 11 respiratory symptom items. These 11 items generate a total score, quantifying respiratory symptom severity overall, and 3 subscale scores assessing breathlessness, cough and sputum, and chest symptoms. The single questionnaire will be used for two functions: quantification of respiratory symptoms in using E-RS total and subscale scores, and the assessment of acute exacerbations using the entire EXACT-PRO instrument.

Electronic Diary (eDiary)

At screening, all patients will be provided with an electronic diary (eDiary). eDiary will record rescue medication, medications intake (dose) after randomization, as well as, the study questionnaires at prespecified timepoints. The patients will be instructed to routinely complete the rescue medication information in the eDiary twice daily at the same time in the morning and evening (before taking the study drug), approximately 12 hours apart. The eDiary is to be reviewed at each clinic visit until study completion: Days −35 to −1 (screening), Day 1 (screening), Day 1 (predose), Day 28±3 (predose), Day 56±3 (predose), Day 84±3 (predose), Day 91±3 (end of study). Sites and patients will receive appropriate training and guidance on the use of the eDiary device

### Pharmacokinetics

PK samples will be collected at treatment visits: Day 1 (predose), Day 1 (1 h, 2 h, 3 h, 4 h, 6 h, 8 h after dose), Day 28±3 (predose), Day 28±3 (1 h, 2 h, 3 h, 4 h, 6 h, 8 h after dose), Day 56±3 (predose), Day 56±3 (3 h after dose), Day 84±3 (predose), and Day 84±3 (3 h after dose). Instructions outlined in the laboratory manual regarding sample collection, numbering, processing and shipment shall be followed.

PK sampling (pre-dose and 3 hour post-dose) will be conducted and evaluated in all subjects on Days 1, 28, 56 and 84 (end of treatment). A subset of all patients enrolled from China sites and approximately 10-20 patients from selected sites in Europe will undergo serial PK sampling up to 8 hours post-dose at Day 1 and Day 28 visits to further characterize the PK profile of Compound A in bronchiectasis patients, which will provide important understanding of the 450 mg b.i.d. dose (or 300 mg b.i.d. if the 450 mg treatment is discontinued) in bronchiectasis patients.

Furthermore, additional PK samples will be collected, where possible, from patients experiencing a treatment-emergent SAE. In case a patient prematurely discontinued treatment but continue participation in the study, the first visit after the discontinuation should have a PK trough assessment and thereafter subsequent PK sampling should be suspended. Plasma PK samples will be evaluated only in subjects who have been administereed Compound A. Compound A concentration will be determined by a validated LC-MS/MS method with an anticipated lower limit of quantification (LLOQ) of 1 ng/mL of Compound A. Concentrations below the LLOQ will be reported as zero and missing data will be labeled as such in the bioanalytical report.

The following pharmacokinetic parameters will be determined using the actual recorded sampling times and non-compartmental method(s) with Phoenix WinNonlin (Version 8 or higher): Cmax, Tmax, AUClast, AUCO-12 h, T1/2,eff. Residual plasma samples remaining after the determination of Compound A may be used for exploratory assessment of metabolites or other bioanalytical purposes (e.g. cross check between different sites, stability assessment).

7.3.10. Study discontinuation and completion

### Discontinuation

Discontinuation of study treatment for a subject occurs when study treatment is stopped earlier than the protocol planned duration and can be initiated by either the subject or the investigator. Subjects may voluntarily discontinue study treatment for any reason at any time. The investigator must discontinue study treatment for a given subject if he/she believes that continuation would negatively impact the subject's well-being. Study treatment must be discontinued under the following circumstances:
Subject/guardian decision
Pregnancy: a positive urine pregnancy test after start study drug requires immediate interruption of study drug until serum hCG is performed and found to be negative. If positive, the subject must discontinue study medication.
Use of prohibited treatment.
Any situation in which study participation might result in a safety risk to the subject.
Following emergency unblinding
Emergence of an Adverse Event (AE) reported as severe and suspected to be related to investigational drug or an SAE reported and suspected to be related to the investigational drug.
Emergence of an SAE not suspected to be related to investigational drug in a patient with a verified exposure above the threshold (AUCO-24 h=91,700 ng*h/ml). The investigator must permanently discontinue the investigational study drug as soon as possible upon receipt of the PK results.
A patient with a verified exposure above the upper range of the individual animal (monkey) model exposure (AUCO-24 h=159,000 ng*h/mL). The investigator must stop study drug immediately and must permanently reduce the investigational study drug to 300 mg b.i.d. as soon as possible upon receipt of the PK results.
Any laboratory abnormalities that in the judgment of the investigator, taking into consideration the subject's overall status, prevents the subject from continuing participation in the study.
Any liver event requiring immediate discontinuation of study treatment (i.e., Potential Hy's Law case, >8×ULN, >3×ULN and INR>1.5, >5 to ≤8×ULN (discontinue immediately if elevation persists for more than 2 weeks), >3×ULN accompanied by symptoms, >2×ULN (in the absence of known Gilbert syndrome)(discontinue immediately if elevation persists), Jaundice, any AE potentially indicative of a liver toxicity)
Individual serum creatinine increase ≥50% compared to baseline (must be confirmed)
Study drug can be temporarily interrupted as a response to the occurrence of adverse events that do not fulfill the requirements above described for permanent discontinuation Discontinuation of study treatment will be at the discretion of the Investigator under the following circumstances:
Any other protocol deviation that results in a significant risk to the subject's safety.
Emergence of adverse event(s) or laboratory abnormalities that in the judgement of the Investigator, taking into account the subjects' overall status, prevent the subject from continuing participation in the study.

The appropriate personnel from the site and Novartis will assess whether study treatment should be discontinued for any subject whose treatment code has been broken inadvertently. Unblinding for emergency reasons requires study drug discontinuation. If discontinuation of study treatment occurs, the investigator should make a reasonable effort to understand the primary reason for the subject's premature discontinuation of study treatment and record this information.

### Dose Reduction

A PK monitoring plan is featured to ensure that patients' exposures are generally consistent with an exposure threshold (AUCO-24 h=91,700 ng*h/ml), which was established based on monkey data.

The following should be considered and the treatment arm may be permanently discontinued if: At predefined safety interim read outs the observed proportion of patients above the threshold (AUCO-24 h=91,700 ng*h/ml) is significantly greater than expected (it is expected that <5% of patients on 300 mg or 450 mg b.i.d. will exceed the threshold based on a conservative scenario) and/or a projected AUCO-24 h above the upper range of the individual monkey exposures (159,000 ng*h/ml) is exhibited.

PK exposure values above the threshold due to unverified sampling (sampling time cannot be confirmed) or analysis or the consequence of an accidental overdosing will not be included in the determination of whether to stop the 450 mg b.i.d. treatment arm. If the dose is reduced to 300 mg b.i.d., an unscheduled Ctrough sample will be collected following the first week of 300 mg b.i.d. administration to ensure the PK exposure is reduced to the expected level.

### Study Completion

Study completion is defined as when the last subject finishes their Study Completion visit and any repeat assessments associated with this visit have been documented and followed-up appropriately by the Investigator or, in the event of an early study termination decision, the date of that decision (e.g. each subject will be required to complete the study in its entirety and thereafter no further study treatment will be made available to them). All randomized and/or treated subjects should have a safety follow-up call conducted 30 days after last administration of study treatment. The information collected is kept as source documentation.

7.3.11. Safety monitoring

### Liver Safety Monitoring

To ensure subject safety and enhance reliability in determining the hepatotoxic potential of an investigational drug, a standardized process for identification, monitoring and evaluation of liver events has to be followed.

The following two categories of abnormalities/adverse events have to be considered during the course of the study (irrespective of whether classified/reported as AE/SAE) and further described in Table 14.
Liver laboratory triggers, which will require repeated assessments of the abnormal laboratory parameter
Liver events, which will require close observation, follow-up monitoring and contributing factors are recorded on the appropriate CRFs
Liver events and liver triggers are detailed in Table 13. Repeat liver chemistry tests (i.e. ALT, AST, TBL, PT/INR, ALP and G-GT) to confirm elevation.
These liver chemistry repeats will be performed using the central laboratory. If results will not be available from the central laboratory, then the repeats can also be performed at a local laboratory to monitor the safety of the subject. If a liver event is subsequently reported, any local liver chemistry tests previously conducted that are associated with this event should have results recorded on the appropriate CRF
If the initial elevation is confirmed, close observation of the subject will be initiated, including consideration of treatment interruption if deemed appropriate.
Discontinuation of the investigational drug, if appropriate
Hospitalization of the subject if appropriate
Causality assessment of the liver event
Thorough follow-up of the liver event should include: serology tests, imaging and pathology assessments, hepatologist's consultancy; obtaining more detailed history of symptoms and prior or concurrent diseases, history of concomitant drug use, exclusion of underlying liver disease and imaging such as abdominal ultrasound, computer tomography (CT) or magnetic resonance imaging (MM), as appropriate.

TABLE 14
Liver event and laboratory trigger definitions

| | |
|---|---|
| Liver Laboratory Triggers | 3 × ULN < ALT/AST ≤ 5 × ULN |
| | 1.5 × ULN < TBL ≤ 2 × ULN |
| Liver Events | ALT or AST > 5 × ULN |
| | ALP > 2 × ULN (in the absence of known bone pathology) |
| | TBL > 2 × ULN (in the absence of known Gilbert syndrome) |
| | ALT or AST > 3 × ULN and INR >1.5 |
| | Potential Hy's Law cases (defined as ALT or AST > 3 × ULN and |
| | TBL > 2 × ULN [mainly conjugated fraction] without notable increase |
| | in ALP to >2 × ULN) |
| | Any clinical event of jaundice (or equivalent term) |
| | ALT or AST > 3 × ULN accompanied by (general) malaise, |
| | fatigue, abdominal pain, nausea, or vomiting, or rash with eosinophilia |
| | Any adverse event potentially indicative of a liver toxicity* |

*These events cover the following: Hepatic failure, fibrosis and cirrhosis, and other liver damage related conditions; the non-infectious hepatitis; the benign, malignant and unspecified liver neoplasms
TBL: total bilirubin;
ULN: upper limit of normal

### Renal Safety Monitoring

Renal safety monitoring for the investigational drug will be performed in the study. This includes baseline measurements of serum creatinine, calcium, potassium and urine dipstick and at subsequent visits at the following timepoints: Days −35 to −1 (screening), Day 1 (predose), Day 28±3 (predose), Day 56±3 (predose), Day 84±3 (predose). Specific renal alert criteria include:
Confirmed serum creatinine increase 25-49%, serum creatinine increase 50+OR if <18 years old, eGFR<35 mL/min/1.73 m²
New onset dipstick proteinuria ≥3+OR protein-creatinine ratio (PCR)≥1 g/g Cr (or mg/mmol equivalent as converted by the measuring laboratory)
New onset hematuria ≥3+ on urine dipstick

7.3.12. Data Analysis and Statistical Methods

The analysis will be conducted on all subject data at the time the trial ends.

### Analysis Sets

For all analysis sets, subjects will be analyzed according to the study treatment(s) received. The Safety Set includes all subjects who received at least one dose of study treatment whether or not being randomized. The safety set will be used in the analysis of all safety variables. The PD analysis set will include all subjects with available PD data at both baseline and at least one post-baseline assessment which are not affected by any protocol deviations. The PK analysis set will include all subjects with at least one available valid (i.e. not flagged for exclusion) PK concentration measurement, who received any study drug and with no protocol deviations that affect PK data.

### Subject Demographics and Other Baseline Characteristics

Demographic and other baseline data, including disease characteristics, will be listed and asummarized descriptively by treatment group for the full analysis set. Categorical data will be presented as frequencies and percentages. For continuous data, mean, standard deviation, median, minimum, and maximum will be presented. Relevant medical histories and current medical conditions at baseline will be summarized by system organ class and preferred term, by treatment group.

### Treatments

The Safety set will be used for the analyses below. Categorical data will be summarized as frequencies and percentages. For continuous data, mean, standard deviation, median, 25th and 75th percentiles, minimum, and maximum will be presented. The duration of exposure in days to each treatment group (Compound A dose or placebo) will be summarized by means of descriptive statistics using the safety set. Concomitant medications and significant non-drug therapies prior to and after the start of the study treatment will be listed and summarized according to the Anatomical Therapeutic Chemical (ATC) classification system, by treatment group.

### Analysis of Primary Endpoint(s)

The primary objective of the study is to assess the change from baseline of Compound A compared to placebo on the total number of bacteria over all strains after 12 weeks of treatment. The PD Analysis Set will be used for analysis of the primary variable, unless otherwise specified.

The primary estimand targets the hypothetical effect as if all patients had stayed on treatment for 12 weeks and as if antibiotics other than macrolide had not been available.
Population: patients are defined by the study inclusion and exclusion criteria. The protocol defines the use of allowed maintenance therapy for this target population, as well as medications that are prohibited.
Variable: change from baseline in bacterial load as measured by the number of colony forming units (CFU/ml) of potentially pathogenic microorganisms in sputum at week 12. A log₁₀ transformation is applied to the CFU counts before forming the change from baseline computing therefore the log ratio to baseline of the bacterial load.
Intervention effect of interest:
   Intake of antibiotics other than macrolide and intake of rescue medication
   Treatment discontinuation or study participation: hypothetical on treatment CFU counts
Summary measure: difference in variable means (Compound A compared with placebo)

The baseline CFU of potentially pathogenic microorganisms in spontaneous sputum is defined as the average of assessment of bacterial load at baseline (on log 10-transformed scale) and the sputum microbiology results at screening. If either assessment is missing, then the result from the other assessment will be considered as baseline. The CFU counts assessed 2-4 weeks after pulmonary exacerbations or during or after last dose of antibiotics will not be included in the primary estimand. Repeated measurements of CFU at week 4, week 8 and week 12 are considered in the primary analysis.

### Statistical Model, Hypothesis, and Method of Analysis

The primary endpoint will be analyzed using a Bayesian repeated measures model with change from baseline in CFU counts as response, adjusting for effect of treatment*visit interaction, status of macrolides use at screening as factor, baseline CFU counts, and any additional covariate as deemed appropriate. In absence of informative data, non-informative priors for the model parameters will be used. The prior for placebo may be updated as a weakly informative prior in the statistical analysis plan, should new relevant data become available.

A comparison of Compound A vs. placebo is of primary interest. Based on the fitted Bayesian model for repeated measures, the posterior probability of Compound A effect over placebo for log₁₀ CFU will be calculated. Statistical evidence will be concluded if there is 90% probability that the true effect over placebo for log₁₀ CFU is >0. The posterior probabilities of efficacy will be assessed according to the following criteria:
Go/success criteria at final: Better than placebo with high confidence (at least 90% probability that the change from baseline in log₁₀ CFU for Compound A is better than placebo, i.e. Posterior Prob (delta >0)>0.90)

Due to the nonconfirmatory nature of this study, no multiplicity adjustment will be applied. To assess the robustness of the results, the PoC criteria will also be evaluated considering a noninformative prior on placebo. In addition, the mean and corresponding two sided 80% credible interval for the Compound A difference to placebo from the posterior distribution will be presented.

### Handling of Missing Values/Censoring/Discontinuations

Since the primary estimand is related to an effect outside of antibiotics intake, the CFU counts assessed within 2-4 weeks after pulmonary exacerbations or during or after last dose of antibiotics will not be included in the primary estimand because potential confounding effect is expected from the use of antibiotics. So the corresponding CFU counts from that visit will be set to be missing. However, the CFU counts at the following visits (if there is no antibiotics use and no pulmonary exacerbation) are assumed not to be confounded and will be included in the primary estimand of interest.

For the primary analysis, only on-treatment data (from date of first randomized dose up to day after date of last randomized dose) will be used as the estimand specifies a hypothetical on treatment effect. Missing on-treatment data related to primary endpoint will not be explicitly imputed. The repeated measures analysis includes all available information in terms of measurements at all times. If endpoint measurements are missing at random, an analysis of the available data provides consistent estimates of model parameters.

Sensitivity and Supportive Analyses

The primary estimand is complemented by two supplementary estimands. This would allow to assess the robustness of the Compound A treatment effect on bacterial load drop with and without antibiotics use/pulmonary exacerbations that would be deemed to affect the outcome of interest.

1. To estimate the effect of study drug versus placebo without potential confounding due to the antibiotics use. All visits after receiving antibiotics other than macrolide will not be included in the CFU analysis (they will be set as missing). Note that this approach will lead to less data being used than the primary estimand.

2. To estimate the effect regardless of antibiotics use. This estimand follows the treatment policy strategy where antibiotics are used as needed basis on top of study treatment as in clinical practice. Thus, the CFU measurements collected during the intake of antibiotics will be included in the analysis. The estimate of treatment actually means (treatment+any antibiotics) in this context.

### Analysis of Secondary Endpoints

The proportion of patients with absence of any CFU or CFU counts below the limit of quantification at week 12 will be analyzed using a logistic regression. The model will include treatment, baseline macrolide use as fixed class effects and the number of bronchiectasis exacerbations in the 12 months prior to screening as a categorical variable. The estimated odds ratios will be displayed along with the associated 80% confidence intervals.

The change from baseline in fibrinogen is assumed normally distributed. An MMRM will be fitted to the changes from baseline in fibrinogen for all time points until Day 84 visit including the following fixed factors: treatment group, visit, treatment group by visit interaction, baseline macrolide use, and baseline fibrinogen value by time interaction.

The mean night-time/day-time number of puffs of rescue medication will be calculated for each subject for each visit interval. The total number of puffs of rescue medication will be divided by the total number of (full or half) days with non-missing rescue data to derive the mean daily number of puffs of rescue medication taken for the patient for each given visit interval. If the number of puffs is missing for part of the day (either morning or evening) then a half day will be used in the denominator. A ‘day without usage of rescue medication’ will be defined from the diary data as any day where the patient took no puffs of rescue medication. The total number of days with ‘no rescue use’ for each given visit interval will be divided by the total number of days where diary recordings have been made in order to derive the proportion of days without usage of rescue medication. The daily mean number of puffs of rescue medication and the proportion of 'day without usage of rescue medication will be also be calculated for each subject for each visit interval by combining records from both night-time/day-time number of puffs of rescue medication.

The descriptive statistics for each baseline in pre- and post-bronchodilator FEV1, FVC, measured by spirometry variable will be provided by treatment. Change from baseline in FEV1 and FVC will be analyzed using the similar statistical method with the same factors of interest described for the secondary efficacy endpoint. A MMRM will be handled in the statistical model accordingly.

All safety endpoints will be analyzed based on safety set and will be summarized by actually received treatment group. Safety summaries (tables, figures) include only data from the on-treatment period with the exception of baseline data which will also be summarized where appropriate (e.g. change from baseline summaries). In addition, a separate summary for death including on treatment and post treatment (30 days after the last actual administration of study treatment) deaths will be provided. In particular, summary tables for adverse events (AEs) will summarize only ontreatment events, with a start date during the on-treatment period (treatment-emergent AEs). The on-treatment period lasts from the date of first administration of study treatment to one week after the date of the last actual administration of any study treatment.

PK analysis set will be used for the analysis of all pharmacokinetic parameters. Descriptive statistics of Compound A plasma concentration data will be provided by treatment and visit/sampling time point, including the frequency (n, %) of concentrations below the lower limit of quantification (LLOQ). Summary statistics of Compound A plasma concentration data and PK parameters will include mean (arithmetic and geometric), SD, CV (arithmetic and geometric), median, minimum, and maximum. An exception to this is Tmax, where median, minimum, and maximum will be presented. Concentrations below LLOQ will be treated as zero in summary statistics and for PK parameter calculations. The PK parameters which will be determined from the blood concentration time data include (but not limited to), where possible: Cmax, Cmin, Tmax, AUClast, AUCO-12 h, and T1/2,eff. Pharmacokinetic parameters will be determined using WinNonlin Phoenix (version 8 or higher). An exploratory analysis of the relationship between pharmacokinetic and pharmacodynamic measures may be performed using a model based approach, if the data measures permits.

### Analysis of Exploratory Endpoints

Change from baseline in airway wall and lumen parameters will be analyzed using the same model as for the efficacy endpoint for fibrinogen plasma concentration. Global and region air trapping changes will be summarized. Contrasts for treatment differences will be provided together with two-sided 80% confidence intervals. Any correlations between changes in air trapping and the lung function parameters measured by spirometry would be explored.

### Bronchiectasis Exacerbation

The following analyses will be performed to explore any differences in the bronchiectasis exacerbation events that occur in Compound A vs placebo:

The time to first bronchiectasis exacerbations analyses will be carried out only upon sufficient number of exacerbation events occur during the study to estimate the median in either of the treatment groups. The time to the first on-treatment bronchiectasis exacerbation (event) is defined as the earliest start date of a bronchiectasis exacerbation minus the date of randomization +1. Patients who do not experience an exacerbation or discontinued earlier without an exacerbation will be considered as censored for analyses purpose at the end of the treatment period. Events which occur after randomization and during the treatment period will be included in the analysis. The hazard ratios for Compound A compared with placebo and their corresponding 80% confidence intervals will be computed using Kaplan-Meier method. The stratification factor may include number of exacerbations in the last 12 months as =1 and >1. The Kaplan-Meier estimates of the survival functions for each treatment will be plotted.

The number of bronchiectasis exacerbations will be analyzed using a generalized linear model assuming a negative binomial distribution. The time at risk for a patient is defined as the length of time the patient is on treatment and the log (length of time) will be used as the offset variable in the model. The model will include treatment, baseline macrolides use, and the number of bronchiectasis exacerbations in the 12 months prior to screening as categorical variables. An estimate of the rate ratio together with 80% confidence intervals and corresponding p-value will be presented.

Exploratory DNA studies are designed to investigate the association between genetic factors (genotypes) and clinical assessments (phenotypes) which are collected during the clinical trial. Without prior evidence of a strong association, a number of possible associations are evaluated with exploratory analyses. A range of statistical tests are used for the analyses. Additional data, from other clinical trials, are often needed to confirm associations. Alternatively, if the number of subjects enrolled in the study is too small to complete proper statistical analyses, the data may be combined, as appropriate, with those from other studies to enlarge the dataset for analysis.

### Sample Size Calculation

This study will enroll approximately of 72 patients who will be randomized in a 1:1 ratio to receive either Compound A 450 mg (or Compound A 300 mg if the 450 mg treatment is discontinued) or placebo in order to achieve 60 patients who complete the treatment period based on the assumption of a 16% drop-out rate. There will be approximately 79% power to show that Compound A is superior to placebo in reducing bacterial load with 10% level of significance, assuming a true difference of 1.5 log 10 CFU count and a standard deviation of 2.8 for the change from baseline to Day 84 on log 10 scale. Regarding the assumption on the standard deviation of the change from baseline in bacterial load (log 10 scale), this is derived from two historical trials in bronchiectasis and COPD patients with a conservative view. There will be about 10% chance to erroneously declare positive PoC (Type 1 error). The sample size assumptions will be reviewed in a blinded manner when approximately 14 patients complete the treatment period. If deemed appropriate, in the case of a higher dropout rate than assumed or a higher variability of the primary endpoint in either region, or if there is foreseen a significant imbalance between sites in Europe and China, up to 108 subjects may be randomized in order to achieve an adequate number of completers.

### REFERENCES

Amaral M D and Kunzelmann K (2007) Molecular targeting of CFTR as a therapeutic approach to cystic fibrosis. TRENDS in Pharmacological Sciences; 28(7): 334-341
Bergougnoux A, Viart V, Miro J, et al (2015) Should diffuse bronchiectasis still be considered a CFTR-related disorder. J Cyst Fibros; 14(5): 646-653
Bienvenu T, Sermet-Gaudelus I, Burgel P R, et al (2010) Cystic fibrosis transmembrane conductance regulator channel dysfunction in non-cystic fibrosis bronchiectasis. Am J Respir Crit Care Med; 181(10):1078-1084
Boucher R C (2007) Cystic fibrosis: a disease of vulnerability to airway surface dehydration. Trends Mol Med; 13(6): 231-240
Chalmers J D, Smith M P, McHugh B J, et al (2012) Short- and Long-Term Antibiotic Treatment Reduces Airway and Systemic Inflammation in Non-Cystic Fibrosis Bronchiectasis. Am J Respir Crit Care Med; 186(7): 657-665
Chalmers J D, Goeminne P, Aliberti S, et al (2014) Derivation and validation of the bronchiectasis severity index: an international multicentre observational study. Am J Respir Crit Care Med; 189: 576-585
Dinakar C, O'Connor G T (2016) The Health Effects of Electronic Cigarettes. N Engl J Med; 375(14):1372-1381
EuroQol Group (1990) EuroQol-a new facility for the measurement of health-related quality of life. Health Policy; 16:199-208
Fahy J V, Dickey B F (2010) Airway mucus function and dysfunction. N Engl J Med; 363(23): 2233-2247
Hogg J C, Chu F, Utokaparch S, et al (2004) The Nature of Small-Airway Obstruction in Chronic Obstructive Pulmonary Disease. N Engl J Med 2004; 350(26):2645-2653 ICH-E2D Guideline 2004. https://www.ich.org/fileadmin/Plublic_Web . . . /Guidelines/ . . . /E2E/ . . . /E2E Guideline.pdf.
Jones P W, Quirk F H, Baveystock C M et al (1992) A self-complete measure of health status for chronic airflow limitation. The St. George's Respiratory Questionnaire. Am Rev Respir Dis. 145(6): 1321-7
Mannino D. M. Singer R. T. Lomas D. A. et al (2015) Plasma Fibrinogen as a Biomarker for Mortality and Hospitalized Exacerbations in People with COPD. Chronic Obstr Pulm Dis.; 2(1): 23-34
Miller M. R, Hankinson J. et al (2005)a Standardisation of spirometry. Eur Respir J. 26(2):319-38
Miller M. R, Crapo R. J. et al (2005)b General considerations for lung function testing. Eur Respir J. 26: 153-161
O'Donnell A E (2008) Bronchiectasis. Chest; 134(4): 815-823
O'Sullivan B P, Freedman, S D (2009) Cystic fibrosis. Lancet; 373(9673), 1891-904
Pezzulo A A, Tang X X, Hoegger M J et al (2012) Reduced airway surface pH impairs bacterial killing in the porcine cystic fibrosis lung. Nature; 487(7405):109-13.
Polverino E, Goeminne P C, McDonnell M J et al (2017) European Respiratory Society guidelines for the management of adult bronchiectasis. Eur Resp J; 50:1700629 Quint J K, Millett E R, Joshi M et al (2016) Changes in the incidence, prevalence and mortality of bronchiectasis in the U K from 2004 to 2013: a population-based cohort study. Eur Respir J; 47(1): 186-93
Quittner A L, O'Donnell A E, Salathe M A et al (2015) Quality of Life Questionnaire-Bronchiectasis: final psychometric analyses and determination of minimal important difference scores. Thorax; 70:12-20
Saleh A D, Chalmers J D, Soyza D A et al (2017) The heterogeneity of systemic inflammation in bronchiectasis. Respir Med; 127:33-39
Solomon G M, Fu L, Rowe S M, Collawn J F, et al (2017) The therapeutic potential of CFTR modulators for COPD and other airway diseases. Curr Opin Pharmacol; 34:132-139

### 8. INCORPORATION BY REFERENCE

The entire disclosure of each of the patent documents and scientific articles cited herein is incorporated by reference for all purposes.

### 9. EMBODIMENTS/CONSISTORY CLAUSES

Embodiment 1. A method for treating bronchiectasis comprising administering at least one compound according to Formula (I):

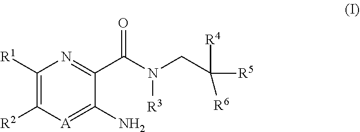

or a pharmaceutically acceptable salt thereof, to a subject in need thereof, wherein:

### A is N or CR^{4a};

R¹ is H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; halogen; SO₂NR⁸R⁹; SO₂R¹⁰; S—C₁-C₈alkyl optionally substituted by one or more halogen atoms; S—C₆-C₁₄ aryl; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; CN; NR¹¹R¹²; CONR¹³R¹⁴; NR¹³SO₂R¹⁵; NR¹³C(O)R¹⁵ and CO₂R¹⁵, wherein the cycloalkyl, cycloalkenyl, aryl and heterocyclyl groups are each optionally substituted by one or more Z substituents;

R² is C₁-C₄ haloalkyl;

R³, R⁴ and R^{4a} are each independently H or C₁-C₈ alkyl optionally substituted by one or more halogen atoms;

R⁵ and R⁶ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; OH; CN; halogen; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; or —(C₀-C₄ alkyl)-CO₂R¹⁵, wherein the cycloalkyl, cycloalkenyl, —(C₀-C₄ alkyl)-C₆-C₁₄ aryl and —(C₀-C₄ alkyl)-3 to 14 membered heterocyclic group groups are each optionally substituted by one or more Z substituents; or

R⁵ and R⁶ are each independently a group of the formula:

—(CH₂)ₘ—NR¹⁷R¹⁸; or

R⁵ and R⁶ are each independently a group of the formula:

—(CH₂)ₘ—OR⁴; or

R⁴ and R⁵ together with the carbon atoms to which they are bound form a 3 to 8 membered carbocyclic ring system; or

R⁵ and R⁶ together with the carbon atoms to which they are bound form a 5 to 8 membered carbocyclic ring system or a 5 to 8 membered heterocyclic ring system containing one or more heteroatoms selected from N, O and S, wherein the ring system is optionally substituted by one or more Z substituents;

R⁴, R⁵ and R⁶ cannot all be the same;

m is 0, 1, 2 or 3;

R⁸, R¹¹, R¹³ and R¹⁷ are each independently H, C₁-C₈ alkyl optionally substituted by one or more halogen atoms, C₃-C₁₀ cycloalkyl or —(C₁-C₄ alkyl)-C₃-C₈ cycloalkyl;

R⁹, R¹⁰, R¹², R¹⁴, R¹⁵, R¹⁶ and R¹⁸ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the cycloalkyl, cycloalkenyl, aryl and heterocyclyl groups are each optionally substituted by one or more Z substituents; or R⁸ and R⁹, R¹¹ and R¹², R¹³ and R¹⁴, and R¹⁷ and R¹⁸ together with the nitrogen atom to which they are attached may form a 4 to 14 membered heterocyclic group optionally substituted by one or more Z substituents;

Z is independently OH, aryl, O-aryl, benzyl, O-benzyl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁸(SO₂)R²¹, (SO₂)NR¹⁹R²¹, (SO₂)R²¹, NR¹⁸C(O)R²¹, C(O)NR¹⁹R²¹, NR¹⁸C(O)NR¹⁹R²¹, NR¹⁸C(O)OR¹⁹, NR¹⁹R²¹, C(O)OR¹⁹, C(O)R¹⁹, SR¹⁹, OR¹⁹, oxo, CN, NO₂, halogen or a 3 to 14 membered heterocyclic group, wherein the heterocyclic group contains at least one heteroatom selected from N, O and S;

R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy.

Embodiment 2. A method for treating bronchiectasis comprising administering at least one compound of Formula (I)

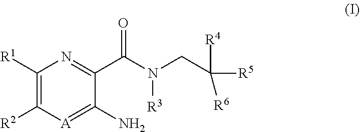

or a pharmaceutically acceptable salt thereof, to a subject in need thereof, wherein:

A is N or CR^{4a};

R¹ is H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; halogen; SO₂NR⁸R⁹; SO₂R¹⁰; S—C₁-C₈alkyl optionally substituted by one or more halogen atoms; S—C₆-C₁₄ aryl; CN; NR¹¹R¹²; C(O)NR¹³R₁₄; NR¹³SO₂R¹⁵; NR¹³C(O)R¹⁵, CO₂R¹⁵, —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, aryl and heterocyclyl groups are each optionally substituted by one or more Z substituents;

R² is C₁-C₄ haloalkyl;

R³ and R^{4a} are each independently H or C₁-C₈ alkyl optionally substituted by one or more halogen atoms;

R⁴ is H, or C₁-C₈ alkyl optional substituted with one or more halogen;

R⁵ is —(CH₂)ₘ—NR¹⁷R¹⁸, —(CH₂)ₘ—OR′; C₁-C₈ alkoxy optionally substituted by one or more halogen atoms; —(C₀-C₄ alkyl)-CO₂R¹⁵; —(C₀-C₄ alkyl)-C₆-C₁₄ aryl or −3 to 14 membered heterocyclic group, wherein the heterocyclic group contains at least one heteroatom selected from N, O and S; wherein the —(C₀-C₄ alkyl)-C₆-C₁₄ aryl and —(C₀-C₄ alkyl)-3 to 14 membered heterocyclic group are each optionally substituted by one or more Z substituents; R⁶ is C₁-C₈ alkyl optionally substituted by one or more halogen atoms; C₃-C₁₀ cycloalkyl; —C₁-C₄ alkyl-C₃-C₈ cycloalkyl; C₁-C₈ alkoxy optionally substituted by one or more halogen atoms; OH; CN; halogen; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, —(C₀-C₄ alkyl)-C₆-C₁₄ aryl and —(C₀-C₄ alkyl)-3 to 14 membered heterocyclic group are each optionally substituted by one or more Z substituents; or R⁶ is H, and R⁵ is —(CH₂)ₘ—NR¹⁷R¹⁸, —(CH₂)ₘ—OR′, C₁-C₈ alkoxy optionally substituted by one or more halogen atoms; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; or —(C₀-C₄ alkyl)-CO₂R¹⁵, wherein —(C₀-C₄ alkyl)-C₆-C₁₄ aryl and —(C₀-C₄ alkyl)-3 to 14 membered heterocyclic group groups are each optionally substituted by one or more Z substituents; or

R⁴ and R⁶ together with the carbon atoms to which they are bound form a 3 to 8 membered carbocyclic ring system; or

R⁴ and R⁵ together form an oxo group (C═O) and R⁶ is C₁-C₄ alkyl optionally substituted by one or more halogen atoms; C₁-C₄ alkoxy optionally substituted by one or more halogen atoms; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the aryl and heterocyclyl groups are each optionally substituted by one or more Z substituents; or

R⁵ and R⁶ together with the carbon atoms to which they are bound a 5 to 8 membered heterocyclic ring system containing one or more heteroatoms selected from N, O and S, wherein the ring system is optionally substituted by one or more Z substituents; or

R⁴ and R⁵ and R⁶ together with the carbon atoms to which they are bound form a 5 to 8 membered heterocyclic ring system containing one or more heteroatoms selected from N, O and S, wherein the ring system is optionally substituted by one or more Z substituents;

R′ is H, or C₁-C₈ alkyl optional substituted with one or more halogen;

m is 0, 1, 2 or 3;

R₈, R¹¹, R¹³ and R¹⁷ are each independently H, C₁-C₈ alkyl optionally substituted by one or more halogen atoms, C₃-C₁₀ cycloalkyl or —(C₁-C₄ alkyl)-C₃-C₈ cycloalkyl;

R⁹, R¹⁰, R¹², R¹⁴, R¹⁵, R¹⁶ and R¹⁸ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the cycloalkyl, cycloalkenyl, aryl and heterocyclyl groups are each optionally substituted by one or more Z substituents; or R⁸ and R⁹, R¹¹ and R¹², R¹³ and R¹⁴, and R¹⁷ and R¹⁸ together with the nitrogen atom to which they are attached may form a 4 to 14 membered heterocyclic group optionally substituted by one or more Z substituents;

Z is independently OH, aryl, O-aryl, benzyl, O-benzyl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁸(SO₂)R²¹, (SO₂)NR¹⁹R²¹, (SO₂)R²¹, NR¹⁸C(O)R²¹, C(O)NR¹⁹R²¹, NR¹⁸C(O)NR¹⁹R²¹, NR¹⁸C(O)OR¹⁹, NR¹⁹R²¹, C(O)OR¹⁹, C(O)R¹⁹, SR¹⁹, OR¹⁹, oxo, CN, NO₂, halogen or a 3 to 14 membered heterocyclic group, wherein the heterocyclic group contains at least one heteroatom selected from N, O and S;

R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy.

Embodiment 3. The method according to Embodiment 1 or Embodiment 2, wherein R¹ is H; C₁-C₄ alkyl optionally substituted by one or more halogen atoms; C₁-C₄ alkoxy optionally substituted by one or more halogen atoms; halogen; C₆-C₁₄ aryl; —(C₀-C₄ alkyl)-3 to 14 membered heterocyclic group, wherein the heterocyclic group contains at least one heteroatom selected from N, O and S; or —NR¹¹R¹², wherein the aryl and heterocyclic groups are each optionally substituted by one or more Z substituents.

Embodiment 4. The method according to one of Embodiment 1 to Embodiment 3, wherein R¹ is C₁-C₄ alkyl optional substituted by one or more halogen atoms.

Embodiment 5. The method according to one of Embodiment 1 to Embodiment 4, wherein R¹ is —CH₃ or CF₃.

Embodiment 6. The method according to one of Embodiment 1 to Embodiment 3, wherein R¹ is C₁-C₄ alkoxy optional substituted by one or more halogen atoms.

Embodiment 7. The method according to any of Embodiment 1 to Embodiment 3 or Embodiment 6, wherein R¹ is —OCH₃, —OCH₂CH₃ or —OCF₃.

Embodiment 8. The method according to one of Embodiment 1 to Embodiment 3, wherein R¹ is aryl, wherein aryl is phenyl optionally substituted by one or more Z substituents,

Embodiment 9. The method according to any of Embodiment 1 to Embodiment 3 or Embodiment 8, wherein R¹ is 4-fluorophenyl, 4-chloro-2-methylphenyl, or 2,4-dichlorophenyl.

Embodiment 10. The method according to one of Embodiment 1 to Embodiment 3, wherein R¹ is pyridyl, oxazole, pyrrolidine or pyrazole and is optionally substituted by one or more Z substituents.

Embodiment 11. The method according to any of Embodiment 1 to Embodiment 3 or Embodiment 10, wherein R¹ is 1-methyl-4-pyridyl, oxzaoyl-2-yl, 1-methyl-1H-pyrazole-4-yl or pyrrolidin-1yl.

Embodiment 12. Embodiment 12: The method according to one of Embodiment 1 to Embodiment 11, wherein R¹ is Br, —CH₃, —CF₃, —OCH₃, —OCH₂CH₃, —OCF₃, 4-fluorophenyl, 4-chloro-2-methylphenyl, 2,4-dichlorophenyl, 1-methyl-4-pyridyl, 1-methyl-1H-pyrazole-4-yl, oxzaoyl-2-yl, or pyrrolidin-1yl.

Embodiment 13. The method according to one of Embodiment 1 to Embodiment 12, wherein R⁵ provides a heteroatom two carbons from the amide nitrogen, wherein the heteroatom is oxygen or nitrogen.

Embodiment 14. The method according to one of Embodiment 1 to Embodiment 13, wherein

R⁴ is H or C₁-C₄ alkyl optionally substituted by one or more halogen atoms;

R⁵ is C₁-C₄ alkoxy optionally substituted by one or more halogen atoms; —(CH₂)ₘ—NR¹⁷R¹⁸; —(CH₂)ₘ—OR″, or OH;

R′ is H, or C₁-C₄ alkyl optional substituted with one or more halogen;

m is 0, 1 or 2;

R⁶ is C₁-C₄ alkyl optionally substituted by one or more halogen atoms; C₁-C₄ alkoxy optionally substituted by one or more halogen atoms; OH; CN; halogen; —(C₀-C₄ alkyl)-C₆ aryl; or —(C₀-C₄ alkyl)-5 to 6 membered heterocyclic group, wherein the heterocyclic group contains at least one heteroatom selected from N, O and S, wherein the aryl and heterocyclyl groups are each optionally substituted by one or more Z substituents; or

R⁴ and R⁶ together with the carbon atoms to which they are bound form a 3 to 8 membered carbocyclic ring system; or

R⁵ and R⁶ together with the carbon atoms to which they are bound a 5 to 8 membered heterocyclic ring system containing one or more heteroatoms selected from N, O and S, wherein the ring system is optionally substituted by one or more Z substituents;

R¹⁷ and R¹⁸ are each independently H; or C₁-C₄ alkyl optionally substituted by one or more halogen atoms.

Embodiment 15. The method according to any proceeding embodiment, wherein

R³ is H;

R⁴ is H or Me;

R^{4a} is H;

R⁵ is —(CH₂)ₘ—NR¹⁷R¹⁸; —(CH₂)ₘ—OR′; or OH;

m is 0, or 1;

R′ is H;

R⁶ is C₁-C₄ alkyl optionally substituted by one or more halogen atoms; or

R⁵ and R⁶ together with the carbon atoms to which they are bound form a 5 to 6 membered heterocyclic ring system containing one or more heteroatoms selected from N, O and S, wherein the ring system is optionally substituted by one or more Z substituents; and

R¹⁷ and R¹⁸ are each independently H; or C₁-C₄ alkyl optionally substituted by one or more halogen atoms.

Embodiment 16. The method according to any proceeding embodiment, wherein

R³ is H;

R⁴ is H or Me;

R^{4a} is H;

R⁵ is —NR¹⁷R¹⁸; or OH;

R⁶ is C₁-C₄ alkyl optionally substituted by one or more halogen atoms; or

R⁵ and R⁶ together with the carbon atoms to which they are bound form a 5 to 6 membered heterocyclic ring system containing one or more heteroatoms selected from N, O and S, wherein the ring system is optionally substituted by one or more Z substituents; and

R¹⁷ and R¹⁸ are each independently H; or C₁-C₄ alkyl optionally substituted by one or more halogen atoms.

Embodiment 17. The method according to any proceeding claim, wherein

R³ is H;

R⁴ is H or Me;

R^{4a} is H;

R⁵ is —NR¹⁷R¹⁸; or OH;

R⁶ is C₁-C₄ alkyl optionally substituted by one or more halogen atoms; and

R¹⁷ and R¹⁸ are each independently H; or C₁-C₄ alkyl optionally substituted by one or more halogen atoms.

Embodiment 18. The method according to one of Embodiment 1 to Embodiment 13, wherein

R³ is H;

R^{4a} is H;

R⁴ and R⁵ form an oxo group;

R⁶ is C₁-C₄ alkyl optionally substituted by one or more halogen atoms; C₁-C₄ alkoxy optionally substituted by one or more halogen atoms; phenyl; or 5 to 6 membered heterocyclic group, wherein the heterocyclic group contains at least one heteroatom selected from N, O and S, wherein the phenyl and heterocyclyl groups are each optionally substituted by one or more Z substituents.

Embodiment 19. The method according to any of Embodiment 1 to Embodiment 13 or Embodiment 18, wherein

R³ is H;

R^{4a} is H;

R⁴ and R⁵ form an oxo group;

R⁶ is C₁-C₄ alkyl optionally substituted by one or more halogen atoms; or phenyl, wherein the phenyl is optionally substituted by one or more Z substituents;

Z is independently OH, C₁-C₄ alkyl optionally substituted by one or more OH groups or NH₂ groups, C₁-C₄ alkyl optionally substituted by one or more halogen atoms, C₁-C₄ alkoxy optionally substituted by one or more OH groups or C₁-C₄ alkoxy, C(O)OR¹⁹, C(O)R¹⁹, OR¹⁹, CN, or halogen;

R¹⁹ is H; C₁-C₄ alkyl; C₃-C₆ cycloalkyl; or C₁-C₄ alkoxy-C₁-C₄ alkyl, wherein all alkyl are optionally substituted with halogens.

Embodiment 20. The method according to any of Embodiment 1 to Embodiment 13 or Embodiment 18 to Embodiment 19, wherein

R³ is H;

R^{4a} is H;

R⁴ and R⁵ form an oxo group;

R⁶ is C₁-C₄ alkyl optionally substituted by one or more halogen atoms; or phenyl, wherein the phenyl is optionally substituted by one or more Z substituents;

Z is independently, C₁-C₄ alkyl optionally substituted by one or more halogen atoms, C₁-C₄ alkoxy or halogen.

Embodiment 21. The method according to one of Embodiment 1 to Embodiment 13, wherein the compound is represented by Formula (II),

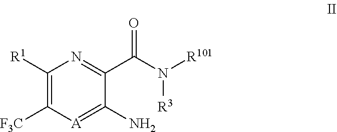

or a pharmaceutically acceptable salt thereof, wherein,

R¹⁰¹ is selected from the following:

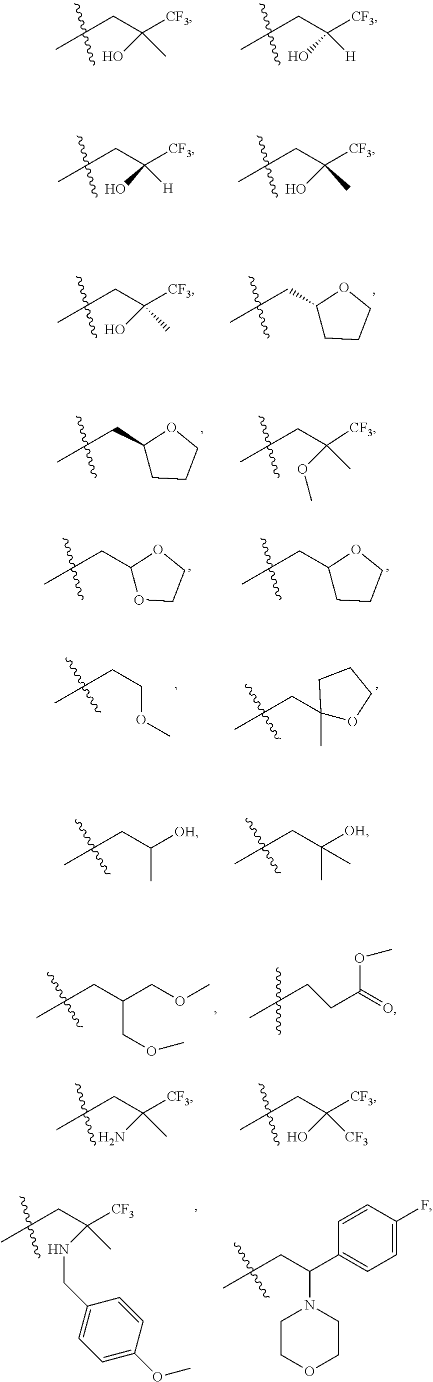

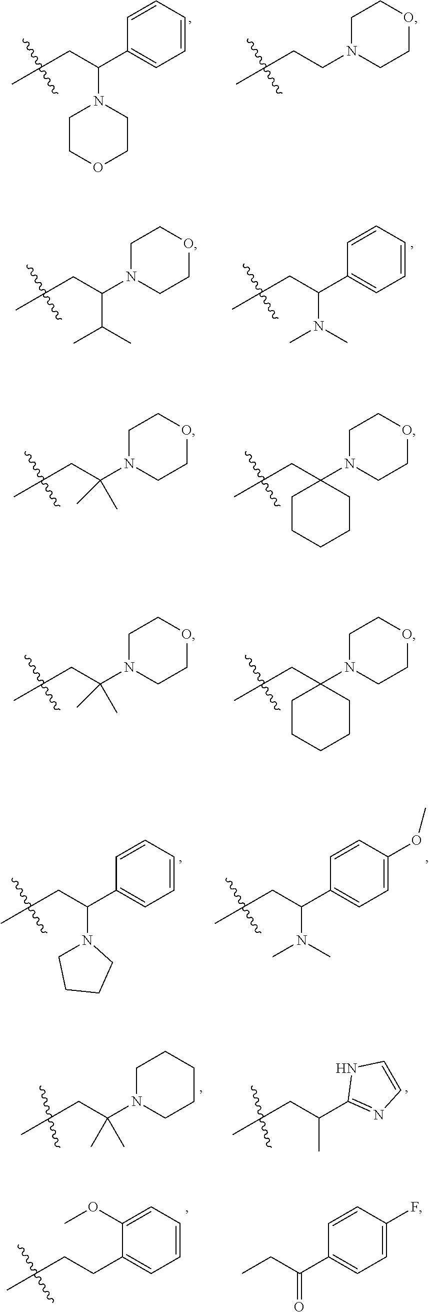

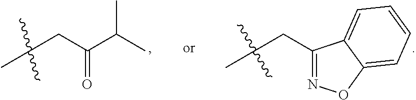

Embodiment 22. The method according to Embodiment 21, wherein

R³ is H;

R¹⁰¹ is

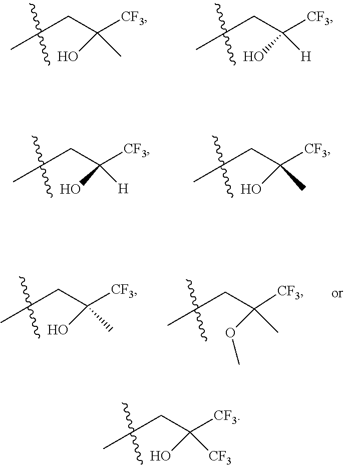

Embodiment 23. The method according to Embodiment 21, wherein

R³ is H;

R¹⁰¹ is

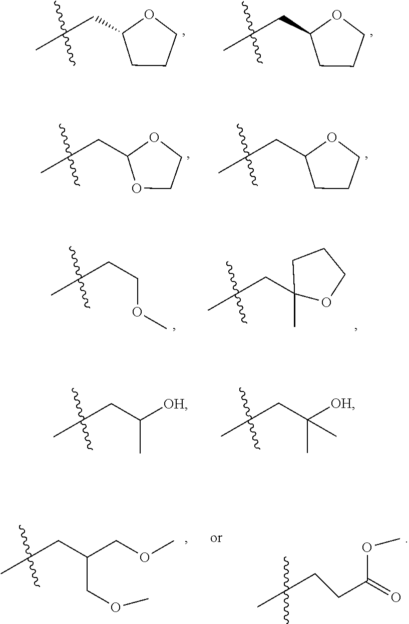

Embodiment 24. The method according to Embodiment 21, wherein

R³ is H;

R¹⁰¹ is

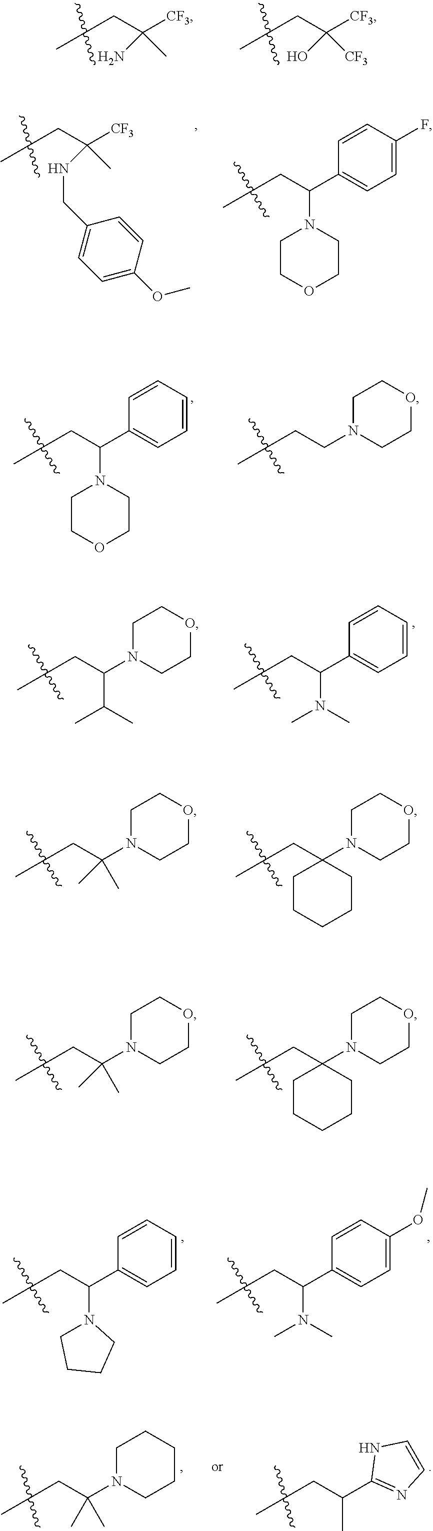

Embodiment 25. The method according to Embodiment 21, wherein

R³ is H;

R¹⁰¹ is

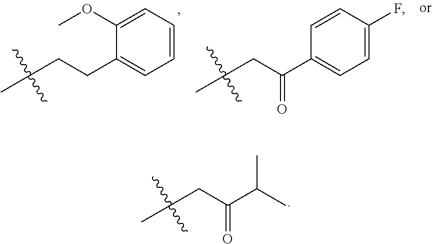

Embodiment 26. The method according to one of Embodiment 1 to Embodiment 13, wherein

R³ is H;

R¹⁰¹ is —(C₁-C₂ alkyl)-5 to 10 membered heterocyclic group, wherein the heterocyclic group contains at least one heteroatom selected from N, O and S, wherein the aryl and heterocyclyl groups are each optionally substituted by one or more Z substituents.

Embodiment 27. The method according to one of Embodiment 21 to Embodiment 26, wherein

R³ is H;

R¹⁰¹ is

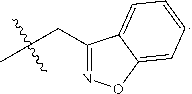 [SMILES: *Cc1noc2ccccc12]

Embodiment 28. A method for treating bronchiectasis comprising administering at least one compound of Formula (III)

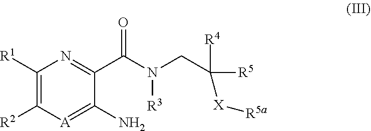

or a pharmaceutically acceptable salt thereof, to a subject in need thereof, wherein:

A is N or CR^{4a};

X is NR^{y} or O;

R¹ is C₁-C₈ alkyl optionally substituted by one or more halogen atoms; C₃-C₁₀ cycloalkyl; —C₁-C₄ alkyl-C₃-C₈ cycloalkyl; C₁-C₈ alkoxy optionally substituted by one or more halogen atoms; halogen; CN; NR¹¹R₁₂; C(O)NR¹³R¹⁴; NR¹³C(O)R¹⁵, CO₂R¹⁵, —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, aryl and heterocyclyl groups are each optionally substituted by one or more Z substituents;

R² is C₁-C₄ haloalkyl;

R³ and R^{4a} are each independently H or C₁-C₈ alkyl optionally substituted by one or more halogen atoms;

R⁴ is H, or C₁-C₈ alkyl optional substituted with one or more halogen;

R^{5a} is H, C₁-C₈ alkyl optional substituted with one or more halogen, —(C₀-C₄ alkyl)-C₆-C₁₄ aryl or −3 to 14 membered heterocyclic group, wherein the heterocyclic group contains at least one heteroatom selected from N, O and S; wherein the —(C₀-C₄ alkyl)-C₆-C₁₄ aryl and —(C₀-C₄ alkyl)-3 to 14 membered heterocyclic group are each optionally substituted by one or more Z substituents;

R^{y} is H, C₁-C₈ alkyl optional substituted with one or more halogen, —(C₀-C₄ alkyl)-C₆-C₁₄ aryl or −3 to 14 membered heterocyclic group, wherein the heterocyclic group contains at least one heteroatom selected from N, O and S; wherein the —(C₀-C₄ alkyl)-C₆-C₁₄ aryl and —(C₀-C₄ alkyl)-3 to 14 membered heterocyclic group are each optionally substituted by one or more Z substituents;

R⁶ is C₁-C₈ alkyl optionally substituted by one or more halogen atoms; C₃-C₁₀ cycloalkyl; —C₁-C₄ alkyl-C₃-C₈ cycloalkyl; C₁-C₈ alkoxy optionally substituted by one or more halogen atoms; OH; CN; halogen; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, —(C₀-C₄ alkyl)-C₆-C₁₄ aryl and —(C₀-C₄ alkyl)-3 to 14 membered heterocyclic group are each optionally substituted by one or more Z substituents; or

R⁴ and R⁶ together with the carbon atoms to which they are bound form a 3 to 8 membered carbocyclic ring system; or

R^{5a} and R⁶ together with the atoms to which they are bound a 5 to 8 membered heterocyclic ring system containing one or more heteroatoms selected from N, O and S, wherein the ring system is optionally substituted by one or more Z substituents; or

R^{5a} and R^{y} together with the atoms to which they are bound a 5 to 8 membered heterocyclic ring system containing one or more heteroatoms selected from N, O and S, wherein the ring system is optionally substituted by one or more Z substituents;

R¹¹ and R¹³ are each independently H, C₁-C₈ alkyl optionally substituted by one or more halogen atoms, C₃-C₁₀ cycloalkyl or —(C₁-C₄ alkyl)-C₃-C₈ cycloalkyl;

R¹², R¹⁴, and R¹⁵ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the cycloalkyl, cycloalkenyl, aryl and heterocyclyl groups are each optionally substituted by one or more Z substituents; or

R¹¹ and R¹², and R¹³ and R¹⁴ together with the nitrogen atom to which they are attached may form a 4 to 14 membered heterocyclic group optionally substituted by one or more Z substituents;

Z is independently OH, aryl, O-aryl, benzyl, O-benzyl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁸(SO₂)R²¹, (SO₂)NR¹⁹R²¹, (SO₂)R²¹, NR¹⁸C(O)R²¹, C(O)NR¹⁹R²¹, NR¹⁸C(O)NR¹⁹R²¹, NR¹⁸C(O)OR¹⁹, NR¹⁹R²¹, C(O)OR¹⁹, C(O)R¹⁹, SR¹⁹, OR¹⁹, oxo, CN, NO₂, halogen or a 3 to 14 membered heterocyclic group, wherein the heterocyclic group contains at least one heteroatom selected from N, O and S;

R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy.

Embodiment 29. The method according to Embodiment 28, wherein

A is N or CR^{4a};

X is NR^{y} or O;

R¹ is C₁-C₄ alkyl optionally substituted by one or more halogen atoms; C₁-C₄ alkoxy optionally substituted by one or more halogen atoms; halogen; —(C₀-C₄ alkyl)-C₆ aryl; or —(C₀-C₄ alkyl)-5 to 6 membered heterocyclic group, wherein the heterocyclic group contains at least one heteroatom selected from N, O and S; wherein the cycloalkyl, aryl and heterocyclyl groups are each optionally substituted by one or more Z substituents;

R² is C₁-C₄ haloalkyl;

R³ and R^{4a} are H;

R⁴ is H, or C₁-C₄ alkyl optional substituted with one or more halogen;

R^{5a} is H, C₁-C₄ alkyl optional substituted with one or more halogen, —(C₀-C₄ alkyl)-C₆ aryl or −5 to 8 membered heterocyclic group, wherein the heterocyclic group contains at least one heteroatom selected from N, O and S; wherein the —(C₀-C₄ alkyl)-C₆ aryl and −5 to 8 membered heterocyclic group are each optionally substituted by one or more Z substituents;

R^{y} is H, C₁-C₄ alkyl optional substituted with one or more halogen, —(C₀-C₄ alkyl)-C₆ aryl or −5 to 8 membered heterocyclic group, wherein the heterocyclic group contains at least one heteroatom selected from N, O and S; wherein the —(C₀-C₄ alkyl)-C₆ aryl and −5 to 8 membered heterocyclic group are each optionally substituted by one or more Z substituents;

R⁶ is C₁-C₄ alkyl optionally substituted by one or more halogen atoms; C₁-C₄ alkoxy optionally substituted by one or more halogen atoms; OH; CN; —(C₀-C₄ alkyl)-C₆ aryl; or −5 to 8 membered heterocyclic group, wherein the heterocyclic group contains at least one heteroatom selected from N, O and S; wherein the —C₆ aryl and −5 to 8 membered heterocyclic group are each optionally substituted by one or more Z substituents; or

R⁴ and R⁶ together with the carbon atoms to which they are bound form a 3 to 8 membered carbocyclic ring system; or

R^{5a} and R⁶ together with the atoms to which they are bound a 5 to 8 membered heterocyclic group containing one or more heteroatoms selected from N, O and S, wherein the heterocyclic group is optionally substituted by one or more Z substituents; or

R^{5a} and R^{y} together with the atoms to which they are bound a 5 to 8 membered heterocyclic ring system containing one or more heteroatoms selected from N, O and S, wherein the ring system is optionally substituted by one or more Z substituents;

Z is independently OH, aryl, O-aryl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, N—R¹⁸C(O)R²¹, C(O)NR¹⁹R²¹, NR¹⁹R²¹, C(O)OR¹⁹, C(O)R¹⁹, SR¹⁹, OR¹⁹, oxo, CN, NO₂, halogen or a 5 to 8 membered heterocyclic group, wherein the heterocyclic group contains at least one heteroatom selected from N, O and S; wherein the heterocyclic group is option substituted by halogen, C₁-C₄ alkyl optionally substituted by halogen, C₁-C₄ alkoxy or —CN;

R¹⁸ is H or C₁-C₄ alkyl;

R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy.

Embodiment 30. The method according to Embodiment 28 or Embodiment 29, wherein

A is N or CR^{4a};

X is NR^{y} or O;

R¹ is C₁-C₄ alkyl optionally substituted by one or more halogen atoms; C₁-C₄ alkoxy optionally substituted by one or more halogen atoms; or halogen;

R² is CF₃;

R³ and R^{4a} are H;

R⁴ is H, or C₁-C₄ alkyl optional substituted with one or more halogen;

R^{5a} is H, C₁-C₄ alkyl optional substituted with one or more halogen,

R⁶ is H, C₁-C₄ alkyl optional substituted with one or more halogen,

R⁶ is C₁-C₄ alkyl optionally substituted by one or more halogen atoms; C₁-C₄ alkoxy optionally substituted by one or more halogen atoms; OH; CN; or

R⁴ and R⁶ together with the carbon atoms to which they are bound form a 3 to 6 membered carbocyclic ring system; or

R^{5a} and R⁶ together with the atoms to which they are bound a 5 to 8 membered heterocyclic group containing one or more heteroatoms selected from N, O and S, wherein the heterocyclic group is optionally substituted by one or more Z substituents; or

R^{5a} and R^{y} together with the atoms to which they are bound a 5 to 8 membered heterocyclic ring system containing one or more heteroatoms selected from N, O and S, wherein the ring system is optionally substituted by one or more Z substituents;

Z is independently OH, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁹R²¹, C(O)OR¹⁹, C(O)R¹⁹, SR¹⁹, OR¹⁹, oxo, CN, NO₂, or halogen;

R¹⁹ is H; C₁-C₈ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

R¹⁹ and R²¹ together with the nitrogen atom to which they attached form a 5- to 6-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy.

Embodiment 31. The method according to one of Embodiment 28 to Embodiment 30, wherein

A is N or CR^{4a};

X is NR^{y} or O;

R¹ is C₁-C₄ alkyl optionally substituted by one or more halogen atoms; C₁-C₄ alkoxy optionally substituted by one or more halogen atoms; or halogen;

R² is CF₃;

R³ and R^{4a} are H;

R⁴ is H, or C₁-C₄ alkyl optional substituted with one or more halogen;

R^{5a} is H, C₁-C₄ alkyl optional substituted with one or more halogen,

R^{y} is H, C₁-C₄ alkyl optional substituted with one or more halogen,

R⁶ is C₁-C₄ alkyl optionally substituted by one or more halogen atoms; C₁-C₄ alkoxy optionally substituted by one or more halogen atoms; OH; CN; or

R^{5a} and R⁶ together with the atoms to which they are bound a 5 to 8 membered heterocyclic group containing one or more heteroatoms selected from N, O and S, wherein the heterocyclic group is optionally substituted by one or more Z substituents; or

R^{5a} and R^{y} together with the atoms to which they are bound a 5 to 8 membered heterocyclic ring system containing one or more heteroatoms selected from N, O and S, wherein the ring system is optionally substituted by one or more Z substituents;

Z is independently OH, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, oxo, CN, NO₂, or halogen;

Embodiment 32. The method according to any of the proceeding embodiments, A is N.

Embodiment 33. The method to one of Embodiment 1 to Embodiment 31, wherein A is CR^{4a}.

Embodiment 34. The method according to Embodiment 33 wherein A is CR^{4a} and R^{4a} is H.

Embodiment 35. The method according to any proceeding embodiment, wherein R² is CF₃CF₂—, (CF₃)₂CH—, CH₃—CF₂—, CF₃CF₂—, CF₃, CF₂H—, CH₃—CCl₂—, CF₃CFCClH—, CBr₃, CBr₂H—CF₃CF₂CHCF₃ or CF₃CF₂CF₂CF₂—.

Embodiment 36. The method according to any proceeding embodiment, wherein R² is CF₃.

Embodiment 37. The method according to any proceeding embodiment, wherein the compound is a substantially pure enantiomer with the S configuration.

Embodiment 38. The method according to one of Embodiment 1 to Embodiment 36, wherein the compound is a substantially pure enantiomer with the R configuration.

Embodiment 39. The method according to any of Embodiment 2, Embodiment 21 or Embodiment 28, wherein the compound is selected from the group consisting of:
3-Amino-6-bromo-5-trifluoromethyl-pyridine-2-carboxylic acid (3,3,3-trifluoro-2-hydroxy-2-methyl-propyl)-amide;
3-Amino-6-bromo-5-trifluoromethyl-pyridine-2-carboxylic acid (3,3,3-trifluoro-2-hydroxy-propyl)-amide;
3-Amino-6-bromo-5-trifluoromethyl-pyridine-2-carboxylic acid ((S)-3,3,3-trifluoro-2-hydroxy-propyl)-amide;
3-Amino-6-bromo-5-trifluoromethyl-pyridine-2-carboxylic acid [(R)-1-(tetrahydro-furan-2-yl)methyl]-amide;
3-Amino-6-bromo-5-trifluoromethyl-pyridine-2-carboxylic acid ([1,3]dioxolan-2-ylmethyl)-amide;
3-Amino-6-bromo-5-trifluoromethyl-pyridine-2-carboxylic acid [(S)-1-(tetrahydro-furan-2-yl)methyl]-amide;
3-Amino-6-bromo-5-trifluoromethyl-pyridine-2-carboxylic acid (tetrahydro-furan-2-ylmethyl)-amide;
3-Amino-6-bromo-5-trifluoromethyl-pyridine-2-carboxylic acid (2-methyl-2-piperidin-1-yl-propyl)-amide;
3-Amino-6-bromo-5-trifluoromethyl-pyridine-2-carboxylic acid (2-hydroxy-propyl)-amide;
3-Amino-6-bromo-5-trifluoromethyl-pyridine-2-carboxylic acid (2-hydroxy-2-methyl-propyl)-amide;
3-Amino-6-bromo-5-trifluoromethyl-pyridine-2-carboxylic acid (2-methyl-tetrahydro-furan-2-yl-methyl)-amide;
3-Amino-6-bromo-5-trifluoromethyl-pyridine-2-carboxylic acid (2-methoxy-ethyl)-amide;
3-Amino-6-bromo-5-trifluoromethyl-pyridine-2-carboxylic acid [2-(4-fluoro-phenyl)-2-morpholin-4-yl-ethyl]-amide;
3-Amino-6-bromo-5-trifluoromethyl-pyridine-2-carboxylic acid (2-morpholin-4-yl-2-phenyl-ethyl)-amide;
3-Amino-6-bromo-5-trifluoromethyl-pyridine-2-carboxylic acid (2-dimethylamino-2-phenyl-ethyl)-amide;
3-Amino-6-(4-fluoro-phenyl)-5-trifluoromethyl-pyridine-2-carboxylic acid (3,3,3-trifluoro-2-hydroxy-2-methyl-propyl)-amide;
3-Amino-5-trifluoromethyl-pyridine-2-carboxylic acid (3,3,3-trifluoro-2-hydroxy-2-methyl-propyl)-amide;
3-Amino-6-(4-chloro-2-methyl-phenyl)-5-trifluoromethyl-pyridine-2-carboxylic acid ((R)-3,3,3-trifluoro-2-hydroxy-propyl)-amide;
3-Amino-6-bromo-5-trifluoromethyl-pyridine-2-carboxylic acid (3,3,3-trifluoro-2-hydroxy-2-trifluoromethyl-propyl)-amide;
5-Amino-6′-methyl-3-trifluoromethyl-[2,3′]bipyridinyl-6-carboxylic acid (3,3,3-trifluoro-2-hydroxy-2-methyl-propyl)-amide;
3-Amino-6-bromo-5-trifluoromethyl-pyridine-2-carboxylic acid ((R)-3,3,3-trifluoro-2-hydroxy-2-methyl-propyl)-amide;
3-Amino-6-bromo-5-trifluoromethyl-pyridine-2-carboxylic acid ((S)-3,3,3-trifluoro-2-hydroxy-2-methyl-propyl)-amide;
3-Amino-6-methoxy-5-trifluoromethyl-pyridine-2-carboxylic acid (3,3,3-trifluoro-2-hydroxy-2-methyl-propyl)-amide;
3-Amino-6-(4-fluoro-phenyl)-5-trifluoromethyl-pyridine-2-carboxylic acid ((S)-3,3,3-trifluoro-2-hydroxy-2-methyl-propyl)-amide;
3-Amino-6-(4-fluoro-phenyl)-5-trifluoromethyl-pyridine-2-carboxylic acid ((R)-3,3,3-trifluoro-2-hydroxy-2-methyl-propyl)-amide;
3-amino-6-(2,4-dichloro-phenyl)-5-trifluoromethyl-pyridine-2-carboxylic acid (3,3,3-trifluoro-2-hydroxy-2-methyl-propyl)-amide;
3-Amino-6-(4-fluoro-phenyl)-5-trifluoromethyl-pyridine-2-carboxylic acid (2-hydroxy-2-methyl-propyl)-amide;
3-Amino-6-methoxy-5-trifluoromethyl-pyridine-2-carboxylic acid (3,3,3-trifluoro-2-hydroxy-propyl)-amide;
5-Amino-6′-methyl-3-trifluoromethyl-[2,3′]bipyridinyl-6-carboxylic acid (3,3,3-trifluoro-2-hydroxy-2-trifluoromethyl-propyl)-amide;
5-Amino-6′-methyl-3-trifluoromethyl-[2,3′]bipyridinyl-6-carboxylic acid (3,3,3-trifluoro-2-hydroxy-2-methyl-propyl)-amide;
3-Amino-5,6-bis-trifluoromethyl-pyridine-2-carboxylic acid ((S)-3,3,3-trifluoro-2-hydroxy-2-methyl-propyl)-amide;
3-Amino-5,6-bis-trifluoromethyl-pyridine-2-carboxylic acid ((R)-3,3,3-trifluoro-2-hydroxy-2-methyl-propyl)-amide;
3-Amino-6-methoxy-5-trifluoromethyl-pyridine-2-carboxylic acid ((S)-3,3,3-trifluoro-2-hydroxy-2-methyl-propyl)-amide;
3-Amino-6-methoxy-5-trifluoromethyl-pyridine-2-carboxylic acid ((R)-3,3,3-trifluoro-2-hydroxy-2-methyl-propyl)-amide;
Methyl 3-(3-amino-6-bromo-5-(trifluoromethyl)picolinamido)propanoate;
3-Amino-N-(benzo[d]isoxazol-3-ylmethyl)-6-bromo-5-(trifluoromethyl) picolinamide;
3-Amino-6-(oxazol-2-yl)-N-(3,3,3-trifluoro-2-hydroxy-2-methylpropyl)-5-(trifluoromethyl)picolinamide;
3-Amino-6-bromo-N-(3,3,3-trifluoro-2-methoxy-2-methylpropyl)-5-(trifluoromethyl)picolinamide;
3-amino-N-(2-hydroxy-3-methyl-2-(trifluoromethyl)butyl)-6-methoxy-5-(trifluoromethyl)picolinamide;
3-Amino-6-cyclopropyl-N-(3,3,3-trifluoro-2-hydroxy-2-methylpropyl)-5-(trifluoromethyl)picolinamide;
3-Amino-6-methoxy-N-(3,3,3-trifluoro-2-hydroxy-2-(trifluoromethyl)propyl)-5-(trifluoro methyl) picolinamide;
5-amino-N-(3,3,3-trifluoro-2-hydroxy-2-methylpropyl)-3-(trifluoromethyl)-2,4′-bipyridine-6-carboxamide;
3-Amino-6-bromo-5-trifluoromethyl-pyridine-2-carboxylic acid (3-methyl-2-oxo-butyl)-amide;
3-Amino-6-(1-methyl-1H-pyrazol-4-yl)-N-(3,3,3-trifluoro-2-hydroxy-2-methylpropyl)-5-(trifluoromethyl)picolinamide;
(S)-3-amino-6-ethoxy-N-(3,3,3-trifluoro-2-hydroxy-2-methylpropyl)-5-(trifluoro methyl)picolinamide;
3-Amino-6-(pyrrolidin-1-yl)-N-(3,3,3-trifluoro-2-hydroxy-2-methylpropyl)-5-(trifluoromethyl)picolinamide;
3-Amino-N-(2-amino-3,3,3-trifluoro-2-methylpropyl)-6-methoxy-5-(trifluoromethyl) picolinamide; and
3-Amino-6-methoxy-N-(3,3,3-trifluoro-2-(4-methoxybenzylamino)-2-methylpropyl)-5-(trifluoromethyl)picolinamide; or
   a pharmaceutically acceptable salt thereof.

Embodiment 40. The method according to Embodiment 39, wherein the compound is selected from the group consisting of:
3-Amino-6-bromo-5-trifluoromethyl-pyridine-2-carboxylic acid [ (R)-1-(tetrahydro-furan-2-yl)methyl]-amide;
3-Amino-6-bromo-5-trifluoromethyl-pyridine-2-carboxylic acid ([1,3]dioxolan-2-ylmethyl)-amide;
3-Amino-6-bromo-5-trifluoromethyl-pyridine-2-carboxylic acid [(S)-1-(tetrahydro-furan-2-yl)methyl]-amide;
3-Amino-6-bromo-5-trifluoromethyl-pyridine-2-carboxylic acid (tetrahydro-furan-2-ylmethyl)-amide; and
3-Amino-6-bromo-5-trifluoromethyl-pyridine-2-carboxylic acid (2-methyl-tetrahydro-furan-2-yl-methyl)-amide; or a pharmaceutically acceptable salt thereof.

Embodiment 41. The method according to any of Embodiment 2, Embodiment 21 or Embodiment 28, wherein the compound is selected from the group consisting of:
3-Amino-6-bromo-5-trifluoromethyl-pyrazine-2-carboxylic acid [2-(4-fluoro-phenyl)-2-morpholin-4-yl-ethyl]-amide;
3-Amino-6-bromo-5-trifluoromethyl-pyridine-2-carboxylic acid [2-(4-fluoro-phenyl)-2-morpholin-4-yl-ethyl]-amide;
3-Amino-6-bromo-5-trifluoromethyl-pyridine-2-carboxylic acid (2-morpholin-4-yl-2-phenyl-ethyl)-amide;
3-Amino-6-bromo-5-trifluoromethyl-pyridine-2-carboxylic acid (2-dimethylamino-2-phenyl-ethyl)-amide;
3-Amino-6-bromo-5-trifluoromethyl-pyrazine-2-carboxylic acid (3-methyl-2-morpholin-4-yl-butyl)-amide;
3-Amino-6-bromo-5-trifluoromethyl-pyrazine-2-carboxylic acid (2-methyl-2-morpholin-4-yl-propyl)-amide;
3-Amino-6-bromo-5-trifluoromethyl-pyrazine-2-carboxylic acid (1-morpholin-4-yl-cyclohexylmethyl)-amide;
3-Amino-6-bromo-5-trifluoromethyl-pyrazine-2-carboxylic acid (2-morpholin-4-yl-2-phenyl-ethyl)-amide;
3-Amino-6-bromo-5-trifluoromethyl-pyrazine-2-carboxylic acid (2-dimethylamino-2-phenyl-ethyl)-amide;
3-Amino-6-bromo-5-trifluoromethyl-pyrazine-2-carboxylic acid [2-(4-methoxy-phenyl)-2-pyrrolidin-1-yl-ethyl]-amide;
3-Amino-N-(2-amino-3,3,3-trifluoro-2-methylpropyl)-6-methoxy-5-(trifluoromethyl) picolinamide; and
3-Amino-6-bromo-5-trifluoromethyl-pyrazine-2-carboxylic acid [2-dimethylamino-2-(4-methoxy-phenyl)-ethyl]-amide; or
   a pharmaceutically acceptable salt thereof.

Embodiment 42. The method according to any of Embodiment 2, Embodiment 21 or Embodiment 28, wherein the compound is selected from the group consisting of:
3-Amino-6-bromo-5-trifluoromethyl-pyrazine-2-carboxylic acid (2-methyl-tetrahydro-furan-2-yl-methyl)-amide;
3-Amino-6-bromo-5-trifluoromethyl-pyrazine-2-carboxylic acid [2-(4-fluoro-phenyl)-2-morpholin-4-yl-ethyl]-amide;
3-Amino-6-bromo-5-trifluoromethyl-pyrazine-2-carboxylic acid (3-methyl-2-morpholin-4-yl-butyl)-amide;
3-Amino-6-bromo-5-trifluoromethyl-pyrazine-2-carboxylic acid (2-methyl-2-morpholin-4-yl-propyl)-amide;
3-Amino-6-bromo-5-trifluoromethyl-pyrazine-2-carboxylic acid (1-morpholin-4-yl-cyclohexylmethyl)-amide;
3-Amino-6-bromo-5-trifluoromethyl-pyrazine-2-carboxylic acid (2-morpholin-4-yl-2-phenyl-ethyl)-amide;
3-Amino-6-bromo-5-trifluoromethyl-pyrazine-2-carboxylic acid (2-dimethylamino-2-phenyl-ethyl)-amide;
3-Amino-6-bromo-5-trifluoromethyl-pyrazine-2-carboxylic acid [2-(4-methoxy-phenyl)-2-pyrrolidin-1-yl-ethyl]-amide;
3-Amino-6-bromo-5-trifluoromethyl-pyrazine-2-carboxylic acid [2-dimethylamino-2-(4-methoxy-phenyl)-ethyl]-amide;
3-Amino-6-bromo-5-trifluoromethyl-pyrazine-2-carboxylic acid [2-(4-fluoro-phenyl)-2-oxo-ethyl]-amide;
3-Amino-6-furan-2-yl-5-trifluoromethyl-pyrazine-2-carboxylic acid [2-(2-methoxy-phenyl)-ethyl]-amide;
3-amino-N-(3,3,3-trifluoro-2-hydroxy-2-methylpropyl)-5,6-bis(trifluoromethyl) pyrazine-2-carboxamide;
N-(2-(1H-imidazol-2-yl)propyl)-3-amino-6-bromo-5-(trifluoromethyl)pyrazine-2-carboxamide;
3-Amino-6-bromo-N-(2-morpholinoethyl)-5-(trifluoromethyl)pyrazine-2-carboxamide; and
3-Amino-6-bromo-5-trifluoromethyl-pyrazine-2-carboxylic acid [2-(4-fluoro-phenyl)-2-oxo-ethyl]-amide; or
   a pharmaceutically acceptable salt thereof.

Embodiment 43. The method according to Embodiment 39, wherein the compound is selected from the group consisting of:
3-Amino-6-bromo-5-trifluoromethyl-pyridine-2-carboxylic acid (3,3,3-trifluoro-2-hydroxy-2-methyl-propyl)-amide;
3-Amino-6-bromo-5-trifluoromethyl-pyridine-2-carboxylic acid (3,3,3-trifluoro-2-hydroxy-propyl)-amide;
3-Amino-6-bromo-5-trifluoromethyl-pyridine-2-carboxylic acid ((S)-3,3,3-trifluoro-2-hydroxy-propyl)-amide;
3-Amino-5,6-bis-trifluoromethyl-pyridine-2-carboxylic acid ((S)-3,3,3-trifluoro-2-hydroxy-2-methyl-propyl)-amide;
3-Amino-5,6-bis-trifluoromethyl-pyridine-2-carboxylic acid ((R)-3,3,3-trifluoro-2-hydroxy-2-methyl-propyl)-amide;
3-Amino-6-methoxy-5-trifluoromethyl-pyridine-2-carboxylic acid ((S)-3,3,3-trifluoro-2-hydroxy-2-methyl-propyl)-amide;
3-Amino-6-(4-fluoro-phenyl)-5-trifluoromethyl-pyridine-2-carboxylic acid ((S)-3,3,3-trifluoro-2-hydroxy-2-methyl-propyl)-amide;
3-Amino-6-(4-fluoro-phenyl)-5-trifluoromethyl-pyridine-2-carboxylic acid ((R)-3,3,3-trifluoro-2-hydroxy-2-methyl-propyl)-amide;
3-amino-6-(2,4-dichloro-phenyl)-5-trifluoromethyl-pyridine-2-carboxylic acid (3,3,3-trifluoro-2-hydroxy-2-methyl-propyl)-amide;
3-Amino-6-(4-fluoro-phenyl)-5-trifluoromethyl-pyridine-2-carboxylic acid (2-hydroxy-2-methyl-propyl)-amide; and
3-Amino-6-methoxy-5-trifluoromethyl-pyridine-2-carboxylic acid ((R)-3,3,3-trifluoro-2-hydroxy-2-methyl-propyl)-amide; or
   a pharmaceutically acceptable salt thereof.

Embodiment 44. The method according to one of Embodiment 1 to Embodiment 43, wherein bronchiectasis is cystic fibrosis bronchiectasis or non-cystic fibrosis bronchiectasis.

Embodiment 45. Use of a compound according to Formula (I):

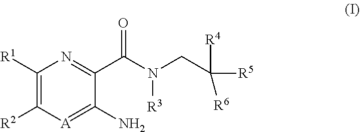

or a pharmaceutically acceptable salt thereof, to a subject in need thereof, wherein:

A is N or CR^{4a};

R¹ is H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; halogen; SO₂NR⁸R⁹; SO₂R¹⁰; S—C₁-C₈alkyl optionally substituted by one or more halogen atoms; S—C₆-C₁₄ aryl; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; CN; NR¹¹R¹²; CONR¹³R¹⁴; NR¹³SO₂R¹⁵; NR¹³C(O)R¹⁵ and CO₂R¹⁵, wherein the cycloalkyl, cycloalkenyl, aryl and heterocyclyl groups are each optionally substituted by one or more Z substituents;

R² is C₁-C₄ haloalkyl;

R³, R⁴ and R^{4a} are each independently H or C₁-C₈ alkyl optionally substituted by one or more halogen atoms;

R⁵ and R⁶ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; OH; CN; halogen; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; or —(C₀-C₄ alkyl)-CO₂R¹⁵, wherein the cycloalkyl, cycloalkenyl, —(C₀-C₄ alkyl)-C₆-C₁₄ aryl and —(C₀-C₄ alkyl)-3 to 14 membered heterocyclic group groups are each optionally substituted by one or more Z substituents; or

R⁵ and R⁶ are each independently a group of the formula:

—(CH₂)ₘ—NR¹⁷R¹⁸; or

R⁵ and R⁶ are each independently a group of the formula:

—(CH₂)ₘ—OR⁴; or

R⁴ and R⁵ together with the carbon atoms to which they are bound form a 3 to 8 membered carbocyclic ring system; or

R⁵ and R⁶ together with the carbon atoms to which they are bound form a 5 to 8 membered carbocyclic ring system or a 5 to 8 membered heterocyclic ring system containing one or more heteroatoms selected from N, O and S, wherein the ring system is optionally substituted by one or more Z substituents;

R⁴, R⁵ and R⁶ cannot all be the same;

m is 0, 1, 2 or 3;

R⁸, R¹¹, R¹³ and R¹⁷ are each independently H, C₁-C₈ alkyl optionally substituted by one or more halogen atoms, C₃-C₁₀ cycloalkyl or —(C₁-C₄ alkyl)-C₃-C₈ cycloalkyl;

R⁹, R¹⁰, R¹², R¹⁴, R¹⁵, R¹⁶ and R¹⁸ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the cycloalkyl, cycloalkenyl, aryl and heterocyclyl groups are each optionally substituted by one or more Z substituents; or

R⁸ and R⁹, R¹¹ and R¹², R¹³ and R¹⁴, and R¹⁷ and R¹⁸ together with the nitrogen atom to which they are attached may form a 4 to 14 membered heterocyclic group optionally substituted by one or more Z substituents;

Z is independently OH, aryl, O-aryl, benzyl, O-benzyl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁸(SO₂)R²¹, (SO₂)NR¹⁹R²¹, (SO₂)R²¹, NR¹⁸C(O)R²¹, C(O)NR¹⁹R²¹, NR¹⁸C(O)NR¹⁹R²¹, NR¹⁸C(O)OR¹⁹, NR¹⁹R²¹, C(O)OR¹⁹, C(O)R¹⁹, SR¹⁹, OR¹⁹, oxo, CN, NO₂, halogen or a 3 to 14 membered heterocyclic group, wherein the heterocyclic group contains at least one heteroatom selected from N, O and S;

R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy,

in the manufacture of a medicament for use in the treatment of an inflammatory or obstructive airways disease or mucosal hydration.

Embodiment 46. Use of a compound according to Formula (I):

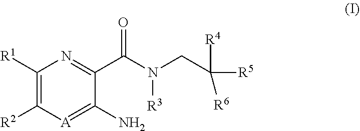

or a pharmaceutically acceptable salt thereof, to a subject in need thereof, wherein:

A is N or CR^{4a};

R¹ is H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; halogen; SO₂NR⁸R⁹; SO₂R¹⁰; S—C₁-C₈alkyl optionally substituted by one or more halogen atoms; S—C₆-C₁₄ aryl; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; CN; NR¹¹R¹²; CONR¹³R¹⁴; NR¹³SO₂R¹⁵; NR¹³C(O)R¹⁵ and CO₂R¹⁵, wherein the cycloalkyl, cycloalkenyl, aryl and heterocyclyl groups are each optionally substituted by one or more Z substituents;

R² is C₁-C₄ haloalkyl;

R³, R⁴ and R^{4a} are each independently H or C₁-C₈ alkyl optionally substituted by one or more halogen atoms;

R⁵ and R⁶ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; OH; CN; halogen; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; or —(C₀-C₄ alkyl)-CO₂R¹⁵, wherein the cycloalkyl, cycloalkenyl, —(C₀-C₄ alkyl)-C₆-C₁₄ aryl and —(C₀-C₄ alkyl)-3 to 14 membered heterocyclic group groups are each optionally substituted by one or more Z substituents; or

R⁵ and R⁶ are each independently a group of the formula:

—(CH₂)ₘ—NR¹⁷R¹⁸; or

R⁵ and R⁶ are each independently a group of the formula:

—(CH₂)ₘ—OR⁴; or

R⁴ and R⁵ together with the carbon atoms to which they are bound form a 3 to 8 membered carbocyclic ring system; or

R⁵ and R⁶ together with the carbon atoms to which they are bound form a 5 to 8 membered carbocyclic ring system or a 5 to 8 membered heterocyclic ring system containing one or more heteroatoms selected from N, O and S, wherein the ring system is optionally substituted by one or more Z substituents;

R⁴, R⁵ and R⁶ cannot all be the same;

m is 0, 1, 2 or 3;

R⁸, R¹¹, R¹³ and R¹⁷ are each independently H, C₁-C₈ alkyl optionally substituted by one or more halogen atoms, C₃-C₁₀ cycloalkyl or —(C₁-C₄ alkyl)-C₃-C₈ cycloalkyl;

R⁹, R¹⁰, R¹², R¹⁴, R¹⁵, R¹⁶ an R¹⁸ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the cycloalkyl, cycloalkenyl, aryl and heterocyclyl groups are each optionally substituted by one or more Z substituents; or

R⁸ and R⁹, R¹¹ and R¹², R¹³ and R¹⁴, and R¹⁷ and R¹⁸ together with the nitrogen atom to which they are attached may form a 4 to 14 membered heterocyclic group optionally substituted by one or more Z substituents;

Z is independently OH, aryl, O-aryl, benzyl, O-benzyl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁸(SO₂)R²¹, (SO₂)NR¹⁹R²¹, (SO₂)R²¹, NR¹⁸C(O)R²¹, C(O)NR¹⁹R²¹, NR¹⁸C(O)NR¹⁹R²¹, NR¹⁸C(O)OR¹⁹, NR¹⁹R²¹, C(O)OR¹⁹, C(O)R¹⁹, SR¹⁹, OR¹⁹, oxo, CN, NO₂, halogen or a 3 to 14 membered heterocyclic group, wherein the heterocyclic group contains at least one heteroatom selected from N, O and S;

R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxyin,

in the manufacture of a medicament for use in the treatment of a disease mediated by CFTR.

Embodiment 47. Use of a compound according to Embodiment 46 in the manufacture of a medicament for use in the treatment of a disease mediated by CFTR, wherein the disease is CF, COPD, or bronchiectasis.

Embodiment 48. Use of a compound according to Formula (I):

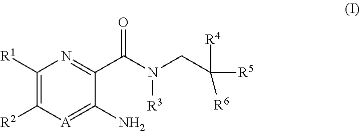

or a pharmaceutically acceptable salt thereof, to a subject in need thereof, wherein:

A is N or CR^{4a};

R¹ is H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; halogen; SO₂NR⁸R⁹; SO₂R¹⁰; S—C₁-C₈alkyl optionally substituted by one or more halogen atoms; S—C₆-C₁₄ aryl; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; CN; NR¹¹R¹²; CONR¹³R¹⁴; NR¹³SO₂R¹⁵; NR¹³C(O)R¹⁵ and CO₂R¹⁵, wherein the cycloalkyl, cycloalkenyl, aryl and heterocyclyl groups are each optionally substituted by one or more Z substituents;

R² is C₁-C₄ haloalkyl;

R³, R⁴ and R^{4a} are each independently H or C₁-C₈ alkyl optionally substituted by one or more halogen atoms;

R⁵ and R⁶ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; OH; CN; halogen; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; or —(C₀-C₄ alkyl)-CO₂R¹⁵, wherein the cycloalkyl, cycloalkenyl, —(C₀-C₄ alkyl)-C₆-C₁₄ aryl and —(C₀-C₄ alkyl)-3 to 14 membered heterocyclic group groups are each optionally substituted by one or more Z substituents; or

R⁵ and R⁶ are each independently a group of the formula:

—(CH₂)ₘ—NR¹⁷R¹⁸; or

R⁵ and R⁶ are each independently a group of the formula:

—(CH₂)ₘ—OR⁴; or

R⁴ and R⁵ together with the carbon atoms to which they are bound form a 3 to 8 membered carbocyclic ring system; or

R⁵ and R⁶ together with the carbon atoms to which they are bound form a 5 to 8 membered carbocyclic ring system or a 5 to 8 membered heterocyclic ring system containing one or more heteroatoms selected from N, O and S, wherein the ring system is optionally substituted by one or more Z substituents;

R⁴, R⁵ and R⁶ cannot all be the same;

m is 0, 1, 2 or 3;

R⁸, R¹¹, R¹³ and R¹⁷ are each independently H, C₁-C₈ alkyl optionally substituted by one or more halogen atoms, C₃-C₁₀ cycloalkyl or —(C₁-C₄ alkyl)-C₃-C₈ cycloalkyl;

R⁹, R¹⁰, R¹², R¹⁴, R¹⁵, R¹⁶ and R¹⁸ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the cycloalkyl, cycloalkenyl, aryl and heterocyclyl groups are each optionally substituted by one or more Z substituents; or

R⁸ and R⁹, R¹¹ and R¹², R¹³ and R¹⁴, and R¹⁷ and R¹⁸ together with the nitrogen atom to which they are attached may form a 4 to 14 membered heterocyclic group optionally substituted by one or more Z substituents;

Z is independently OH, aryl, O-aryl, benzyl, O-benzyl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁸(SO₂)R²¹, (SO₂)NR¹⁹R²¹, (SO₂)R²¹, NR¹⁸C(O)R²¹, C(O)NR¹⁹R²¹, NR¹⁸C(O)NR¹⁹R²¹, NR¹⁸C(O)OR¹⁹, NR¹⁹R²¹, C(O)OR¹⁹, C(O)R¹⁹, SR¹⁹, OR¹⁹, oxo, CN, NO₂, halogen or a 3 to 14 membered heterocyclic group, wherein the heterocyclic group contains at least one heteroatom selected from N, O and S;

R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxyin,

in the manufacture of a medicament for use in the treatment of bronchiectasis.

Embodiment 49. A pharmaceutical composition for treating a disease or disorder mediated by CFTR, comprising the compound as claimed in one of Embodiment 1 to Embodiment 44, and one or more pharmaceutically acceptable excipients.

Embodiment 50. The pharmaceutical composition according to Embodiment 49, wherein the disease or disorder is cystic fibrosis, COPD, or bronchiectasis.

Embodiment 51. The pharmaceutical composition according to Embodiment 49 or Embodiment 50, wherein the disease or disorder is bronchiectasis.

Embodiment 52. The pharmaceutical composition according to one of Embodiment 49 to Embodiment 51, wherein bronchiectasis is cystic fibrosis bronchiectasis or non-cystic fibrosis bronchiectasis.

Embodiment 53. A pharmaceutical combination, comprising:

a first active comprising the compound as claimed in one of Embodiment 1 to Embodiment 44, and

a second active selected from osmotic agents, ENaC blockers, anti-inflammatory agents, bronchodilatory agents, antihistamine agents, anti-tussive agents, antibiotic agents and DNase drug substances, wherein the first and second actives may be in the same or different pharmaceutical composition.

Embodiment 54. The pharmaceutical combination according to Embodiment 53, wherein the second active agent is an EnaC blocker.

Embodiment 55. A method for treating bronchiectasis, comprising administering an effective amount of 3-Amino-6-methoxy-5-trifluoromethyl-pyridine-2-carboxylic acid ((S)-3,3,3-trifluoro-2-hydroxy-2-methyl-propyl)-amide, or a pharmaceutically acceptable salt thereof, to a subject in need thereof.

Embodiment 56. A method for treating bronchiectasis, comprising administering an effective amount of 3-Amino-6-methoxy-5-trifluoromethyl-pyridine-2-carboxylic acid ((R)-3,3,3-trifluoro-2-hydroxy-2-methyl-propyl)-amide, or a pharmaceutically acceptable salt thereof, to a subject in need thereof.

Embodiment 57. A method for treating bronchiectasis, comprising administering an effective amount of 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to a subject in need thereof.

Embodiment 58. The method for treating bronchiectasis according to one of Embodiment 55 to Embodiment 57, wherein bronchiectasis is cystic fibrosis bronchiectasis or non-cystic fibrosis bronchiectasis.

Embodiment 59. A compound for use in treating bronchiectasis comprising administering an effective amount of said compound to a subject in need thereof, wherein said compound is 3-Amino-6-methoxy-5-trifluoromethyl-pyridine-2-carboxylic acid ((S)-3,3,3-trifluoro-2-hydroxy-2-methyl-propyl)-amide, or a pharmaceutically acceptable salt thereof.

Embodiment 60. A compound for use in treating bronchiectasis comprising administering an effective amount of said compound to a subject in need thereof, wherein said compound is 3-Amino-6-methoxy-5-trifluoromethyl-pyridine-2-carboxylic acid ((R)-3,3,3-trifluoro-2-hydroxy-2-methyl-propyl)-amide, or a pharmaceutically acceptable salt thereof.

Embodiment 61. A compound for use in treating bronchiectasis comprising administering an effective amount of said compound to a subject in need thereof, wherein said compound is 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof.

Embodiment 62. Use of a compound for treating bronchiectasis, comprising administering an effective amount of said compound to a subject in need thereof, wherein said compound is 3-Amino-6-methoxy-5-trifluoromethyl-pyridine-2-carboxylic acid ((S)-3,3,3-trifluoro-2-hydroxy-2-methyl-propyl)-amide or a pharmaceutically acceptable salt thereof.

Embodiment 63. Use of a compound for treating bronchiectasis, comprising administering an effective amount of said compound to a subject in need thereof, wherein said compound is 3-Amino-6-methoxy-5-trifluoromethyl-pyridine-2-carboxylic acid ((R)-3,3,3-trifluoro-2-hydroxy-2-methyl-propyl)-amide or a pharmaceutically acceptable salt thereof.

Embodiment 64. Use of a compound for treating bronchiectasis, comprising administering an effective amount of said compound to a subject in need thereof, wherein said compound is 3-Amino-6-methoxy-5-trifluoromethyl-pyridine-2-carboxylic acid (3,3,3-trifluoro-2-hydroxy-2-methyl-propyl)-amide or a pharmaceutically acceptable salt thereof.

Embodiment 65. Use of a compound in the manufacture of a medicament for use in treating bronchiectasis comprising administering an effective amount of said compound to a subject in need thereof, wherein said compound is 3-Amino-6-methoxy-5-trifluoromethyl-pyridine-2-carboxylic acid ((S)-3,3,3-trifluoro-2-hydroxy-2-methyl-propyl)-amide, or a pharmaceutically acceptable salt thereof.

Embodiment 66. Use of a compound in the manufacture of a medicament for use in treating bronchiectasis comprising administering an effective amount of said compound to a subject in need thereof, wherein said compound is 3-Amino-6-methoxy-5-trifluoromethyl-pyridine-2-carboxylic acid ((R)-3,3,3-trifluoro-2-hydroxy-2-methyl-propyl)-amide, or a pharmaceutically acceptable salt thereof.

Embodiment 67. Use of a compound in the manufacture of a medicament for use in treating bronchiectasis comprising administering an effective amount of said compound to a subject in need thereof, wherein said compound is 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof.

Embodiment 68. A method for inhibiting or reducing the level of colonization of at least one pathogenic bacteria in the lungs of a subject comprising administering a compound of Formula (I),

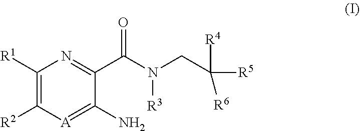

or pharmaceutically acceptable salts thereof, to a subject in need thereof, wherein:

A is N or CR^{4a};

R¹ is H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; halogen; SO₂NR⁸R⁹; SO₂R¹⁰; S—C₁-C₈alkyl optionally substituted by one or more halogen atoms; S—C₆-C₁₄ aryl; CN; NR¹¹R¹²; C(O)NR¹³R¹⁴; N¹³SO₂R¹⁵; NR¹³C(O)R¹⁵, CO₂R¹⁵, —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, aryl and heterocyclyl groups are each optionally substituted by one or more Z substituents;

R² is C₁-C₄ haloalkyl;

R³ and R^{4a} are each independently H or C₁-C₈ alkyl optionally substituted by one or more halogen atoms;

R⁴ is H, or C₁-C₈ alkyl optional substituted with one or more halogen;

R⁵ is —(CH₂)ₘ—NR¹⁷R¹⁸, —(CH₂)ₘ—OR′; C₁-C₈ alkoxy optionally substituted by one or more halogen atoms; —(C₀-C₄ alkyl)-CO₂R¹⁵; —(C₀-C₄ alkyl)-C₆-C₁₄ aryl or −3 to 14 membered heterocyclic group, wherein the heterocyclic group contains at least one heteroatom selected from N, O and S; wherein the —(C₀-C₄ alkyl)-C₆-C₁₄ aryl and —(C₀-C₄ alkyl)-3 to 14 membered heterocyclic group are each optionally substituted by one or more Z substituents;

R⁶ is C₁-C₈ alkyl optionally substituted by one or more halogen atoms; C₃-C₁₀ cycloalkyl; —C₁-C₄ alkyl-C₃-C₈ cycloalkyl; C₁-C₈ alkoxy optionally substituted by one or more halogen atoms; OH; CN; halogen; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, —(C₀-C₄ alkyl)-C₆-C₁₄ aryl and —(C₀-C₄ alkyl)-3 to 14 membered heterocyclic group are each optionally substituted by one or more Z substituents; or

R⁶ is H, and R⁵ is —(CH₂)ₘ—NR¹⁷R¹⁸, —(CH₂)ₘ—OR′, C₁-C₈ alkoxy optionally substituted by one or more halogen atoms; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; or —(C₀-C₄ alkyl)-CO₂R¹⁵, wherein —(C₀-C₄ alkyl)-C₆-C₁₄ aryl and —(C₀-C₄ alkyl)-3 to 14 membered heterocyclic group groups are each optionally substituted by one or more Z substituents; or

R⁴ and R⁶ together with the carbon atoms to which they are bound form a 3 to 8 membered carbocyclic ring system; or

R⁴ and R⁵ together form an oxo group (C═O) and R⁶ is C₁-C₄ alkyl optionally substituted by one or more halogen atoms; C₁-C₄ alkoxy optionally substituted by one or more halogen atoms; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the aryl and heterocyclyl groups are each optionally substituted by one or more Z substituents; or

R⁵ and R⁶ together with the carbon atoms to which they are bound a 5 to 8 membered heterocyclic ring system containing one or more heteroatoms selected from N, O and S, wherein the ring system is optionally substituted by one or more Z substituents; or

R⁴ and R⁵ and R⁶ together with the carbon atoms to which they are bound form a 5 to 8 membered heterocyclic ring system containing one or more heteroatoms selected from N, O and S, wherein the ring system is optionally substituted by one or more Z substituents;

R′ is H, or C₁-C₈ alkyl optional substituted with one or more halogen;

m is 0, 1, 2 or 3;

R⁸, R¹¹, R¹³ and R¹⁷ are each independently H, C₁-C₈ alkyl optionally substituted by one or more halogen atoms, C₃-C₁₀ cycloalkyl or —(C₁-C₄ alkyl)-C₃-C₈ cycloalkyl;

R⁹, R¹⁰, R¹², R¹⁴, R¹⁵, R¹⁶ and R¹⁸ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the cycloalkyl, cycloalkenyl, aryl and heterocyclyl groups are each optionally substituted by one or more Z substituents; or

R⁸ and R⁹, R¹¹ and R¹², R¹³ and R¹⁴, and R¹⁷ and R¹⁸ together with the nitrogen atom to which they are attached may form a 4 to 14 membered heterocyclic group optionally substituted by one or more Z substituents;

Z is independently OH, aryl, O-aryl, benzyl, O-benzyl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁸(SO₂)R²¹, (SO₂)NR¹⁹R²¹, (SO₂)R²¹, NR¹⁸C(O)R²¹, C(O)NR¹⁹R²¹, NR¹⁸C(O)NR¹⁹R²¹, NR¹⁸C(O)OR¹⁹, NR¹⁹R²¹, C(O)OR¹⁹, C(O)R¹⁹, SR¹⁹, OR¹⁹, oxo, CN, NO₂, halogen or a 3 to 14 membered heterocyclic group, wherein the heterocyclic group contains at least one heteroatom selected from N, O and S;

R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or

R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy.

Embodiment 69. A method for inhibiting or reducing the level of colonization of at least one pathogenic bacteria in the lungs of a subject in need thereof, comprising administering an effective amount of 3-Amino-6-methoxy-5-trifluoromethyl-pyridine-2-carboxylic acid ((S)-3,3,3-trifluoro-2-hydroxy-2-methyl-propyl)-amide, or a pharmaceutically acceptable salt thereof, to the subject.

Embodiment 70. A method for inhibiting or reducing the level of colonization of at least one pathogenic bacteria in the lungs of a subject in need thereof, comprising administering an effective amount of 3-Amino-6-methoxy-5-trifluoromethyl-pyridine-2-carboxylic acid ((R)-3,3,3-trifluoro-2-hydroxy-2-methyl-propyl)-amide, or a pharmaceutically acceptable salt thereof, to the subject.

Embodiment 71. A method for inhibiting or reducing the level of colonization of at least one pathogenic bacteria in the lungs of a subject in need thereof, comprising administering an effective amount of 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to the subject.

Embodiment 72. The method of one of Embodiment 68 to Embodiment 71, wherein the pathogenic bacteria is selected from the group consisting of Hemophilus parainfluenza, Hemophilus influenza, Pseudomonas aeruginosa, Moraxella, and Streptococcus pneumonia.

Embodiment 73. The method of one of Embodiment 68 to Embodiment 72, wherein the level of colonization of pathogenic bacteria is reduced by at least one log.

We claim:

1. A method for treating bronchiectasis, comprising:
administering an effective amount of at least one compound of Formula (I), 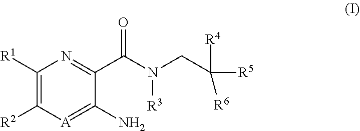
or a pharmaceutically acceptable salt thereof, to a subject in need thereof, wherein:
A is N or CR^{4a};
R¹ is H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; halogen; SO₂NR⁸R⁹; SO₂R¹⁰; S—C₁-C₈alkyl optionally substituted by one or more halogen atoms; S—C₆-C₁₄ aryl; CN; NR¹¹R¹²; C(O)NR¹³R¹⁴; NR¹³SO₂R¹⁵; NR¹³C(O)R¹⁵, CO₂CO₂R¹⁵, —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, aryl and heterocyclyl groups are each optionally substituted by one or more Z substituents;
R² is C₁-C₄ haloalkyl;
R³ and R^{4a} are each independently H or C₁-C₈ alkyl optionally substituted by one or more halogen atoms;
R⁴ is H, or C₁-C₈ alkyl optional substituted with one or more halogen;
R⁵ is —(CH₂)ₘ—NR¹⁷R¹⁸, —(CH₂)ₘ—OR′; C₁-C₈ alkoxy optionally substituted by one or more halogen atoms; —(C₀-C₄ alkyl)-CO₂R¹⁵; —(C₀-C₄ alkyl)-C₆-C₁₄ aryl or −3 to 14 membered heterocyclic group, wherein the heterocyclic group contains at least one heteroatom selected from N, O and S; wherein the —(C₀-C₄ alkyl)-C₆-C₁₄ aryl and —(C₀-C₄ alkyl)-3 to 14 membered heterocyclic group are each optionally substituted by one or more Z substituents;
R⁶ is C₁-C₈ alkyl optionally substituted by one or more halogen atoms; C₃-C₁₀ cycloalkyl; —C₁-C₄ alkyl-C₃-C₈ cycloalkyl; C₁-C₈ alkoxy optionally substituted by one or more halogen atoms; OH; CN; halogen; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, —(C₀-C₄ alkyl)-C₆-C₁₄ aryl and —(C₀-C₄ alkyl)-3 to 14 membered heterocyclic group are each optionally substituted by one or more Z substituents; or
R⁶ is H, and R⁵ is —(CH₂)ₘ—NR¹⁷R¹⁸, —(CH₂)ₘ—OR′, C₁-C₈ alkoxy optionally substituted by one or more halogen atoms; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; or —(C₀-C₄ alkyl)-CO₂R¹⁵, wherein —(C₀-C₄ alkyl)-C₆-C₁₄ aryl and —(C₀-C₄ alkyl)-3 to 14 membered heterocyclic group groups are each optionally substituted by one or more Z substituents; or
R⁴ and R⁶ together with the carbon atoms to which they are bound form a 3 to 8 membered carbocyclic ring system; or
R⁴ and R⁵ together form an oxo group (C═O) and R⁶ is C₁-C₄ alkyl optionally substituted by one or more halogen atoms; C₁-C₄ alkoxy optionally substituted by one or more halogen atoms; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the aryl and heterocyclyl groups are each optionally substituted by one or more Z substituents; or
R⁵ and R⁶ together with the carbon atoms to which they are bound a 5 to 8 membered heterocyclic ring system containing one or more heteroatoms selected from N, O and S, wherein the ring system is optionally substituted by one or more Z substituents; or
R⁴ and R⁵ and R⁶ together with the carbon atoms to which they are bound form a 5 to 8 membered heterocyclic ring system containing one or more heteroatoms selected from N, O and S, wherein the ring system is optionally substituted by one or more Z substituents;
R′ is H, or C₁-C₈ alkyl optional substituted with one or more halogen;
m is 0, 1, 2 or 3;
R⁸, R¹¹, R¹³ and R¹⁷ are each independently H, C₁-C₈ alkyl optionally substituted by one or more halogen atoms, C₃-C₁₀ cycloalkyl or —(C₁-C₄ alkyl)-C₃-C₈ cycloalkyl;
R⁹, R¹⁰, R¹², R¹⁴, R¹⁵, R¹⁶ and R¹⁸ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the cycloalkyl, cycloalkenyl, aryl and heterocyclyl groups are each optionally substituted by one or more Z substituents; or
R⁸ and R⁹, R¹¹ and R¹², R¹³ and R¹⁴, and R¹⁷ and R¹⁸ together with the nitrogen atom to which they are attached may form a 4 to 14 membered heterocyclic group optionally substituted by one or more Z substituents;
Z is independently OH, aryl, O-aryl, benzyl, O-benzyl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁸(SO₂)R²¹, (SO₂)NR¹⁹R²¹, (SO₂)R²¹, NR¹⁸C(O)R²¹, C(O)NR¹⁹R²¹, NR¹⁸C(O)NR¹⁹R²¹, NR¹⁸C(O)OR¹⁹, NR¹⁹R²¹, C(O)OR¹⁹, C(O)R¹⁹, SR¹⁹, OR¹⁹, oxo, CN, NO₂, halogen or a 3 to 14 membered heterocyclic group, wherein the heterocyclic group contains at least one heteroatom selected from N, O and S;
R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or
R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy.

2. The method according to claim 1, wherein A is CR^{4a}.

3. The method according to claim 1 or claim 2, wherein R¹ is C₁-C₈ alkyl optionally substituted by one or more halogen atoms; C₁-C₈ alkoxy optionally substituted by one or more halogen atoms; halogen; NR¹¹R¹², C₆-C₁₄ aryl; or —(C₀-C₄ alkyl)-5 to 6 membered heterocyclic group, wherein the heterocyclic group contains at least one heteroatom selected from N, O and S, wherein the aryl and heterocyclic groups are each optionally substituted by one or more Z substituents.

4. The method according to any preceding claim, wherein R¹ is C₁-C₄ alkyl optionally substituted by one or more halogen atoms; C₁-C₄ alkoxy optionally substituted by one or more halogen atoms; or halogen.

5. The method according to any of claims 1 to 3, wherein R¹ is aryl, wherein aryl is phenyl optionally substituted by one or more Z substituents.

6. The method according to any preceding claim, wherein R² is CF₃.

7. The method according to any preceding claim, wherein
R⁴ is H or C₁-C₄ alkyl optionally substituted by one or more halogen atoms;
R⁵ is C₁-C₄ alkoxy optionally substituted by one or more halogen atoms; —(CH₂)ₘ—NR¹⁷R¹⁸, —(CH₂)ₘ—OR′; or —(C₀-C₄ alkyl)-3 to 14 membered heterocyclic group, wherein the heterocyclic group contains at least one heteroatom selected from N, O and S, wherein the aryl heterocyclyl groups is optionally substituted by one or more Z substituents;
R⁶ is C₁-C₄ alkyl optionally substituted by one or more halogen atoms; C₁-C₄ alkoxy optionally substituted by one or more halogen atoms; or —(C₀-C₄ alkyl)-C₆-C₁₄ aryl wherein the aryl is optionally substituted by one or more Z substituents; or
R⁴ and R⁶ together with the carbon atoms to which they are bound form a 3 to 6 membered carbocyclic ring system; or
R⁵ and R⁶ together with the carbon atoms to which they are bound form a 5 to 8 membered heterocyclic ring system containing one or more heteroatoms selected from N, O and S, wherein the ring system is optionally substituted by one or more Z substituents;
m is 0 or 1;
R¹⁷ and R¹⁸ are each independently H; C₁-C₈ alkyl optionally substituted by one or more halogen atoms.

8. The method according to any of the preceding claims, wherein
A is CR^{4a};
R¹ is C₁-C₄ alkyl optionally substituted by one or more halogen atoms; or C₁-C₄ alkoxy optionally substituted by one or more halogen atoms;
R² is CF₃,
R³ is H, CH₃ or CF₃;
R⁴ is H or Me;
R^{4a} is H;
R⁵ is —NR¹⁷R¹⁸ or OH, and
R⁶ is C₁-C₄ alkyl optionally substituted by one or more halogen atoms.

9. The method according to claim 1, wherein the compound is represented by Formula (II), 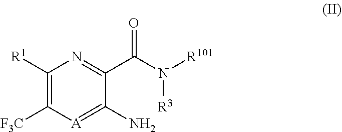
or a pharmaceutically acceptable salt;
wherein
A is N or CR^{4a};
R^{a} is H or C₁-C₄ alkyl;
R¹ is C₁-C₈ alkyl optionally substituted by one or more halogen atoms; C₁-C₈ alkoxy optionally substituted by one or more halogen atoms; halogen; NR¹¹R¹², C₆-C₁₄ aryl; or —(C₀-C₄ alkyl)-5 to 6 membered heterocyclic group, wherein the heterocyclic group contains at least one heteroatom selected from N, O and S, wherein the aryl and heterocyclic groups are each optionally substituted by one or more Z substituents;
R₃ is H or CH₃;
R¹⁰¹ is

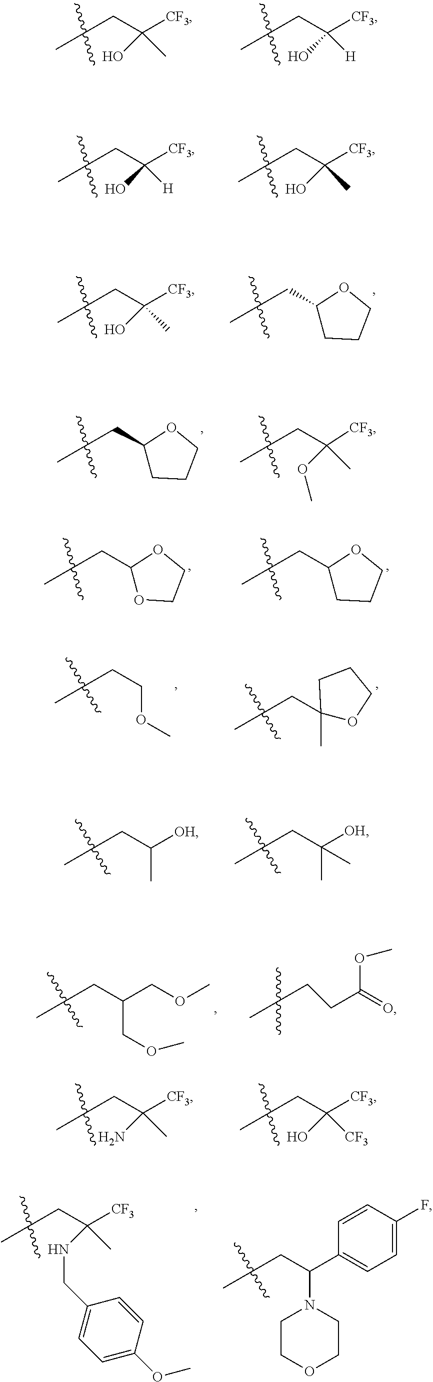

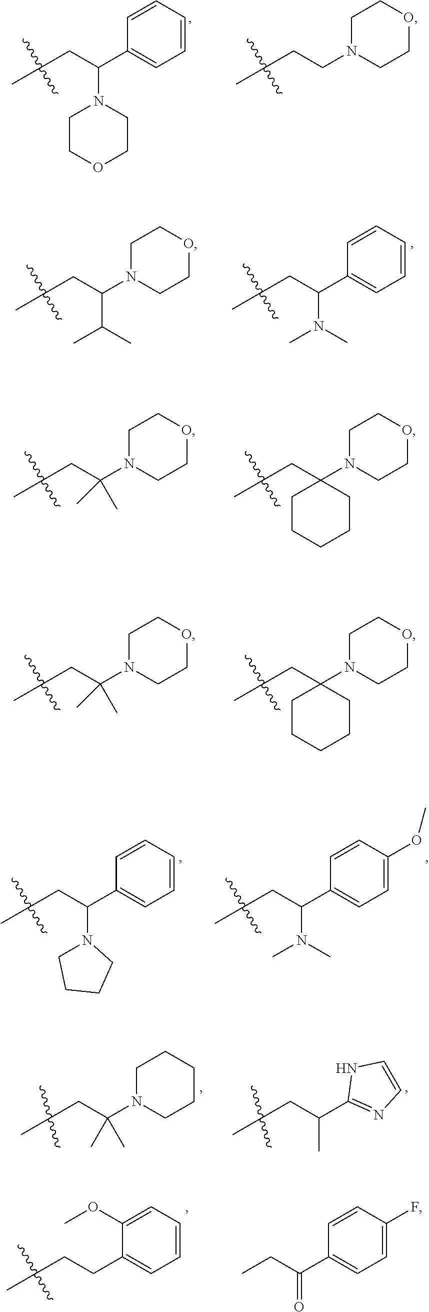

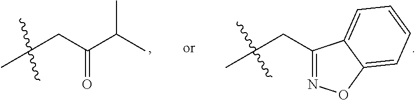

10. The method according to any of the preceding claims, wherein the bronchiectasis is cystic fibrosis bronchiectasis or non-cystic fibrosis bronchiectasis.

11. A method for treating bronchiectasis comprising administering an effective amount of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to a subject in need thereof.

12. The method of claim 11, wherein the compound is 3-Amino-6-methoxy-5-trifluoromethyl-pyridine-2-carboxylic acid ((S)-3,3,3-trifluoro-2-hydroxy-2-methyl-propyl)-amide or a pharmaceutically acceptable salt thereof.

13. The method according to claim 11 or claim 12, wherein the bronchiectasis is cystic fibrosis bronchiectasis or non-cystic fibrosis bronchiectasis.

14. A method for inhibiting or reducing the level of colonization of at least one pathogenic bacteria in the lungs of a subject comprising administering a compound of Formula (I), 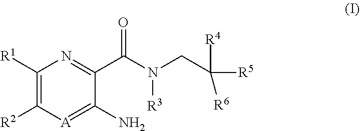
or pharmaceutically acceptable salts thereof, to a subject in need thereof, wherein:
A is N or CR^{4a};
R¹ is H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; C₁-C₈ alkoxy optionally substituted by one or more halogen atoms; halogen; SO₂NR⁸R⁹; SO₂R¹⁰; S—C₁-C₈alkyl optionally substituted by one or more halogen atoms; S—C₆-C₁₄ aryl; CN; NR¹¹R¹²; C(O)NR¹³R¹⁴; NR¹³SO₂R¹⁵; NR¹³C(O)R¹⁵, CO₂R¹⁵, —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, aryl and heterocyclyl groups are each optionally substituted by one or more Z substituents;
R² is C₁-C₄ haloalkyl;
R³ and R^{4a} are each independently H or C₁-C₈ alkyl optionally substituted by one or more halogen atoms;
R⁴ is H, or C₁-C₈ alkyl optional substituted with one or more halogen;
R⁵ is —(CH₂)ₘ—NR¹⁷R¹⁸, —(CH₂)ₘ—OR′; (CH₂)ₘ—OR′; C₁-C₈ alkoxy optionally substituted by one or more halogen atoms; —(C₀-C₄ alkyl)-CO₂R¹⁵; —(C₀-C₄ alkyl)-C₆-C₁₄ aryl or −3 to 14 membered heterocyclic group, wherein the heterocyclic group contains at least one heteroatom selected from N, O and S; wherein the —(C₀-C₄ alkyl)-C₆-C₁₄ aryl and —(C₀-C₄ alkyl)-3 to 14 membered heterocyclic group are each optionally substituted by one or more Z substituents;
R⁶ is C₁-C₈ alkyl optionally substituted by one or more halogen atoms; C₃-C₁₀ cycloalkyl; —C₁-C₄ alkyl-C₃-C₈ cycloalkyl; C₁-C₈ alkoxy optionally substituted by one or more halogen atoms; OH; CN; halogen; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S; wherein the cycloalkyl, cycloalkenyl, —(C₀-C₄ alkyl)-C₆-C₁₄ aryl and —(C₀-C₄ alkyl)-3 to 14 membered heterocyclic group are each optionally substituted by one or more Z substituents; or
R⁶ is H, and R⁵ is —(CH₂)ₘ—NR¹⁷R¹⁸, —(CH₂)ₘ—OR′, C₁-C₈ alkoxy optionally substituted by one or more halogen atoms; —(C₀-C₄ alkyl)-C₆-C₁₄ aryl; —(C₀-C₄ alkyl)-3 to 14 membered heterocyclic group, wherein the heterocyclic group contains at least one heteroatom selected from N, O and S; or —(C₀-C₄ alkyl)-CO₂R¹⁵, wherein —(C₀-C₄ alkyl)-C₆-C₁₄ aryl and —(C₀-C₄ alkyl)-3 to 14 membered heterocyclic group groups are each optionally substituted by one or more Z substituents; or
R⁴ and R⁶ together with the carbon atoms to which they are bound form a 3 to 8 membered carbocyclic ring system; or
R⁴ and R⁵ together form an oxo group (C═O) and R⁶ is C₁-C₄ alkyl optionally substituted by one or more halogen atoms; C₁-C₄ alkoxy optionally substituted by one or more halogen atoms; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the aryl and heterocyclyl groups are each optionally substituted by one or more Z substituents; or
R⁵ and R⁶ together with the carbon atoms to which they are bound a 5 to 8 membered heterocyclic ring system containing one or more heteroatoms selected from N, O and S, wherein the ring system is optionally substituted by one or more Z substituents; or
R⁴ and R⁵ and R⁶ together with the carbon atoms to which they are bound form a 5 to 8 membered heterocyclic ring system containing one or more heteroatoms selected from N, O and S, wherein the ring system is optionally substituted by one or more Z substituents;
R′ is H, or C₁-C₈ alkyl optional substituted with one or more halogen;
m is 0, 1, 2 or 3;
R⁸, R¹¹, R¹³ and R¹⁷ are each independently H, C₁-C₈ alkyl optionally substituted by one or more halogen atoms, C₃-C₁₀ cycloalkyl or —(C₁-C₄ alkyl)-C₃-C₈ cycloalkyl;
R⁹, R¹⁰, R¹², R¹⁴, R¹⁵, R¹⁶ and R¹⁸ are each independently H; C₁-C₈ alkyl optionally substituted by one or more halogen atoms; C₂-C₈ alkenyl; C₂-C₈ alkynyl; C₃-C₁₀ cycloalkyl; C₅-C₁₀ cycloalkenyl; —C₁-C₄ alkyl-C₃-C₈ cycloalkyl; —(C₀-C₄ alkyl)-C₆-C₁₄ aryl; or —(C₀-C₄ alkyl)-3 to 14 membered heterocyclic group, wherein the heterocyclic group contains at least one heteroatom selected from N, O and S, wherein the cycloalkyl, cycloalkenyl, aryl and heterocyclyl groups are each optionally substituted by one or more Z substituents; or
R⁸ and R⁹, R¹¹ and R¹², R¹³ and R¹⁴, and R¹⁷ and R¹⁸ together with the nitrogen atom to which they are attached may form a 4 to 14 membered heterocyclic group optionally substituted by one or more Z substituents;
Z is independently OH, aryl, O-aryl, benzyl, O-benzyl, C₁-C₆ alkyl optionally substituted by one or more OH groups or NH₂ groups, C₁-C₆ alkyl optionally substituted by one or more halogen atoms, C₁-C₆ alkoxy optionally substituted by one or more OH groups or C₁-C₄ alkoxy, NR¹⁸(SO₂)R²¹, (SO₂)NR¹⁹R²¹, (SO₂)R²¹, NR¹⁸C(O)R²¹, C(O)NR¹⁹R²¹, NR¹⁸C(O)NR¹⁹R²¹, NR¹⁸C(O)OR¹⁹, NR¹⁹R²¹, C(O)OR¹⁹, C(O)R¹⁹, SR¹⁹, OR¹⁹, oxo, CN, NO₂, halogen or a 3 to 14 membered heterocyclic group, wherein the heterocyclic group contains at least one heteroatom selected from N, O and S;
R¹⁹ and R²¹ are each independently H; C₁-C₈ alkyl; C₃-C₈ cycloalkyl; C₁-C₄ alkoxy-C₁-C₄ alkyl; (C₀-C₄ alkyl)-aryl optionally substituted by one or more groups selected from C₁-C₆ alkyl, C₁-C₆ alkoxy and halogen; (C₀-C₄ alkyl)-3- to 14-membered heterocyclic group, the heterocyclic group including one or more heteroatoms selected from N, O and S, optionally substituted by one or more groups selected from halogen, oxo, C₁-C₆ alkyl and C(O)C₁-C₆ alkyl; (C₀-C₄ alkyl)-O-aryl optionally substituted by one or more groups selected from C₁-C₆ alkyl, C₁-C₆ alkoxy and halogen; and (C₀-C₄ alkyl)-O-3- to 14-membered heterocyclic group, the heterocyclic group including one or more heteroatoms selected from N, O and S, optionally substituted by one or more groups selected from halogen, C₁-C₆ alkyl or C(O)C₁-C₆ alkyl; wherein the alkyl groups are optionally substituted by one or more halogen atoms, C₁-C₄ alkoxy, C(O)NH₂, C(O)NHC₁-C₆ alkyl or C(O)N(C₁-C₆ alkyl)₂; or
R¹⁹ and R²¹ together with the nitrogen atom to which they attached form a 5- to 10-membered heterocyclic group, the heterocyclic group including one or more further heteroatoms selected from N, O and S, the heterocyclic group being optionally substituted by one or more substituents selected from OH; halogen; aryl; 5- to 10-membered heterocyclic group including one or more heteroatoms selected from N, O and S; S(O)₂-aryl; S(O)₂—C₁-C₆ alkyl; C₁-C₆ alkyl optionally substituted by one or more halogen atoms; C₁-C₆ alkoxy optionally substituted by one or more OH groups or C₁-C₄ alkoxy; and C(O)OC₁-C₆ alkyl, wherein the aryl and heterocyclic substituent groups are themselves optionally substituted by C₁-C₆ alkyl, C₁-C₆ haloalkyl or C₁-C₆ alkoxy.

15. A method for inhibiting or reducing the level of colonization of at least one pathogenic bacteria in the lungs of a subject in need thereof comprising administering an effective amount of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to the subject, optionally wherein the level of at least one pathogenic bacteria is measured from a sputum sample obtained from the subject, optionally wherein the level of at least one pathogenic bacteria is measured by 16S rRNA PCR.

16. The method of claim 14 or claim 15, wherein the pathogenic bacteria is a non-fermenting Gram negative bacteria.

17. The method of any one of claim 14 to claim 16, wherein the pathogenic bacteria is selected from the group consisting of M catarrhalis, S. aureus, Enterobacteriaceae, Stenotrophomonous Maltophilia Hemophilus parainfluenza, Hemophilus influenza, Pseudomonas aeruginosa, Moraxella, and Streptococcus pneumonia.

18. The method of any one of claim 14 to claim 17, wherein the level of colonization of pathogenic bacteria is reduced by at least one log.

19. A method for reducing the level of fibrinogen in the blood of a subject in need thereof, e.g., a bronchiectasis subject, comprising administering an effective amount of a compound selected from the group consisting of: 3-Amino-6-methoxy-5-trifluoromethyl-pyridine-2-carboxylic acid ((S)-3,3,3-trifluoro-2-hydroxy-2-methyl-propyl)-amide, 3-Amino-6-methoxy-5-trifluoromethyl-pyridine-2-carboxylic acid ((R)-3,3,3-trifluoro-2-hydroxy-2-methyl-propyl)-amide, and 3-Amino-6-methoxy-5-trifluoromethyl-pyridine-2-carboxylic acid (3,3,3-trifluoro-2-hydroxy-2-methyl-propyl)-amide, or a pharmaceutically acceptable salt thereof, to the subject.

20. The method according to any of the preceding claims, wherein the method further comprises:
a. reducing the use of rescue medication (e.g., salbutamol/albuterol or systemic antibiotics) in a subject when compared to a subject who is not administered the compound;
b. reducing the severity of exacerbations in a subject when compared to a subject who is not administered the compound;
c. increasing one or more of improved lung function or forced vital capacity in a subject, e.g., as measured by spirometry, when compared to a patient who is not administered the compound; or
d. any combination thereof.

21. The method of any one of claim 14 to claim 20, wherein the subject in need thereof is a bronchiectasis subject.

22. The method according to any of the preceding claims, wherein the compound is administered in an amount of between about 300 mg b.i.d. and about 450 mg b.i.d., e.g., is administered in an amount of 300 mg b.i.d. or 450 mg b.i.d.

23. The method according to any of the preceding claims, wherein the compound is administered to the subject in an amount of about 300 mg b.i.d.

24. The method according to any of the preceding claims, wherein the compound is administered orally.

25. The method according to any of the preceding claims, wherein the compound is administered to the subject without a high fat meal.

26. The method according to any of the preceding claims wherein the compound is administered in combination with at least one additional therapy.

27. The method according to claim 26, wherein the additional therapy comprises:
a. a long-acting beta-agonist (LABA);
b. a long-acting muscarinic antagonists (LAMA);
c. an inhaled corticosteroid (ICS);
d. macrolides;
e. an antibiotic;
f. a short-acting muscarinic antagonist (SAMA); or
g. any combination thereof.

28. The method according to any of the preceding claims, wherein the bronchiecstasis is characterized by deterioration in three or more symptoms for at least 48 hours.

29. The method according to claim 28, wherein the symptoms are selected from the group consisting of: cough, sputum volume and/or consistency, sputum purulence, breathlessness and/or exercise tolerance, fatigue and/or malaise, and haemoptysis.
